SEQUENCE LISTING (59 sequences)

SEQ ID NO: 1 - DNA, 888 bp, Artificial Sequence
  Description of Artificial Sequence Synthetic polynucleotide
atgaatgcaaaactgaagaaattgttccagcagaaagtagacggcaagaccatcatcgtgaccggtgcaagcagcggtattggcttgaccgtgagcaaatacctggctcaggcgggtgcacacgtgctgctgctggcgcgtacgaaagagaaactggatgaggtcaaggcggagattgaagcggaaggcggtaaggctactgttttcccgtgcgatttgaatgacatggaatccattgacgcagtcagcaaagagatcctggcagccgttgatcatatcgacattctggtgaataacgcgggtcgcagcatccgtcgcgcggtccacgaaagcgtggatcgcttccatgactttgagcgtaccatgcaactgaattacttcggtgccgttcgtctggtcctgaatgttctgccgcacatgatgcagcgcaaagatggccaaatcattaacattagcagcattggcgttttggcgaacgcgacgcgtttcagcgcgtatgtggcgagcaaggctgcactggatgccttctcccgttgtctgagcgccgaggtccattcgcacaagattgcgattacctctatctatatgccgctggttcgtaccccgatgattgcgccgacgaagatctacaagtatgtcccaacgttgtccccggaagaggcggctgacctgattgcttatgcgatcgttaaacgtccgaaaaagatcgccaccaatctgggtcgcctggcaagcatcacctacgcgattgccccggacatcaacaacatcctgatgagcatcggctttaacctgtttccgtctagcacggcgagcgtgggtgagcaagaaaagctgaacctgattcaacgtgcctacgcacgtctgtttcctggtgaacactggtaa

SEQ ID NO: 2 - DNA, 6215 bp, Artificial Sequence
  Description of Artificial Sequence Synthetic polynucleotide
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatgaatgcaaaactgaagaaattgttccagcagaaagtagacggcaagaccatcatcgtgaccggtgcaagcagcggtattggcttgaccgtgagcaaatacctggctcaggcgggtgcacacgtgctgctgctggcgcgtacgaaagagaaactggatgaggtcaaggcggagattgaagcggaaggcggtaaggctactgttttcccgtgcgatttgaatgacatggaatccattgacgcagtcagcaaagagatcctggcagccgttgatcatatcgacattctggtgaataacgcgggtcgcagcatccgtcgcgcggtccacgaaagcgtggatcgcttccatgactttgagcgtaccatgcaactgaattacttcggtgccgttcgtctggtcctgaatgttctgccgcacatgatgcagcgcaaagatggccaaatcattaacattagcagcattggcgttttggcgaacgcgacgcgtttcagcgcgtatgtggcgagcaaggctgcactggatgccttctcccgttgtctgagcgccgaggtccattcgcacaagattgcgattacctctatctatatgccgctggttcgtaccccgatgattgcgccgacgaagatctacaagtatgtcccaacgttgtccccggaagaggcggctgacctgattgcttatgcgatcgttaaacgtccgaaaaagatcgccaccaatctgggtcgcctggcaagcatcacctacgcgattgccccggacatcaacaacatcctgatgagcatcggctttaacctgtttccgtctagcacggcgagcgtgggtgagcaagaaaagctgaacctgattcaacgtgcctacgcacgtctgtttcctggtgaacactggtaagaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 3 - DNA, 3549 bp, Artificial Sequence
  Description of Artificial Sequence Synthetic polynucleotide
gagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtacc

SEQ ID NO: 4 - DNA, 705 bp, Artificial Sequence
  Description of Artificial Sequence Synthetic polynucleotide
catatgcaagaactggccctgagaagcgagctggacttcaatagcgaaacctataaagatgcgtatagccgtattaacgccattgtgatcgaaggcgagcaagaagcataccaaaactacctggacatggcgcaactgctgccggaggacgaggctgagctgattcgtttgagcaagatggagaaccgtcacaaaaagggttttcaagcgtgcggcaagaacctcaatgtgactccggatatggattatgcacagcagttctttgcggagctgcacggcaattttcagaaggctaaagccgagggtaagattgttacctgcctgctcatccaaagcctgatcatcgaggcgtttgcgattgcagcctacaacatttacattccagtggctgatccgtttgcacgtaaaatcaccgagggtgtcgtcaaggatgagtatacccacctgaatttcggcgaagtttggttgaaggaacattttgaagcaagcaaggcggagttggaggacgccaacaaagagaacttaccgctggtctggcagatgttgaaccaggtcgaaaaggatgccgaagtgctgggtatggagaaagaggctctggtggaggactttatgattagctatggtgaggcactgagcaacatcggcttttctacgagagaaatcatgaagatgagcgcgtacggtctgcgtgcagcataagagctc

SEQ ID NO: 5 - DNA, 4774 bp, Artificial Sequence
  Description of Artificial Sequence Synthetic polynucleotide
gagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtaccaggaggtttttacaatggctgatactttgttgattttgggtgattctctctctgcaggctaccgtatgtccgcgagcgcggcatggccggctctgctgaacgataagtggcagagcaagaccagcgtggtcaatgcgagcatcagcggcgataccagccagcagggtctggcacgtctgccagcgctgctgaagcaacaccagccgcgttgggtgctggttgaactgggcggcaatgacggtctgcgtggttttcagccgcagcagaccgaacaaacgttgcgtcagattctgcaggacgtcaaggcggctaacgcggaaccgctgctgatgcaaattcgcctgccggcgaattatggtcgtcgttacaacgaggctttcagcgccatttatcctaaactggctaaagagtttgacgtgccgctgctgccgttcttcatggaagaggtctacctgaaaccgcaatggatgcaagacgacggtattcatccgaatcgtgatgcacaacctttcatcgcggattggatggcgaagcaattgcaaccgctggtgaaccatgactcgtaaaagcttgttgctgcatgcaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattc

SEQ ID NO: 6 - DNA, 11239 bp, Artificial Sequence
  Description of Artificial Sequence Synthetic polynucleotide
aaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcaggcgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatgcaagaactggccctgagaagcgagctggacttcaatagcgaaacctataaagatgcgtatagccgtattaacgccattgtgatcgaaggcgagcaagaagcataccaaaactacctggacatggcgcaactgctgccggaggacgaggctgagctgattcgtttgagcaagatggagaaccgtcacaaaaagggttttcaagcgtgcggcaagaacctcaatgtgactccggatatggattatgcacagcagttctttgcggagctgcacggcaattttcagaaggctaaagccgagggtaagattgttacctgcctgctcatccaaagcctgatcatcgaggcgtttgcgattgcagcctacaacatttacattccagtggctgatccgtttgcacgtaaaatcaccgagggtgtcgtcaaggatgagtatacccacctgaatttcggcgaagtttggttgaaggaacattttgaagcaagcaaggcggagttggaggacgccaacaaagagaacttaccgctggtctggcagatgttgaaccaggtcgaaaaggatgccgaagtgctgggtatggagaaagaggctctggtggaggactttatgattagctatggtgaggcactgagcaacatcggcttttctacgagagaaatcatgaagatgagcgcgtacggtctgcgtgcagcataagagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtaccaggaggtttttacaatggctgatactttgttgattttgggtgattctctctctgcaggctaccgtatgtccgcgagcgcggcatggccggctctgctgaacgataagtggcagagcaagaccagcgtggtcaatgcgagcatcagcggcgataccagccagcagggtctggcacgtctgccagcgctgctgaagcaacaccagccgcgttgggtgctggttgaactgggcggcaatgacggtctgcgtggttttcagccgcagcagaccgaacaaacgttgcgtcagattctgcaggacgtcaaggcggctaacgcggaaccgctgctgatgcaaattcgcctgccggcgaattatggtcgtcgttacaacgaggctttcagcgccatttatcctaaactggctaaagagtttgacgtgccgctgctgccgttcttcatggaagaggtctacctgaaaccgcaatggatgcaagacgacggtattcatccgaatcgtgatgcacaacctttcatcgcggattggatggcgaagcaattgcaaccgctggtgaaccatgactcgtaaaagcttgttgctgcatgcaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggt

SEQ ID NO: 7 - DNA, 940 bp, Artificial Sequence
  Description of Artificial Sequence Synthetic polynucleotide
ggtaccaggaggtttttacatggaccgtaaaagcaagcgtccggacatgctggttgattcctttggtctggaaagcaccgtgcaggacggtctggttttccgtcagtctttctccattcgtagctatgagattggtactgatcgtaccgcctctatcgaaaccctgatgaatcacctgcaagaaacctctctgaaccattgtaagtctactggcatcctgctggacggtttcggtcgtaccctggagatgtgcaaacgcgacctgatttgggtagtgatcaaaatgcagatcaaagttaaccgttatccggcatggggtgataccgttgaaatcaacacccgcttttctcgtctgggcaaaatcggtatgggccgtgactggctgatctctgactgtaacactggtgaaattctggttcgtgctactagcgcatacgcgatgatgaaccagaaaacccgtcgcctgagcaagctgccgtacgaggtccaccaggagattgttccgctgtttgtagacagcccagtgattgaggattctgacctgaaagtgcataaattcaaagtgaagaccggtgacagcatccaaaaaggcctgaccccaggttggaacgatctggacgttaaccagcacgtttccaacgtgaagtatatcggttggattctggagagcatgccgaccgaggtcctggaaacccaggagctgtgttccctggcgctggagtaccgccgtgagtgcggccgtgacagcgtgctggagtctgtgaccgctatggacccaagcaaagttggtgttcgtagccagtaccagcacctgctgcgtctggaagacggtactgctatcgtgaacggtgcaactgaatggcgtcctaaaaacgcgggtgcaaacggtgctatcagcaccggtaaaacctctaacggtaactccgtgagctaaaagctt

SEQ ID NO: 8 - DNA, 11601 bp, Artificial Sequence
  Description of Artificial Sequence Synthetic polynucleotide
aaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcaggcgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatgcaagaactggccctgagaagcgagctggacttcaatagcgaaacctataaagatgcgtatagccgtattaacgccattgtgatcgaaggcgagcaagaagcataccaaaactacctggacatggcgcaactgctgccggaggacgaggctgagctgattcgtttgagcaagatggagaaccgtcacaaaaagggttttcaagcgtgcggcaagaacctcaatgtgactccggatatggattatgcacagcagttctttgcggagctgcacggcaattttcagaaggctaaagccgagggtaagattgttacctgcctgctcatccaaagcctgatcatcgaggcgtttgcgattgcagcctacaacatttacattccagtggctgatccgtttgcacgtaaaatcaccgagggtgtcgtcaaggatgagtatacccacctgaatttcggcgaagtttggttgaaggaacattttgaagcaagcaaggcggagttggaggacgccaacaaagagaacttaccgctggtctggcagatgttgaaccaggtcgaaaaggatgccgaagtgctgggtatggagaaagaggctctggtggaggactttatgattagctatggtgaggcactgagcaacatcggcttttctacgagagaaatcatgaagatgagcgcgtacggtctgcgtgcagcataagagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtaccaggaggtttttacatggaccgtaaaagcaagcgtccggacatgctggttgattcctttggtctggaaagcaccgtgcaggacggtctggttttccgtcagtctttctccattcgtagctatgagattggtactgatcgtaccgcctctatcgaaaccctgatgaatcacctgcaagaaacctctctgaaccattgtaagtctactggcatcctgctggacggtttcggtcgtaccctggagatgtgcaaacgcgacctgatttgggtagtgatcaaaatgcagatcaaagttaaccgttatccggcatggggtgataccgttgaaatcaacacccgcttttctcgtctgggcaaaatcggtatgggccgtgactggctgatctctgactgtaacactggtgaaattctggttcgtgctactagcgcatacgcgatgatgaaccagaaaacccgtcgcctgagcaagctgccgtacgaggtccaccaggagattgttccgctgtttgtagacagcccagtgattgaggattctgacctgaaagtgcataaattcaaagtgaagaccggtgacagcatccaaaaaggcctgaccccaggttggaacgatctggacgttaaccagcacgtttccaacgtgaagtatatcggttggattctggagagcatgccgaccgaggtcctggaaacccaggagctgtgttccctggcgctggagtaccgccgtgagtgcggccgtgacagcgtgctggagtctgtgaccgctatggacccaagcaaagttggtgttcgtagccagtaccagcacctgctgcgtctggaagacggtactgctatcgtgaacggtgcaactgaatggcgtcctaaaaacgcgggtgcaaacggtgctatcagcaccggtaaaacctctaacggtaactccgtgagctaaaagcttgttgctgcatgcaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggt

SEQ ID NO: 9 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 10 - protein, 206 aa, Escherichia coli
MKTTHTSLPFAGHTLHFVEFDPANFCEQDLLWLPHYAQLQHAGRKRKTEHLAGRIAAVYALREYGYKCVPAIGELRQPVWPAEVYGSISHCGTTALAVVSRQPIGIDIEEIFSVQTARELTDNIITPAEHERLADCGLAFSLALTLAFSAKESAFKASEIQTDAGFLDYQIISWNKQQVIIHRENEMFAVHWQIKEKIVITLCQHD

SEQ ID NO: 11 - protein, 295 aa, Acinetobacter sp.
MNAKLKKLFQQKVDGKTIIVTGASSGIGLTVSKYLAQAGAHVLLLARTKEKLDEVKAEIEAEGGKATVFPCDLNDMESIDAVSKEILAAVDHIDILVNNAGRSIRRAVHESVDRFHDFERTMQLNYFGAVRLVLNVLPHMMQRKDGQIINISSIGVLANATRFSAYVASKAALDAFSRCLSAEVHSHKIAITSIYMPLVRTPMIAPTKIYKYVPTLSPEEAADLIAYAIVKRPKKIATNLGRLASITYAIAPDINNILMSIGFNLFPSSTASVGEQEKLNLIQRAYARLFPGEHW

SEQ ID NO: 12 - protein, 561 aa, Escherichia coli
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 13 - protein, 382 aa, Umbellularia californica
MATTSLASAFCSMKAVMLARDGRGMKPRSSDLQLRAGNAPTSLKMINGTKFSYTESLKRLPDWSMLFAVITTIFSAAEKQWTNLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 14 - protein, 299 aa, Umbellularia californica
MEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 15 - protein, 415 aa, Cuphea hookeriana
MVAAAASSAFFPVPAPGASPKPGKFGNWPSSLSPSFKPKSIPNGGFQVKANDSAHPKANGSAVSLKSGSLNTQEDTSSSPPPRTFLHQLPDWSRLLTAITTVFVKSKRPDMHDRKSKRPDMLVDSFGLESTVQDGLVFRQSFSIRSYEIGTDRTASIETLMNHLQETSLNHCKSTGILLDGFGRTLEMCKRDLIWVVIKMQIKVNRYPAWGDTVEINTRFSRLGKIGMGRDWLISDCNTGEILVRATSAYAMMNQKTRRLSKLPYEVHQEIVPLFVDSPVIEDSDLKVHKFKVKTGDSIQKGLTPGWNDLDVNQHVSNVKYIGWILESMPTEVLETQELCSLALEYRRECGRDSVLESVTAMDPSKVGVRSQYQHLLRLEDGTAIVNGATEWRPKNAGANGAISTGKTSNGNSVS

SEQ ID NO: 16 - protein, 304 aa, Cuphea hookeriana
MDRKSKRPDMLVDSFGLESTVQDGLVFRQSFSIRSYEIGTDRTASIETLMNHLQETSLNHCKSTGILLDGFGRTLEMCKRDLIWVVIKMQIKVNRYPAWGDTVEINTRFSRLGKIGMGRDWLISDCNTGEILVRATSAYAMMNQKTRRLSKLPYEVHQEIVPLFVDSPVIEDSDLKVHKFKVKTGDSIQKGLTPGWNDLDVNQHVSNVKYIGWILESMPTEVLETQELCSLALEYRRECGRDSVLESVTAMDPSKVGVRSQYQHLLRLEDGTAIVNGATEWRPKNAGANGAISTGKTSNGNSVS

SEQ ID NO: 17 - protein, 548 aa, Lactococcus lactis
MYTVGDYLLDRLHELGIEEIFGVPGDYNLQFLDQIISRKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENPTSNTSDQEILNKIQESLKNAKKPIVITGHEIISFGLENTVTQFISKTKLPITTLNFGKSSVDETLPSFLGIYNGKLSEPNLKEFVESADFILMLGVKLTDSSTGAFTHHLNENKMISLNIDEGKIFNESIQNFDFESLISSLLDLSGIEYKGKYIDKKQEDFVPSNALLSQDRLWQAVENLTQSNETIVAEQGTSFFGASSIFLKPKSHFIGQPLWGSIGYTFPAALGSQIADKESRHLLFIGDGSLQLTVQELGLAIREKINPICFIINNDGYTVEREIHGPNQSYNDIPMWNYSKLPESFGATEERVVSKIVRTENEFVSVMKEAQADPNRMYWIELVLAKEDAPKVLKKMGKLFAEQNKS

SEQ ID NO: 18 - DNA, 3522 bp, Artificial Sequence
  Description of Artificial Sequence Synthetic polynucleotide
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctga

SEQ ID NO: 19 - DNA, 699 bp, Artificial Sequence
  Description of Artificial Sequence Synthetic polynucleotide
atgcagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaa

SEQ ID NO: 20 - DNA, 900 bp, Artificial Sequence
  Description of Artificial Sequence Synthetic polynucleotide
atggagtggaaaccaaaaccgaaactgcctcagctgctggatgaccacttcggtctgcacggcctggttttccgtcgtaccttcgctatccgttcttacgaagtcggccctgatcgctccacctccatcctggcggtaatgaaccacatgcaggaagcaactctgaaccatgcgaaaagcgtaggtatcctgggcgatggtttcggcactactctggagatgtccaaacgtgatctgatgtgggttgttcgccgtacccatgtcgcggttgaacgctacccgacctggggcgatacggttgaagtggaatgctggatcggcgcgtccggcaacaacggcatgcgtcgcgatttcctggttcgcgattgtaagacgggcgagattctgacccgttgcacgtccctgagcgttctgatgaatacccgtacccgtcgtctgagcaccatcccggacgaagttcgcggtgaaattggcccggcattcatcgataacgttgcagtaaaagacgatgaaatcaagaaactgcagaaactgaatgactctaccgcggactacatccagggtggtctgaccccgcgctggaacgacctggacgtgaaccagcacgtcaacaacctgaaatacgtagcttgggtattcgaaacggtcccggattctatcttcgaatctcaccacatcagctccttcaccctggaataccgtcgtgagtgtacccgtgactccgttctgcgctctctgaccacggtatccggcggtagctctgaagccggtctggtttgcgatcacctgctgcagctggaaggcggcagcgaggttctgcgtgctcgtactgagtggcgtccgaagctgactgactctttccgcggcatctctgttatcccggcagagcctcgtgtgtaa

SEQ ID NO: 21 - DNA, 621 bp, Artificial Sequence
  Description of Artificial Sequence Synthetic polynucleotide
atgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactga

SEQ ID NO: 22 - DNA, 6538 bp, Artificial Sequence
  Description of Artificial Sequence Synthetic polynucleotide
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaaggcctcactggcccctgcagggatggtggaatgctggttatctggtggggattaagtggtgttttactaaagcttgaacaactcaagaaagattatattcgcaataactgccaataatcccagcatcttgagaaaatccagcaaaccgggggcaaaacaccagcaagaagccagcagactatcaccaaatccccagcgtacagctagaaataactgagcagttgtattcaattaccttctggtcaagccgaggaaatttccccacaccttatacacctctggaaggtttttttgacgaagcgcaaaatatccacaatcggctggggacttcttctgtcagaaaatggcagaaatttttgaatgtgttggcgatcgccctcatcaatgattattagagaacttttgtccctgatgttgggaatactcttgatgacaattgtgattgctcaaagaagaaagaaatttggagtaaatctctaaaaggggactgaaatatttgtatggtcagcatgaccactgaaatggagagaagtctaagacagtagatgtcttagatataagcctcattagaagccatgccataaaacagattttgtggatgaaacaacttgaaatagttcagttgtagaccatgttataaacatttattcttaacacagtgacacattaatgactcatatatccgtccaaaaaaaactaaaatgtttgtaaatttagttttgcggccgcgtcgacttcgttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaatcatatgcagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaactcgagcaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagttttaattaacgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtggcgcgcccagtttcctttactggccctaaagtcgctgtggctagggttccgaaggggcattattggctcgcggctttacaaccttgataaggagagagatgacagttttttttctcttttgcttagtaaaacagcaaatttaaggcatgttaaagagcagtagaacgaaatggttgagccggcctcgatacactcaattaactactaatagcttcaataaattttgggacgattgaagctatttttttgaaaatcaactcttaatatctcctgtctcaaaagagttaattgctaaacaaaagccagtttcagcgaaaaatctagagttttataggttcgttctcagtacaggacaaaaagtttgaaaaggatagagggagagggtttgatggaaataagcacaaatcaatcaagccctcatgaatcagattagcgaaattcgccgccaattgcgacctcatctcggatggcatggagccagactgtcatttatcgccctcttcctggtggcactgttccgagcaaaaaccgtcaatctcgccaaactcgccaccgtctggggaggcaatgcagcagaagagtctaattacaaacgcatgcagcgattctttcagtcctttgacgtcaacatggacaaaatcgccaggatggtaatgaatatcgcggctatcccgcaaccttgggtcttaagcatcgaccgcaccaacggccggcctacatggcccgtcaatcgaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 23 - DNA, 10863 bp, Artificial Sequence
  Description of Artificial Sequence Synthetic polynucleotide
aaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcaggcgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatggagtggaaaccaaaaccgaaactgcctcagctgctggatgaccacttcggtctgcacggcctggttttccgtcgtaccttcgctatccgttcttacgaagtcggccctgatcgctccacctccatcctggcggtaatgaaccacatgcaggaagcaactctgaaccatgcgaaaagcgtaggtatcctgggcgatggtttcggcactactctggagatgtccaaacgtgatctgatgtgggttgttcgccgtacccatgtcgcggttgaacgctacccgacctggggcgatacggttgaagtggaatgctggatcggcgcgtccggcaacaacggcatgcgtcgcgatttcctggttcgcgattgtaagacgggcgagattctgacccgttgcacgtccctgagcgttctgatgaatacccgtacccgtcgtctgagcaccatcccggacgaagttcgcggtgaaattggcccggcattcatcgataacgttgcagtaaaagacgatgaaatcaagaaactgcagaaactgaatgactctaccgcggactacatccagggtggtctgaccccgcgctggaacgacctggacgtgaaccagcacgtcaacaacctgaaatacgtagcttgggtattcgaaacggtcccggattctatcttcgaatctcaccacatcagctccttcaccctggaataccgtcgtgagtgtacccgtgactccgttctgcgctctctgaccacggtatccggcggtagctctgaagccggtctggtttgcgatcacctgctgcagctggaaggcggcagcgaggttctgcgtgctcgtactgagtggcgtccgaagctgactgactctttccgcggcatctctgttatcccggcagagcctcgtgtgtaagagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtacccacaaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggt

SEQ ID NO: 24 - DNA, 3522 bp, Artificial Sequence; identical to SEQ ID NO: 18
  Description of Artificial Sequence Synthetic polynucleotide

SEQ ID NO: 25 - DNA, 699 bp, Artificial Sequence; identical to SEQ ID NO: 19
  Description of Artificial Sequence Synthetic polynucleotide

SEQ ID NO: 26 - DNA, 900 bp, Artificial Sequence; identical to SEQ ID NO: 20
  Description of Artificial Sequence Synthetic polynucleotide

SEQ ID NO: 27 - DNA, 915 bp, Artificial Sequence
  Description of Artificial Sequence Synthetic polynucleotide
atggaccgtaaaagcaagcgtccggacatgctggttgattcctttggtctggaaagcaccgtgcaggacggtctggttttccgtcagtctttctccattcgtagctatgagattggtactgatcgtaccgcctctatcgaaaccctgatgaatcacctgcaagaaacctctctgaaccattgtaagtctactggcatcctgctggacggtttcggtcgtaccctggagatgtgcaaacgcgacctgatttgggtagtgatcaaaatgcagatcaaagttaaccgttatccggcatggggtgataccgttgaaatcaacacccgcttttctcgtctgggcaaaatcggtatgggccgtgactggctgatctctgactgtaacactggtgaaattctggttcgtgctactagcgcatacgcgatgatgaaccagaaaacccgtcgcctgagcaagctgccgtacgaggtccaccaggagattgttccgctgtttgtagacagcccagtgattgaggattctgacctgaaagtgcataaattcaaagtgaagaccggtgacagcatccaaaaaggcctgaccccaggttggaacgatctggacgttaaccagcacgtttccaacgtgaagtatatcggttggattctggagagcatgccgaccgaggtcctggaaacccaggagctgtgttccctggcgctggagtaccgccgtgagtgcggccgtgacagcgtgctggagtctgtgaccgctatggacccaagcaaagttggtgttcgtagccagtaccagcacctgctgcgtctggaagacggtactgctatcgtgaacggtgcaactgaatggcgtcctaaaaacgcgggtgcaaacggtgctatcagcaccggtaaaacctctaacggtaactccgtgagctaa

SEQ ID NO: 28 - DNA, 621 bp, Artificial Sequence; identical to SEQ ID NO: 21
  Description of Artificial Sequence Synthetic polynucleotide

SEQ ID NO: 29 - DNA, 6538 bp, Artificial Sequence; identical to SEQ ID NO: 22
  Description of Artificial Sequence Synthetic polynucleotide

SEQ ID NO: 30 - DNA, 10863 bp, Artificial Sequence; identical to SEQ ID NO: 23
  Description of Artificial Sequence Synthetic polynucleotide

SEQ ID NO: 31 - DNA, 10878 bp, Artificial Sequence
  Description of Artificial Sequence Synthetic polynucleotide
aaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcaggcgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatggaccgtaaaagcaagcgtccggacatgctggttgattcctttggtctggaaagcaccgtgcaggacggtctggttttccgtcagtctttctccattcgtagctatgagattggtactgatcgtaccgcctctatcgaaaccctgatgaatcacctgcaagaaacctctctgaaccattgtaagtctactggcatcctgctggacggtttcggtcgtaccctggagatgtgcaaacgcgacctgatttgggtagtgatcaaaatgcagatcaaagttaaccgttatccggcatggggtgataccgttgaaatcaacacccgcttttctcgtctgggcaaaatcggtatgggccgtgactggctgatctctgactgtaacactggtgaaattctggttcgtgctactagcgcatacgcgatgatgaaccagaaaacccgtcgcctgagcaagctgccgtacgaggtccaccaggagattgttccgctgtttgtagacagcccagtgattgaggattctgacctgaaagtgcataaattcaaagtgaagaccggtgacagcatccaaaaaggcctgaccccaggttggaacgatctggacgttaaccagcacgtttccaacgtgaagtatatcggttggattctggagagcatgccgaccgaggtcctggaaacccaggagctgtgttccctggcgctggagtaccgccgtgagtgcggccgtgacagcgtgctggagtctgtgaccgctatggacccaagcaaagttggtgttcgtagccagtaccagcacctgctgcgtctggaagacggtactgctatcgtgaacggtgcaactgaatggcgtcctaaaaacgcgggtgcaaacggtgctatcagcaccggtaaaacctctaacggtaactccgtgagctaagagctcgaggaggtttttacaatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctgaggtacccacaaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggt

SEQ ID NO: 32 - DNA, 3549 bp, Artificial Sequence; identical to SEQ ID NO: 3
  Description of Artificial Sequence Synthetic polynucleotide

SEQ ID NO: 33 - DNA, 705 bp, Artificial Sequence; identical to SEQ ID NO: 4
  Description of Artificial Sequence Synthetic polynucleotide

SEQ ID NO: 34 - DNA, 4774 bp, Artificial Sequence; identical to SEQ ID NO: 5
  Description of Artificial Sequence Synthetic polynucleotide

SEQ ID NO: 35 - DNA, 11239 bp, Artificial Sequence; identical to SEQ ID NO: 6
  Description of Artificial Sequence Synthetic polynucleotide

SEQ ID NO: 36 - protein, 232 aa, Nostoc punctiforme
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 37 - DNA, 699 bp, Artificial Sequence; identical to SEQ ID NO: 19
  Description of Artificial Sequence Synthetic polynucleotide

SEQ ID NO: 38 - protein, 246 aa, Artificial Sequence
  Description of Artificial Sequence Synthetic polypeptide
MGSSHHHHHHSQDPMQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 39 - DNA, 738 bp, Artificial Sequence
  Description of Artificial Sequence Synthetic polynucleotide
atgggcagcagccatcaccatcatcaccacagccaggatccgatgcagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcc

SEQ ID NO: 40 - DNA, 60 bp, Artificial Sequence
  Description of Artificial Sequence Synthetic primer
catcaccacagccaggatccgatgcagcaactgaccgatcaaagcaaagaactggacttc

SEQ ID NO: 41 - DNA, 57 bp, Artificial Sequence
  Description of Artificial Sequence Synthetic primer
cggcccgccaagcttttaggcaccgatcaggccataggcgctcagacgcatgatatc

SEQ ID NO: 42 - DNA, 4449 bp, Artificial Sequence
  Description of Artificial Sequence Synthetic polynucleotide
ggggaattgtgagcggataacaattcccctgtagaaataattttgtttaactttaataaggagatataccatgggcagcagccatcaccatcatcaccacagccaggatccgatgcagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 43 - protein, 548 aa, Lactococcus lactis; identical to SEQ ID NO: 17

SEQ ID NO: 44 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 45 - DNA, 1707 bp, Zymomonas mobilis
atgagttatactgtcggtacctatttagcggagcggcttgtccagattggtctcaagcatcacttcgcagtcgcgggcgactacaacctcgtccttcttgacaacctgcttttgaacaaaaacatggagcaggtttattgctgtaacgaactgaactgcggtttcagtgcagaaggttatgctcgtgccaaaggcgcagcagcagccgtcgttacctacagcgtcggtgcgctttccgcatttgatgctatcggtggcgcctatgcagaaaaccttccggttatcctgatctccggtgctccgaacaacaatgatcacgctgctggtcacgtgttgcatcacgctcttggcaaaaccgactatcactatcagttggaaatggccaagaacatcacggccgcagctgaagcgatttacaccccagaagaagctccggctaaaatcgatcacgtgattaaaactgctcttcgtgagaagaagccggtttatctcgaaatcgcttgcaacattgcttccatgccctgcgccgctcctggaccggcaagcgcattgttcaatgacgaagccagcgacgaagcttctttgaatgcagcggttgaagaaaccctgaaattcatcgccaaccgcgacaaagttgccgtcctcgtcggcagcaagctgcgcgcagctggtgctgaagaagctgctgtcaaatttgctgatgctctcggtggcgcagttgctaccatggctgctgcaaaaagcttcttcccagaagaaaacccgcattacatcggtacctcatggggtgaagtcagctatccgggcgttgaaaagacgatgaaagaagccgatgcggttatcgctctggctcctgtcttcaacgactactccaccactggttggacggatattcctgatcctaagaaactggttctcgctgaaccgcgttctgtcgtcgttaacggcgttcgcttccccagcgttcatctgaaagactatctgacccgtttggctcagaaagtttccaagaaaaccggtgctttggacttcttcaaatccctcaatgcaggtgaactgaagaaagccgctccggctgatccgagtgctccgttggtcaacgcagaaatcgcccgtcaggtcgaagctcttctgaccccgaacacgacggttattgctgaaaccggtgactcttggttcaatgctcagcgcatgaagctcccgaacggtgctcgcgttgaatatgaaatgcagtggggtcacatcggttggtccgttcctgccgccttcggttatgccgtcggtgctccggaacgtcgcaacatcctcatggttggtgatggttccttccagctgacggctcaggaagtcgctcagatggttcgcctgaaactgccggttatcatcttcttgatcaataactatggttacaccatcgaagttatgatccatgatggtccgtacaacaacatcaagaactgggattatgccggtctgatggaagtgttcaacggtaacggtggttatgacagcggtgctggtaaaggcctgaaggctaaaaccggtggcgaactggcagaagctatcaaggttgctctggcaaacaccgacggcccaaccctgatcgaatgcttcatcggtcgtgaagactgcactgaagaattggtcaaatggggtaagcgcgttgctgccgccaacagccgtaagcctgttaacaagctcctctag

SEQ ID NO: 46 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 47 - DNA, 3522 bp, Artificial Sequence; identical to SEQ ID NO: 18
  Description of Artificial Sequence Synthetic polynucleotide

SEQ ID NO: 48 - DNA, 726 bp, Artificial Sequence
  Description of Artificial Sequence Synthetic polynucleotide
atgcaccatcaccaccatcatggaggcggacagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaa

SEQ ID NO: 49 - DNA, 900 bp, Artificial Sequence; identical to SEQ ID NO: 20
  Description of Artificial Sequence Synthetic polynucleotide

SEQ ID NO: 50 - DNA, 621 bp, Artificial Sequence; identical to SEQ ID NO: 21
  Description of Artificial Sequence Synthetic polynucleotide

SEQ ID NO: 51 - DNA, 6571 bp, Artificial Sequence
  Description of Artificial Sequence Synthetic polynucleotide
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgccctttacacgtacttagtcgctgaaggcctcactggcccctgcagggatggtggaatgctggttatctggtggggattaagtggtgttttactaaagcttgaacaactcaagaaagattatattcgcaataactgccaataatcccagcatcttgagaaaatccagcaaaccgggggcaaaacaccagcaagaagccagcagactatcaccaaatccccagcgtacagctagaaataactgagcagttgtattcaattaccttctggtcaagccgaggaaatttccccacaccttatacacctctggaaggtttttttgacgaagcgcaaaatatccacaatcggctggggacttcttctgtcagaaaatggcagaaatttttgaatgtgttggcgatcgccctcatcaatgattattagagaacttttgtccctgatgttgggaatactcttgatgacaattgtgattgctcaaagaagaaagaaatttggagtaaatctctaaaaggggactgaaatatttgtatggtcagcatgaccactgaaatggagagaagtctaagacagtagatgtcttagatataagcctcattagaagccatgccataaaacagattttgtggatgaaacaacttgaaatagttcagttgtagaccatgttataaacatttattcttaacacagtgacacattaatgactcatatatccgtccaaaaaaaactaaaatgtttgtaaatttagttttgcggccgcgtcgacttcgttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaatcatatgcaccatcaccaccatcatggaggcggacagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaagagctcctcgaggaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagttttaattaacgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtggcgcgcccagtttcctttactggccctaaagtcgctgtggctagggttccgaaggggcattattggctcgcggctttacaaccttgataaggagagagatgacagttttttttctcttttgcttagtaaaacagcaaatttaaggcatgttaaagagcagtagaacgaaatggttgagccggcctcgatacactcaattaactactaatagcttcaataaattttgggacgattgaagctatttttttgaaaatcaactcttaatatctcctgtctcaaaagagttaattgctaaacaaaagccagtttcagcgaaaaatctagagttttataggttcgttctcagtacaggacaaaaagtttgaaaaggatagagggagagggtttgatggaaataagcacaaatcaatcaagccctcatgaatcagattagcgaaattcgccgccaattgcgacctcatctcggatggcatggagccagactgtcatttatcgccctcttcctggtggcactgttccgagcaaaaaccgtcaatctcgccaaactcgccaccgtctggggaggcaatgcagcagaagagtctaattacaaacgcatgcagcgattctttcagtcctttgacgtcaacatggacaaaatcgccaggatggtaatgaatatcgcggctatcccgcaaccttgggtcttaagcatcgaccgcaccaacggccggcctacatggcccgtcaatcgaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 52 - DNA, 10863 bp, Artificial Sequence; identical to SEQ ID NO: 23
  Description of Artificial Sequence Synthetic polynucleotide

SEQ ID NO: 53 - DNA, 3522 bp, Artificial Sequence; identical to SEQ ID NO: 18
  Description of Artificial Sequence Synthetic polynucleotide

SEQ ID NO: 54 - DNA, 726 bp, Artificial Sequence; identical to SEQ ID NO: 48
  Description of Artificial Sequence Synthetic polynucleotide

SEQ ID NO: 55 - DNA, 621 bp, Artificial Sequence; identical to SEQ ID NO: 21
  Description of Artificial Sequence Synthetic polynucleotide

SEQ ID NO: 56 - DNA, 6893 bp, Artificial Sequence
  Description of Artificial Sequence Synthetic polynucleotide
aaaagcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttcgctgaacctgcaggcgagcatttcaacgatgatgaatgggacggcgaacccactgaacccgtcgccattgacccagaaccgcgcaaagaacgggaaaaaattgatctcgatctggaggatgaaccagaggaaaaccgcaaaccgcaaaaaatcaaagtgaagttagccgatgggaaagagcgggaactcgcccatactcaaaccacaactttttgggatgctgatggtaaacccatttccgcccaagaatttatcgaaaagctatttggcgacctgcccgacctcttcaaggatgaagccgaactacgcaccatctgggggaaacccgatacccgtaaatcgttcctgaccggactcgcggaaaaaggctacggtgacacccaactgaaggcgatcgcacgcattgccgaagcggaaaaaagtgatgtctatgatgtcctgacttgggttgcctacaacaccaaacccattagcagagaagagcgagtaattaagcatcgagatctgattttctcgaagtacaccggaaagcagcaagaatttttagattttgtcctagaccaatacattcgagaaggagtggaggaacttgatcgggggaaactgcctaccctcatcgaaatcaaataccaaaccgttaatgaaggtttagtgatcttgggtcaggatatcggtcaagtattcgcagattttcaggcggatttatataccgaagatgtggcataaaaaaggacggcgatcgccgggggcgttgcctgccttgagcggccgcgtcgacttcgttataaaataaacttaacaaatctatacccacctgtagagaagagtccctgaatatcaaaatggtgggataaaaagctcaaaaaggaaagtaggctgtggttccctaggcaacagtcttccctaccccactggaaactaaaaaaacgagaaaagttcgcaccgaacatcaattgcataattttagccctaaaacataagctgaacgaaactggttgtcttcccttcccaatccaggacaatctgagaatcccctgcaacattacttaacaaaaaagcaggaataaaattaacaagatgtaacagacataagtcccatcaccgttgtataaagttaactgtgggattgcaaaagcattcaagcctaggcgctgagctgtttgagcatcccggtggcccttgtcgctgcctccgtgtttctccctggatttatttaggtaatatctctcataaatccccgggtagttaacgaaagttaatggagatcagtaacaataactctagggtcattactttggactccctcagtttatccgggggaattgtgtttaagaaaatcccaactcataaagtcaagtaggagattaatcatatgcaccatcaccaccatcatggaggcggacagcaactgaccgatcaaagcaaagaactggacttcaagagcgagacgtacaaagacgcctatagccgcattaacgcgatcgtcattgaaggcgaacaagaggcgcatgaaaactacatcaccctggcgcagctgctgcctgagagccacgacgaactgattcgcctgagcaaaatggagagccgtcacaagaaaggttttgaggcgtgtggccgcaatctggcggtgaccccggacctgcaatttgcgaaggagttctttagcggtctgcaccagaatttccagacggccgcagccgagggcaaagtcgtcacttgtttgttgatccagagcctgattattgaatgctttgctattgcggcgtacaacatttacattccggtcgccgatgactttgcgcgtaaaatcacggaaggtgttgtcaaagaggagtattcccacctgaatttcggtgaagtgtggttgaaggaacattttgcggaatctaaagccgaattggaactggcaaatcgccagaacctgccgatcgtttggaagatgctgaaccaagtggaaggtgatgcacatacgatggcgatggagaaggacgcattggttgaggactttatgattcagtatggcgaagcactgtccaatatcggtttcagcacccgtgatatcatgcgtctgagcgcctatggcctgatcggtgcctaagagctcctcgaggaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaggcgcgccacgagaaagagttatgacaaattaaaattctgactcttagattatttccagagaggctgattttcccaatctttgggaaagcctaagtttttagattctatttctggatacatctcaaaagttctttttaaatgctgtgcaaaattatgctctggtttaattctgtctaagagatactgaatacaacataagccagtgaaaattttacggctgtttctttgattaatatcctccaatacttctctagagagccattttccttttaacctatcaggcaatttaggtgattctcctagctgtatattccagagccttgaatgatgagcgcaaatatttctaatatgcgacaaagaccgtaaccaagatataaaaaacttgttaggtaattggaaatgagtatgtattttttgtcgtgtcttagatggtaataaatttgtgtacattctagataactgcccaaaggcgattatctccaaagccatatatgacggcggtagtagaggatttgtgtacttgtttcgataatgcccgataaattcttctacttttttagattggcaatattgagtaatcgaatcgattaattcttgatgcttcccagtgtcataaaataaacttttattcagataccaatgaggatcataatcatgggagtagtgataaatcatttgagttctgactgctacttctatcgactccgtagcattaaaaataagcattctcaaggatttatcaaacttgtatagatttggccggcccgtcaaaagggcgacaccccataattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcgatggcaacaacgttgcgcaaactattaactggcgaactacttactctagcttcccggcaacaattaatagactggatggaggcggataaagttgcaggaccacttctgcgctcggcccttccggctggctggtttattgctgataaatccggagccggtgagcgtggttctcgcggtatcatcgcagcgctggggccagatggtaagccctcccgtatcgtagttatctacacgacggggagtcaggcaactatggatgaacgaaatagacagatcgctgagataggtgcctcactgattaagcattggt

SEQ ID NO: 57 - DNA, 6571 bp, Artificial Sequence; identical to SEQ ID NO: 51
  Description of Artificial Sequence Synthetic polynucleotide

SEQ ID NO: 58 - DNA, 9794 bp, Artificial Sequence
  Description of Artificial Sequence Synthetic polynucleotide
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttattcgactctatagtgaagttcctattctctagaaagtataggaacttctgaagtggggcctgcagggccaccacagccaaattcatcgttaatgtggacttgccgacgcccccttttcgactaacaatcgcaatttttttcatagacatttcccacagaccacatcaaattacagcaattgatctagctgaaagtttaacccacttccccccagacccagaagaccagaggcgcttaagcttccccgaacaaactcaactgaccgagggggagggagccgtagcggcgttggtgttggcgtaaatgacaggccgagcaaagagcgatgagattttcccgacgattgtcttcggggatgtaatttttgtggtggacgcttaaggttaaaacagcccgcaggtgacgatcaatgcctttgaccttcacatccgacggaatacaaaccaagccacagagttcacagcgccagtctgcatcctcttttacttgtaaggcgatcgcctgccaatcatcagaatatcgagaagaatgtttcatctaaacctagcgccgcaagataatcctgaaatcgctacagtattaaaaaattctggccaacatcacagccaatactgcggccgctactcattagttaagtgtaatgcagaaaacgcatattctctattaaacttacgcattaatacgagaattttgtagctacttatactattttacctgagatcccgacataaccttagaagtatcgaaatcgttacataaacattcacacaaaccacttgacaaatttagccaatgtaaaagactacagtttctccccggtttagttctagagttaccttcagtgaaacatcggcggcgtgtcagtcattgaagtagcataaatcaattcaaaataccctgcgggaaggctgcgccaacaaaattaaatatttggtttttcactattagagcatcgattcattaatcaaaaaccttaccccccagcccccttcccttgtagggaagtgggagccaaactcccctctccgcgtcggagcgaaaagtctgagcggaggtttcctccgaacagaacttttaaagagagaggggttgggggagaggttctttcaagattactaaattgctatcactagacctcgtagaactagcaaagactacgggtggattgatcttgagcaaaaaaactttatgagaactttagcaggaggaaaaccatatgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctcgagctcgtgaggtacccacaaggaggtttttacaatgaaaacgacccacaccagcttaccatttgccggccacacgttacatttcgtcgaatttgatccggcgaacttttgtgaacaagacctgttgtggctgccgcattatgcccagctgcagcacgcaggccgtaagcgtaaaactgaacatctggccggtcgcattgcggcagtgtatgccctgcgcgagtacggctacaaatgcgtgccggccattggtgaactgcgtcaaccggtttggccggcagaagtttacggttccatctcccactgcggtactaccgcgttggcggttgtgtctcgccagccgatcggtattgatattgaagagatattctctgtccagacggcacgcgagctgacggacaacatcattaccccggcagagcacgagcgtctggcggactgtggtctggcgttcagcctggcgctgaccctggcattcagcgcaaaagagagcgcgttcaaggcttccgagatccaaaccgatgcgggcttcctggattatcaaatcatcagctggaacaagcaacaggttatcattcaccgtgagaatgagatgtttgccgtccattggcagattaaagagaaaatcgttatcaccctgtgccagcacgactgagaattcggttttccgtcctgtcttgattttcaagcaaacaatgcctccgatttctaatcggaggcatttgtttttgtttattgcaaaaacaaaaaatattgttacaaatttttacaggctattaagcctaccgtcataaataatttgccatttactagtttttaattaaacccctatttgtttatttttctaaatacattcaaatatgtatccgctcatgagacaataaccctgataaatgcttcaataatattgaaaaaggaagagtatgattgaacaagatggcctgcatgctggttctccggctgcttgggtggaacgcctgtttggttacgactgggctcagctgactattggctgtagcgatgcagcggttttccgtctgtctgcacagggtcgtccggttctgtttgtgaaaaccgacctgtccggcgcactgaacgaactgcaggacgaagcggcccgtctgtcctggctcgcgacgactggtgttccgtgcgcggcagttctggacgtagttactgaagccggtcgcgattggctgctgctgggtgaagttccgggtcaggatctgctgagcagccacctcgctccggcagaaaaagtttccatcatggcggacgcgatgcgccgtctgcacaccctggacccggcaacttgcccgtttgaccatcaggctaaacaccgtattgaacgtgcacgcactcgtatggaagcgggtctggttgatcaggacgacctggatgaagagcaccagggcctcgcaccggcggaactgtttgcacgtctgaaagcccgcatgccggacggcgaagacctggtggtaacgcatggcgacgcttgtctgccaaacattatggtggaaaacggccgcttctctggttttattgactgtggccgtctgggtgtagctgatcgctatcaggatatcgccctcgctacccgcgatattgcagaagaactgggtggtgaatgggctgaccgtttcctggtgctgtacggtatcgcagcgccggattctcagcgcattgccttctaccgtctgctggatgagttcttctaaggcgcgccgaaactgcgccaagaatagctcacttcaaatcagtcacggttttgtttagggcttgtctggcgattttggtgacatagacagtcacagcaacagtagccacaaaaccaagaatccggatcgaccactgggcaatggggttggcgctggtgctttctgtgccgagggtcgcaagatttccggccagggagccaatgtagacatacatgatggtgccagggatcatccccacagagccgaggacatagtcttttagggaaacgcccgtgaccccataggcatagttaagcagattaaagggaaatacaggtgagagacgcgtcaggagaacaatcttcaggccttccttgcccacagcttcgtcgatggcgcgaaatttcgggttgtcggcgattttttggctcacccattggcgggccagataacgacccactaggaaagcagcgatcgctcctagggttgcgccaacaaagacgtaaattgatcctaaagcgacaccaaaaacaaccccggctcccaaggtcagaatcgaccccggtagaaaagccaccgtcgccaccacataaagcaccataaaggcgatggccggccaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 59 - protein, 6 aa, Artificial Sequence
  Description of Artificial Sequence Synthetic 6xHis tag
HHHHHH

(19) United States
(12) Patent Application Publication
(10) Pub. No.: US 2015/0152438 A1
(43) Pub. Date: Jun. 4, 2015

(54) Recombinant Synthesis of Alkanes

(71) Applicant: Joule Unlimited Technologies, Inc., Bedford, MA (US)

(72) Inventors: Frank Anthony Skraly, Watertown, MA (US); Ning Li, Bedford, MA (US)

(21) Appl. No.: 14/562,294

(22) Filed: Dec. 5, 2014

Related U.S. Application Data
(63) Continuation of application No. PCT/US2014/013189, filed on Jan. 27, 2014.
(60) Provisional application No. 61/756,973, filed on Jan. 25, 2013.
(60) Provisional application No. 61/826,637, filed on May 23, 2013.

(51) Int. Cl. C12P 5/02; C12N 15/82

(52) U.S. Cl. CPC C12P 5/02; C12N 15/8247

(57) ABSTRACT

The present disclosure identifies methods and compositions for modifying photoautotrophic organisms as hosts, such that the organisms efficiently produce alkanes, and in particular the use of such organisms for the commercial production of alkanes and related molecules. Other materials, methods, and compositions are also described.

### CROSS-REFERENCE TO RELATED APPLICATIONS

This application is a continuation of PCT/US2014/013189, filed on Jan. 27, 2014, which is related to U.S. Provisional Application No. 61/756,973, filed Jan. 25, 2013 and U.S. Provisional Application No. 61/826,637, filed May 23, 2013; each of which is herein incorporated by reference, in its entirety, for all purposes.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### BACKGROUND

Many existing photoautotrophic organisms (i.e., plants, algae, and photosynthetic bacteria) are poorly suited for industrial bioprocessing and have therefore not demonstrated commercial viability. Recombinant photosynthetic microorganisms have been engineered to produce hydrocarbons and alcohols in amounts that exceed the levels produced naturally by the organism.

### SUMMARY

Described herein is an engineered microorganism, wherein said engineered microorganism comprises one or more recombinant genes encoding one or more enzymes having enzyme activities which catalyze the production of alkanes, wherein the enzyme activities comprise: an alkane deformylative monooxygenase activity, a thioesterase activity, a carboxylic acid reductase activity, and a phosphopanthetheinyl transferase activity; an alkane deformylative monooxygenase activity, a thioesterase activity, a long-chain fatty acid CoA-ligase activity, and a long-chain acyl-CoA reductase activity; and/or an alkane deformylative monooxygenase activity, a pyruvate decarboxylase activity and a 2-ketoacid decarboxylase activity.

In some aspects, the enzymes comprise an alkane deformylative monooxygenase, a thioesterase, a carboxylic acid reductase, and a phosphopanthetheinyl transferase. In some aspects, the alkane deformylative monooxygenase has EC number 4.1.99.5, the thioesterase has EC number 3.1.2.14, the carboxylic acid reductase has EC number 1.2.99.6, and the phosphopanthetheinyl transferase has EC number 2.7.8.7. In some aspects, the alkane deformylative monooxygenase is encoded by adm, the thioesterase is encoded by fatB or fatB2, the carboxylic acid reductase is encoded by carB, and the phosphopanthetheinyl transferase is encoded by entD.

In some aspects, the enzyme having alkane deformylative monooxygenase activity has EC number 4.1.99.5. In some aspects, the enzyme having thioesterase activity has EC number 3.1.2.14. In some aspects, the enzyme having carboxylic acid reductase activity has EC number 1.2.99.6. In some aspects, the enzyme having phosphopanthetheinyl transferase activity has EC number 2.7.8.7.

In some aspects, the enzymes comprise an alkane deformylative monooxygenase, a thioesterase, a long-chain fatty acid CoA-ligase, and a long-chain acyl-CoA reductase. In some aspects, the alkane deformylative monooxygenase has EC number 4.1.99.5, the thioesterase has EC number 3.1.2.14, the long-chain fatty acid CoA-ligase has EC number 6.2.1.3, and the long-chain acyl-CoA reductase has EC number 1.2.1.50. In some aspects, the alkane deformylative monooxygenase is encoded by adm, the thioesterase is encoded by fatB or fatB2, the long-chain fatty acid CoA-ligase is encoded by fatD, and the long-chain acyl-CoA reductase is encoded by acrM.

In some aspects, the enzyme having alkane deformylative monooxygenase activity has EC number 4.1.99.5. In some aspects, the enzyme having thioesterase activity has EC number 3.1.2.14. In some aspects, the enzyme having long-chain fatty acid CoA-ligase activity has EC number 6.2.1.3. In some aspects, the enzyme having long-chain acyl-CoA reductase activity has EC number 1.2.1.50.

In some aspects, the one or more recombinant genes comprise a recombinant gene encoding a thioesterase that catalyzes the conversion of acyl-ACP to a fatty acid. In some aspects, the one or more recombinant genes comprises a recombinant gene encoding a phosphopanthetheinyl transferase that phosphopatetheinylates the ACP moiety of a protein encoded by a carboxylic acid reductase gene. In some aspects, the one or more recombinant genes comprise a recombinant gene encoding a carboxylic acid reductase that catalyzes the conversion of fatty acid to fatty aldehyde. In some aspects, the one or more recombinant genes comprise a recombinant gene encoding a alkane deformylative monooxygenase that catalyzes the conversion of fatty aldehyde to an alkane or alkene. In some aspects, the one or more recombinant genes comprise a recombinant gene encoding a fatty acid CoA-ligase that catalyzes the conversion of fatty acid to acyl-CoA. In some aspects, the one or more recombinant genes comprise a recombinant gene encoding an acyl-CoA reductase that catalyzes the conversion of acyl-CoA to fatty aldehyde.

In some aspects, the enzymes comprise an alkane deformylative monooxygenase, a pyruvate decarboxylase and a 2-ketoacid decarboxylase.

In some aspects, said microorganism is a bacterium. In some aspects, said microorganism is a gram-negative bacterium. In some aspects, said microorganism is E. coli.

In some aspects, said microorganism is a photosynthetic microorganism. In some aspects, said microorganism is a cyanobacterium. In some aspects, said microorganism is a thermotolerant cyanobacterium. In some aspects, said microorganism is a Synechococcus species.

In some aspects, expression of an operon comprising the one or more recombinant genes is controlled by a recombinant promoter, and wherein the promoter is constitutive or inducible. In some aspects, said operon is integrated into the genome of said microorganism. In some aspects, said operon is extrachromosomal.

In some aspects, said alkanes are less than or equal to 11 carbon atoms in length. In some aspects, said alkanes are 7 to 11 carbon atoms in length. In some aspects, said alkanes are 7, 8, 9, 10, or 11 carbon atoms in length. In some aspects, said alkanes are less than or equal to 18 carbon atoms in length. In some aspects, said alkanes are 7 to 18 carbon atoms in length. In some aspects, said alkanes are 5, 6, 7, 8, 9, 10, 11, 12, 13, 14, 15, 16, 17, or 18 carbon atoms in length.

In some aspects, said recombinant genes are at least 90% or at least 95% identical to a sequence shown in the Tables.

Also described herein is a cell culture comprising a culture medium and a microorganism described herein.

Also described herein is a method for producing hydrocarbons, comprising: culturing an engineered microorganism described herein in a culture medium, wherein said engineered microorganism produces increased amounts of alkanes relative to an otherwise identical microorganism, cultured under identical conditions, but lacking said recombinant genes. In some aspects, the method further includes allowing alkanes to accumulate in the culture medium or in the organism. In some aspects, the method further includes isolating at least a portion of the alkanes. In some aspects, the method further includes processing the isolated alkanes to produce a processed material.

Also described herein is a method for producing hydrocarbons, comprising: (i) culturing an engineered microorganism described herein in a culture medium; and (ii) exposing said engineered microorganism to light and inorganic carbon, wherein said exposure results in the conversion of said inorganic carbon by said microorganism into alkanes, wherein said alkanes are produced in an amount greater than that produced by an otherwise identical microorganism, cultured under identical conditions, but lacking said recombinant genes. In some aspects, the method further includes allowing alkanes to accumulate in the culture medium or in the organism. In some aspects, the method further includes isolating at least a portion of the alkanes. In some aspects, the method further includes processing the isolated alkanes to produce a processed material.

Also described herein is a composition comprising alkanes, wherein said alkanes are produced by a method described herein. In some aspects, the composition comprises at least 50%, at least 60%, at least 70%, at least 80%, at least 90%, at least 95%, or at least 99% alkanes.

The present invention provides, in certain embodiments a method of producing a short-chain alkane or alkene from an engineered organism, the method comprising: expressing a recombinant alkanal deformylative monooxygenase (“ADM”) in the engineered microorganism; culturing the engineered microorganism in a culture medium containing a carbon source under conditions effective to produce a short-chain alkane or alkene.

In an embodiment, ADM catalyzes the conversion of an aldehyde into an alkane or alkene, wherein the aldehyde is selected from the group consisting of acetaldehyde, butanal, propanal, isobutanal, butanal, 3-methyl-1-butanal and 2-phenylethanal. In an embodiment, the alkane or alkene is selected from the group consisting of methane, propane, ethane, butane, propane, isobutane and toluene. In an embodiment, the method of producing a short-chain alkane or alkene from an engineered organism comprises expressing a recombinant pyruvate decarboxylase (“Pdc”) in the engineered microorganism. In certain embodiments, the Pdc is at least 90% identical SEQ ID NO: 46. In an embodiment, the method of producing a short-chain alkane or alkene from an engineered organism comprises expressing a 2-ketoacid decarboxylase in the engineered microorganism. In certain embodiments, the Pdc or the 2-ketoacid decarboxylase are expressed in an operon under the control of a single promoter.

In an embodiment, the operon comprises ADM. In certain embodiments, the ADM is at least 90% identical to SEQ ID NO: 36.

Also provided herein, are embodiments comprising an engineered microorganism, wherein the engineered microorganism comprises a recombinant gene encoding an alkanal deformylative monooxygenase (“ADM”), and wherein the engineered microorganism further comprises a recombinant gene encoding an enzyme selected from the group consisting of: pyruvate decarboxylase and 2-ketoacid decarboxylase.

In one embodiment, the ADM catalyzes the conversion of an aldehyde into an alkane or alkene, wherein the aldehyde is selected from the group consisting of acetaldehyde, butanal, propanal, isobutanal, 2-methyl-1-butanal, butanal, 3-methyl-1-butanal and 2-phenylethanal. In certain embodiments, the alkane or alkene is selected from the group consisting of methane, propane, ethane, butane, propane, isobutane and toluene.

In one embodiment, the engineered microorganism comprises a recombinant pyruvate decarboxylase (“Pdc”). In certain embodiments, the Pdc is at least 90% identical to SEQ ID NO: 46. In one embodiment, the engineered microorganism comprises a 2-ketoacid decarboxylase. In certain embodiments, the Pdc or the 2-ketoacid decarboxylase are expressed in an operons under the control of a single promoter.

In one embodiment, the operon comprises ADM. In some embodiments, the engineered microorganism is an engineered cyanobacterium. In certain embodiments, the ADM is at least 90% identical to SEQ ID NO: 36.

Also provided herein, are embodiments comprising a cell culture comprising a recombinant microorganism and a culture medium containing a carbon source, wherein a polypeptide that catalyzes the conversion of an aldehyde to an alkane is overexpressed in the recombinant microorganism and an alkane or alkene is produced in the cell culture when the recombinant microorganism is cultured in the culture medium under conditions effective to express the polypeptide. In an embodiment, the polypeptide has alkanal deformylative monooxygenase activity. In an embodiment, the polypeptide comprises an amino acid sequence having at least 90% identity to SEQ ID NO: 36. In some embodiments, the aldehyde is selected from the group consisting of acetaldehyde, butanal, propanal, isobutanal, butanal, 3-methyl-1-butanal, and 2-phenylethanal.

In an embodiment, the alkane or alkene is selected from the group consisting of methane, propane, ethane, butane, propane, isobutane, and toluene. In an embodiment, the alkane is a short-chain alkane. In certain embodiments, the alkane comprises a C₂ to C₄ alkane. In some embodiments, the alkane comprises a C₂ to C₇ alkane. In an embodiment, the alkane or the alkene is secreted into the culture medium.

In an embodiment, the recombinant microorganism further comprises a recombinant polypeptide comprising a pyruvate decarboxylase (“Pdc”) activity. In certain embodiments, the Pdc is at least 90% identical to SEQ ID NO: 46. In an embodiment, the recombinant microorganism further comprises a recombinant 2-ketoacid decarboxylase. In some embodiments, the Pdc or the 2-ketoacid decarboxylase are expressed in an operon under the control of a single promoter. In an embodiment, the operon comprises ADM.

In an embodiment, the recombinant microorganism is selected from the group consisting of yeast, fungi, filamentous fungi, algae, and bacterium. In some embodiments, the bacterium is a cyanobacterium.

Also provided herein, are embodiments comprising a method for producing isobutane or a derivative of isobutane, comprising contacting ADM with an aldehyde in vitro. In an embodiment, the ADM is at least 90% identical to SEQ ID NO: 36. In certain embodiments, the ADM is Nostoc punctiforme ADM. In an embodiment, the aldehyde is 3-methylbutyraldehyde.

These and other embodiments of the invention are further described in the Figures, Description, Examples and Claims, herein.

### BRIEF DESCRIPTION OF THE FIGURES

FIG. 1. SDS-PAGE gel showing the overexpression of AcrM protein in E. coli.

FIG. 2. TIC chromatograms of assays with (A) decanoyl-CoA, (B) lauroyl-CoA. Solid line: wild type BL21(DE3); dotted line: acrM-expressing BL21(DE3).

FIG. 3. GC/FID chromatogram showing the detection of C13 and C15 alkanes produced by Synechococcus sp. PCC 7002 strain expressing Adm, CarB, TesA and EntD proteins. Grey trace: control strain (does not express CarB protein); solid black trace: Standards of C13, C14, and C15 n-alkanes; dashed black trace: Synechococcus sp. PCC 7002 strain expressi-ng Adm, CarB, TesA, and EntD proteins.

FIG. 4. TIC chromatograms of samples from acid-fed (dashed lines) or control (solid lines) Synechococcus sp. PCC 7002 expressing Adm and CarB. A and D: octanoic acid feeding, B and E: decanoic acid feeding, C and F: dodecanoic acid feeding.

FIG. 5. GC/FID chromatogram showing the detection of nonane produced by Synechococcus sp. PCC 7002 strain expressing Adm, CarB, FatB2 and EntD proteins at 12 h and 72 h. Solid trace: control strain (wild type); dotted trace: Synechococcus sp. PCC 7002 strain expressing Adm, CarB, FatB2, and EntD proteins.

FIG. 6. Examples of pathways for production of alkanes. Note that the use of carB can be facilitated by the product of entD (phosphopanthetheinyl transferase), which phosphopatetheinylates the ACP moiety of the CarB protein. For example, one can use the Bacillus entD, whose enzyme product has a wide substrate spectrum that includes CarB.

FIG. 7. Detection of nonane (A) and undecane (B) produced by Synechococcus sp. PCC 7002 strain expressing Adm, thioesterase, CarB, and EntD proteins when fed with decanoic acid and dodecanoic acid. Circles: alkane detected in the cell pellet; triangles: alkane detected in the hexadecane overlay.

FIG. 8. GC/FID chromatograms showing the biosynthesis of nonane (A) and undecane (B) from CO₂, by Synechococcus sp. PCC 7002 strain expressing Adm, thioesterase, CarB, and EntD proteins, secreted into the hexadecane overlay. Solid trace: samples from day 0; dotted trace: samples from day 5.

FIG. 9. Time course of the biosynthesis of undecane (triangle) and nonane (circle) from CO₂, by Synechococcus sp. PCC 7002 strain expressing Adm, thioesterase, CarB, and EntD proteins, secreted into the hexadecane overlay.

FIG. 10. GC/FID chromatogram showing the detection of C13 and C15 alkanes produced by 7002 strain expressing Adm, CarB, TesAₘ and EntD proteins. Solid line: control strain; dotted line: ALK-C13C15 (experimental strain).

FIG. 11. The growth curve of ALK-C13C15 over 10 days.

FIG. 12. The production curve of tridecane and pentadecane by ALK-C13C15 over 10 days.

FIG. 13. Depicts fractions from Ni-NTA purification of His₆-tagged ADM enzyme. The collected fractions pooled for assay use are indicated.

FIG. 14. Time course of the biosynthesis of undecane (triangle) from CO₂ by JCC6036.

FIG. 15. Detection of nonane produced by 7002 strain expressing Adm, CarB, and EntD proteins when fed with decanoic acid. By expressing Nhistagged Adm on pAQ3, the initial activity was increased significantly compared to that on pAQ4.

### DETAILED DESCRIPTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native intemucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

As used herein, an “isolated” organic molecule (e.g., an alkane) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, intemucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

Deletion:

The removal of one or more nucleotides from a nucleic acid molecule or one or more amino acids from a protein, the regions on either side being joined together.

Knock-Out:

A gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present invention may be used to produce an equivalent effect and are therefore envisioned to be part of the present invention.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred embodiment, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

“Carbon-based Products of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, Docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Biofuel: A biofuel refers to any fuel that derives from a biological source. Biofuel can refer to one or more hydrocarbons, one or more alcohols (such as ethanol), one or more fatty esters, or a mixture thereof.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Unless otherwise defined, all technical and scientific terms used herein have the same meaning as commonly understood by one of ordinary skill in the art to which this present invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Throughout this specification and claims, the word “comprise” or variations such as “comprises” or “comprising”, will be understood to imply the inclusion of a stated integer or group of integers but not the exclusion of any other integer or group of integers.

### Nucleic Acid Sequences

The present invention provides isolated nucleic acid molecules for genes encoding enzymes, and variants thereof. Exemplary full-length nucleic acid sequences for genes encoding enzymes and the corresponding amino acid sequences are presented in Tables 1 and 2.

In one embodiment, the present invention provides an isolated nucleic acid molecule having a nucleic acid sequence comprising or consisting of a gene coding for an alkane deformylative monooxygenase, a thioesterase, a carboxylic acid reductase, a phosphopanthetheinyl transferase, a long-chain fatty acid CoA-ligase, and/or a long-chain acyl-CoA reductase and homologs, variants and derivatives thereof expressed in a host cell of interest. The present invention also provides a nucleic acid molecule comprising or consisting of a sequence which is a codon-optimized version of the alkane deformylative monooxygenase, a thioesterase, a carboxylic acid reductase, a phosphopanthetheinyl transferase, a long-chain fatty acid CoA-ligase, and/or a long-chain acyl-CoA reductase genes described herein. In a further embodiment, the present invention provides a nucleic acid molecule and homologs, variants and derivatives of the molecule comprising or consisting of a sequence which is a variant of the alkane deformylative monooxygenase, a thioesterase, a carboxylic acid reductase, a phosphopanthetheinyl transferase, a long-chain fatty acid CoA-ligase, and/or a long-chain acyl-CoA reductase gene having at least 80% identity to the wild-type gene. The nucleic acid sequence can be preferably greater than 80%, 85%, 90%, 95%, 98%, 99%, 99.9% or even higher identity to the wild-type gene.

In another embodiment, the nucleic acid molecule of the present invention encodes a polypeptide having an amino acid sequence disclosed in Tables 1 and 2. Preferably, the nucleic acid molecule of the present invention encodes a polypeptide sequence of at least 50%, 60, 70%, 80%, 85%, 90% or 95% identity to the amino acid sequences shown in Tables 1 and 2 and the identity can even more preferably be 96%, 97%, 98%, 99%, 99.9% or even higher.

The present invention also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The nucleic acid sequence fragments of the present invention display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hydridization to target sequences can be detected and optionally quantified. One of skill in the art will appreciate that the nucleic acid fragments of the present invention may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosure of each of which is incorporated herein by reference in its entirety.

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf. M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present invention. Other methods and techniques may also be suitable for the measurement of enzyme activity, as would be known by one of skill in the art.

### Vectors

Also provided are vectors, including expression vectors, which comprise the above nucleic acid molecules of the present invention, as described further herein. In a first embodiment, the vectors include the isolated nucleic acid molecules described above. In an alternative embodiment, the vectors of the present invention include the above-described nucleic acid molecules operably linked to one or more expression control sequences. The vectors of the instant invention may thus be used to express a polypeptide contributing to alkane producing activity by a host cell.

Vectors useful for expression of nucleic acids in prokaryotes are well known in the art.

### Isolated Polypeptides

According to another aspect of the present invention, isolated polypeptides (including muteins, allelic variants, fragments, derivatives, and analogs) encoded by the nucleic acid molecules of the present invention are provided. In one embodiment, the isolated polypeptide comprises the polypeptide sequence corresponding to a polypeptide sequence shown in Table 1 or 2. In an alternative embodiment of the present invention, the isolated polypeptide comprises a polypeptide sequence at least 85% identical to a polypeptide sequence shown in Table 1 or 2. Preferably the isolated polypeptide of the present invention has at least 50%, 60, 70%, 80%, 85%, 90%, 95%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9% or even higher identity to a polypeptide sequence shown in Table 1 or 2.

According to other embodiments of the present invention, isolated polypeptides comprising a fragment of the above-described polypeptide sequences are provided. These fragments preferably include at least 20 contiguous amino acids, more preferably at least 25, 30, 35, 40, 45, 50, 60, 70, 80, 90, 100 or even more contiguous amino acids.

The polypeptides of the present invention also include fusions between the above-described polypeptide sequences and heterologous polypeptides. The heterologous sequences can, for example, include sequences designed to facilitate purification, e.g. histidine tags, and/or visualization of recombinantly-expressed proteins. Other non-limiting examples of protein fusions include those that permit display of the encoded protein on the surface of a phage or a cell, fusions to intrinsically fluorescent proteins, such as green fluorescent protein (GFP), and fusions to the IgG Fc region.

### Host Cell Transformants

In another aspect of the present invention, host cells transformed with the nucleic acid molecules or vectors of the present invention, and descendants thereof, are provided. In some embodiments of the present invention, these cells carry the nucleic acid sequences of the present invention on vectors, which may but need not be freely replicating vectors. In other embodiments of the present invention, the nucleic acids have been integrated into the genome of the host cells.

In an alternative embodiment, the host cells of the present invention can be mutated by recombination with a disruption, deletion or mutation of the isolated nucleic acid of the present invention so that the activity of one or more enzyme(s) in the host cell is reduced or eliminated compared to a host cell lacking the mutation.

### Selected or Engineered Microorganisms for the Production of Carbon-Based Products of Interest

Microorganism: Includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chiysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium. Cyanobacteria include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochiorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera:

Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Preferred organisms for the manufacture of alkanes according to the methods disclosed herein include: Arabidopsis thaliana, Panicum virgatum, Miscanthus giganteus, and Zea mays (plants); Botryococcus braunii, Chlamydomonas reinhardtii and Dunaliela salina (algae); Synechococcus sp PCC 7002, Synechococcus sp. PCC 7942, Synechocystis sp. PCC 6803, Thermosynechococcus elongatus BP-1 (cyanobacteria); Chlorobium tepidum (green sulfur bacteria), Chloroflexus auranticus (green non-sulfur bacteria); Chromatium tepidum and Chromatium vinosum (purple sulfur bacteria); Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A suitable organism for selecting or engineering is capable of autotrophic fixation of CO₂ to products. This would cover photosynthesis and methanogenesis. Acetogenesis, encompassing the three types of CO₂ fixation; Calvin cycle, acetyl-CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. See, e.g., Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

Alkane production via engineered cyanobacteria, e.g., a Synechococcus or Thermosynechococcus species, is preferred. Other preferred organisms include Synechocystis, Klebsiella oxytoca, Escherichia coli or Saccharomyces cerevisiae. Other prokaryotic, archaea and eukaryotic host cells are also encompassed within the scope of the present invention.

In some aspects, alkane production via a photosynthetic organism can be carried out using the compositions, materials, and methods described in: PCT/US2009/035937 (filed Mar. 3, 2009); and PCT/US2009/055949 (filed Sep. 3, 2009); each of which is herein incorporated by reference in its entirety, for all purposes.

### Carbon-Based Products of Interest: Hydrocarbons & Alcohols

In various embodiments of the invention, desired hydrocarbons and/or alcohols of certain chain length or a mixture thereof can be produced. In certain aspects, the host cell produces at least one of the following carbon-based products of interest: alkanes such as heptane, nonane, tridecane, pentadecane, and/or undecane. In other aspects, the carbon chain length ranges from C₂ to C₂₀, e.g., C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀. Accordingly, the invention provides production of various chain lengths of alkanes suitable for use as fuels & chemicals.

In preferred aspects, the methods provide culturing host cells for direct product secretion for easy recovery without the need to extract biomass. These carbon-based products of interest are secreted directly into the medium. Since the invention enables production of various defined chain length of hydrocarbons and alcohols, the secreted products are easily recovered or separated. The products of the invention, therefore, can be used directly or used with minimal processing.

### Fuel Compositions

In various embodiments, compositions produced by the methods of the invention are used as fuels. Such fuels comply with ASTM standards, for instance, standard specifications for diesel fuel oils D 975-09b, and Jet A, Jet A-1 and Jet B as specified in ASTM Specification D. 1655-68. Fuel compositions may require blending of several products to produce a uniform product. The blending process is relatively straightforward, but the determination of the amount of each component to include in a blend is much more difficult. Fuel compositions may, therefore, include aromatic and/or branched hydrocarbons, for instance, 75% saturated and 25% aromatic, wherein some of the saturated hydrocarbons are branched and some are cyclic. Preferably, the methods of the invention produce an array of hydrocarbons, such as C₂-C₁₇ or C₁₀-C₁₅ to alter cloud point. Furthermore, the compositions may comprise fuel additives, which are used to enhance the performance of a fuel or engine. For example, fuel additives can be used to alter the freezing/gelling point, cloud point, lubricity, viscosity, oxidative stability, ignition quality, octane level, and flash point. Fuels compositions may also comprise, among others, antioxidants, static dissipater, corrosion inhibitor, icing inhibitor, biocide, metal deactivator and thermal stability improver.

In addition to many environmental advantages of the invention such as CO₂ conversion and renewable source, other advantages of the fuel compositions disclosed herein include low sulfur content, low emissions, being free or substantially free of alcohol and having high cetane number.

### Example 1

### Crude Extract of E. coli Cells Overexpressing acrM Convert Lauroyl-CoA to Dodecanal and Decanoyl-CoA to Decanal

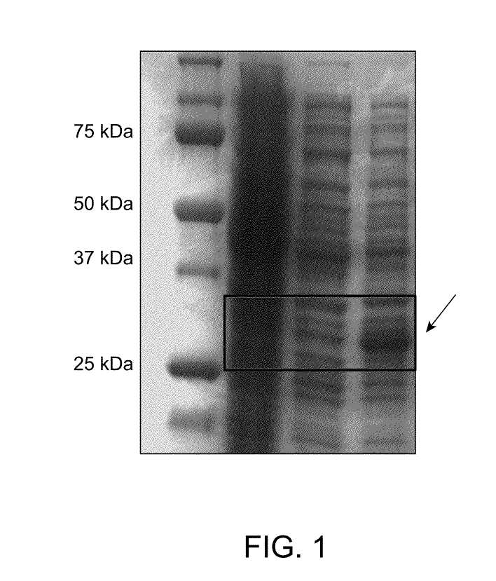

Acinetobacter sp. M-1 acyl coenzyme A reductase, acrM, was codon-optimized for E. coli expression and synthesized by DNA2.0 (Menlo Park, Calif.; SEQ ID NO. 1) with a NdeI site on the 5′ end and an EcoRI site on the 3′end. The obtained gene was subcloned into a pET28a vector (Novagen) by digestion with NdeI and EcoRI and subsequent ligation. The resulting plasmid, pET28a-acrM (SEQ ID NO. 2), containing an N-terminal His₆-tagged acrM, was transformed into a BL21(DE3) E. coli strain purchased from New England Biolabs, which was subsequently grown with shaking in Luria-Bertani medium supplemented with 100 μg/mL of kanomycin in a volume of 1 L to OD₆₀₀=0.8 before induction with 0.25 mM Isopropyl β-D-1-thiogalactopyranoside for 5 hours in a 2-L shaker flask at 37° C. An SDS-PAGE gel demonstrating the overexpression of AcrM protein in pET28a-acrM containing BL21(DE3) E. coli cells is shown in FIG. 1.

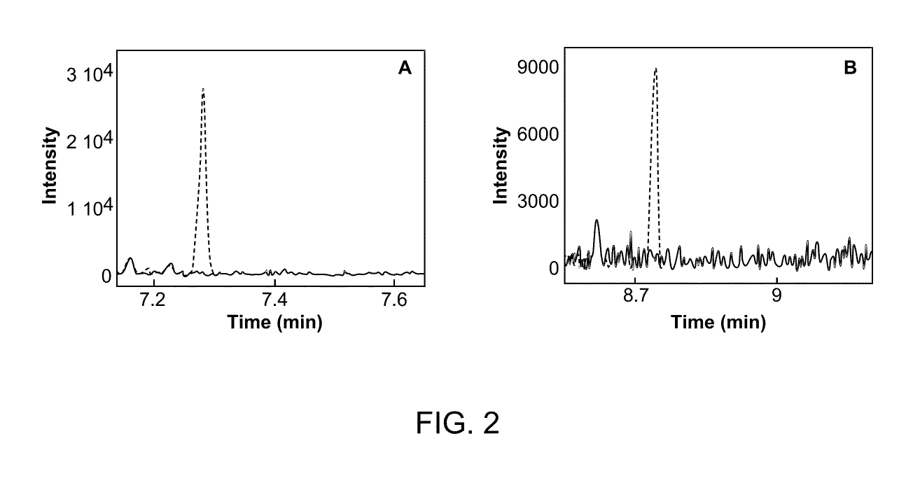

The E. coli cells containing overexpressed AcrM were collected by centrifugation, resuspended in HEPES buffer (100 mM HEPES, 10% glycerol, pH 7.5) at a 1:3 (w/v) ratio and lysed by sonication. 200 μL of buffer solution containing 100 μL total lysate, 1 mM acyl-CoA, 3 mM NADH (Sigma-Aldrich), 100 mM HEPES, 10% glycerol at pH 7.5 was incubated at 37° C. for 30 min, extracted with 100 μL ethyl acetate and analyzed by GC/MS equipped with a HP-5 ms column (Agilent, Santa Clara, Calif.). Total ion chromatography (TIC) indicated the detection of aldehydes produced from corresponding acyl-CoA substrates by the AcrM-containing cell extract in the presence of supplemented NADH, as shown in FIG. 2, indicating that AcrM is able to convert lauroyl-CoA to dodecanal and decanoyl-CoA to decanal.

### Example 2

### Feeding Fatty Acid to Synechococcus Sp. PCC 7002 Strain Expressing Adm-carB-entD Results in Detection of Corresponding Aldehyde and Alkane

The carboxylic acid reductase (carB) gene (SEQ ID NO. 3) was PCR-amplified from Mycobacterium smegmatis and verified by sequencing with multiple primers by Genewiz (South Plainfield, N.J.). Cyanothece adm, E. coli leaderless tesA and E. coli entD genes were codon-optimized for E. coli overexpression and synthesized by DNA 2.0 (Menlo Park, Calif.; SEQ ID NO. 4 and 5) with an individual ribosome binding site in front of each gene. All four genes were subcloned into a pUC19 vector containing an ammonia-repressible P(nir07) promoter (U.S. Pat. No. 7,955,820), upstream/downstream homology regions, and a spectinomycin marker. The resulting plasmid, pAQ3::P(nir07)-adm-carB-tesA-entD-SpecR (SEQ ID NO. 6), was transformed into wild-type Synechococcus sp. PCC 7002 and segregated in the presence of spectinomycin.

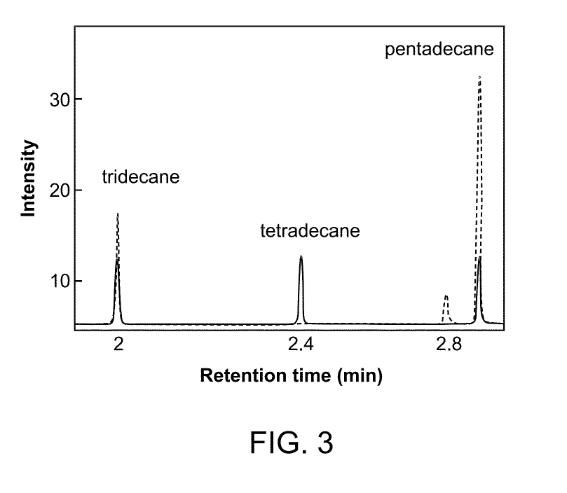

The expression and activity of the Adm, CarB, TesA, and EntD proteins were demonstrated by detection of tridecane and pentadecane in the transformed Synechococcus sp. PCC 7002 strain by GC/FID (FIG. 3).

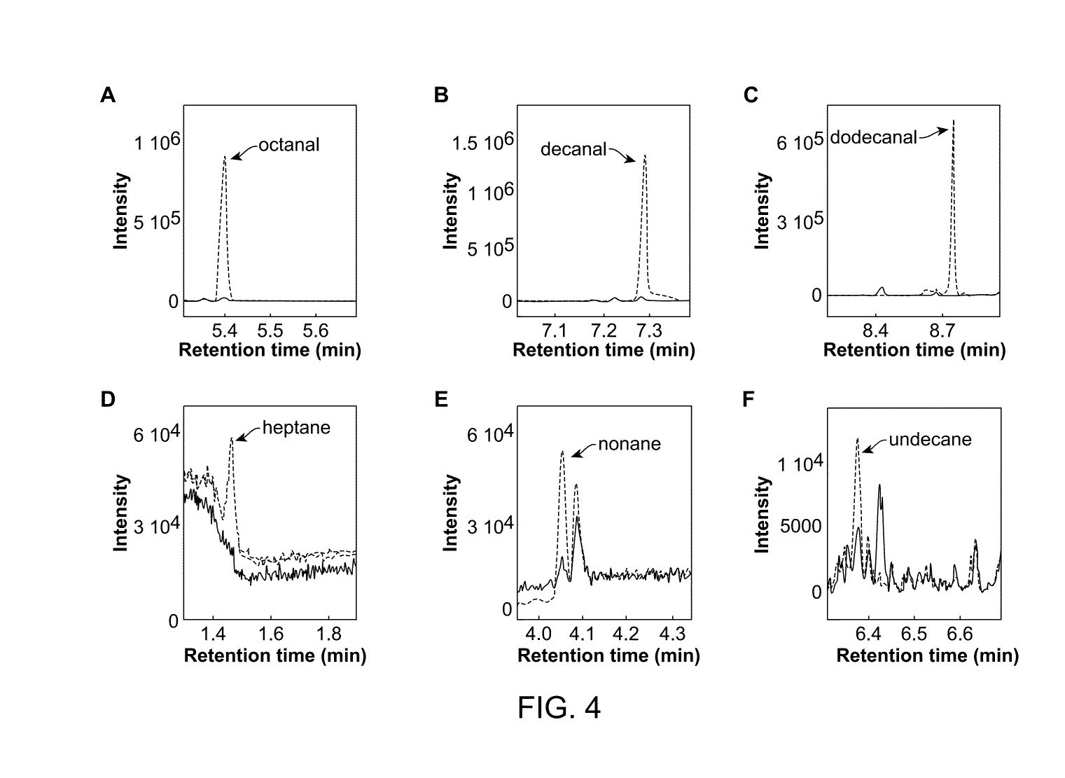

The Synechococcus sp. PCC 7002 cultures were grown to OD₇₃₀˜5 before 1 mM fatty acid (100 mM stock in ethanol) was added and were then shaken at 150 rpm, 37° C. for ˜3 hours in the absence (lauric acid feeding) or presence (octanoic acid and decanoic acid feeding) of a pentadecane overlay (6 mL culture with 1 mL overlay). The pentadecane overlay from the octanoic acid-fed culture (FIGS. 4A and 4D), or decanoic acid culture (FIGS. 4B and 4E) was analyzed by GC/MS equipped with an HP-5 ms column. For the lauric acid feeding assay, 1 mL culture was extracted with 400 μL hexane by vortexing for 1 min before being analyzed by GC/MS (FIG. 4C, 4F). Note that the pAQ3::P(nir07)-adm-carB-tesA-entD-SpecR expressing Synechococcus sp. PCC 7002 strain can produce a detectable level of undecane even without feeding dodecanoic acid. Adm and carB together is able to produce undecane in vivo.

### Example 3

### Synechococcus sp. PCC 7002 Strain Expressing Adm-carB-fatB2-entD Results in Increased Detection of Nonane in Pentadecane Overlay

The E. coli leaderless tesA of pAQ3::P(nir07)-adm-carB-tesA-entD-SpecR, was replaced by Cuphea hookeriana leaderless fatB2 (a medium-chain acyl-ACP thioesterase), which was codon-optimized for E. coli overexpression and synthesized by DNA 2.0 (Menlo Park, Calif.; SEQ ID NO. 7), with an individual ribosome binding site in front of the gene, a 5′ Kpn I restriction site and a 3′ Hind III restriction site. The resulting plasmid, pAQ3::P(nir07)-adm-carB-fatB2-entD-SpecR (SEQ ID NO. 8), was transformed into wild-type Synechococcus sp. PCC 7002 and segregated in the presence of spectinomycin.

The wild type Synechococcus sp. PCC 7002 and pAQ3::P(nir07)-adm-carB-fatB2-entD-SpecR expressing Synechococcus sp. PCC 7002 cultures (35 mL) were grown in JB3.0 media (Table A below) to OD₇₃₀˜3 (in the presence of 2 mM urea) before a 10 mL pentadecane overlay was added.

TABLE A
JB3.0 Media

| | Amount | | Calculated |
|---|---|---|---|
| Ingredient | per liter | Units | Amount |
| NaCl | 18 | g | 36 |
| Citric Acid | 1 | g | 2 |
| KCl | 0.6 | g | 1.2 |
| NaNO₃ | 5.1 | g | 10.2 |
| 500 g/l | 10 | mL | 20 |
| MgSO₄•7H₂O | | | |
| 50 g/l KH₂PO₄ | 4.6 | mL | 9.2 |
| 17.76 g/l CaCl₂ | 15 | mL | 30 |
| 3 g/l NaEDTAₜₑₜᵣₐ | 10 | mL | 20 |
| 3.52 g/l Ferric | 4.83 | mL | 9.66 |
| Citrate (in 0.1N HCl) | | | |
| 0.88M Tris (pH 8.2) | 9.375 | mL | 18.75 |
| P1 Metals | 1 | mL | 2 |
| Solution | | | |
| MilliQ H₂O | 950 | mL | 1900 |
| 4 mg/l Vitamin B₁₂ | 1 | mL | 2 |

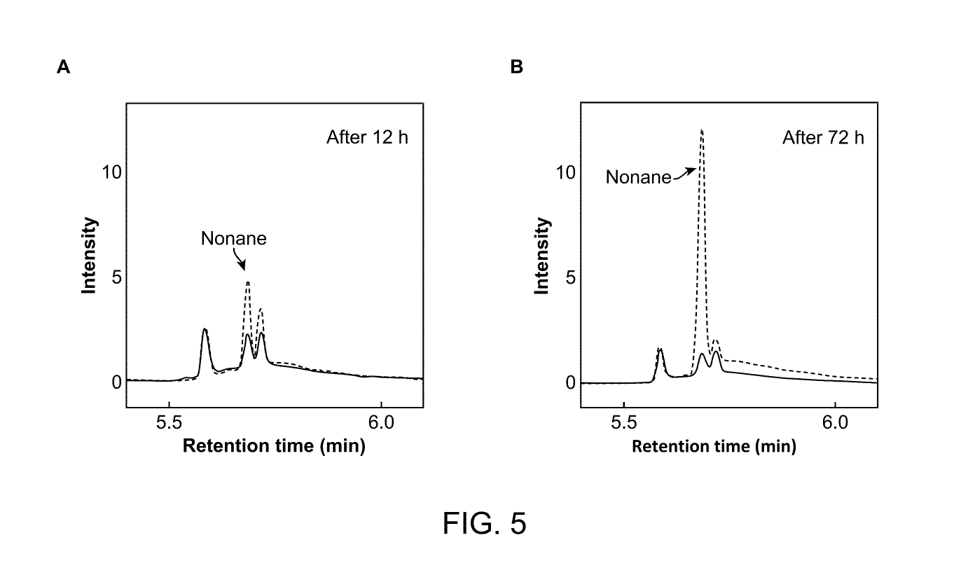

The cultures were shaken at 150 rpm, 37° C. for 3 more days continuously. 100 μL pentadecane overlay samples from each flask were taken 12 hours (FIG. 5A) or 72 hours (FIG. 5B) after pentadecane addition, respectively, and analyzed directly by GC/FID equipped with a 20 meter hp-5 ms column. An increase of nonane production was detected in the pAQ3::P(nir07)-adm-carB-fatB2-entD-SpecR expressing Synechococcus sp. PCC 7002 cultures but not in the wild type control ones. A relative increase in octane and heptanes production was also detected in the pAQ3::P(nir07)-adm-carB-fatB2-entD-SpecR expressing Synechococcus sp. PCC 7002 cultures. Adm, CarB and FatB2 together produced nonane in vivo. Shorter alkanes can also be produced via Adm-CarB pathway if shorter fatty acids are provided in vivo.

### Example 4

### Alkane Production

One or more recombinant genes encoding one or more enzymes having enzyme activities which catalyze the production of alkanes are identified and selected. The enzyme activities include: an alkane deformylative monooxygenase activity, a thioesterase activity, a carboxylic acid reductase activity, and a phosphopanthetheinyl transferase activity, a long-chain fatty acid CoA-ligase activity, and/or a long-chain acyl-CoA reductase activity. Such genes and enzymes can be those described in Tables 1 and 2.

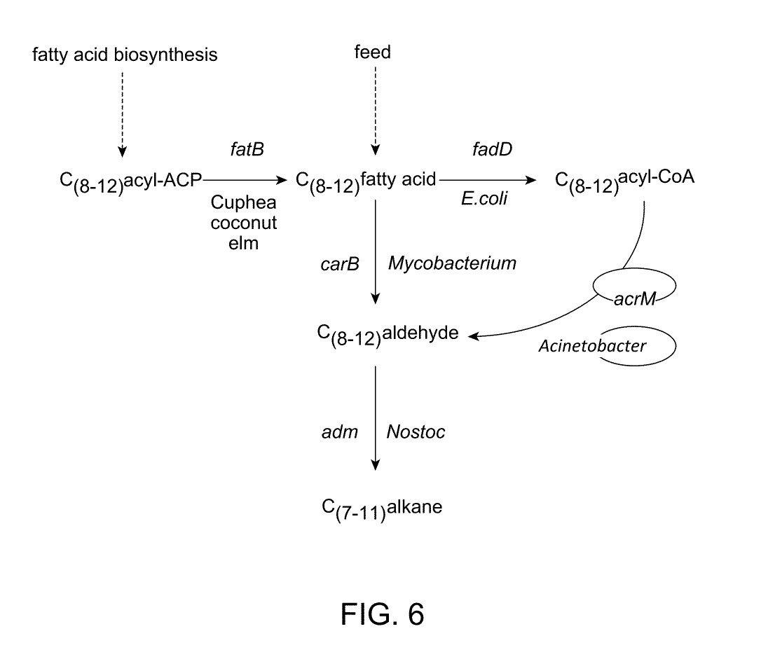

The selected genes are cloned into an expression vector. For example, adm-carB-entD-fatB or adm-acrM-fadD-fatB (or combinations of homologs thereof) are cloned into one or more vectors. See FIG. 6. The genes can be under inducible control (such as the urea-repressible nir07 promoter or the cumate-inducible cum02 promoter). The genes may or may not be expressed operonically; and one or more of the genes can be placed under constitutive control such that when the other gene(s) are induced, the genes under constitutive control are already expressed. For example, one might express adm, carB, and entD constitutively while placing fatty-acid-generating fatB under inducible control; thus when fatty acids are made by fatB after induction, the remainder of the pathway is already present.

One or more vectors are selected and transformed into a microorganism (e.g., cyanobacteria). The cells are grown to a suitable optical density. In some instances cells are grown to a suitable optical density in an uninduced state, and then an induction signal is applied to commence alkane production.

Alkanes are produced by the transformed cells. The alkanes generally have 7, 8, 9, 10, 11 or more carbon atoms. In some instances, alkanes are detected. In some instances, alkanes are quantified. In some instances, alkanes are collected.

In some aspects, a thioesterase such as fatB can be used. To test downstream of fatB, fatty acids of various chain lengths are fed along with inorganic carbon (e.g., CO₂) to cells, and alkane production is monitored. After fatB addition, cells are provided with inorganic carbon (e.g., CO₂) and alkane production is monitored.

### Example 5

### Feeding Decanoic Acid and Dodecanoic Acid to Adm, Thioesterase and carB/entD Expressing Synechococcus Sp. PCC 7002 Strain Results in Detection of Corresponding Nonane and Undecane with Secretion

Carboxylic acid reductase (carB) (SEQ ID NO. 18) was PCR amplified from Mycobacterium smegmatis and verified by sequencing with multiple primers by Genewiz. Nostoc punctiforme adm, Umbellularia californicia fatBₘ (where subscript “m” indicates mature protein, i.e., without leader sequence), and E. coli entD genes were codon-optimized for E. coli overexpression and synthesized by DNA 2.0 (Menlo Park, Calif.; SEQ ID NOs. 19, 20, and 21). The adm gene was subcloned into a pUC19 vector with a P(cpcB) promoter (U.S. Pat. No. 7,794,969), upstream/downstream homology regions, and an erythromycin marker. The resulting plasmid (pAQ4::P(cpcB)-adm_{Npu}-ermC (SEQ ID NO. 22)) was transformed into wild-type Synechococcus sp. PCC 7002 strain and segregated in the presence of erythromycin (which resulted in strain ADM). The fatBₘ, carB, and entD genes were subcloned into a pUC19 vector containing a P(nir07) promoter, upstream/downstream homology regions, and a spectinomycin marker. The resulting plasmid (pAQ3::P(nir07)-fatBₘ-carB-entD-SpecR (SEQ ID NO. 23)) was transformed into the strain ADM and segregated in the presence of the antibiotic spectinomycin.

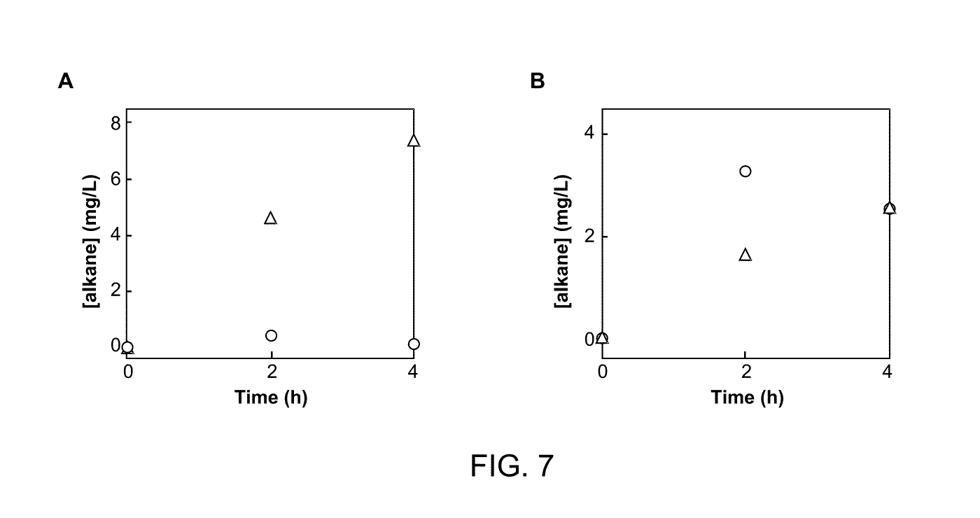

The culture of the above final strain was grown in JB3.0 media till OD₇₃₀˜6 at 37° C., 150 rpm, and with 2% CO₂, in the presence of 15 mM urea. The cells were spun down, resuspended in fresh media without urea, and grown overnight to allow the expression of proteins regulated under the P(nir07) promoter. An overlay of 1.5 mL hexadecane was then added onto the 6 mL culture before 0.1 mM decanoic acid or dodecanoic acid (200 mM stock, dissolved in 100% ethanol) was fed into the culture every 2 hours. At 2 and 4 hours, 0.15 mL of the overlay (triangle) and 0.6 mL of the aqueous culture sample (circle) were collected and analyzed by GC/FID equipped with an hp-5 ms column. When fed with decanoic acid, nonane was produced in vivo with an initial rate of >2.2 mg/L/h, >90% of which was secreted into the overlay (FIG. 7A). When fed with dodecanoic acid, undecane was produced in vivo with an initial rate of 1.2 mg/L/h, ˜50% of which was secreted after 4 hours (FIG. 7B). This indicates that the undecane product is spontaneously secreted to the overlay outside the cells overtime.

### Example 6

### Biosynthesis of Nonane and Undecane by Synechococcus Sp. PCC 7002 Strain Expressing Adm, Thioesterase and carB/entD with Secretion

Carboxylic acid reductase (carB) (SEQ ID NO. 24) was PCR amplified from Mycobacterium smegmatis and verified by sequencing with multiple primers by Genewiz. Nostoc punctiforme adm, Umbellularia californicia fatBₘ (where subscript “m” indicates mature protein, i.e. without leader sequence), Cuphea hookeriana fatB2ₘ, and E. coli entD genes were codon-optimized for E. coli overexpression and synthesized by DNA 2.0 (Menlo Park, Calif.; SEQ ID NOs. 25, 26, 27, and 28). The adm gene was subcloned into a pUC19 vector with P(cpcB) promoter, upstream/downstream homology regions, and an erythromycin marker. The resulting plasmid (pAQ4::P(cpcB)-adm_{Npu}-ermC (SEQ ID NO. 29)) was transformed into wild-type Synechococcus sp. PCC 7002 strain and segregated in the presence of erythromycin (which resulted in strain ADM). The fatBₘ, carB, and entD genes were subcloned into a pUC19 vector containing a P(nir07) promoter, upstream/downstream homology regions, and a spectinomycin marker. The resulting plasmid (pAQ3::P(nir07)-fatBₘ-carB-entD-SpecR (SEQ ID NO. 30)) was transformed into the strain ADM and segregated in the presence of the antibiotic spectinomycin, resulting in strain ALK-C11. The fatB2ₘ, carB, and entD genes were subcloned into a pUC19 vector containing a P(nir07) promoter, upstream/downstream homology regions, and a spectinomycin marker. The resulting plasmid (pAQ3::P(nir07)-fatB2ₘ-carB-entD-SpecR (SEQ ID NO. 31)) was transformed into the strain ADM and segregated in the presence of the antibiotic spectinomycin, resulting in strain ALK-C9.

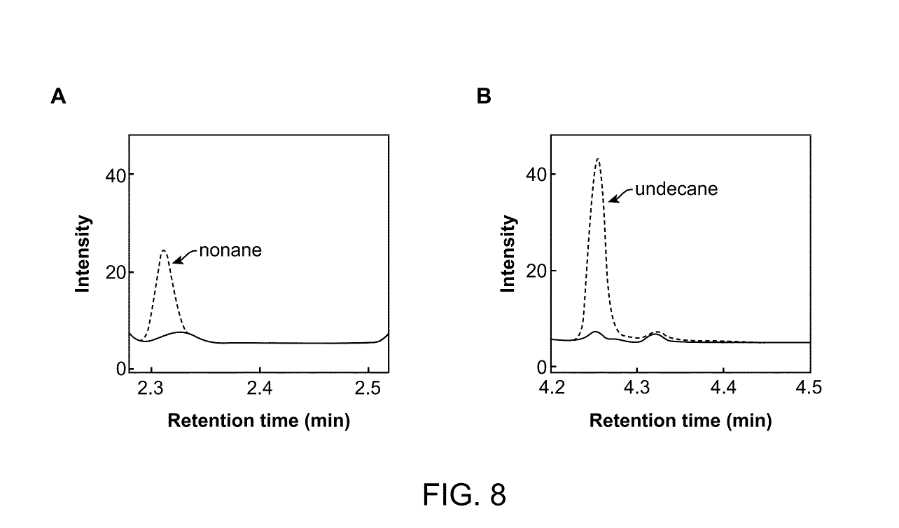

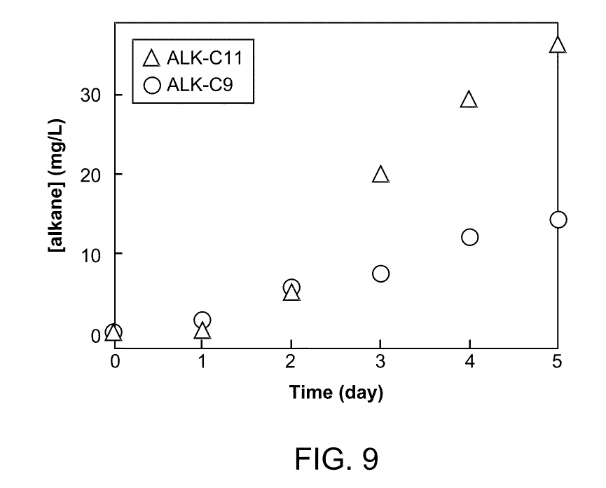

ALK-C9 (FIG. 8A) and ALK-C11 (FIG. 8B) were grown in JB3.0 media till OD₂₃₀˜3 at 37° C., 150 rpm and with 2% CO₂, in the presence of 15 mM urea. The cells were spun down, resuspended in fresh media without urea and 8 mL hexadecane overlay was then added onto the 32 mL culture. Each day, 0.1 mL of the overlay was collected and analyzed by GC/FID equipped with an hp-5 ms column. An increasing amount of nonane was detected in the overlay for ALK-C9 (FIG. 9, circle), and an increasing amount of undecane was detected in the overlay for ALK-C11 (FIG. 9, triangle). Nonane and undecane are produced continuously by ALK-C9 and ALK-C11 from CO₂.

### Example 7

### Biosynthesis of Tridecane and Pentadecane by Synechococcus Sp. PCC 7002 Strain Expressing Adm, tesA (Thioesterase), and carB/entD

Carboxylic acid reductase (carB) (SEQ ID NO. 32) was PCR amplified from Mycobacterium smegmatis and verified by sequencing with multiple primers by Genewiz. Cyanothece sp. ATCC 51142 adm, E. coli tesAₘ (where subscript “m” indicates mature protein, i.e. without leader sequence), and E. coli entD genes were codon-optimized for E. coli overexpression and synthesized by DNA 2.0 (Menlo Park, Calif.; SEQ ID NO. 33 and 34, respectively) with individual ribosome binding sites in front of each gene. All four genes were subcloned into a pUC19 vector containing a P(nir07) promoter, upstream/downstream homology regions, and a spectinomycin marker. The resulting plasmid (pAQ3::P(nir07)-adm-carB-tesAₘ-entD-SpecR (SEQ ID NO. 35)) was transformed into wild-type 7002 strain and segregated in the presence of the antibiotic spectinomycin resulting in strain ALK-C13C15.

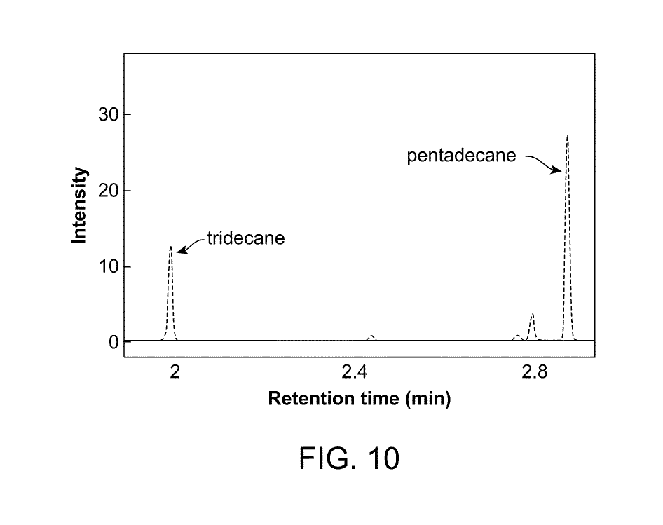

ALK-C13C15 of OD₇₃₀˜0.5 was grown in a shaker flask at 37° C., 150 rpm with 2% CO₂ in the presence of 2 mM urea in JB3.0 medium. After 48 h, 0.5 mL sample of the culture was collected and centrifuged for 5 min at 15,000 rpm. The cell pellet was extracted with acetone and analyzed by GC/FID equipped with an hp-5 ms column. FIG. 10. A control strain that did not express tesAₘ, carB, or entD proteins was treated similarly, and the sample was prepared and analyzed by the same method.

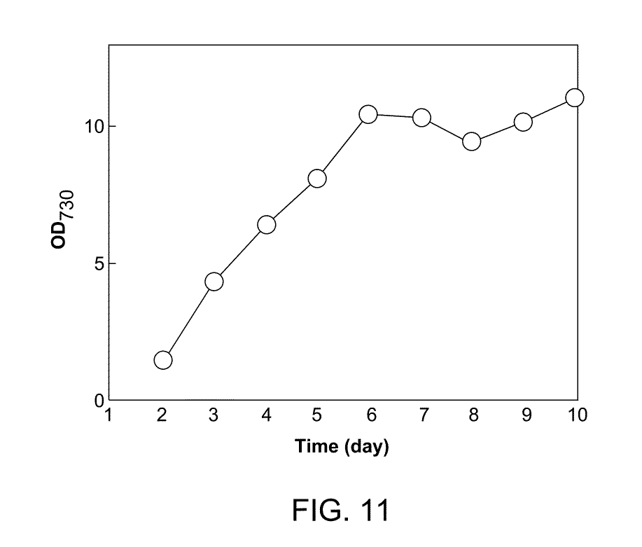

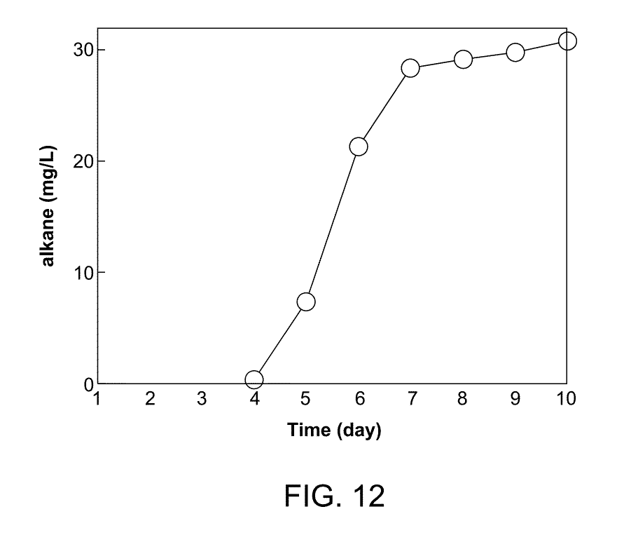

The growth and alkane production of ALK-C13C15 was also analyzed over a ten day period of time. FIG. 11 shows the growth curve of ALK-C13C15 over 10 days. FIG. 12 shows the production curve of tridecane and pentadecane by ALK-C13C15 over 10 days.

Nonane and undecane are produced continuously by ALK-C9 and ALK-C11 from in vivo using CO₂ and sunlight.

### Example 8

### A Pathway for the Enzymatic Synthesis of Short-Chain Alkanes

Organisms are constructed which express both adm (alkanal deformylative monooxygenase) and a pathway leading to the formation of a short-chain aldehyde. Examples of such aldehyde-generating pathways are shown in Table 3.

TABLE 3
Pathways for production of an aldehyde and subsequent conversion
to an alkane/alkene via alkanal deformylative monooxygenase.

| | Resultant | |
|---|---|---|
| Pathway | aldehyde | Alkane product |
| pdc, Zymomonas mobilis | acetaldehyde | methane |
| (EC 4.1.1.1) | | |
| 2-ketoacid decarboxylase | propanal | ethane |
| (EC 4.1.1.72) | isobutanal | propane |
| | 2-methyl-1-butanal | butane |
| | butanal | propane |
| | 3-methyl-1-butanal | isobutane |
| | 2-phenylethanal | toluene |

For example, an organism (e.g., cyanobacterium) is engineered according to standard genetic engineering techniques to express Pdc from Zymomonas mobilis (SEQ ID NO: 46) and Adm from N. punctiforme (SEQ ID NO: 36). The Pdc polypeptide converts pyruvate to acetaldehyde. The Adm polypeptide converts acetaldehyde to the short-chain alkane, methane. The genes of the invention may be constructed synthetically or isolated by PCR.

Alternatively, ketoacid decarboxylase and Adm are recombinantly expressed by the organism. The ketoacid decarboxylase is KivD from Lactococcus lactis subsp. lactis KF147 (SEQ ID NO: 43). Alternatively, the ketoacid decarboxylase is ARO10 from Saccharomyces cerevisiae S288c (SEQ ID NO: 44).

The resulting organism comprises an operon coexpressing an adm gene and pdc and/or a 2-ketoacid decarboxylase gene. Cells will be cultured and the presence of the expected product in Table 3 will be measured by gas chromatography analysis.

### Example 9

### Purified ADM from Nostoc punctiforme PCC 73102 Deformylates Isovaleraldehyde and Forms Isobutane In Vitro

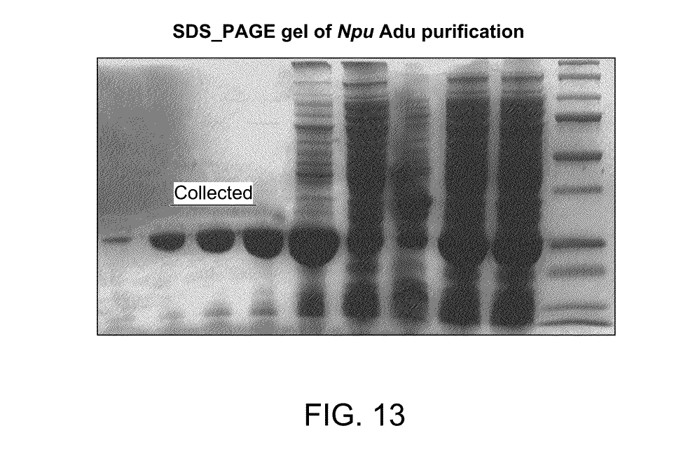

N. punctiforme PCC73102 adm was amplified from the codon-optimized gene obtained from DNA2.0 (Menlo Park, Calif.; SEQ ID NO. 37) by PCR using primers UN19 (5′-CAT CAC CAC AGC CAG GAT CCG ATG CAG CAA CTG ACC GAT CAA AGC AAA GAA CTG GAC TTC-3′) (SEQ ID NO: 40) and UN20 (5′-CGG CCC GCC AAG CTT TTA GGC ACC GAT CAG GCC ATA GGC GCT CAG ACG CAT GAT ATC-3′) (SEQ ID NO: 41), allowing the introduction of 5′ BamHI and 3′ HindIII restriction sites. The resulting PCR product was inserted into the E. coli vector pCDF-Duet1 (Merck; Darmstadt, Germany) by digestion with BamHI and HindIII and subsequent ligation. The resulting plasmid, pCDF-npu (SEQ ID NO. 42), containing N-terminal His₆-tagged N. punctiforme adm, was transformed into E. coli strain BL21(DE3), which was subsequently grown with shaking in Luria-Bertani medium supplemented with 100 ng/mL of spectinomycin in a volume of 1 L to OD₆₀₀=0.8 before induction with 0.25 mM IPTG for 4 hours in a 2-L shaker flask at 37° C. The ADM protein was purified by affinity chromatography using a Ni-NTA agarose (Qiagen; Valencia, Calif.) column, eluting the purified protein with a buffer solution of pH 7.5, which contained 100 mM HEPES, 10% glycerol and 250 mM imidazole. An SDS-PAGE gel of the collected fractions is shown in FIG. 13.

The activity of the purified ADM was tested on various short-chain aldehydes: isobutyraldehyde, 2-methylbutyraldehyde, and 3-methylbutyraldehyde, among which the 3-methylbutyraldehyde (isovaleraldehyde) is converted to isobutane; whereas the other two showed no detectable deformylation to the corresponding alkane. The activity of purified ADM was also tested on butanal, valeraldehyde, and isovaleraldehyde, as shown in Table 4. The assay conditions were as follows: ˜0.2 mM N. punctiforme Adm (N-His₆-tagged), 0.3 mM 1-methoxy-5-methylphenazinium methyl sulfate (Sigma-Aldrich; St. Louis, Mo.), 10 mM NADH (Sigma-Aldrich), 10 mM aldehyde (stock of 250 mM, dissolved in dimethyl sulfoxide), in a buffer solution containing 100 mM HEPES, 10% glycerol at pH 7.4. Each assay was run at 25° C. for 5 minutes, after which it was immediately analyzed by headspace gas chromatography using a 20-m HP-5MS column (Agilent Technologies; Santa Clara, Calif.). The column was kept at 40° C. for 3 min before being heated to 100° C. at 15 C.°/min. Species were identified according to retention time, compared to corresponding standards, which were purchased from Sigma-Aldrich. Results are shown in Table 4. The expression of ADM results in an increase in peak area for each product.

TABLE 4
Results of chromatagram assays.

| | | Product | | Product |
|---|---|---|---|---|
| | | retention | Reaction | peak area |
| Substrate | Product | time (min) | condition | (arbitrary unit) |
| Butanal | Propane | 1.33 | No ADM | 4.1 |
| | | | With ADM | 11.2 |
| Valeraldehyde | Butane | 1.42 | No ADM | 2.5 |
| | | | With ADM | 32 |
| Isovaleraldehyde | Isobutane | 1.36 | No ADM | 3.4 |
| | | | With ADM | 17.4 |

### Example 10

### Biosynthesis of Undecane by Synechococcus Sp. PCC 7002 Strain Expressing Adm, Thioesterase and carB/entD with Secretion

Carboxylic acid reductase (carB) (SEQ ID NO. 47) was PCR amplified from Mycobacterium smegmatis and verified by sequencing with multiple primers by Genewiz. Hexahistidine-tagged Nostoc punctiforme adm, Umbellularia californicia fatBₘ (without leader sequence), and E. coli entD genes were codon-optimized for E. coli overexpression and synthesized by DNA2.0 (Menlo Park, Calif.; SEQ ID NO. 48, 49, and 50). The adm gene with an N-terminal hexahistidine tag was subcloned into a pUC19 vector with P(cpcB) promoter, upstream and downstream homologous regions, and a erythromycin marker. The resulting plasmid (pAQ4::P(cpcB)-Nhistag_adm(Npu)-ErmC (SEQ ID NO. 51)) was transformed into wild-type Synechococcus sp. PCC 7002 and segregated in the presence of erythromycin (which resulted in strain ADM). The fatBₘ, carB and entD genes were subcloned into a pUC19 vector containing a P(nir07) promoter, upstream and downstream homologous regions, and a spectinomycin marker. The resulting plasmid (pAQ3::P(nir07)-fatBₘ-carB-entD-SpecR (SEQ ID NO. 52)) was transformed into the strain ADM and segregated in the presence of the antibiotic spectinomycin, resulting in strain JCC6036.

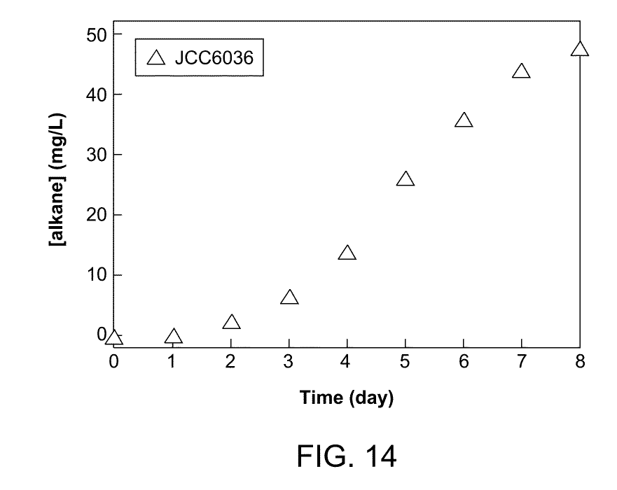

JCC6036 was grown up in JB3.0 media to OD₇₃₀˜3 at 37° C., 150 rpm and with 2% CO₂, in the presence of 15 mM urea. The cells were spun down, resuspended in fresh JB3.0 media with 3 mM urea and a 6 mL pentadecane overlay was then added onto 30 mL culture. 0.06 mL of the overlay was collected everyday and analyzed by GC/FID equipped with an hp-5 ms column. An increased amount of undecane was detected in the overlay for JCC6036 (FIG. 14).

### Example 11

### Feeding Decanoic Acid to Adm and carB/entD-Expressing Synechococcus Sp. PCC 7002 Strain Results in Detection of Corresponding Nonane with Secretion. His-Tagged Adm on pAQ3 Showed Significantly Higher Activity In Vivo

Carboxylic acid reductase (carB) (SEQ ID NO. 53) was PCR amplified from Mycobacterium smegmatis and verified by sequencing with multiple primers by Genewiz. Hexahistidine-tagged Nostoc punctiforme adm and E. coli entD genes codon-optimized for E. coli overexpression were synthesized by DNA 2.0 (Menlo Park, Calif.; SEQ ID NO. 54 and 55). The adm gene was subcloned into a pUC19 vector with P(cpcB) promoter, upstream and downstream homologous regions of pAQ3 or pAQ4, and a spectinomycin marker. The resulting plasmids (pAQ3::P(cpcB)-Nhistag_adm(Npu)-SpecR (SEQ ID NO. 56) and pAQ4::P(cpcB)-Nhistag_adm(Npu)-EmrC (SEQ ID NO. 57)) were transformed into wild-type Synechococcus sp. PCC 7002 strain and segregated in the presence of spectinomycin (resulting in strains ADM3 and ADM4). The carB and entD genes were subcloned into a pUC19 vector containing a P(nir07) promoter, upstream and downstream homologous regions of pAQ7, and a kanamycin marker. The resulting plasmid (pAQ7::P(nir07)-carB-entD-KanR (SEQ ID NO. 58)) was transformed into strains ADM3 and ADM4 and segregated in the presence of the antibiotic spectinomycin (resulting in strains ADM3CARB and ADM4CARB).

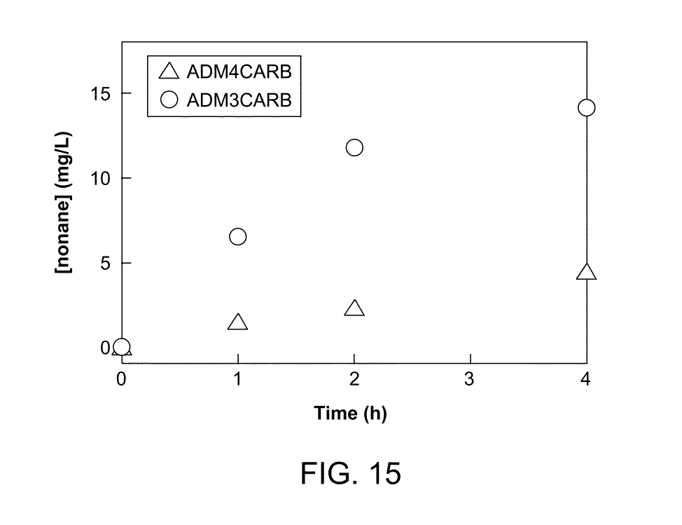

The ADM3CARB and ADM4CARB strains were grown in JB3.0 media to OD₇₃₀˜4 at 37° C., 150 rpm and with 2% CO₂, in the presence of 15 mM urea. The cells were spun down, resuspended in fresh JB3.0 media without urea, and grown overnight to allow the expression of proteins regulated by the P(nir07) promoter. 1.5 mL pentadecane overlay was then added onto 6 mL of culture before 4 mM decanoic acid (500 mM stock, dissolved in 100% ethanol) was fed into the culture at the beginning. 0.08 mL of the overlay was collected at 1 and 2 hours after feeding and analyzed by GC/FID equipped with an hp-5 ms column. When fed with decanoic acid, nonane was produced in vivo by the strain ADM3CARB with an initial rate of ˜6 mg/L/h (FIG. 15).

A number of embodiments of the invention have been described. Nevertheless, it will be understood that various modifications may be made without departing from the spirit and scope of the invention. All publications, patents and other references mentioned herein are hereby incorporated by reference in their entirety.

TABLE 1

| SEQ | | |
|---|---|---|
| ID NO | DESCRIPTION | SEQUENCE |
| 1 | acrM (from | ATGAATGCAAAACTGAAGAAATTGTTCCAGCAGAAAGTAGACGGCAAGACCATCATCGTGACCGGTGCAA |
| | Acinetobacter | GCAGCGGTATTGGCTTGACCGTGAGCAAATACCTGGCTCAGGCGGGTGCACACGTGCTGCTGCTGGCGCG |
| | sp. M-1), | TACGAAAGAGAAACTGGATGAGGTCAAGGCGGAGATTGAAGCGGAAGGCGGTAAGGCTACTGTTTTCCCG |
| | codon- | TGCGATTTGAATGACATGGAATCCATTGACGCAGTCAGCAAAGAGATCCTGGCAGCCGTTGATCATATCG |
| | optimized for | ACATTCTGGTGAATAACGCGGGTCGCAGCATCCGTCGCGCGGTCCACGAAAGCGTGGATCGCTTCCATGA |
| | E. coli | CTTTGAGCGTACCATGCAACTGAATTACTTCGGTGCCGTTCGTCTGGTCCTGAATGTTCTGCCGCACATG |
| | | ATGCAGCGCAAAGATGGCCAAATCATTAACATTAGCAGCATTGGCGTTTTGGCGAACGCGACGCGTTTCA |
| | | GCGCGTATGTGGCGAGCAAGGCTGCACTGGATGCCTTCTCCCGTTGTCTGAGCGCCGAGGTCCATTCGCA |
| | | CAAGATTGCGATTACCTCTATCTATATGCCGCTGGTTCGTACCCCGATGATTGCGCCGACGAAGATCTAC |
| | | AAGTATGTCCCAACGTTGTCCCCGGAAGAGGCGGCTGACCTGATTGCTTATGCGATCGTTAAACGTCCGA |
| | | AAAAGATCGCCACCAATCTGGGTCGCCTGGCAAGCATCACCTACGCGATTGCCCCGGACATCAACAACAT |
| | | CCTGATGAGCATCGGCTTTAACCTGTTTCCGTCTAGCACGGCGAGCGTGGGTGAGCAAGAAAAGCTGAAC |
| | | CTGATTCAACGTGCCTACGCACGTCTGTTTCCTGGTGAACACTGGTAA |
| 2 | Plasmid | TGGCGAATGGGACGCGCCCTGTAGCGGCGCATTAAGCGCGGCGGGTGTGGTGGTTACGCGCAGCGTGACC |
| | pET28a-acrM | GCTACACTTGCCAGCGCCCTAGCGCCCGCTCCTTTCGCTTTCTTCCCTTCCTTTCTCGCCACGTTCGCCG |
| | | GCTTTCCCCGTCAAGCTCTAAATCGGGGGCTCCCTTTAGGGTTCCGATTTAGTGCTTTACGGCACCTCGA |
| | | CCCCAAAAAACTTGATTAGGGTGATGGTTCACGTAGTGGGCCATCGCCCTGATAGACGGTTTTTCGCCCT |
| | | TTGACGTTGGAGTCCACGTTCTTTAATAGTGGACTCTTGTTCCAAACTGGAACAACACTCAACCCTATCT |
| | | CGGTCTATTCTTTTGATTTATAAGGGATTTTGCCGATTTCGGCCTATTGGTTAAAAAATGAGCTGATTTA |
| | | ACAAAAATTTAACGCGAATTTTAACAAAATATTAACGTTTACAATTTCAGGTGGCACTTTTCGGGGAAAT |
| | | GTGCGCGGAACCCCTATTTGTTTATTTTTCTAAATACATTCAAATATGTATCCGCTCATGAATTAATTCT |
| | | TAGAAAAACTCATCGAGCATCAAATGAAACTGCAATTTATTCATATCAGGATTATCAATACCATATTTTT |
| | | GAAAAAGCCGTTTCTGTAATGAAGGAGAAAACTCACCGAGGCAGTTCCATAGGATGGCAAGATCCTGGTA |
| | | TCGGTCTGCGATTCCGACTCGTCCAACATCAATACAACCTATTAATTTCCCCTCGTCAAAAATAAGGTTA |
| | | TCAAGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATTCAT |
| | | TCGTGATTGCGCCTGAGCGAGACGAAATACGCGATCGCTGTTAAAAGGACAATTACAAACAGGAATCGAA |
| | | TGCAACCGGCGCAGGAACACTGCCAGCGCATCAACAATATTTTCACCTGAATCAGGATATTCTTCTAATA |
| | | CCTGGAATGCTGTTTTCCCGGGGATCGCAGTGGTGAGTAACCATGCATCATCAGGAGTACGGATAAAATG |
| | | CTTGATGGTCGGAAGAGGCATAAATTCCGTCAGCCAGTTTAGTCTGACCATCTCATCTGTAACATCATTG |
| | | GCAACGCTACCTTTGCCATGTTTCAGAAACAACTCTGGCGCATCGGGCTTCCCATACAATCGATAGATTG |
| | | TCGCACCTGATTGCCCGACATTATCGCGAGCCCATTTATACCCATATAAATCAGCATCCATGTTGGAATT |
| | | TAATCGCGGCCTAGAGCAAGACGTTTCCCGTTGAATATGGCTCATAACACCCCTTGTATTACTGTTTATG |
| | | TAAGCAGACAGTTTTATTGTTCATGACCAAAATCCCTTAACGTGAGTTTTCGTTCCACTGAGCGTCAGAC |
| | | CCCGTAGAAAAGATCAAAGGATCTTCTTGAGATCCTTTTTTTCTGCGCGTAATCTGCTGCTTGCAAACAA |
| | | AAAAACCACCGCTACCAGCGGTGGTTTGTTTGCCGGATCAAGAGCTACCAACTCTTTTTCCGAAGGTAAC |
| | | TGGCTTCAGCAGAGCGCAGATACCAAATACTGTCCTTCTAGTGTAGCCGTAGTTAGGCCACCACTTCAAG |
| | | AACTCTGTAGCACCGCCTACATACCTCGCTCTGCTAATCCTGTTACCAGTGGCTGCTGCCAGTGGCGATA |
| | | AGTCGTGTCTTACCGGGTTGGACTCAAGACGATAGTTACCGGATAAGGCGCAGCGGTCGGGCTGAACGGG |
| | | GGGTTCGTGCACACAGCCCAGCTTGGAGCGAACGACCTACACCGAACTGAGATACCTACAGCGTGAGCTA |
| | | TGAGAAAGCGCCACGCTTCCCGAAGGGAGAAAGGCGGACAGGTATCCGGTAAGCGGCAGGGTCGGAACAG |
| | | GAGAGCGCACGAGGGAGCTTCCAGGGGGAAACGCCTGGTATCTTTATAGTCCTGTCGGGTTTCGCCACCT |
| | | CTGACTTGAGCGTCGATTTTTGTGATGCTCGTCAGGGGGGCGGAGCCTATGGAAAAACGCCAGCAACGCG |
| | | GCCTTTTTACGGTTCCTGGCCTTTTGCTGGCCTTTTGCTCACATGTTCTTTCCTGCGTTATCCCCTGATT |
| | | CTGTGGATAACCGTATTACCGCCTTTGAGTGAGCTGATACCGCTCGCCGCAGCCGAACGACCGAGCGCAG |
| | | CGAGTCAGTGAGCGAGGAAGCGGAAGAGCGCCTGATGCGGTATTTTCTCCTTACGCATCTGTGCGGTATT |
| | | TCACACCGCATATATGGTGCACTCTCAGTACAATCTGCTCTGATGCCGCATAGTTAAGCCAGTATACACT |
| | | CCGCTATCGCTACGTGACTGGGTCATGGCTGCGCCCCGACACCCGCCAACACCCGCTGACGCGCCCTGAC |
| | | GGGCTTGTCTGCTCCCGGCATCCGCTTACAGACAAGCTGTGACCGTCTCCGGGAGCTGCATGTGTCAGAG |
| | | GTTTTCACCGTCATCACCGAAACGCGCGAGGCAGCTGCGGTAAAGCTCATCAGCGTGGTCGTGAAGCGAT |
| | | TCACAGATGTCTGCCTGTTCATCCGCGTCCAGCTCGTTGAGTTTCTCCAGAAGCGTTAATGTCTGGCTTC |
| | | TGATAAAGCGGGCCATGTTAAGGGCGGTTTTTTCCTGTTTGGTCACTGATGCCTCCGTGTAAGGGGGATT |
| | | TCTGTTCATGGGGGTAATGATACCGATGAAACGAGAGAGGATGCTCACGATACGGGTTACTGATGATGAA |
| | | CATGCCCGGTTACTGGAACGTTGTGAGGGTAAACAACTGGCGGTATGGATGCGGCGGGACCAGAGAAAAA |
| | | TCACTCAGGGTCAATGCCAGCGCTTCGTTAATACAGATGTAGGTGTTCCACAGGGTAGCCAGCAGCATCC |
| | | TGCGATGCAGATCCGGAACATAATGGTGCAGGGCGCTGACTTCCGCGTTTCCAGACTTTACGAAACACGG |
| | | AAACCGAAGACCATTCATGTTGTTGCTCAGGTCGCAGACGTTTTGCAGCAGCAGTCGCTTCACGTTCGCT |
| | | CGCGTATCGGTGATTCATTCTGCTAACCAGTAAGGCAACCCCGCCAGCCTAGCCGGGTCCTCAACGACAG |
| | | GAGCACGATCATGCGCACCCGTGGGGCCGCCATGCCGGCGATAATGGCCTGCTTCTCGCCGAAACGTTTG |
| | | GTGGCGGGACCAGTGACGAAGGCTTGAGCGAGGGCGTGCAAGATTCCGAATACCGCAAGCGACAGGCCGA |
| | | TCATCGTCGCGCTCCAGCGAAAGCGGTCCTCGCCGAAAATGACCCAGAGCGCTGCCGGCACCTGTCCTAC |
| | | GAGTTGCATGATAAAGAAGACAGTCATAAGTGCGGCGACGATAGTCATGCCCCGCGCCCACCGGAAGGAG |
| | | CTGACTGGGTTGAAGGCTCTCAAGGGCATCGGTCGAGATCCCGGTGCCTAATGAGTGAGCTAACTTACAT |
| | | TAATTGCGTTGCGCTCACTGCCCGCTTTCCAGTCGGGAAACCTGTCGTGCCAGCTGCATTAATGAATCGG |
| | | CCAACGCGCGGGGAGAGGCGGTTTGCGTATTGGGCGCCAGGGTGGTTTTTCTTTTCACCAGTGAGACGGG |
| | | CAACAGCTGATTGCCCTTCACCGCCTGGCCCTGAGAGAGTTGCAGCAAGCGGTCCACGCTGGTTTGCCCC |
| | | AGCAGGCGAAAATCCTGTTTGATGGTGGTTAACGGCGGGATATAACATGAGCTGTCTTCGGTATCGTCGT |
| | | ATCCCACTACCGAGATATCCGCACCAACGCGCAGCCCGGACTCGGTAATGGCGCGCATTGCGCCCAGCGC |
| | | CATCTGATCGTTGGCAACCAGCATCGCAGTGGGAACGATGCCCTCATTCAGCATTTGCATGGTTTGTTGA |
| | | AAACCGGACATGGCACTCCAGTCGCCTTCCCGTTCCGCTATCGGCTGAATTTGATTGCGAGTGAGATATT |
| | | TATGCCAGCCAGCCAGACGCAGACGCGCCGAGACAGAACTTAATGGGCCCGCTAACAGCGCGATTTGCTG |
| | | GTGACCCAATGCGACCAGATGCTCCACGCCCAGTCGCGTACCGTCTTCATGGGAGAAAATAATACTGTTG |
| | | ATGGGTGTCTGGTCAGAGACATCAAGAAATAACGCCGGAACATTAGTGCAGGCAGCTTCCACAGCAATGG |
| | | CATCCTGGTCATCCAGCGGATAGTTAATGATCAGCCCACTGACGCGTTGCGCGAGAAGATTGTGCACCGC |
| | | CGCTTTACAGGCTTCGACGCCGCTTCGTTCTACCATCGACACCACCACGCTGGCACCCAGTTGATCGGCG |
| | | CGAGATTTAATCGCCGCGACAATTTGCGACGGCGCGTGCAGGGCCAGACTGGAGGTGGCAACGCCAATCA |
| | | GCAACGACTGTTTGCCCGCCAGTTGTTGTGCCACGCGGTTGGGAATGTAATTCAGCTCCGCCATCGCCGC |
| | | TTCCACTTTTTCCCGCGTTTTCGCAGAAACGTGGCTGGCCTGGTTCACCACGCGGGAAACGGTCTGATAA |
| | | GAGACACCGGCATACTCTGCGACATCGTATAACGTTACTGGTTTCACATTCACCACCCTGAATTGACTCT |
| | | CTTCCGGGCGCTATCATGCCATACCGCGAAAGGTTTTGCGCCATTCGATGGTGTCCGGGATCTCGACGCT |
| | | CTCCCTTATGCGACTCCTGCATTAGGAAGCAGCCCAGTAGTAGGTTGAGGCCGTTGAGCACCGCCGCCGC |
| | | AAGGAATGGTGCATGCAAGGAGATGGCGCCCAACAGTCCCCCGGCCACGGGGCCTGCCACCATACCCACG |
| | | CCGAAACAAGCGCTCATGAGCCCGAAGTGGCGAGCCCGATCTTCCCCATCGGTGATGTCGGCGATATAGG |
| | | CGCCAGCAACCGCACCTGTGGCGCCGGTGATGCCGGCCACGATGCGTCCGGCGTAGAGGATCGAGATCTC |
| | | GATCCCGCGAAATTAATACGACTCACTATAGGGGAATTGTGAGCGGATAACAATTCCCCTCTAGAAATAA |
| | | TTTTGTTTAACTTTAAGAAGGAGATATACCATGGGCAGCAGCCATCATCATCATCATCACAGCAGCGGCC |
| | | TGGTGCCGCGCGGCAGCCATATGAATGCAAAACTGAAGAAATTGTTCCAGCAGAAAGTAGACGGCAAGAC |
| | | CATCATCGTGACCGGTGCAAGCAGCGGTATTGGCTTGACCGTGAGCAAATACCTGGCTCAGGCGGGTGCA |
| | | CACGTGCTGCTGCTGGCGCGTACGAAAGAGAAACTGGATGAGGTCAAGGCGGAGATTGAAGCGGAAGGCG |
| | | GTAAGGCTACTGTTTTCCCGTGCGATTTGAATGACATGGAATCCATTGACGCAGTCAGCAAAGAGATCCT |
| | | GGCAGCCGTTGATCATATCGACATTCTGGTGAATAACGCGGGTCGCAGCATCCGTCGCGCGGTCCACGAA |
| | | AGCGTGGATCGCTTCCATGACTTTGAGCGTACCATGCAACTGAATTACTTCGGTGCCGTTCGTCTGGTCC |
| | | TGAATGTTCTGCCGCACATGATGCAGCGCAAAGATGGCCAAATCATTAACATTAGCAGCATTGGCGTTTT |
| | | GGCGAACGCGACGCGTTTCAGCGCGTATGTGGCGAGCAAGGCTGCACTGGATGCCTTCTCCCGTTGTCTG |
| | | AGCGCCGAGGTCCATTCGCACAAGATTGCGATTACCTCTATCTATATGCCGCTGGTTCGTACCCCGATGA |
| | | TTGCGCCGACGAAGATCTACAAGTATGTCCCAACGTTGTCCCCGGAAGAGGCGGCTGACCTGATTGCTTA |
| | | TGCGATCGTTAAACGTCCGAAAAAGATCGCCACCAATCTGGGTCGCCTGGCAAGCATCACCTACGCGATT |
| | | GCCCCGGACATCAACAACATCCTGATGAGCATCGGCTTTAACCTGTTTCCGTCTAGCACGGCGAGCGTGG |
| | | GTGAGCAAGAAAAGCTGAACCTGATTCAACGTGCCTACGCACGTCTGTTTCCTGGTGAACACTGGTAAGA |
| | | ATTCGAGCTCCGTCGACAAGCTTGCGGCCGCACTCGAGCACCACCACCACCACCACTGAGATCCGGCTGC |
| | | TAACAAAGCCCGAAAGGAAGCTGAGTTGGCTGCTGCCACCGCTGAGCAATAACTAGCATAACCCCTTGGG |
| | | GCCTCTAAACGGGTCTTGAGGGGTTTTTTGCTGAAAGGAGGAACTATATCCGGAT |
| 3 | carboxylic | GAGCTCGAGGAGGTTTTTACAATGACCAGCGATGTTCACGACGCCACAGACGGCGTCACCGAAACCGCAC |
| | acid | TCGACGACGAGCAGTCGACCCGCCGCATCGCCGAGCTGTACGCCACCGATCCCGAGTTCGCCGCCGCCGC |
| | reductase | ACCGTTGCCCGCCGTGGTCGACGCGGCGCACAAACCCGGGCTGCGGCTGGCAGAGATCCTGCAGACCCTG |
| | amplified | TTCACCGGCTACGGTGACCGCCCGGCGCTGGGATACCGCGCCCGTGAACTGGCCACCGACGAGGGCGGGC |
| | from | GCACCGTGACGCGTCTGCTGCCGCGGTTCGACACCCTCACCTACGCCCAGGTGTGGTCGCGCGTGCAAGC |
| | Mycobacterium | GGTCGCCGCGGCCCTGCGCCACAACTTCGCGCAGCCGATCTACCCCGGCGACGCCGTCGCGACGATCGGT |
| | smegmatis. | TTCGCGAGTCCCGATTACCTGACGCTGGATCTCGTATGCGCCTACCTGGGCCTCGTGAGTGTTCCGCTGC |
| | | AGCACAACGCACCGGTCAGCCGGCTCGCCCCGATCCTGGCCGAGGTCGAACCGCGGATCCTCACCGTGAG |
| | | CGCCGAATACCTCGACCTCGCAGTCGAATCCGTGCGGGACGTCAACTCGGTGTCGCAGCTCGTGGTGTTC |
| | | GACCATCACCCCGAGGTCGACGACCACCGCGACGCACTGGCCCGCGCGCGTGAACAACTCGCCGGCAAGG |
| | | GCATCGCCGTCACCACCCTGGACGCGATCGCCGACGAGGGCGCCGGGCTGCCGGCCGAACCGATCTACAC |
| | | CGCCGACCATGATCAGCGCCTCGCGATGATCCTGTACACCTCGGGTTCCACCGGCGCACCCAAGGGTGCG |
| | | ATGTACACCGAGGCGATGGTGGCGCGGCTGTGGACCATGTCGTTCATCACGGGTGACCCCACGCCGGTCA |
| | | TCAACGTCAACTTCATGCCGCTCAACCACCTGGGCGGGCGCATCCCCATTTCCACCGCCGTGCAGAACGG |
| | | TGGAACCAGTTACTTCGTACCGGAATCCGACATGTCCACGCTGTTCGAGGATCTCGCGCTGGTGCGCCCG |
| | | ACCGAACTCGGCCTGGTTCCGCGCGTCGCCGACATGCTCTACCAGCACCACCTCGCCACCGTCGACCGCC |
| | | TGGTCACGCAGGGCGCCGACGAACTGACCGCCGAGAAGCAGGCCGGTGCCGAACTGCGTGAGCAGGTGCT |
| | | CGGCGGACGCGTGATCACCGGATTCGTCAGCACCGCACCGCTGGCCGCGGAGATGAGGGCGTTCCTCGAC |
| | | ATCACCCTGGGCGCACACATCGTCGACGGCTACGGGCTCACCGAGACCGGCGCCGTGACACGCGACGGTG |
| | | TGATCGTGCGGCCACCGGTGATCGACTACAAGCTGATCGACGTTCCCGAACTCGGCTACTTCAGCACCGA |
| | | CAAGCCCTACCCGCGTGGCGAACTGCTGGTCAGGTCGCAAACGCTGACTCCCGGGTACTACAAGCGCCCC |
| | | GAGGTCACCGCGAGCGTCTTCGACCGGGACGGCTACTACCACACCGGCGACGTCATGGCCGAGACCGCAC |
| | | CCGACCACCTGGTGTACGTGGACCGTCGCAACAACGTCCTCAAACTCGCGCAGGGCGAGTTCGTGGCGGT |
| | | CGCCAACCTGGAGGCGGTGTTCTCCGGCGCGGCGCTGGTGCGCCAGATCTTCGTGTACGGCAACAGCGAG |
| | | CGCAGTTTCCTTCTGGCCGTGGTGGTCCCGACGCCGGAGGCGCTCGAGCAGTACGATCCGGCCGCGCTCA |
| | | AGGCCGCGCTGGCCGACTCGCTGCAGCGCACCGCACGCGACGCCGAACTGCAATCCTACGAGGTGCCGGC |
| | | CGATTTCATCGTCGAGACCGAGCCGTTCAGCGCCGCCAACGGGCTGCTGTCGGGTGTCGGAAAACTGCTG |
| | | CGGCCCAACCTCAAAGACCGCTACGGGCAGCGCCTGGAGCAGATGTACGCCGATATCGCGGCCACGCAGG |
| | | CCAACCAGTTGCGCGAACTGCGGCGCGCGGCCGCCACACAACCGGTGATCGACACCCTCACCCAGGCCGC |
| | | TGCCACGATCCTCGGCACCGGGAGCGAGGTGGCATCCGACGCCCACTTCACCGACCTGGGCGGGGATTCC |
| | | CTGTCGGCGCTGACACTTTCGAACCTGCTGAGCGATTTCTTCGGTTTCGAAGTTCCCGTCGGCACCATCG |
| | | TGAACCCGGCCACCAACCTCGCCCAACTCGCCCAGCACATCGAGGCGCAGCGCACCGCGGGTGACCGCAG |
| | | GCCGAGTTTCACCACCGTGCACGGCGCGGACGCCACCGAGATCCGGGCGAGTGAGCTGACCCTGGACAAG |
| | | TTCATCGACGCCGAAACGCTCCGGGCCGCACCGGGTCTGCCCAAGGTCACCACCGAGCCACGGACGGTGT |
| | | TGCTCTCGGGCGCCAACGGCTGGCTGGGCCGGTTCCTCACGTTGCAGTGGCTGGAACGCCTGGCACCTGT |
| | | CGGCGGCACCCTCATCACGATCGTGCGGGGCCGCGACGACGCCGCGGCCCGCGCACGGCTGACCCAGGCC |
| | | TACGACACCGATCCCGAGTTGTCCCGCCGCTTCGCCGAGCTGGCCGACCGCCACCTGCGGGTGGTCGCCG |
| | | GTGACATCGGCGACCCGAATCTGGGCCTCACACCCGAGATCTGGCACCGGCTCGCCGCCGAGGTCGACCT |
| | | GGTGGTGCATCCGGCAGCGCTGGTCAACCACGTGCTCCCCTACCGGCAGCTGTTCGGCCCCAACGTCGTG |
| | | GGCACGGCCGAGGTGATCAAGCTGGCCCTCACCGAACGGATCAAGCCCGTCACGTACCTGTCCACCGTGT |
| | | CGGTGGCCATGGGGATCCCCGACTTCGAGGAGGACGGCGACATCCGGACCGTGAGCCCGGTGCGCCCGCT |
| | | CGACGGCGGATACGCCAACGGCTACGGCAACAGCAAGTGGGCCGGCGAGGTGCTGCTGCGGGAGGCCCAC |
| | | GATCTGTGCGGGCTGCCCGTGGCGACGTTCCGCTCGGACATGATCCTGGCGCATCCGCGCTACCGCGGTC |
| | | AGGTCAACGTGCCAGACATGTTCACGCGACTCCTGTTGAGCCTCTTGATCACCGGCGTCGCGCCGCGGTC |
| | | GTTCTACATCGGAGACGGTGAGCGCCCGCGGGCGCACTACCCCGGCCTGACGGTCGATTTCGTGGCCGAG |
| | | GCGGTCACGACGCTCGGCGCGCAGCAGCGCGAGGGATACGTGTCCTACGACGTGATGAACCCGCACGACG |
| | | ACGGGATCTCCCTGGATGTGTTCGTGGACTGGCTGATCCGGGCGGGCCATCCGATCGACCGGGTCGACGA |
| | | CTACGACGACTGGGTGCGTCGGTTCGAGACCGCGTTGACCGCGCTTCCCGAGAAGCGCCGCGCACAGACC |
| | | GTACTGCCGCTGCTGCACGCGTTCCGCGCTCCGCAGGCACCGTTGCGCGGCGCACCCGAACCCACGGAGG |
| | | TGTTCCACGCCGCGGTGCGCACCGCGAAGGTGGGCCCGGGAGACATCCCGCACCTCGACGAGGCGCTGAT |
| | | CGACAAGTACATACGCGATCTGCGTGAGTTCGGTCTGATCTGAGGTACC |
| 4 | codon- | CATATGCAAGAACTGGCCCTGAGAAGCGAGCTGGACTTCAATAGCGAAACCTATAAAGATGCGTATAGCC |
| | optimized | GTATTAACGCCATTGTGATCGAAGGCGAGCAAGAAGCATACCAAAACTACCTGGACATGGCGCAACTGCT |
| | Cyanothece | GCCGGAGGACGAGGCTGAGCTGATTCGTTTGAGCAAGATGGAGAACCGTCACAAAAAGGGTTTTCAAGCG |
| | adm. | TGCGGCAAGAACCTCAATGTGACTCCGGATATGGATTATGCACAGCAGTTCTTTGCGGAGCTGCACGGCA |
| | | ATTTTCAGAAGGCTAAAGCCGAGGGTAAGATTGTTACCTGCCTGCTCATCCAAAGCCTGATCATCGAGGC |
| | | GTTTGCGATTGCAGCCTACAACATTTACATTCCAGTGGCTGATCCGTTTGCACGTAAAATCACCGAGGGT |
| | | GTCGTCAAGGATGAGTATACCCACCTGAATTTCGGCGAAGTTTGGTTGAAGGAACATTTTGAAGCAAGCA |
| | | AGGCGGAGTTGGAGGACGCCAACAAAGAGAACTTACCGCTGGTCTGGCAGATGTTGAACCAGGTCGAAAA |
| | | GGATGCCGAAGTGCTGGGTATGGAGAAAGAGGCTCTGGTGGAGGACTTTATGATTAGCTATGGTGAGGCA |
| | | CTGAGCAACATCGGCTTTTCTACGAGAGAAATCATGAAGATGAGCGCGTACGGTCTGCGTGCAGCATAAG |
| | | AGCTC |
| 5 | codon- | GAGCTCGAGGAGGTTTTTACAATGACCAGCGATGTTCACGACGCCACAGACGGCGTCACCGAAACCGCAC |
| | optimized E. | TCGACGACGAGCAGTCGACCCGCCGCATCGCCGAGCTGTACGCCACCGATCCCGAGTTCGCCGCCGCCGC |
| | coli tesA and | ACCGTTGCCCGCCGTGGTCGACGCGGCGCACAAACCCGGGCTGCGGCTGGCAGAGATCCTGCAGACCCTG |
| | E. coli entD | TTCACCGGCTACGGTGACCGCCCGGCGCTGGGATACCGCGCCCGTGAACTGGCCACCGACGAGGGCGGGC |
| | genes. | GCACCGTGACGCGTCTGCTGCCGCGGTTCGACACCCTCACCTACGCCCAGGTGTGGTCGCGCGTGCAAGC |
| | | GGTCGCCGCGGCCCTGCGCCACAACTTCGCGCAGCCGATCTACCCCGGCGACGCCGTCGCGACGATCGGT |
| | | TTCGCGAGTCCCGATTACCTGACGCTGGATCTCGTATGCGCCTACCTGGGCCTCGTGAGTGTTCCGCTGC |
| | | AGCACAACGCACCGGTCAGCCGGCTCGCCCCGATCCTGGCCGAGGTCGAACCGCGGATCCTCACCGTGAG |
| | | CGCCGAATACCTCGACCTCGCAGTCGAATCCGTGCGGGACGTCAACTCGGTGTCGCAGCTCGTGGTGTTC |
| | | GACCATCACCCCGAGGTCGACGACCACCGCGACGCACTGGCCCGCGCGCGTGAACAACTCGCCGGCAAGG |
| | | GCATCGCCGTCACCACCCTGGACGCGATCGCCGACGAGGGCGCCGGGCTGCCGGCCGAACCGATCTACAC |
| | | CGCCGACCATGATCAGCGCCTCGCGATGATCCTGTACACCTCGGGTTCCACCGGCGCACCCAAGGGTGCG |
| | | ATGTACACCGAGGCGATGGTGGCGCGGCTGTGGACCATGTCGTTCATCACGGGTGACCCCACGCCGGTCA |
| | | TCAACGTCAACTTCATGCCGCTCAACCACCTGGGCGGGCGCATCCCCATTTCCACCGCCGTGCAGAACGG |
| | | TGGAACCAGTTACTTCGTACCGGAATCCGACATGTCCACGCTGTTCGAGGATCTCGCGCTGGTGCGCCCG |
| | | ACCGAACTCGGCCTGGTTCCGCGCGTCGCCGACATGCTCTACCAGCACCACCTCGCCACCGTCGACCGCC |
| | | TGGTCACGCAGGGCGCCGACGAACTGACCGCCGAGAAGCAGGCCGGTGCCGAACTGCGTGAGCAGGTGCT |
| | | CGGCGGACGCGTGATCACCGGATTCGTCAGCACCGCACCGCTGGCCGCGGAGATGAGGGCGTTCCTCGAC |
| | | ATCACCCTGGGCGCACACATCGTCGACGGCTACGGGCTCACCGAGACCGGCGCCGTGACACGCGACGGTG |
| | | TGATCGTGCGGCCACCGGTGATCGACTACAAGCTGATCGACGTTCCCGAACTCGGCTACTTCAGCACCGA |
| | | CAAGCCCTACCCGCGTGGCGAACTGCTGGTCAGGTCGCAAACGCTGACTCCCGGGTACTACAAGCGCCCC |
| | | GAGGTCACCGCGAGCGTCTTCGACCGGGACGGCTACTACCACACCGGCGACGTCATGGCCGAGACCGCAC |
| | | CCGACCACCTGGTGTACGTGGACCGTCGCAACAACGTCCTCAAACTCGCGCAGGGCGAGTTCGTGGCGGT |
| | | CGCCAACCTGGAGGCGGTGTTCTCCGGCGCGGCGCTGGTGCGCCAGATCTTCGTGTACGGCAACAGCGAG |
| | | CGCAGTTTCCTTCTGGCCGTGGTGGTCCCGACGCCGGAGGCGCTCGAGCAGTACGATCCGGCCGCGCTCA |
| | | AGGCCGCGCTGGCCGACTCGCTGCAGCGCACCGCACGCGACGCCGAACTGCAATCCTACGAGGTGCCGGC |
| | | CGATTTCATCGTCGAGACCGAGCCGTTCAGCGCCGCCAACGGGCTGCTGTCGGGTGTCGGAAAACTGCTG |
| | | CGGCCCAACCTCAAAGACCGCTACGGGCAGCGCCTGGAGCAGATGTACGCCGATATCGCGGCCACGCAGG |
| | | CCAACCAGTTGCGCGAACTGCGGCGCGCGGCCGCCACACAACCGGTGATCGACACCCTCACCCAGGCCGC |
| | | TGCCACGATCCTCGGCACCGGGAGCGAGGTGGCATCCGACGCCCACTTCACCGACCTGGGCGGGGATTCC |
| | | CTGTCGGCGCTGACACTTTCGAACCTGCTGAGCGATTTCTTCGGTTTCGAAGTTCCCGTCGGCACCATCG |
| | | TGAACCCGGCCACCAACCTCGCCCAACTCGCCCAGCACATCGAGGCGCAGCGCACCGCGGGTGACCGCAG |
| | | GCCGAGTTTCACCACCGTGCACGGCGCGGACGCCACCGAGATCCGGGCGAGTGAGCTGACCCTGGACAAG |
| | | TTCATCGACGCCGAAACGCTCCGGGCCGCACCGGGTCTGCCCAAGGTCACCACCGAGCCACGGACGGTGT |
| | | TGCTCTCGGGCGCCAACGGCTGGCTGGGCCGGTTCCTCACGTTGCAGTGGCTGGAACGCCTGGCACCTGT |
| | | CGGCGGCACCCTCATCACGATCGTGCGGGGCCGCGACGACGCCGCGGCCCGCGCACGGCTGACCCAGGCC |
| | | TACGACACCGATCCCGAGTTGTCCCGCCGCTTCGCCGAGCTGGCCGACCGCCACCTGCGGGTGGTCGCCG |
| | | GTGACATCGGCGACCCGAATCTGGGCCTCACACCCGAGATCTGGCACCGGCTCGCCGCCGAGGTCGACCT |
| | | GGTGGTGCATCCGGCAGCGCTGGTCAACCACGTGCTCCCCTACCGGCAGCTGTTCGGCCCCAACGTCGTG |
| | | GGCACGGCCGAGGTGATCAAGCTGGCCCTCACCGAACGGATCAAGCCCGTCACGTACCTGTCCACCGTGT |
| | | CGGTGGCCATGGGGATCCCCGACTTCGAGGAGGACGGCGACATCCGGACCGTGAGCCCGGTGCGCCCGCT |
| | | CGACGGCGGATACGCCAACGGCTACGGCAACAGCAAGTGGGCCGGCGAGGTGCTGCTGCGGGAGGCCCAC |
| | | GATCTGTGCGGGCTGCCCGTGGCGACGTTCCGCTCGGACATGATCCTGGCGCATCCGCGCTACCGCGGTC |
| | | AGGTCAACGTGCCAGACATGTTCACGCGACTCCTGTTGAGCCTCTTGATCACCGGCGTCGCGCCGCGGTC |
| | | GTTCTACATCGGAGACGGTGAGCGCCCGCGGGCGCACTACCCCGGCCTGACGGTCGATTTCGTGGCCGAG |
| | | GCGGTCACGACGCTCGGCGCGCAGCAGCGCGAGGGATACGTGTCCTACGACGTGATGAACCCGCACGACG |
| | | ACGGGATCTCCCTGGATGTGTTCGTGGACTGGCTGATCCGGGCGGGCCATCCGATCGACCGGGTCGACGA |
| | | CTACGACGACTGGGTGCGTCGGTTCGAGACCGCGTTGACCGCGCTTCCCGAGAAGCGCCGCGCACAGACC |
| | | GTACTGCCGCTGCTGCACGCGTTCCGCGCTCCGCAGGCACCGTTGCGCGGCGCACCCGAACCCACGGAGG |
| | | TGTTCCACGCCGCGGTGCGCACCGCGAAGGTGGGCCCGGGAGACATCCCGCACCTCGACGAGGCGCTGAT |
| | | CGACAAGTACATACGCGATCTGCGTGAGTTCGGTCTGATCTGAGGTACCAGGAGGTTTTTACAATGGCTG |
| | | ATACTTTGTTGATTTTGGGTGATTCTCTCTCTGCAGGCTACCGTATGTCCGCGAGCGCGGCATGGCCGGC |
| | | TCTGCTGAACGATAAGTGGCAGAGCAAGACCAGCGTGGTCAATGCGAGCATCAGCGGCGATACCAGCCAG |
| | | CAGGGTCTGGCACGTCTGCCAGCGCTGCTGAAGCAACACCAGCCGCGTTGGGTGCTGGTTGAACTGGGCG |
| | | GCAATGACGGTCTGCGTGGTTTTCAGCCGCAGCAGACCGAACAAACGTTGCGTCAGATTCTGCAGGACGT |
| | | CAAGGCGGCTAACGCGGAACCGCTGCTGATGCAAATTCGCCTGCCGGCGAATTATGGTCGTCGTTACAAC |
| | | GAGGCTTTCAGCGCCATTTATCCTAAACTGGCTAAAGAGTTTGACGTGCCGCTGCTGCCGTTCTTCATGG |
| | | AAGAGGTCTACCTGAAACCGCAATGGATGCAAGACGACGGTATTCATCCGAATCGTGATGCACAACCTTT |
| | | CATCGCGGATTGGATGGCGAAGCAATTGCAACCGCTGGTGAACCATGACTCGTAAAAGCTTGTTGCTGCA |
| | | TGCAGGAGGTTTTTACAATGAAAACGACCCACACCAGCTTACCATTTGCCGGCCACACGTTACATTTCGT |
| | | CGAATTTGATCCGGCGAACTTTTGTGAACAAGACCTGTTGTGGCTGCCGCATTATGCCCAGCTGCAGCAC |
| | | GCAGGCCGTAAGCGTAAAACTGAACATCTGGCCGGTCGCATTGCGGCAGTGTATGCCCTGCGCGAGTACG |
| | | GCTACAAATGCGTGCCGGCCATTGGTGAACTGCGTCAACCGGTTTGGCCGGCAGAAGTTTACGGTTCCAT |
| | | CTCCCACTGCGGTACTACCGCGTTGGCGGTTGTGTCTCGCCAGCCGATCGGTATTGATATTGAAGAGATA |
| | | TTCTCTGTCCAGACGGCACGCGAGCTGACGGACAACATCATTACCCCGGCAGAGCACGAGCGTCTGGCGG |
| | | ACTGTGGTCTGGCGTTCAGCCTGGCGCTGACCCTGGCATTCAGCGCAAAAGAGAGCGCGTTCAAGGCTTC |
| | | CGAGATCCAAACCGATGCGGGCTTCCTGGATTATCAAATCATCAGCTGGAACAAGCAACAGGTTATCATT |
| | | CACCGTGAGAATGAGATGTTTGCCGTCCATTGGCAGATTAAAGAGAAAATCGTTATCACCCTGTGCCAGC |
| | | ACGACTGAGAATTC |
| 6 | plasmid | AAAAGCAGAGCATTACGCTGACTTGACGGGACGGCGCAAGCTCATGACCAAAATCCCTTAACGTGAGTTA |
| | pAQ3::Pnir07_ | CGCGCGCGTCGTTCCACTGAGCGTCAGACCCCGTAGAAAAGATCAAAGGATCTTCTTGAGATCCTTTTTT |
| | adm_carB_tesA_ | TCTGCGCGTAATCTGCTGCTTGCAAACAAAAAAACCACCGCTACCAGCGGTGGTTTGTTTGCCGGATCAA |
| | entD_SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | CAAGAACTGGCCCTGAGAAGCGAGCTGGACTTCAATAGCGAAACCTATAAAGATGCGTATAGCCGTATTA |
| | | ACGCCATTGTGATCGAAGGCGAGCAAGAAGCATACCAAAACTACCTGGACATGGCGCAACTGCTGCCGGA |
| | | GGACGAGGCTGAGCTGATTCGTTTGAGCAAGATGGAGAACCGTCACAAAAAGGGTTTTCAAGCGTGCGGC |
| | | AAGAACCTCAATGTGACTCCGGATATGGATTATGCACAGCAGTTCTTTGCGGAGCTGCACGGCAATTTTC |
| | | AGAAGGCTAAAGCCGAGGGTAAGATTGTTACCTGCCTGCTCATCCAAAGCCTGATCATCGAGGCGTTTGC |
| | | GATTGCAGCCTACAACATTTACATTCCAGTGGCTGATCCGTTTGCACGTAAAATCACCGAGGGTGTCGTC |
| | | AAGGATGAGTATACCCACCTGAATTTCGGCGAAGTTTGGTTGAAGGAACATTTTGAAGCAAGCAAGGCGG |
| | | AGTTGGAGGACGCCAACAAAGAGAACTTACCGCTGGTCTGGCAGATGTTGAACCAGGTCGAAAAGGATGC |
| | | CGAAGTGCTGGGTATGGAGAAAGAGGCTCTGGTGGAGGACTTTATGATTAGCTATGGTGAGGCACTGAGC |
| | | AACATCGGCTTTTCTACGAGAGAAATCATGAAGATGAGCGCGTACGGTCTGCGTGCAGCATAAGAGCTCG |
| | | AGGAGGTTTTTACAATGACCAGCGATGTTCACGACGCCACAGACGGCGTCACCGAAACCGCACTCGACGA |
| | | CGAGCAGTCGACCCGCCGCATCGCCGAGCTGTACGCCACCGATCCCGAGTTCGCCGCCGCCGCACCGTTG |
| | | CCCGCCGTGGTCGACGCGGCGCACAAACCCGGGCTGCGGCTGGCAGAGATCCTGCAGACCCTGTTCACCG |
| | | GCTACGGTGACCGCCCGGCGCTGGGATACCGCGCCCGTGAACTGGCCACCGACGAGGGCGGGCGCACCGT |
| | | GACGCGTCTGCTGCCGCGGTTCGACACCCTCACCTACGCCCAGGTGTGGTCGCGCGTGCAAGCGGTCGCC |
| | | GCGGCCCTGCGCCACAACTTCGCGCAGCCGATCTACCCCGGCGACGCCGTCGCGACGATCGGTTTCGCGA |
| | | GTCCCGATTACCTGACGCTGGATCTCGTATGCGCCTACCTGGGCCTCGTGAGTGTTCCGCTGCAGCACAA |
| | | CGCACCGGTCAGCCGGCTCGCCCCGATCCTGGCCGAGGTCGAACCGCGGATCCTCACCGTGAGCGCCGAA |
| | | TACCTCGACCTCGCAGTCGAATCCGTGCGGGACGTCAACTCGGTGTCGCAGCTCGTGGTGTTCGACCATC |
| | | ACCCCGAGGTCGACGACCACCGCGACGCACTGGCCCGCGCGCGTGAACAACTCGCCGGCAAGGGCATCGC |
| | | CGTCACCACCCTGGACGCGATCGCCGACGAGGGCGCCGGGCTGCCGGCCGAACCGATCTACACCGCCGAC |
| | | CATGATCAGCGCCTCGCGATGATCCTGTACACCTCGGGTTCCACCGGCGCACCCAAGGGTGCGATGTACA |
| | | CCGAGGCGATGGTGGCGCGGCTGTGGACCATGTCGTTCATCACGGGTGACCCCACGCCGGTCATCAACGT |
| | | CAACTTCATGCCGCTCAACCACCTGGGCGGGCGCATCCCCATTTCCACCGCCGTGCAGAACGGTGGAACC |
| | | AGTTACTTCGTACCGGAATCCGACATGTCCACGCTGTTCGAGGATCTCGCGCTGGTGCGCCCGACCGAAC |
| | | TCGGCCTGGTTCCGCGCGTCGCCGACATGCTCTACCAGCACCACCTCGCCACCGTCGACCGCCTGGTCAC |
| | | GCAGGGCGCCGACGAACTGACCGCCGAGAAGCAGGCCGGTGCCGAACTGCGTGAGCAGGTGCTCGGCGGA |
| | | CGCGTGATCACCGGATTCGTCAGCACCGCACCGCTGGCCGCGGAGATGAGGGCGTTCCTCGACATCACCC |
| | | TGGGCGCACACATCGTCGACGGCTACGGGCTCACCGAGACCGGCGCCGTGACACGCGACGGTGTGATCGT |
| | | GCGGCCACCGGTGATCGACTACAAGCTGATCGACGTTCCCGAACTCGGCTACTTCAGCACCGACAAGCCC |
| | | TACCCGCGTGGCGAACTGCTGGTCAGGTCGCAAACGCTGACTCCCGGGTACTACAAGCGCCCCGAGGTCA |
| | | CCGCGAGCGTCTTCGACCGGGACGGCTACTACCACACCGGCGACGTCATGGCCGAGACCGCACCCGACCA |
| | | CCTGGTGTACGTGGACCGTCGCAACAACGTCCTCAAACTCGCGCAGGGCGAGTTCGTGGCGGTCGCCAAC |
| | | CTGGAGGCGGTGTTCTCCGGCGCGGCGCTGGTGCGCCAGATCTTCGTGTACGGCAACAGCGAGCGCAGTT |
| | | TCCTTCTGGCCGTGGTGGTCCCGACGCCGGAGGCGCTCGAGCAGTACGATCCGGCCGCGCTCAAGGCCGC |
| | | GCTGGCCGACTCGCTGCAGCGCACCGCACGCGACGCCGAACTGCAATCCTACGAGGTGCCGGCCGATTTC |
| | | ATCGTCGAGACCGAGCCGTTCAGCGCCGCCAACGGGCTGCTGTCGGGTGTCGGAAAACTGCTGCGGCCCA |
| | | ACCTCAAAGACCGCTACGGGCAGCGCCTGGAGCAGATGTACGCCGATATCGCGGCCACGCAGGCCAACCA |
| | | GTTGCGCGAACTGCGGCGCGCGGCCGCCACACAACCGGTGATCGACACCCTCACCCAGGCCGCTGCCACG |
| | | ATCCTCGGCACCGGGAGCGAGGTGGCATCCGACGCCCACTTCACCGACCTGGGCGGGGATTCCCTGTCGG |
| | | CGCTGACACTTTCGAACCTGCTGAGCGATTTCTTCGGTTTCGAAGTTCCCGTCGGCACCATCGTGAACCC |
| | | GGCCACCAACCTCGCCCAACTCGCCCAGCACATCGAGGCGCAGCGCACCGCGGGTGACCGCAGGCCGAGT |
| | | TTCACCACCGTGCACGGCGCGGACGCCACCGAGATCCGGGCGAGTGAGCTGACCCTGGACAAGTTCATCG |
| | | ACGCCGAAACGCTCCGGGCCGCACCGGGTCTGCCCAAGGTCACCACCGAGCCACGGACGGTGTTGCTCTC |
| | | GGGCGCCAACGGCTGGCTGGGCCGGTTCCTCACGTTGCAGTGGCTGGAACGCCTGGCACCTGTCGGCGGC |
| | | ACCCTCATCACGATCGTGCGGGGCCGCGACGACGCCGCGGCCCGCGCACGGCTGACCCAGGCCTACGACA |
| | | CCGATCCCGAGTTGTCCCGCCGCTTCGCCGAGCTGGCCGACCGCCACCTGCGGGTGGTCGCCGGTGACAT |
| | | CGGCGACCCGAATCTGGGCCTCACACCCGAGATCTGGCACCGGCTCGCCGCCGAGGTCGACCTGGTGGTG |
| | | CATCCGGCAGCGCTGGTCAACCACGTGCTCCCCTACCGGCAGCTGTTCGGCCCCAACGTCGTGGGCACGG |
| | | CCGAGGTGATCAAGCTGGCCCTCACCGAACGGATCAAGCCCGTCACGTACCTGTCCACCGTGTCGGTGGC |
| | | CATGGGGATCCCCGACTTCGAGGAGGACGGCGACATCCGGACCGTGAGCCCGGTGCGCCCGCTCGACGGC |
| | | GGATACGCCAACGGCTACGGCAACAGCAAGTGGGCCGGCGAGGTGCTGCTGCGGGAGGCCCACGATCTGT |
| | | GCGGGCTGCCCGTGGCGACGTTCCGCTCGGACATGATCCTGGCGCATCCGCGCTACCGCGGTCAGGTCAA |
| | | CGTGCCAGACATGTTCACGCGACTCCTGTTGAGCCTCTTGATCACCGGCGTCGCGCCGCGGTCGTTCTAC |
| | | ATCGGAGACGGTGAGCGCCCGCGGGCGCACTACCCCGGCCTGACGGTCGATTTCGTGGCCGAGGCGGTCA |
| | | CGACGCTCGGCGCGCAGCAGCGCGAGGGATACGTGTCCTACGACGTGATGAACCCGCACGACGACGGGAT |
| | | CTCCCTGGATGTGTTCGTGGACTGGCTGATCCGGGCGGGCCATCCGATCGACCGGGTCGACGACTACGAC |
| | | GACTGGGTGCGTCGGTTCGAGACCGCGTTGACCGCGCTTCCCGAGAAGCGCCGCGCACAGACCGTACTGC |
| | | CGCTGCTGCACGCGTTCCGCGCTCCGCAGGCACCGTTGCGCGGCGCACCCGAACCCACGGAGGTGTTCCA |
| | | CGCCGCGGTGCGCACCGCGAAGGTGGGCCCGGGAGACATCCCGCACCTCGACGAGGCGCTGATCGACAAG |
| | | TACATACGCGATCTGCGTGAGTTCGGTCTGATCTGAGGTACCAGGAGGTTTTTACAATGGCTGATACTTT |
| | | GTTGATTTTGGGTGATTCTCTCTCTGCAGGCTACCGTATGTCCGCGAGCGCGGCATGGCCGGCTCTGCTG |
| | | AACGATAAGTGGCAGAGCAAGACCAGCGTGGTCAATGCGAGCATCAGCGGCGATACCAGCCAGCAGGGTC |
| | | TGGCACGTCTGCCAGCGCTGCTGAAGCAACACCAGCCGCGTTGGGTGCTGGTTGAACTGGGCGGCAATGA |
| | | CGGTCTGCGTGGTTTTCAGCCGCAGCAGACCGAACAAACGTTGCGTCAGATTCTGCAGGACGTCAAGGCG |
| | | GCTAACGCGGAACCGCTGCTGATGCAAATTCGCCTGCCGGCGAATTATGGTCGTCGTTACAACGAGGCTT |
| | | TCAGCGCCATTTATCCTAAACTGGCTAAAGAGTTTGACGTGCCGCTGCTGCCGTTCTTCATGGAAGAGGT |
| | | CTACCTGAAACCGCAATGGATGCAAGACGACGGTATTCATCCGAATCGTGATGCACAACCTTTCATCGCG |
| | | GATTGGATGGCGAAGCAATTGCAACCGCTGGTGAACCATGACTCGTAAAAGCTTGTTGCTGCATGCAGGA |
| | | GGTTTTTACAATGAAAACGACCCACACCAGCTTACCATTTGCCGGCCACACGTTACATTTCGTCGAATTT |
| | | GATCCGGCGAACTTTTGTGAACAAGACCTGTTGTGGCTGCCGCATTATGCCCAGCTGCAGCACGCAGGCC |
| | | GTAAGCGTAAAACTGAACATCTGGCCGGTCGCATTGCGGCAGTGTATGCCCTGCGCGAGTACGGCTACAA |
| | | ATGCGTGCCGGCCATTGGTGAACTGCGTCAACCGGTTTGGCCGGCAGAAGTTTACGGTTCCATCTCCCAC |
| | | TGCGGTACTACCGCGTTGGCGGTTGTGTCTCGCCAGCCGATCGGTATTGATATTGAAGAGATATTCTCTG |
| | | TCCAGACGGCACGCGAGCTGACGGACAACATCATTACCCCGGCAGAGCACGAGCGTCTGGCGGACTGTGG |
| | | TCTGGCGTTCAGCCTGGCGCTGACCCTGGCATTCAGCGCAAAAGAGAGCGCGTTCAAGGCTTCCGAGATC |
| | | CAAACCGATGCGGGCTTCCTGGATTATCAAATCATCAGCTGGAACAAGCAACAGGTTATCATTCACCGTG |
| | | AGAATGAGATGTTTGCCGTCCATTGGCAGATTAAAGAGAAAATCGTTATCACCCTGTGCCAGCACGACTG |
| | | AGAATTCGGTTTTCCGTCCTGTCTTGATTTTCAAGCAAACAATGCCTCCGATTTCTAATCGGAGGCATTT |
| | | GTTTTTGTTTATTGCAAAAACAAAAAATATTGTTACAAATTTTTACAGGCTATTAAGCCTACCGTCATAA |
| | | ATAATTTGCCATTTACTAGTTTTTAATTAACCAGAACCTTGACCGAACGCAGCGGTGGTAACGGCGCAGT |
| | | GGCGGTTTTCATGGCTTGTTATGACTGTTTTTTTGGGGTACAGTCTATGCCTCGGGCATCCAAGCAGCAA |
| | | GCGCGTTACGCCGTGGGTCGATGTTTGATGTTATGGAGCAGCAACGATGTTACGCAGCAGGGCAGTCGCC |
| | | CTAAAACAAAGTTAAACATCATGAGGGAAGCGGTGATCGCCGAAGTATCGACTCAACTATCAGAGGTAGT |
| | | TGGCGTCATCGAGCGCCATCTCGAACCGACGTTGCTGGCCGTACATTTGTACGGCTCCGCAGTGGATGGC |
| | | GGCCTGAAGCCACACAGTGATATTGATTTGCTGGTTACGGTGACCGTAAGGCTTGATGAAACAACGCGGC |
| | | GAGCTTTGATCAACGACCTTTTGGAAACTTCGGCTTCCCCTGGAGAGAGCGAGATTCTCCGCGCTGTAGA |
| | | AGTCACCATTGTTGTGCACGACGACATCATTCCGTGGCGTTATCCAGCTAAGCGCGAACTGCAATTTGGA |
| | | GAATGGCAGCGCAATGACATTCTTGCAGGTATCTTCGAGCCAGCCACGATCGACATTGATCTGGCTATCT |
| | | TGCTGACAAAAGCAAGAGAACATAGCGTTGCCTTGGTAGGTCCAGCGGCGGAGGAACTCTTTGATCCGGT |
| | | TCCTGAACAGGATCTATTTGAGGCGCTAAATGAAACCTTAACGCTATGGAACTCGCCGCCCGACTGGGCT |
| | | GGCGATGAGCGAAATGTAGTGCTTACGTTGTCCCGCATTTGGTACAGCGCAGTAACCGGCAAAATCGCGC |
| | | CGAAGGATGTCGCTGCCGACTGGGCAATGGAGCGCCTGCCGGCCCAGTATCAGCCCGTCATACTTGAAGC |
| | | TAGACAGGCTTATCTTGGACAAGAAGAAGATCGCTTGGCCTCGCGCGCAGATCAGTTGGAAGAATTTGTC |
| | | CACTACGTGAAAGGCGAGATCACCAAGGTAGTCGGCAAATAATGTCTAACAATTCGTTCAAGCCGACGCC |
| | | GCTTCGCGGCGCGGCTTAACTCAAGCGTTAGATGCACTAAGCACATAATTGCTCACAGCCAAACTATCAG |
| | | GTCAAGTCTGCTTTTATTATTTTTAAGCGTGCATAATAAGCCCTACACAAATTGGGAGATATATCATGAG |
| | | GCGCGCCACGAGAAAGAGTTATGACAAATTAAAATTCTGACTCTTAGATTATTTCCAGAGAGGCTGATTT |
| | | TCCCAATCTTTGGGAAAGCCTAAGTTTTTAGATTCTATTTCTGGATACATCTCAAAAGTTCTTTTTAAAT |
| | | GCTGTGCAAAATTATGCTCTGGTTTAATTCTGTCTAAGAGATACTGAATACAACATAAGCCAGTGAAAAT |
| | | TTTACGGCTGTTTCTTTGATTAATATCCTCCAATACTTCTCTAGAGAGCCATTTTCCTTTTAACCTATCA |
| | | GGCAATTTAGGTGATTCTCCTAGCTGTATATTCCAGAGCCTTGAATGATGAGCGCAAATATTTCTAATAT |
| | | GCGACAAAGACCGTAACCAAGATATAAAAAACTTGTTAGGTAATTGGAAATGAGTATGTATTTTTTGTCG |
| | | TGTCTTAGATGGTAATAAATTTGTGTACATTCTAGATAACTGCCCAAAGGCGATTATCTCCAAAGCCATA |
| | | TATGACGGCGGTAGTAGAGGATTTGTGTACTTGTTTCGATAATGCCCGATAAATTCTTCTACTTTTTTAG |
| | | ATTGGCAATATTGAGTAATCGAATCGATTAATTCTTGATGCTTCCCAGTGTCATAAAATAAACTTTTATT |
| | | CAGATACCAATGAGGATCATAATCATGGGAGTAGTGATAAATCATTTGAGTTCTGACTGCTACTTCTATC |
| | | GACTCCGTAGCATTAAAAATAAGCATTCTCAAGGATTTATCAAACTTGTATAGATTTGGCCGGCCCGTCA |
| | | AAAGGGCGACACCCCATAATTAGCCCGGGCGAAAGGCCCAGTCTTTCGACTGAGCCTTTCGTTTTATTTG |
| | | ATGCCTGGCAGTTCCCTACTCTCGCATGGGGAGTCCCCACACTACCATCGGCGCTACGGCGTTTCACTTC |
| | | TGAGTTCGGCATGGGGTCAGGTGGGACCACCGCGCTACTGCCGCCAGGCAAACAAGGGGTGTTATGAGCC |
| | | ATATTCAGGTATAAATGGGCTCGCGATAATGTTCAGAATTGGTTAATTGGTTGTAACACTGACCCCTATT |
| | | TGTTTATTTTTCTAAATACATTCAAATATGTATCCGCTCATGAGACAATAACCCTGATAAATGCTTCAAT |
| | | AATATTGAAAAAGGAAGAATATGAGTATTCAACATTTCCGTGTCGCCCTTATTCCCTTTTTTGCGGCATT |
| | | TTGCCTTCCTGTTTTTGCTCACCCAGAAACGCTGGTGAAAGTAAAAGATGCTGAAGATCAGTTGGGTGCA |
| | | CGAGTGGGTTACATCGAACTGGATCTCAACAGCGGTAAGATCCTTGAGAGTTTTCGCCCCGAAGAACGTT |
| | | TTCCAATGATGAGCACTTTTAAAGTTCTGCTATGTGGCGCGGTATTATCCCGTATTGACGCCGGGCAAGA |
| | | GCAACTCGGTCGCCGCATACACTATTCTCAGAATGACTTGGTTGAGTACTCACCAGTCACAGAAAAGCAT |
| | | CTTACGGATGGCATGACAGTAAGAGAATTATGCAGTGCTGCCATAACCATGAGTGATAACACTGCGGCCA |
| | | ACTTACTTCTGACAACGATCGGAGGACCGAAGGAGCTAACCGCTTTTTTGCACAACATGGGGGATCATGT |
| | | AACTCGCCTTGATCGTTGGGAACCGGAGCTGAATGAAGCCATACCAAACGACGAGCGTGACACCACGATG |
| | | CCTGTAGCGATGGCAACAACGTTGCGCAAACTATTAACTGGCGAACTACTTACTCTAGCTTCCCGGCAAC |
| | | AATTAATAGACTGGATGGAGGCGGATAAAGTTGCAGGACCACTTCTGCGCTCGGCCCTTCCGGCTGGCTG |
| | | GTTTATTGCTGATAAATCCGGAGCCGGTGAGCGTGGTTCTCGCGGTATCATCGCAGCGCTGGGGCCAGAT |
| | | GGTAAGCCCTCCCGTATCGTAGTTATCTACACGACGGGGAGTCAGGCAACTATGGATGAACGAAATAGAC |
| | | AGATCGCTGAGATAGGTGCCTCACTGATTAAGCATTGGT |
| 7 | codon- | GGTACCAGGAGGTTTTTACATGGACCGTAAAAGCAAGCGTCCGGACATGCTGGTTGATTCCTTTGGTCTG |
| | optimized | GAAAGCACCGTGCAGGACGGTCTGGTTTTCCGTCAGTCTTTCTCCATTCGTAGCTATGAGATTGGTACTG |
| | Cuphea | ATCGTACCGCCTCTATCGAAACCCTGATGAATCACCTGCAAGAAACCTCTCTGAACCATTGTAAGTCTAC |
| | hookeriana | TGGCATCCTGCTGGACGGTTTCGGTCGTACCCTGGAGATGTGCAAACGCGACCTGATTTGGGTAGTGATC |
| | leaderless | AAAATGCAGATCAAAGTTAACCGTTATCCGGCATGGGGTGATACCGTTGAAATCAACACCCGCTTTTCTC |
| | fatB2 gene. | GTCTGGGCAAAATCGGTATGGGCCGTGACTGGCTGATCTCTGACTGTAACACTGGTGAAATTCTGGTTCG |
| | | TGCTACTAGCGCATACGCGATGATGAACCAGAAAACCCGTCGCCTGAGCAAGCTGCCGTACGAGGTCCAC |
| | | CAGGAGATTGTTCCGCTGTTTGTAGACAGCCCAGTGATTGAGGATTCTGACCTGAAAGTGCATAAATTCA |
| | | AAGTGAAGACCGGTGACAGCATCCAAAAAGGCCTGACCCCAGGTTGGAACGATCTGGACGTTAACCAGCA |
| | | CGTTTCCAACGTGAAGTATATCGGTTGGATTCTGGAGAGCATGCCGACCGAGGTCCTGGAAACCCAGGAG |
| | | CTGTGTTCCCTGGCGCTGGAGTACCGCCGTGAGTGCGGCCGTGACAGCGTGCTGGAGTCTGTGACCGCTA |
| | | TGGACCCAAGCAAAGTTGGTGTTCGTAGCCAGTACCAGCACCTGCTGCGTCTGGAAGACGGTACTGCTAT |
| | | CGTGAACGGTGCAACTGAATGGCGTCCTAAAAACGCGGGTGCAAACGGTGCTATCAGCACCGGTAAAACC |
| | | TCTAACGGTAACTCCGTGAGCTAAAAGCTT |
| 8 | plasmid | AAAAGCAGAGCATTACGCTGACTTGACGGGACGGCGCAAGCTCATGACCAAAATCCCTTAACGTGAGTTA |
| | pAQ3::P(nir07)_ | CGCGCGCGTCGTTCCACTGAGCGTCAGACCCCGTAGAAAAGATCAAAGGATCTTCTTGAGATCCTTTTTT |
| | adm_carB_ | TCTGCGCGTAATCTGCTGCTTGCAAACAAAAAAACCACCGCTACCAGCGGTGGTTTGTTTGCCGGATCAA |
| | fatB2_entD_ | GAGCTACCAACTCTTTTTCCGAAGGTAACTGGCTTCAGCAGAGCGCAGATACCAAATACTGTTCTTCTAG |
| | SpecR. | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | CAAGAACTGGCCCTGAGAAGCGAGCTGGACTTCAATAGCGAAACCTATAAAGATGCGTATAGCCGTATTA |
| | | ACGCCATTGTGATCGAAGGCGAGCAAGAAGCATACCAAAACTACCTGGACATGGCGCAACTGCTGCCGGA |
| | | GGACGAGGCTGAGCTGATTCGTTTGAGCAAGATGGAGAACCGTCACAAAAAGGGTTTTCAAGCGTGCGGC |
| | | AAGAACCTCAATGTGACTCCGGATATGGATTATGCACAGCAGTTCTTTGCGGAGCTGCACGGCAATTTTC |
| | | AGAAGGCTAAAGCCGAGGGTAAGATTGTTACCTGCCTGCTCATCCAAAGCCTGATCATCGAGGCGTTTGC |
| | | GATTGCAGCCTACAACATTTACATTCCAGTGGCTGATCCGTTTGCACGTAAAATCACCGAGGGTGTCGTC |
| | | AAGGATGAGTATACCCACCTGAATTTCGGCGAAGTTTGGTTGAAGGAACATTTTGAAGCAAGCAAGGCGG |
| | | AGTTGGAGGACGCCAACAAAGAGAACTTACCGCTGGTCTGGCAGATGTTGAACCAGGTCGAAAAGGATGC |
| | | CGAAGTGCTGGGTATGGAGAAAGAGGCTCTGGTGGAGGACTTTATGATTAGCTATGGTGAGGCACTGAGC |
| | | AACATCGGCTTTTCTACGAGAGAAATCATGAAGATGAGCGCGTACGGTCTGCGTGCAGCATAAGAGCTCG |
| | | AGGAGGTTTTTACAATGACCAGCGATGTTCACGACGCCACAGACGGCGTCACCGAAACCGCACTCGACGA |
| | | CGAGCAGTCGACCCGCCGCATCGCCGAGCTGTACGCCACCGATCCCGAGTTCGCCGCCGCCGCACCGTTG |
| | | CCCGCCGTGGTCGACGCGGCGCACAAACCCGGGCTGCGGCTGGCAGAGATCCTGCAGACCCTGTTCACCG |
| | | GCTACGGTGACCGCCCGGCGCTGGGATACCGCGCCCGTGAACTGGCCACCGACGAGGGCGGGCGCACCGT |
| | | GACGCGTCTGCTGCCGCGGTTCGACACCCTCACCTACGCCCAGGTGTGGTCGCGCGTGCAAGCGGTCGCC |
| | | GCGGCCCTGCGCCACAACTTCGCGCAGCCGATCTACCCCGGCGACGCCGTCGCGACGATCGGTTTCGCGA |
| | | GTCCCGATTACCTGACGCTGGATCTCGTATGCGCCTACCTGGGCCTCGTGAGTGTTCCGCTGCAGCACAA |
| | | CGCACCGGTCAGCCGGCTCGCCCCGATCCTGGCCGAGGTCGAACCGCGGATCCTCACCGTGAGCGCCGAA |
| | | TACCTCGACCTCGCAGTCGAATCCGTGCGGGACGTCAACTCGGTGTCGCAGCTCGTGGTGTTCGACCATC |
| | | ACCCCGAGGTCGACGACCACCGCGACGCACTGGCCCGCGCGCGTGAACAACTCGCCGGCAAGGGCATCGC |
| | | CGTCACCACCCTGGACGCGATCGCCGACGAGGGCGCCGGGCTGCCGGCCGAACCGATCTACACCGCCGAC |
| | | CATGATCAGCGCCTCGCGATGATCCTGTACACCTCGGGTTCCACCGGCGCACCCAAGGGTGCGATGTACA |
| | | CCGAGGCGATGGTGGCGCGGCTGTGGACCATGTCGTTCATCACGGGTGACCCCACGCCGGTCATCAACGT |
| | | CAACTTCATGCCGCTCAACCACCTGGGCGGGCGCATCCCCATTTCCACCGCCGTGCAGAACGGTGGAACC |
| | | AGTTACTTCGTACCGGAATCCGACATGTCCACGCTGTTCGAGGATCTCGCGCTGGTGCGCCCGACCGAAC |
| | | TCGGCCTGGTTCCGCGCGTCGCCGACATGCTCTACCAGCACCACCTCGCCACCGTCGACCGCCTGGTCAC |
| | | GCAGGGCGCCGACGAACTGACCGCCGAGAAGCAGGCCGGTGCCGAACTGCGTGAGCAGGTGCTCGGCGGA |
| | | CGCGTGATCACCGGATTCGTCAGCACCGCACCGCTGGCCGCGGAGATGAGGGCGTTCCTCGACATCACCC |
| | | TGGGCGCACACATCGTCGACGGCTACGGGCTCACCGAGACCGGCGCCGTGACACGCGACGGTGTGATCGT |
| | | GCGGCCACCGGTGATCGACTACAAGCTGATCGACGTTCCCGAACTCGGCTACTTCAGCACCGACAAGCCC |
| | | TACCCGCGTGGCGAACTGCTGGTCAGGTCGCAAACGCTGACTCCCGGGTACTACAAGCGCCCCGAGGTCA |
| | | CCGCGAGCGTCTTCGACCGGGACGGCTACTACCACACCGGCGACGTCATGGCCGAGACCGCACCCGACCA |
| | | CCTGGTGTACGTGGACCGTCGCAACAACGTCCTCAAACTCGCGCAGGGCGAGTTCGTGGCGGTCGCCAAC |
| | | CTGGAGGCGGTGTTCTCCGGCGCGGCGCTGGTGCGCCAGATCTTCGTGTACGGCAACAGCGAGCGCAGTT |
| | | TCCTTCTGGCCGTGGTGGTCCCGACGCCGGAGGCGCTCGAGCAGTACGATCCGGCCGCGCTCAAGGCCGC |
| | | GCTGGCCGACTCGCTGCAGCGCACCGCACGCGACGCCGAACTGCAATCCTACGAGGTGCCGGCCGATTTC |
| | | ATCGTCGAGACCGAGCCGTTCAGCGCCGCCAACGGGCTGCTGTCGGGTGTCGGAAAACTGCTGCGGCCCA |
| | | ACCTCAAAGACCGCTACGGGCAGCGCCTGGAGCAGATGTACGCCGATATCGCGGCCACGCAGGCCAACCA |
| | | GTTGCGCGAACTGCGGCGCGCGGCCGCCACACAACCGGTGATCGACACCCTCACCCAGGCCGCTGCCACG |
| | | ATCCTCGGCACCGGGAGCGAGGTGGCATCCGACGCCCACTTCACCGACCTGGGCGGGGATTCCCTGTCGG |
| | | CGCTGACACTTTCGAACCTGCTGAGCGATTTCTTCGGTTTCGAAGTTCCCGTCGGCACCATCGTGAACCC |
| | | GGCCACCAACCTCGCCCAACTCGCCCAGCACATCGAGGCGCAGCGCACCGCGGGTGACCGCAGGCCGAGT |
| | | TTCACCACCGTGCACGGCGCGGACGCCACCGAGATCCGGGCGAGTGAGCTGACCCTGGACAAGTTCATCG |
| | | ACGCCGAAACGCTCCGGGCCGCACCGGGTCTGCCCAAGGTCACCACCGAGCCACGGACGGTGTTGCTCTC |
| | | GGGCGCCAACGGCTGGCTGGGCCGGTTCCTCACGTTGCAGTGGCTGGAACGCCTGGCACCTGTCGGCGGC |
| | | ACCCTCATCACGATCGTGCGGGGCCGCGACGACGCCGCGGCCCGCGCACGGCTGACCCAGGCCTACGACA |
| | | CCGATCCCGAGTTGTCCCGCCGCTTCGCCGAGCTGGCCGACCGCCACCTGCGGGTGGTCGCCGGTGACAT |
| | | CGGCGACCCGAATCTGGGCCTCACACCCGAGATCTGGCACCGGCTCGCCGCCGAGGTCGACCTGGTGGTG |
| | | CATCCGGCAGCGCTGGTCAACCACGTGCTCCCCTACCGGCAGCTGTTCGGCCCCAACGTCGTGGGCACGG |
| | | CCGAGGTGATCAAGCTGGCCCTCACCGAACGGATCAAGCCCGTCACGTACCTGTCCACCGTGTCGGTGGC |
| | | CATGGGGATCCCCGACTTCGAGGAGGACGGCGACATCCGGACCGTGAGCCCGGTGCGCCCGCTCGACGGC |
| | | GGATACGCCAACGGCTACGGCAACAGCAAGTGGGCCGGCGAGGTGCTGCTGCGGGAGGCCCACGATCTGT |
| | | GCGGGCTGCCCGTGGCGACGTTCCGCTCGGACATGATCCTGGCGCATCCGCGCTACCGCGGTCAGGTCAA |
| | | CGTGCCAGACATGTTCACGCGACTCCTGTTGAGCCTCTTGATCACCGGCGTCGCGCCGCGGTCGTTCTAC |
| | | ATCGGAGACGGTGAGCGCCCGCGGGCGCACTACCCCGGCCTGACGGTCGATTTCGTGGCCGAGGCGGTCA |
| | | CGACGCTCGGCGCGCAGCAGCGCGAGGGATACGTGTCCTACGACGTGATGAACCCGCACGACGACGGGAT |
| | | CTCCCTGGATGTGTTCGTGGACTGGCTGATCCGGGCGGGCCATCCGATCGACCGGGTCGACGACTACGAC |
| | | GACTGGGTGCGTCGGTTCGAGACCGCGTTGACCGCGCTTCCCGAGAAGCGCCGCGCACAGACCGTACTGC |
| | | CGCTGCTGCACGCGTTCCGCGCTCCGCAGGCACCGTTGCGCGGCGCACCCGAACCCACGGAGGTGTTCCA |
| | | CGCCGCGGTGCGCACCGCGAAGGTGGGCCCGGGAGACATCCCGCACCTCGACGAGGCGCTGATCGACAAG |
| | | TACATACGCGATCTGCGTGAGTTCGGTCTGATCTGAGGTACCAGGAGGTTTTTACATGGACCGTAAAAGC |
| | | AAGCGTCCGGACATGCTGGTTGATTCCTTTGGTCTGGAAAGCACCGTGCAGGACGGTCTGGTTTTCCGTC |
| | | AGTCTTTCTCCATTCGTAGCTATGAGATTGGTACTGATCGTACCGCCTCTATCGAAACCCTGATGAATCA |
| | | CCTGCAAGAAACCTCTCTGAACCATTGTAAGTCTACTGGCATCCTGCTGGACGGTTTCGGTCGTACCCTG |
| | | GAGATGTGCAAACGCGACCTGATTTGGGTAGTGATCAAAATGCAGATCAAAGTTAACCGTTATCCGGCAT |
| | | GGGGTGATACCGTTGAAATCAACACCCGCTTTTCTCGTCTGGGCAAAATCGGTATGGGCCGTGACTGGCT |
| | | GATCTCTGACTGTAACACTGGTGAAATTCTGGTTCGTGCTACTAGCGCATACGCGATGATGAACCAGAAA |
| | | ACCCGTCGCCTGAGCAAGCTGCCGTACGAGGTCCACCAGGAGATTGTTCCGCTGTTTGTAGACAGCCCAG |
| | | TGATTGAGGATTCTGACCTGAAAGTGCATAAATTCAAAGTGAAGACCGGTGACAGCATCCAAAAAGGCCT |
| | | GACCCCAGGTTGGAACGATCTGGACGTTAACCAGCACGTTTCCAACGTGAAGTATATCGGTTGGATTCTG |
| | | GAGAGCATGCCGACCGAGGTCCTGGAAACCCAGGAGCTGTGTTCCCTGGCGCTGGAGTACCGCCGTGAGT |
| | | GCGGCCGTGACAGCGTGCTGGAGTCTGTGACCGCTATGGACCCAAGCAAAGTTGGTGTTCGTAGCCAGTA |
| | | CCAGCACCTGCTGCGTCTGGAAGACGGTACTGCTATCGTGAACGGTGCAACTGAATGGCGTCCTAAAAAC |
| | | GCGGGTGCAAACGGTGCTATCAGCACCGGTAAAACCTCTAACGGTAACTCCGTGAGCTAAAAGCTTGTTG |
| | | CTGCATGCAGGAGGTTTTTACAATGAAAACGACCCACACCAGCTTACCATTTGCCGGCCACACGTTACAT |
| | | TTCGTCGAATTTGATCCGGCGAACTTTTGTGAACAAGACCTGTTGTGGCTGCCGCATTATGCCCAGCTGC |
| | | AGCACGCAGGCCGTAAGCGTAAAACTGAACATCTGGCCGGTCGCATTGCGGCAGTGTATGCCCTGCGCGA |
| | | GTACGGCTACAAATGCGTGCCGGCCATTGGTGAACTGCGTCAACCGGTTTGGCCGGCAGAAGTTTACGGT |
| | | TCCATCTCCCACTGCGGTACTACCGCGTTGGCGGTTGTGTCTCGCCAGCCGATCGGTATTGATATTGAAG |
| | | AGATATTCTCTGTCCAGACGGCACGCGAGCTGACGGACAACATCATTACCCCGGCAGAGCACGAGCGTCT |
| | | GGCGGACTGTGGTCTGGCGTTCAGCCTGGCGCTGACCCTGGCATTCAGCGCAAAAGAGAGCGCGTTCAAG |
| | | GCTTCCGAGATCCAAACCGATGCGGGCTTCCTGGATTATCAAATCATCAGCTGGAACAAGCAACAGGTTA |
| | | TCATTCACCGTGAGAATGAGATGTTTGCCGTCCATTGGCAGATTAAAGAGAAAATCGTTATCACCCTGTG |
| | | CCAGCACGACTGAGAATTCGGTTTTCCGTCCTGTCTTGATTTTCAAGCAAACAATGCCTCCGATTTCTAA |
| | | TCGGAGGCATTTGTTTTTGTTTATTGCAAAAACAAAAAATATTGTTACAAATTTTTACAGGCTATTAAGC |
| | | CTACCGTCATAAATAATTTGCCATTTACTAGTTTTTAATTAACCAGAACCTTGACCGAACGCAGCGGTGG |
| | | TAACGGCGCAGTGGCGGTTTTCATGGCTTGTTATGACTGTTTTTTTGGGGTACAGTCTATGCCTCGGGCA |
| | | TCCAAGCAGCAAGCGCGTTACGCCGTGGGTCGATGTTTGATGTTATGGAGCAGCAACGATGTTACGCAGC |
| | | AGGGCAGTCGCCCTAAAACAAAGTTAAACATCATGAGGGAAGCGGTGATCGCCGAAGTATCGACTCAACT |
| | | ATCAGAGGTAGTTGGCGTCATCGAGCGCCATCTCGAACCGACGTTGCTGGCCGTACATTTGTACGGCTCC |
| | | GCAGTGGATGGCGGCCTGAAGCCACACAGTGATATTGATTTGCTGGTTACGGTGACCGTAAGGCTTGATG |
| | | AAACAACGCGGCGAGCTTTGATCAACGACCTTTTGGAAACTTCGGCTTCCCCTGGAGAGAGCGAGATTCT |
| | | CCGCGCTGTAGAAGTCACCATTGTTGTGCACGACGACATCATTCCGTGGCGTTATCCAGCTAAGCGCGAA |
| | | CTGCAATTTGGAGAATGGCAGCGCAATGACATTCTTGCAGGTATCTTCGAGCCAGCCACGATCGACATTG |
| | | ATCTGGCTATCTTGCTGACAAAAGCAAGAGAACATAGCGTTGCCTTGGTAGGTCCAGCGGCGGAGGAACT |
| | | CTTTGATCCGGTTCCTGAACAGGATCTATTTGAGGCGCTAAATGAAACCTTAACGCTATGGAACTCGCCG |
| | | CCCGACTGGGCTGGCGATGAGCGAAATGTAGTGCTTACGTTGTCCCGCATTTGGTACAGCGCAGTAACCG |
| | | GCAAAATCGCGCCGAAGGATGTCGCTGCCGACTGGGCAATGGAGCGCCTGCCGGCCCAGTATCAGCCCGT |
| | | CATACTTGAAGCTAGACAGGCTTATCTTGGACAAGAAGAAGATCGCTTGGCCTCGCGCGCAGATCAGTTG |
| | | GAAGAATTTGTCCACTACGTGAAAGGCGAGATCACCAAGGTAGTCGGCAAATAATGTCTAACAATTCGTT |
| | | CAAGCCGACGCCGCTTCGCGGCGCGGCTTAACTCAAGCGTTAGATGCACTAAGCACATAATTGCTCACAG |
| | | CCAAACTATCAGGTCAAGTCTGCTTTTATTATTTTTAAGCGTGCATAATAAGCCCTACACAAATTGGGAG |
| | | ATATATCATGAGGCGCGCCACGAGAAAGAGTTATGACAAATTAAAATTCTGACTCTTAGATTATTTCCAG |
| | | AGAGGCTGATTTTCCCAATCTTTGGGAAAGCCTAAGTTTTTAGATTCTATTTCTGGATACATCTCAAAAG |
| | | TTCTTTTTAAATGCTGTGCAAAATTATGCTCTGGTTTAATTCTGTCTAAGAGATACTGAATACAACATAA |
| | | GCCAGTGAAAATTTTACGGCTGTTTCTTTGATTAATATCCTCCAATACTTCTCTAGAGAGCCATTTTCCT |
| | | TTTAACCTATCAGGCAATTTAGGTGATTCTCCTAGCTGTATATTCCAGAGCCTTGAATGATGAGCGCAAA |
| | | TATTTCTAATATGCGACAAAGACCGTAACCAAGATATAAAAAACTTGTTAGGTAATTGGAAATGAGTATG |
| | | TATTTTTTGTCGTGTCTTAGATGGTAATAAATTTGTGTACATTCTAGATAACTGCCCAAAGGCGATTATC |
| | | TCCAAAGCCATATATGACGGCGGTAGTAGAGGATTTGTGTACTTGTTTCGATAATGCCCGATAAATTCTT |
| | | CTACTTTTTTAGATTGGCAATATTGAGTAATCGAATCGATTAATTCTTGATGCTTCCCAGTGTCATAAAA |
| | | TAAACTTTTATTCAGATACCAATGAGGATCATAATCATGGGAGTAGTGATAAATCATTTGAGTTCTGACT |
| | | GCTACTTCTATCGACTCCGTAGCATTAAAAATAAGCATTCTCAAGGATTTATCAAACTTGTATAGATTTG |
| | | GCCGGCCCGTCAAAAGGGCGACACCCCATAATTAGCCCGGGCGAAAGGCCCAGTCTTTCGACTGAGCCTT |
| | | TCGTTTTATTTGATGCCTGGCAGTTCCCTACTCTCGCATGGGGAGTCCCCACACTACCATCGGCGCTACG |
| | | GCGTTTCACTTCTGAGTTCGGCATGGGGTCAGGTGGGACCACCGCGCTACTGCCGCCAGGCAAACAAGGG |
| | | GTGTTATGAGCCATATTCAGGTATAAATGGGCTCGCGATAATGTTCAGAATTGGTTAATTGGTTGTAACA |
| | | CTGACCCCTATTTGTTTATTTTTCTAAATACATTCAAATATGTATCCGCTCATGAGACAATAACCCTGAT |
| | | AAATGCTTCAATAATATTGAAAAAGGAAGAATATGAGTATTCAACATTTCCGTGTCGCCCTTATTCCCTT |
| | | TTTTGCGGCATTTTGCCTTCCTGTTTTTGCTCACCCAGAAACGCTGGTGAAAGTAAAAGATGCTGAAGAT |
| | | CAGTTGGGTGCACGAGTGGGTTACATCGAACTGGATCTCAACAGCGGTAAGATCCTTGAGAGTTTTCGCC |
| | | CCGAAGAACGTTTTCCAATGATGAGCACTTTTAAAGTTCTGCTATGTGGCGCGGTATTATCCCGTATTGA |
| | | CGCCGGGCAAGAGCAACTCGGTCGCCGCATACACTATTCTCAGAATGACTTGGTTGAGTACTCACCAGTC |
| | | ACAGAAAAGCATCTTACGGATGGCATGACAGTAAGAGAATTATGCAGTGCTGCCATAACCATGAGTGATA |
| | | ACACTGCGGCCAACTTACTTCTGACAACGATCGGAGGACCGAAGGAGCTAACCGCTTTTTTGCACAACAT |
| | | GGGGGATCATGTAACTCGCCTTGATCGTTGGGAACCGGAGCTGAATGAAGCCATACCAAACGACGAGCGT |
| | | GACACCACGATGCCTGTAGCGATGGCAACAACGTTGCGCAAACTATTAACTGGCGAACTACTTACTCTAG |
| | | CTTCCCGGCAACAATTAATAGACTGGATGGAGGCGGATAAAGTTGCAGGACCACTTCTGCGCTCGGCCCT |
| | | TCCGGCTGGCTGGTTTATTGCTGATAAATCCGGAGCCGGTGAGCGTGGTTCTCGCGGTATCATCGCAGCG |
| | | CTGGGGCCAGATGGTAAGCCCTCCCGTATCGTAGTTATCTACACGACGGGGAGTCAGGCAACTATGGATG |
| | | AACGAAATAGACAGATCGCTGAGATAGGTGCCTCACTGATTAAGCATTGGT |
| 9 | carB | MTSDVHDATDGVTETALDDEQSTRRIAELYATDPEFAAAAPLPAVVDAAHKPGLRLAEILQTLFTGYGDR |
| | Mycobacterium | PALGYRARELATDEGGRTVTRLLPREDTLTYAQVWSRVQAVAAALRHNFAQPIYPGDAVATIGFASPDYL |
| | smegmatis | TLDLVCAYLGLVSVPLQHNAPVSRLAPILAEVEPRILTVSAEYLDLAVESVRDVNSVSQLVVEDHHPEVD |
| | | DHRDALARAREQLAGKGIAVTTLDAIADEGAGLPAEPIYTADHDQRLAMILYTSGSTGAPKGAMYTEAMV |
| | | ARLWTMSFITGDPTPVINVNFMPLNHLGGRIPISTAVQNGGTSYFVPESDMSTLFEDLALVRPTELGLVP |
| | | RVADMLYQHHLATVDRLVTQGADELTAEKQAGAELREQVLGGRVITGFVSTAPLAAEMRAFLDITLGAHI |
| | | VDGYGLTETGAVTRDGVIVRPPVIDYKLIDVPELGYFSTDKPYPRGELLVRSQTLTPGYYKRPEVTASVF |
| | | DRDGYYHTGDVMAETAPDHLVYVDRRNNVLKLAQGEFVAVANLEAVESGAALVRQIFVYGNSERSFLLAV |
| | | VVPTPEALEQYDPAALKAALADSLQRTARDAELQSYEVPADFIVETEPFSAANGLLSGVGKLLRPNLKDR |
| | | YGQRLEQMYADIAATQANQLRELRRAAATQPVIDTLTQAAATILGTGSEVASDAHFTDLGGDSLSALTLS |
| | | NLLSDFFGFEVPVGTIVNPATNLAQLAQHIEAQRTAGDRRPSFTTVHGADATEIRASELTLDKFIDAETL |
| | | RAAPGLPKVTTEPRTVLLSGANGWLGRFLTLQWLERLAPVGGTLITIVRGRDDAAARARLTQAYDTDPEL |
| | | SRRFAELADRHLRVVAGDIGDPNLGLTPEIWHRLAAEVDLVVHPAALVNHVLPYRQLFGPNVVGTAEVIK |
| | | LALTERIKPVTYLSTVSVAMGIPDFEEDGDIRTVSPVRPLDGGYANGYGNSKWAGEVLLREAHDLCGLPV |
| | | ATFRSDMILAHPRYRGQVNVPDMFTRLLLSLLITGVAPRSFYIGDGERPRAHYPGLTVDFVAEAVTTLGA |
| | | QQREGYVSYDVMNPHDDGISLDVFVDWLIRAGHPIDRVDDYDDWVRRFETALTALPEKRRAQTVLPLLHA |
| | | FRAPQAPLRGAPEPTEVFHAAVRTAKVGPGDIPHLDEALIDKYIRDLREFGLI |
| 10 | entD E.coli | MKTTHTSLPFAGHTLHFVEFDPANFCEQDLLWLPHYAQLQHAGRKRKTEHLAGRIAAVYALREYGYKCVP |
| | | AIGELRQPVWPAEVYGSISHCGTTALAVVSRQPIGIDIEEIFSVQTARELTDNIITPAEHERLADCGLAF |
| | | SLALTLAFSAKESAFKASEIQTDAGFLDYQIISWNKQQVIIHRENEMFAVHWQIKEKIVITLCQHD |
| 11 | acrM | MNAKLKKLFQQKVDGKTIIVTGASSGIGLTVSKYLAQAGAHVLLLARTKEKLDEVKAEIEAEGGKATVFP |
| | Acinetobacter | CDLNDMESIDAVSKEILAAVDHIDILVNNAGRSIRRAVHESVDRFHDFERTMQLNYFGAVRLVLNVLPHM |
| | sp. M-1 | MQRKDGQIINISSIGVLANATRFSAYVASKAALDAFSRCLSAEVHSHKIAITSIYMPLVRTPMIAPTKIY |
| | | KYVPTLSPEEAADLIAYAIVKRPKKIATNLGRLASITYAIAPDINNILMSIGFNLFPSSTASVGEQEKLN |
| | | LIQRAYARLFPGEHW |
| 12 | fadD E.coli | MKKVWLNRYPADVPTEINPDRYQSLVDMFEQSVARYADQPAFVNMGEVMTFRKLEERSRAFAAYLQQGLG |
| | | LKKGDRVALMMPNLLQYPVALEGILRAGMIVVNVNPLYTPRELEHQLNDSGASAIVIVSNFAHTLEKVVD |
| | | KTAVQHVILTRMGDQLSTAKGTVVNEVVKYIKRLVPKYHLPDAISFRSALHNGYRMQYVKPELVPEDLAF |
| | | LQYTGGTTGVAKGAMLTHRNMLANLEQVNATYGPLLHPGKELVVTALPLYHIFALTINCLLFIELGGQNL |
| | | LITNPRDIPGLVKELAKYPFTAITGVNTLFNALLNNKEFQQLDFSSLHLSAGGGMPVQQVVAERWVKLTG |
| | | QYLLEGYGLTECAPLVSVNPYDIDYHSGSIGLPVPSTEAKLVDDDDNEVPPGQPGELCVKGPQVMLGYWQ |
| | | RPDATDEIIKNGWLHTGDIAVMDEEGFLRIVDRKKDMILVSGENVYPNEIEDVVMQHPGVQEVAAVGVPS |
| | | GSSGEAVKIFVVKKDPSLTEESLVTFCRRQLTGYKVPKLVEFRDELPKSNVGKILRRELRDEARGKVDNK |
| | | A |
| 13 | fatB (C12 | MATTSLASAFCSMKAVMLARDGRGMKPRSSDLQLRAGNAPTSLKMINGTKFSYTESLKRLPDWSMLFAVI |
| | fatty acid) | TTIFSAAEKQWTNLEWKPKPKLPQLLDDHEGLHGLVERRTFAIRSYEVGPDRSTSILAVMNHMQEATLNH |
| | Umbellularia | AKSVGILGDGFGTTLEMSKRDLMWVVRRTHVAVERYPTWGDTVEVECWIGASGNNGMRRDFLVRDCKTGE |
| | californica | ILTRCTSLSVLMNTRTRRLSTIPDEVRGEIGPAFIDNVAVKDDEIKKLQKLNDSTADYIQGGLTPRWNDL |
| | | DVNQHVNNLKYVAWVFETVPDSIFESHHISSFTLEYRRECTRDSVLRSLTTVSGGSSEAGLVCDHLLQLE |
| | | GGSEVLRARTEWRPKLTDSFRGISVIPAEPRV |
| 14 | fatBmat (fatB | MEWKPKPKLPQLLDDHEGLHGLVERRTFAIRSYEVGPDRSTSILAVMNHMQEATLNHAKSVGILGDGFGT |
| | without | TLEMSKRDLMWVVRRTHVAVERYPTWGDTVEVECWIGASGNNGMRRDFLVRDCKTGEILTRCTSLSVLMN |
| | leader | TRTRRLSTIPDEVRGEIGPAFIDNVAVKDDEIKKLQKLNDSTADYIQGGLTPRWNDLDVNQHVNNLKYVA |
| | sequence) | WVFETVPDSIFESHHISSFTLEYRRECTRDSVLRSLTTVSGGSSEAGLVCDHLLQLEGGSEVLRARTEWR |
| | Umbellularia | PKLTDSFRGISVIPAEPRV |
| | californica | |
| 15 | fatB2 (C8 C10 | MVAAAASSAFFPVPAPGASPKPGKEGNWPSSLSPSFKPKSIPNGGFQVKANDSAHPKANGSAVSLKSGSL |
| | fatty acid) | NTQEDTSSSPPPRTFLHQLPDWSRLLTAITTVEVKSKRPDMHDRKSKRPDMLVDSFGLESTVQDGLVFRQ |
| | Cuphea | SFSIRSYEIGTDRTASIETLMNHLQETSLNHCKSTGILLDGFGRTLEMCKRDLIWVVIKMQIKVNRYPAW |
| | hookeriana | GDTVEINTRFSRLGKIGMGRDWLISDCNTGEILVRATSAYAMMNQKTRRLSKLPYEVHQEIVPLFVDSPV |
| | | IEDSDLKVHKEKVKTGDSIQKGLTPGWNDLDVNQHVSNVKYIGWILESMPTEVLETQELCSLALEYRREC |
| | | GRDSVLESVTAMDPSKVGVRSQYQHLLRLEDGTAIVNGATEWRPKNAGANGAISTGKTSNGNSVS |
| 16 | fatB2mat (fatB | MDRKSKRPDMLVDSFGLESTVQDGLVFRQSFSIRSYEIGTDRTASIETLMNHLQETSLNHCKSTGILLDG |
| | 2 without | FGRTLEMCKRDLIWVVIKMQIKVNRYPAWGDTVEINTRFSRLGKIGMGRDWLISDCNTGEILVRATSAYA |
| | leader | MMNQKTRRLSKLPYEVHQEIVPLFVDSPVIEDSDLKVHKEKVKTGDSIQKGLTPGWNDLDVNQHVSNVKY |
| | sequence) | IGWILESMPTEVLETQELCSLALEYRRECGRDSVLESVTAMDPSKVGVRSQYQHLLRLEDGTAIVNGATE |
| | Cuphea | WRPKNAGANGAISTGKTSNGNSVS |
| | hookeriana | |
| 17 | kivd | MYTVGDYLLDRLHELGIEEIFGVPGDYNLQFLDQIISRKDMKWVGNANELNASYMADGYARTKKAAAFLT |
| | Lactococcus | TFGVGELSAVNGLAGSYAENLPVVEIVGSPTSKVQNEGKEVHHTLADGDFKHFMKMHEPVTAARTLLTAE |
| | lactis | NATVEIDRVLSALLKERKPVYINLPVDVAAAKAEKPSLPLKKENPTSNTSDQEILNKIQESLKNAKKPIV |
| | | ITGHEIISFGLENTVTQFISKTKLPITTLNFGKSSVDETLPSFLGIYNGKLSEPNLKEFVESADFILMLG |
| | | VKLTDSSTGAFTHHLNENKMISLNIDEGKIFNESIQNFDFESLISSLLDLSGIEYKGKYIDKKQEDFVPS |
| | | NALLSQDRLWQAVENLTQSNETIVAEQGTSFFGASSIFLKPKSHFIGQPLWGSIGYTFPAALGSQIADKE |
| | | SRHLLFIGDGSLQLTVQELGLATREKINPICFIINNDGYTVEREIHGPNQSYNDIPMWNYSKLPESFGAT |
| | | EERVVSKIVRTENEFVSVMKEAQADPNRMYWIELVLAKEDAPKVLKKMGKLFAEQNKS |
| 18 | carboxylic | ATGACCAGCGATGTTCACGACGCCACAGACGGCGTCACCGAAACCGCACTCGACGACGAGCAGTCGACCC |
| | acid | GCCGCATCGCCGAGCTGTACGCCACCGATCCCGAGTTCGCCGCCGCCGCACCGTTGCCCGCCGTGGTCGA |
| | reductase | CGCGGCGCACAAACCCGGGCTGCGGCTGGCAGAGATCCTGCAGACCCTGTTCACCGGCTACGGTGACCGC |
| | amplified | CCGGCGCTGGGATACCGCGCCCGTGAACTGGCCACCGACGAGGGCGGGCGCACCGTGACGCGTCTGCTGC |
| | from | CGCGGTTCGACACCCTCACCTACGCCCAGGTGTGGTCGCGCGTGCAAGCGGTCGCCGCGGCCCTGCGCCA |
| | Mycobacterium | CAACTTCGCGCAGCCGATCTACCCCGGCGACGCCGTCGCGACGATCGGTTTCGCGAGTCCCGATTACCTG |
| | smegmatis. | ACGCTGGATCTCGTATGCGCCTACCTGGGCCTCGTGAGTGTTCCGCTGCAGCACAACGCACCGGTCAGCC |
| | | GGCTCGCCCCGATCCTGGCCGAGGTCGAACCGCGGATCCTCACCGTGAGCGCCGAATACCTCGACCTCGC |
| | | AGTCGAATCCGTGCGGGACGTCAACTCGGTGTCGCAGCTCGTGGTGTTCGACCATCACCCCGAGGTCGAC |
| | | GACCACCGCGACGCACTGGCCCGCGCGCGTGAACAACTCGCCGGCAAGGGCATCGCCGTCACCACCCTGG |
| | | ACGCGATCGCCGACGAGGGCGCCGGGCTGCCGGCCGAACCGATCTACACCGCCGACCATGATCAGCGCCT |
| | | CGCGATGATCCTGTACACCTCGGGTTCCACCGGCGCACCCAAGGGTGCGATGTACACCGAGGCGATGGTG |
| | | GCGCGGCTGTGGACCATGTCGTTCATCACGGGTGACCCCACGCCGGTCATCAACGTCAACTTCATGCCGC |
| | | TCAACCACCTGGGCGGGCGCATCCCCATTTCCACCGCCGTGCAGAACGGTGGAACCAGTTACTTCGTACC |
| | | GGAATCCGACATGTCCACGCTGTTCGAGGATCTCGCGCTGGTGCGCCCGACCGAACTCGGCCTGGTTCCG |
| | | CGCGTCGCCGACATGCTCTACCAGCACCACCTCGCCACCGTCGACCGCCTGGTCACGCAGGGCGCCGACG |
| | | AACTGACCGCCGAGAAGCAGGCCGGTGCCGAACTGCGTGAGCAGGTGCTCGGCGGACGCGTGATCACCGG |
| | | ATTCGTCAGCACCGCACCGCTGGCCGCGGAGATGAGGGCGTTCCTCGACATCACCCTGGGCGCACACATC |
| | | GTCGACGGCTACGGGCTCACCGAGACCGGCGCCGTGACACGCGACGGTGTGATCGTGCGGCCACCGGTGA |
| | | TCGACTACAAGCTGATCGACGTTCCCGAACTCGGCTACTTCAGCACCGACAAGCCCTACCCGCGTGGCGA |
| | | ACTGCTGGTCAGGTCGCAAACGCTGACTCCCGGGTACTACAAGCGCCCCGAGGTCACCGCGAGCGTCTTC |
| | | GACCGGGACGGCTACTACCACACCGGCGACGTCATGGCCGAGACCGCACCCGACCACCTGGTGTACGTGG |
| | | ACCGTCGCAACAACGTCCTCAAACTCGCGCAGGGCGAGTTCGTGGCGGTCGCCAACCTGGAGGCGGTGTT |
| | | CTCCGGCGCGGCGCTGGTGCGCCAGATCTTCGTGTACGGCAACAGCGAGCGCAGTTTCCTTCTGGCCGTG |
| | | GTGGTCCCGACGCCGGAGGCGCTCGAGCAGTACGATCCGGCCGCGCTCAAGGCCGCGCTGGCCGACTCGC |
| | | TGCAGCGCACCGCACGCGACGCCGAACTGCAATCCTACGAGGTGCCGGCCGATTTCATCGTCGAGACCGA |
| | | GCCGTTCAGCGCCGCCAACGGGCTGCTGTCGGGTGTCGGAAAACTGCTGCGGCCCAACCTCAAAGACCGC |
| | | TACGGGCAGCGCCTGGAGCAGATGTACGCCGATATCGCGGCCACGCAGGCCAACCAGTTGCGCGAACTGC |
| | | GGCGCGCGGCCGCCACACAACCGGTGATCGACACCCTCACCCAGGCCGCTGCCACGATCCTCGGCACCGG |
| | | GAGCGAGGTGGCATCCGACGCCCACTTCACCGACCTGGGCGGGGATTCCCTGTCGGCGCTGACACTTTCG |
| | | AACCTGCTGAGCGATTTCTTCGGTTTCGAAGTTCCCGTCGGCACCATCGTGAACCCGGCCACCAACCTCG |
| | | CCCAACTCGCCCAGCACATCGAGGCGCAGCGCACCGCGGGTGACCGCAGGCCGAGTTTCACCACCGTGCA |
| | | CGGCGCGGACGCCACCGAGATCCGGGCGAGTGAGCTGACCCTGGACAAGTTCATCGACGCCGAAACGCTC |
| | | CGGGCCGCACCGGGTCTGCCCAAGGTCACCACCGAGCCACGGACGGTGTTGCTCTCGGGCGCCAACGGCT |
| | | GGCTGGGCCGGTTCCTCACGTTGCAGTGGCTGGAACGCCTGGCACCTGTCGGCGGCACCCTCATCACGAT |
| | | CGTGCGGGGCCGCGACGACGCCGCGGCCCGCGCACGGCTGACCCAGGCCTACGACACCGATCCCGAGTTG |
| | | TCCCGCCGCTTCGCCGAGCTGGCCGACCGCCACCTGCGGGTGGTCGCCGGTGACATCGGCGACCCGAATC |
| | | TGGGCCTCACACCCGAGATCTGGCACCGGCTCGCCGCCGAGGTCGACCTGGTGGTGCATCCGGCAGCGCT |
| | | GGTCAACCACGTGCTCCCCTACCGGCAGCTGTTCGGCCCCAACGTCGTGGGCACGGCCGAGGTGATCAAG |
| | | CTGGCCCTCACCGAACGGATCAAGCCCGTCACGTACCTGTCCACCGTGTCGGTGGCCATGGGGATCCCCG |
| | | ACTTCGAGGAGGACGGCGACATCCGGACCGTGAGCCCGGTGCGCCCGCTCGACGGCGGATACGCCAACGG |
| | | CTACGGCAACAGCAAGTGGGCCGGCGAGGTGCTGCTGCGGGAGGCCCACGATCTGTGCGGGCTGCCCGTG |
| | | GCGACGTTCCGCTCGGACATGATCCTGGCGCATCCGCGCTACCGCGGTCAGGTCAACGTGCCAGACATGT |
| | | TCACGCGACTCCTGTTGAGCCTCTTGATCACCGGCGTCGCGCCGCGGTCGTTCTACATCGGAGACGGTGA |
| | | GCGCCCGCGGGCGCACTACCCCGGCCTGACGGTCGATTTCGTGGCCGAGGCGGTCACGACGCTCGGCGCG |
| | | CAGCAGCGCGAGGGATACGTGTCCTACGACGTGATGAACCCGCACGACGACGGGATCTCCCTGGATGTGT |
| | | TCGTGGACTGGCTGATCCGGGCGGGCCATCCGATCGACCGGGTCGACGACTACGACGACTGGGTGCGTCG |
| | | GTTCGAGACCGCGTTGACCGCGCTTCCCGAGAAGCGCCGCGCACAGACCGTACTGCCGCTGCTGCACGCG |
| | | TTCCGCGCTCCGCAGGCACCGTTGCGCGGCGCACCCGAACCCACGGAGGTGTTCCACGCCGCGGTGCGCA |
| | | CCGCGAAGGTGGGCCCGGGAGACATCCCGCACCTCGACGAGGCGCTGATCGACAAGTACATACGCGATCT |
| | | GCGTGAGTTCGGTCTGATCTGA |
| 19 | codon- | ATGCAGCAACTGACCGATCAAAGCAAAGAACTGGACTTCAAGAGCGAGACGTACAAAGACGCCTATAGCC |
| | optimized | GCATTAACGCGATCGTCATTGAAGGCGAACAAGAGGCGCATGAAAACTACATCACCCTGGCGCAGCTGCT |
| | Nostoc | GCCTGAGAGCCACGACGAACTGATTCGCCTGAGCAAAATGGAGAGCCGTCACAAGAAAGGTTTTGAGGCG |
| | punctiforme | TGTGGCCGCAATCTGGCGGTGACCCCGGACCTGCAATTTGCGAAGGAGTTCTTTAGCGGTCTGCACCAGA |
| | adm. | ATTTCCAGACGGCCGCAGCCGAGGGCAAAGTCGTCACTTGTTTGTTGATCCAGAGCCTGATTATTGAATG |
| | | CTTTGCTATTGCGGCGTACAACATTTACATTCCGGTCGCCGATGACTTTGCGCGTAAAATCACGGAAGGT |
| | | GTTGTCAAAGAGGAGTATTCCCACCTGAATTTCGGTGAAGTGTGGTTGAAGGAACATTTTGCGGAATCTA |
| | | AAGCCGAATTGGAACTGGCAAATCGCCAGAACCTGCCGATCGTTTGGAAGATGCTGAACCAAGTGGAAGG |
| | | TGATGCACATACGATGGCGATGGAGAAGGACGCATTGGTTGAGGACTTTATGATTCAGTATGGCGAAGCA |
| | | CTGTCCAATATCGGTTTCAGCACCCGTGATATCATGCGTCTGAGCGCCTATGGCCTGATCGGTGCCTAA |
| 20 | codon- | ATGGAGTGGAAACCAAAACCGAAACTGCCTCAGCTGCTGGATGACCACTTCGGTCTGCACGGCCTGGTTT |
| | optimized | TCCGTCGTACCTTCGCTATCCGTTCTTACGAAGTCGGCCCTGATCGCTCCACCTCCATCCTGGCGGTAAT |
| | Umbellularia | GAACCACATGCAGGAAGCAACTCTGAACCATGCGAAAAGCGTAGGTATCCTGGGCGATGGTTTCGGCACT |
| | californicia | ACTCTGGAGATGTCCAAACGTGATCTGATGTGGGTTGTTCGCCGTACCCATGTCGCGGTTGAACGCTACC |
| | fatBₘ (without | CGACCTGGGGCGATACGGTTGAAGTGGAATGCTGGATCGGCGCGTCCGGCAACAACGGCATGCGTCGCGA |
| | leader | TTTCCTGGTTCGCGATTGTAAGACGGGCGAGATTCTGACCCGTTGCACGTCCCTGAGCGTTCTGATGAAT |
| | sequence). | ACCCGTACCCGTCGTCTGAGCACCATCCCGGACGAAGTTCGCGGTGAAATTGGCCCGGCATTCATCGATA |
| | | ACGTTGCAGTAAAAGACGATGAAATCAAGAAACTGCAGAAACTGAATGACTCTACCGCGGACTACATCCA |
| | | GGGTGGTCTGACCCCGCGCTGGAACGACCTGGACGTGAACCAGCACGTCAACAACCTGAAATACGTAGCT |
| | | TGGGTATTCGAAACGGTCCCGGATTCTATCTTCGAATCTCACCACATCAGCTCCTTCACCCTGGAATACC |
| | | GTCGTGAGTGTACCCGTGACTCCGTTCTGCGCTCTCTGACCACGGTATCCGGCGGTAGCTCTGAAGCCGG |
| | | TCTGGTTTGCGATCACCTGCTGCAGCTGGAAGGCGGCAGCGAGGTTCTGCGTGCTCGTACTGAGTGGCGT |
| | | CCGAAGCTGACTGACTCTTTCCGCGGCATCTCTGTTATCCCGGCAGAGCCTCGTGTGTAA |
| 21 | codon- | ATGAAAACGACCCACACCAGCTTACCATTTGCCGGCCACACGTTACATTTCGTCGAATTTGATCCGGCGA |
| | optimized | ACTTTTGTGAACAAGACCTGTTGTGGCTGCCGCATTATGCCCAGCTGCAGCACGCAGGCCGTAAGCGTAA |
| | E. coli entD. | AACTGAACATCTGGCCGGTCGCATTGCGGCAGTGTATGCCCTGCGCGAGTACGGCTACAAATGCGTGCCG |
| | | GCCATTGGTGAACTGCGTCAACCGGTTTGGCCGGCAGAAGTTTACGGTTCCATCTCCCACTGCGGTACTA |
| | | CCGCGTTGGCGGTTGTGTCTCGCCAGCCGATCGGTATTGATATTGAAGAGATATTCTCTGTCCAGACGGC |
| | | ACGCGAGCTGACGGACAACATCATTACCCCGGCAGAGCACGAGCGTCTGGCGGACTGTGGTCTGGCGTTC |
| | | AGCCTGGCGCTGACCCTGGCATTCAGCGCAAAAGAGAGCGCGTTCAAGGCTTCCGAGATCCAAACCGATG |
| | | CGGGCTTCCTGGATTATCAAATCATCAGCTGGAACAAGCAACAGGTTATCATTCACCGTGAGAATGAGAT |
| | | GTTTGCCGTCCATTGGCAGATTAAAGAGAAAATCGTTATCACCCTGTGCCAGCACGACTGA |
| 22 | plasmid | TAGAAAAACTCATCGAGCATCAAATGAAACTGCAATTTATTCATATCAGGATTATCAATACCATATTTTT |
| | pAQ4::P(cpcB)- | GAAAAAGCCGTTTCTGTAATGAAGGAGAAAACTCACCGAGGCAGTTCCATAGGATGGCAAGATCCTGGTA |
| | adm_{Npu}-ermC. | TCGGTCTGCGATTCCGACTCGTCCAACATCAATACAACCTATTAATTTCCCCTCGTCAAAAATAAGGTTA |
| | | TCAAGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATTCAT |
| | | TCGTGATTGCGCCTGAGCGAGGCGAAATACGCGATCGCTGTTAAAAGGACAATTACAAACAGGAATCGAG |
| | | TGCAACCGGCGCAGGAACACTGCCAGCGCATCAACAATATTTTCACCTGAATCAGGATATTCTTCTAATA |
| | | CCTGGAACGCTGTTTTTCCGGGGATCGCAGTGGTGAGTAACCATGCATCATCAGGAGTACGGATAAAATG |
| | | CTTGATGGTCGGAAGTGGCATAAATTCCGTCAGCCAGTTTAGTCTGACCATCTCATCTGTAACATCATTG |
| | | GCAACGCTACCTTTGCCATGTTTCAGAAACAACTCTGGCGCATCGGGCTTCCCATACAAGCGATAGATTG |
| | | TCGCACCTGATTGCCCGACATTATCGCGAGCCCATTTATACCCATATAAATCAGCATCCATGTTGGAATT |
| | | TAATCGCGGCCTCGACGTTTCCCGTTGAATATGGCTCATATTCTTCCTTTTTCAATATTATTGAAGCATT |
| | | TATCAGGGTTATTGTCTCATGAGCGGATACATATTTGAATGTATTTAGAAAAATAAACAAATAGGGGTCA |
| | | GTGTTACAACCAATTAACCAATTCTGAACATTATCGCGAGCCCATTTATACCTGAATATGGCTCATAACA |
| | | CCCCTTGTTTGCCTGGCGGCAGTAGCGCGGTGGTCCCACCTGACCCCATGCCGAACTCAGAAGTGAAACG |
| | | CCGTAGCGCCGATGGTAGTGTGGGGACTCCCCATGCGAGAGTAGGGAACTGCCAGGCATCAAATAAAACG |
| | | AAAGGCTCAGTCGAAAGACTGGGCCTTTCGCCCGGGCTAATTAGGGGGTGTCGCCCTTTACACGTACTTA |
| | | GTCGCTGAAGGCCTCACTGGCCCCTGCAGGGATGGTGGAATGCTGGTTATCTGGTGGGGATTAAGTGGTG |
| | | TTTTACTAAAGCTTGAACAACTCAAGAAAGATTATATTCGCAATAACTGCCAATAATCCCAGCATCTTGA |
| | | GAAAATCCAGCAAACCGGGGGCAAAACACCAGCAAGAAGCCAGCAGACTATCACCAAATCCCCAGCGTAC |
| | | AGCTAGAAATAACTGAGCAGTTGTATTCAATTACCTTCTGGTCAAGCCGAGGAAATTTCCCCACACCTTA |
| | | TACACCTCTGGAAGGTTTTTTTGACGAAGCGCAAAATATCCACAATCGGCTGGGGACTTCTTCTGTCAGA |
| | | AAATGGCAGAAATTTTTGAATGTGTTGGCGATCGCCCTCATCAATGATTATTAGAGAACTTTTGTCCCTG |
| | | ATGTTGGGAATACTCTTGATGACAATTGTGATTGCTCAAAGAAGAAAGAAATTTGGAGTAAATCTCTAAA |
| | | AGGGGACTGAAATATTTGTATGGTCAGCATGACCACTGAAATGGAGAGAAGTCTAAGACAGTAGATGTCT |
| | | TAGATATAAGCCTCATTAGAAGCCATGCCATAAAACAGATTTTGTGGATGAAACAACTTGAAATAGTTCA |
| | | GTTGTAGACCATGTTATAAACATTTATTCTTAACACAGTGACACATTAATGACTCATATATCCGTCCAAA |
| | | AAAAACTAAAATGTTTGTAAATTTAGTTTTGCGGCCGCGTCGACTTCGTTATAAAATAAACTTAACAAAT |
| | | CTATACCCACCTGTAGAGAAGAGTCCCTGAATATCAAAATGGTGGGATAAAAAGCTCAAAAAGGAAAGTA |
| | | GGCTGTGGTTCCCTAGGCAACAGTCTTCCCTACCCCACTGGAAACTAAAAAAACGAGAAAAGTTCGCACC |
| | | GAACATCAATTGCATAATTTTAGCCCTAAAACATAAGCTGAACGAAACTGGTTGTCTTCCCTTCCCAATC |
| | | CAGGACAATCTGAGAATCCCCTGCAACATTACTTAACAAAAAAGCAGGAATAAAATTAACAAGATGTAAC |
| | | AGACATAAGTCCCATCACCGTTGTATAAAGTTAACTGTGGGATTGCAAAAGCATTCAAGCCTAGGCGCTG |
| | | AGCTGTTTGAGCATCCCGGTGGCCCTTGTCGCTGCCTCCGTGTTTCTCCCTGGATTTATTTAGGTAATAT |
| | | CTCTCATAAATCCCCGGGTAGTTAACGAAAGTTAATGGAGATCAGTAACAATAACTCTAGGGTCATTACT |
| | | TTGGACTCCCTCAGTTTATCCGGGGGAATTGTGTTTAAGAAAATCCCAACTCATAAAGTCAAGTAGGAGA |
| | | TTAATCATATGCAGCAACTGACCGATCAAAGCAAAGAACTGGACTTCAAGAGCGAGACGTACAAAGACGC |
| | | CTATAGCCGCATTAACGCGATCGTCATTGAAGGCGAACAAGAGGCGCATGAAAACTACATCACCCTGGCG |
| | | CAGCTGCTGCCTGAGAGCCACGACGAACTGATTCGCCTGAGCAAAATGGAGAGCCGTCACAAGAAAGGTT |
| | | TTGAGGCGTGTGGCCGCAATCTGGCGGTGACCCCGGACCTGCAATTTGCGAAGGAGTTCTTTAGCGGTCT |
| | | GCACCAGAATTTCCAGACGGCCGCAGCCGAGGGCAAAGTCGTCACTTGTTTGTTGATCCAGAGCCTGATT |
| | | ATTGAATGCTTTGCTATTGCGGCGTACAACATTTACATTCCGGTCGCCGATGACTTTGCGCGTAAAATCA |
| | | CGGAAGGTGTTGTCAAAGAGGAGTATTCCCACCTGAATTTCGGTGAAGTGTGGTTGAAGGAACATTTTGC |
| | | GGAATCTAAAGCCGAATTGGAACTGGCAAATCGCCAGAACCTGCCGATCGTTTGGAAGATGCTGAACCAA |
| | | GTGGAAGGTGATGCACATACGATGGCGATGGAGAAGGACGCATTGGTTGAGGACTTTATGATTCAGTATG |
| | | GCGAAGCACTGTCCAATATCGGTTTCAGCACCCGTGATATCATGCGTCTGAGCGCCTATGGCCTGATCGG |
| | | TGCCTAACTCGAGCAATTCGGTTTTCCGTCCTGTCTTGATTTTCAAGCAAACAATGCCTCCGATTTCTAA |
| | | TCGGAGGCATTTGTTTTTGTTTATTGCAAAAACAAAAAATATTGTTACAAATTTTTACAGGCTATTAAGC |
| | | CTACCGTCATAAATAATTTGCCATTTACTAGTTTTAATTAACGTGCTATAATTATACTAATTTTATAAGG |
| | | AGGAAAAAATATGGGCATTTTTAGTATTTTTGTAATCAGCACAGTTCATTATCAACCAAACAAAAAATAA |
| | | GTGGTTATAATGAATCGTTAATAAGCAAAATTCATATAACCAAATTAAAGAGGGTTATAATGAACGAGAA |
| | | AAATATAAAACACAGTCAAAACTTTATTACTTCAAAACATAATATAGATAAAATAATGACAAATATAAGA |
| | | TTAAATGAACATGATAATATCTTTGAAATCGGCTCAGGAAAAGGCCATTTTACCCTTGAATTAGTAAAGA |
| | | GGTGTAATTTCGTAACTGCCATTGAAATAGACCATAAATTATGCAAAACTACAGAAAATAAACTTGTTGA |
| | | TCACGATAATTTCCAAGTTTTAAACAAGGATATATTGCAGTTTAAATTTCCTAAAAACCAATCCTATAAA |
| | | ATATATGGTAATATACCTTATAACATAAGTACGGATATAATACGCAAAATTGTTTTTGATAGTATAGCTA |
| | | ATGAGATTTATTTAATCGTGGAATACGGGTTTGCTAAAAGATTATTAAATACAAAACGCTCATTGGCATT |
| | | ACTTTTAATGGCAGAAGTTGATATTTCTATATTAAGTATGGTTCCAAGAGAATATTTTCATCCTAAACCT |
| | | AAAGTGAATAGCTCACTTATCAGATTAAGTAGAAAAAAATCAAGAATATCACACAAAGATAAACAAAAGT |
| | | ATAATTATTTCGTTATGAAATGGGTTAACAAAGAATACAAGAAAATATTTACAAAAAATCAATTTAACAA |
| | | TTCCTTAAAACATGCAGGAATTGACGATTTAAACAATATTAGCTTTGAACAATTCTTATCTCTTTTCAAT |
| | | AGCTATAAATTATTTAATAAGTAAGTTAAGGGATGCATAAACTGCATCCCTTAACTTGTTTTTCGTGTGC |
| | | CTATTTTTTGTGGCGCGCCCAGTTTCCTTTACTGGCCCTAAAGTCGCTGTGGCTAGGGTTCCGAAGGGGC |
| | | ATTATTGGCTCGCGGCTTTACAACCTTGATAAGGAGAGAGATGACAGTTTTTTTTCTCTTTTGCTTAGTA |
| | | AAACAGCAAATTTAAGGCATGTTAAAGAGCAGTAGAACGAAATGGTTGAGCCGGCCTCGATACACTCAAT |
| | | TAACTACTAATAGCTTCAATAAATTTTGGGACGATTGAAGCTATTTTTTTGAAAATCAACTCTTAATATC |
| | | TCCTGTCTCAAAAGAGTTAATTGCTAAACAAAAGCCAGTTTCAGCGAAAAATCTAGAGTTTTATAGGTTC |
| | | GTTCTCAGTACAGGACAAAAAGTTTGAAAAGGATAGAGGGAGAGGGTTTGATGGAAATAAGCACAAATCA |
| | | ATCAAGCCCTCATGAATCAGATTAGCGAAATTCGCCGCCAATTGCGACCTCATCTCGGATGGCATGGAGC |
| | | CAGACTGTCATTTATCGCCCTCTTCCTGGTGGCACTGTTCCGAGCAAAAACCGTCAATCTCGCCAAACTC |
| | | GCCACCGTCTGGGGAGGCAATGCAGCAGAAGAGTCTAATTACAAACGCATGCAGCGATTCTTTCAGTCCT |
| | | TTGACGTCAACATGGACAAAATCGCCAGGATGGTAATGAATATCGCGGCTATCCCGCAACCTTGGGTCTT |
| | | AAGCATCGACCGCACCAACGGCCGGCCTACATGGCCCGTCAATCGAAGGGCGACACAAAATTTATTCTAA |
| | | ATGCATAATAAATACTGATAACATCTTATAGTTTGTATTATATTTTGTATTATCGTTGACATGTATAATT |
| | | TTGATATCAAAAACTGATTTTCCCTTTATTATTTTCGAGATTTATTTTCTTAATTCTCTTTAACAAACTA |
| | | GAAATATTGTATATACAAAAAATCATAAATAATAGATGAATAGTTTAATTATAGGTGTTCATCAATCGAA |
| | | AAAGCAACGTATCTTATTTAAAGTGCGTTGCTTTTTTCTCATTTATAAGGTTAAATAATTCTCATATATC |
| | | AAGCAAAGTGACAGGCGCCCTTAAATATTCTGACAAATGCTCTTTCCCTAAACTCCCCCCATAAAAAAAC |
| | | CCGCCGAAGCGGGTTTTTACGTTATTTGCGGATTAACGATTACTCGTTATCAGAACCGCCCAGGGGGCCC |
| | | GAGCTTAAGACTGGCCGTCGTTTTACAACACAGAAAGAGTTTGTAGAAACGCAAAAAGGCCATCCGTCAG |
| | | GGGCCTTCTGCTTAGTTTGATGCCTGGCAGTTCCCTACTCTCGCCTTCCGCTTCCTCGCTCACTGACTCG |
| | | CTGCGCTCGGTCGTTCGGCTGCGGCGAGCGGTATCAGCTCACTCAAAGGCGGTAATACGGTTATCCACAG |
| | | AATCAGGGGATAACGCAGGAAAGAACATGTGAGCAAAAGGCCAGCAAAAGGCCAGGAACCGTAAAAAGGC |
| | | CGCGTTGCTGGCGTTTTTCCATAGGCTCCGCCCCCCTGACGAGCATCACAAAAATCGACGCTCAAGTCAG |
| | | AGGTGGCGAAACCCGACAGGACTATAAAGATACCAGGCGTTTCCCCCTGGAAGCTCCCTCGTGCGCTCTC |
| | | CTGTTCCGACCCTGCCGCTTACCGGATACCTGTCCGCCTTTCTCCCTTCGGGAAGCGTGGCGCTTTCTCA |
| | | TAGCTCACGCTGTAGGTATCTCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTCCAACCCGGTAAGACACGACT |
| | | TATCGCCACTGGCAGCAGCCACTGGTAACAGGATTAGCAGAGCGAGGTATGTAGGCGGTGCTACAGAGTT |
| | | CTTGAAGTGGTGGGCTAACTACGGCTACACTAGAAGAACAGTATTTGGTATCTGCGCTCTGCTGAAGCCA |
| | | GTTACCTTCGGAAAAAGAGTTGGTAGCTCTTGATCCGGCAAACAAACCACCGCTGGTAGCGGTGGTTTTT |
| | | TTGTTTGCAAGCAGCAGATTACGCGCAGAAAAAAAGGATCTCAAGAAGATCCTTTGATCTTTTCTACGGG |
| | | GTCTGACGCTCAGTGGAACGACGCGCGCGTAACTCACGTTAAGGGATTTTGGTCATGAGCTTGCGCCGTC |
| | | CCGTCAAGTCAGCGTAATGCTCTGCTTT |
| 23 | plasmid | AAAAGCAGAGCATTACGCTGACTTGACGGGACGGCGCAAGCTCATGACCAAAATCCCTTAACGTGAGTTA |
| | pAQ3::P(nir07- | CGCGCGCGTCGTTCCACTGAGCGTCAGACCCCGTAGAAAAGATCAAAGGATCTTCTTGAGATCCTTTTTT |
| | fatBₘ-carB- | TCTGCGCGTAATCTGCTGCTTGCAAACAAAAAAACCACCGCTACCAGCGGTGGTTTGTTTGCCGGATCAA |
| | entD-SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGOCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCG<+GCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | G<AAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGG<+TAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | GAGTGGAAACCAAAACCGAAACTGCCTCAGCTGCTGGATGACCACTTCGGTCTGCACGGCCTGGTTTTCC |
| | | GTCGTACCTTCGCTATCCGTTCTTACGAAGTCGGCCCTGATCGCTCCACCTCCATCCTGGCGGTAATGAA |
| | | CCACATGCAGGAAGCAACTCTGAACCATGCGAAAAGCGTAGGTATCCTGGGCGATGGTTTCGGCACTACT |
| | | CTGGAGATGTCCAAACGTGATCTGATGTGGGTTGTTCGCCGTACCCATGTCGCGGTTGAACGCTACCCGA |
| | | CCTGGGGCGATACGGTTGAAGTGGAATGCTGGATCGGCGCGTCCGGCAACAACGGCATGCGTCGCGATTT |
| | | CCTGGTTCGCGATTGTAAGACGGGCGAGATTCTGACCCGTTGCACGTCCCTGAGCGTTCTGATGAATACC |
| | | CGTACCCGTCGTCTGAGCACCATCCCGGACGAAGTTCGCGGTGAAATTGGCCCGGCATTCATCGATAACG |
| | | TTGCAGTAAAAGACGATGAAATCAAGAAACTGCAGAAACTGAATGACTCTACCGCGGACTACATCCAGGG |
| | | TGGTCTGACCCCGCGCTGGAACGACCTGGACGTGAACCAGCACGTCAACAACCTGAAATACGTAGCTTGG |
| | | GTATTCGAAACGGTCCCGGATTCTATCTTCGAATCTCACCACATCAGCTCCTTCACCCTGGAATACCGTC |
| | | AAGCTGACTGACTCTTTCCGCGGCATCTCTGTTATCCCGGCAGAGCCTCGTGTGTAAGAGCTCGAGGAGG |
| | | TTTTTACAATGACCAGCGATGTTCACGACGCCACAGACGGCGTCACCGAAACCGCACTCGACGACGAGCA |
| | | GTCGACCCGCCGCATCGCCGAGCTGTACGCCACCGATCCCGAGTTCGCCGCCGCCGCACCGTTGCCCGCC |
| | | GTGGTCGACGCGGCGCACAAACCCGGGCTGCGGCTGGCAGAGATCCTGCAGACCCTGTTCACCGGCTACG |
| | | GTGACCGCCCGGCGCTGGGATACCGCGCCCGTGAACTGGCCACCGACGAGGGCGGGCGCACCGTGACGCG |
| | | TCTGCTGCCGCGGTTCGACACCCTCACCTACGCCCAGGTGTGGTCGCGCGTGCAAGCGGTCGCCGCGGCC |
| | | CTGCGCCACAACTTCGCGCAGCCGATCTACCCCGGCGACGCCGTCGCGACGATCGGTTTCGCGAGTCCCG |
| | | ATTACCTGACGCTGGATCTCGTATGCGCCTACCTGGGCCTCGTGAGTGTTCCGCTGCAGCACAACGCACC |
| | | GGTCAGCCGGCTCGCCCCGATCCTGGCCGAGGTCGAACCGCGGATCCTCACCGTGAGCGCCGAATACCTC |
| | | GACCTCGCAGTCGAATCCGTGCGGGACGTCAACTCGGTGTCGCAGCTCGTGGTGTTCGACCATCACCCCG |
| | | AGGTCGACGACCACCGCGACGCACTGGCCCGCGCGCGTGAACAACTCGCCGGCAAGGGCATCGCCGTCAC |
| | | CACCCTGGACGCGATCGCCGACGAGGGCGCCGGGCTGCCGGCCGAACCGATCTACACCGCCGACCATGAT |
| | | CAGCGCCTCGCGATGATCCTGTACACCTCGGGTTCCACCGGCGCACCCAAGGGTGCGATGTACACCGAGG |
| | | CGATGGTGGCGCGGCTGTGGACCATGTCGTTCATCACGGGTGACCCCACGCCGGTCATCAACGTCAACTT |
| | | CATGCCGCTCAACCACCTGGGCGGGCGCATCCCCATTTCCACCGCCGTGCAGAACGGTGGAACCAGTTAC |
| | | TTCGTACCGGAATCCGACATGTCCACGCTGTTCGAGGATCTCGCGCTGGTGCGCCCGACCGAACTCGGCC |
| | | TGGTTCCGCGCGTCGCCGACATGCTCTACCAGCACCACCTCGCCACCGTCGACCGCCTGGTCACGCAGGG |
| | | CGCCGACGAACTGACCGCCGAGAAGCAGGCCGGTGCCGAACTGCGTGAGCAGGTGCTCGGCGGACGCGTG |
| | | ATCACCGGATTCGTCAGCACCGCACCGCTGGCCGCGGAGATGAGGGCGTTCCTCGACATCACCXTGGGCG |
| | | CACACATCGTCGACGGCTACGGGCTCACCGAGACCGGCGCCGTGACACGCGACGGTGTGATCGTGCGGCC |
| | | ACCGGTGATCGACTACAAGCTGATCGACGTTCCCGAACTCGGCTACTTCAGCACCGACAAGCCCTACCCG |
| | | CGTGGCGAACTGCTGCTCAGGTCGCAAACGCTGACTCCCGGGTACTACAAGCGCCCCGAGGTCACCGCGA |
| | | GCGTCTTCGACCGGGACGGCTACTACCACACCGGCGACGTCATGGCCGAGACCGCACCCGACCACCTGGT |
| | | GTACGTGGACCGTCGCAACAACGTCCTCAAACTCGCGCAGGGCGAGTTCGTGGCGGTCGCCAACCTGGAG |
| | | GCGGTGTTCTCCGGCGCGGCGCTGOTGCGCCAGATCTTCGTGTACGGCAACAGCGAGCGCAGTTTCCTTC |
| | | TGGCCGTGGTGGTCCCGACGCCGGAGGCGCTCGAGCAGTACGATCCGGCCGCGCTCAAGGCCGCGCTGGC |
| | | CGACTCGCTGCAGCGCACCGCACGCGACGCCGAACTGCAATCCTACGAGGTGCCGGCCGATTTCATCGTC |
| | | GAGACCGAGCCGTTCAGCGCCGCCAACGGGCTGCTGTCGGGTGTCGGAAAACTGCTGCGGCCCAACCTCA |
| | | AAGACCGCTACGGGCAGCGCCTGGAGCAGATGTACGCCGATATCGCGGCCACGCAGGCCAACCAGTTGCG |
| | | CGAACTGCGGCGCGCGGCCGCCACACAACCGGTGATCGACACCCTCACCCAGGCCGCTGCCACGATCCTC |
| | | GGCACCGGGAGCGAGGTGGCATCCGACGCCCACTTCACCGACCTGGGCGGGGATTCCCTGTCGGCGCTGA |
| | | CACTTTCGAACCTGCTGAGCGATTTCTTCGGTTTCGAAGTTCCCGTCGGCACCATCGTGAACCCGGCCAC |
| | | CAACCTCGCCCAACTCGCCCAGCACATCGAGGCGCAGCGCACCGCGGGTGACCGCAGGCCGAGTTTCACC |
| | | ACCGTGCACGGCGCGGACGCCACCGAGATCCGGGCGAGTGAGCTGACCCTGGACAAGTTCATCGACGCCG |
| | | AAACGCTCCGGGCCGCACCGGGTCTGCCCAAGGTCACCACCGAGCCACGGACGGTGTTGCTCTCGGGCGC |
| | | CAACGGCTGGCTGGGCCGGTTCCTCACGTTGCAGTGGCTGGAACGCCTGGCACCTGTCGGCGGCACCCTC |
| | | ATCACGATCGTGCGGGGCCGCGACGACGCCGCGGCCCGCGCACGGCTGACCCAGGCCTACGACACCGATC |
| | | CCGAGTTGTCCCGCCGCTTCGCCGAGCTGGCCGACCGCCACCTGCGGGTGGTCGCCGGTGACATCGGCGA |
| | | CCCGAATCTGGGCCTCACACCCGAGATCTGGCACCGGCTCGCCGCCGAGGTCGACCTGGTGGTGCATCCG |
| | | GCAGCGCTGGTCAACCACGTGCTCCCCTACCGGCAGCTGTTCGGCCCCAACGTCGTGGGCACGGCCGAGG |
| | | TGATCAAGCTGGCCCTCACCGAACGGATCAAGCCCGTCACGTACCTGTCCACCGTGTCGGTGGCCATGGG |
| | | GATCCCCGACTTCGAGGAGGACGGCGACATCCGGACCGTGAGCCCGGTGCGCCCGCTCGACGGCGGATAC |
| | | GCCAACGGCTACGGCAACAGCAAGTGGGCCGGCGAGGTGCTGCTGCGGGAGGCCCACGATCTGTGCGGGC |
| | | TGCCCGTGGCGACGTTCCGCTCGGACATGATCCTGGCGCATCCGCGCTACCGCGGTCAGGTCAACGTGCC |
| | | AGACATGTTCACGCGACTCCTGTTGAGCCTCTTGATCACCGGCGTCGCGCCGCGGTCGTTCTACATCGGA |
| | | GACGGTGAGCGCCCGCGGGCGCACTACCCCGGCCTGACGGTCGATTTCGTGGCCGAGGCGGTCACGACGC |
| | | TCGGCGCGCAGCAGCGCGAGGGATACGTGTCCTACGACGTGATGAACCCGCACGACGACGGGATCTCCCT |
| | | GGATGTGTTCGTGGACTGGCTGATCCGGGCGGGCCATCCGATCGACCGGGTCGACGACTACGACGACTGG |
| | | GTGCGTCGGTTCGAGACCGCGTTGACCGCGCTTCCCGAGAAGCGCCGCGCACAGACCGTACTGCCGCTGC |
| | | TGCACGCGTTCCGCGCTCCGCAGGCACCGTTGCGCGGCGCACCCGAACCCACGGAGGTGTTCCACGCCGC |
| | | GGTGCGCACCGCGAAGGTGGGCCCGGGAGACATCCCGCACCTCGACGAGGCGCTGATCGACAAGTACATA |
| | | CGCGATCTGCGTGAGTTCGGTCTGATCTGAGGTACCCACAAGGAGGTTTTTACAATGAAAACGACCCACA |
| | | CCAGCTTACCATTTGCCGGCCACACGTTACATTTCGTCGAATTTGATCCGGCGAACTTTTGTGAACAAGA |
| | | CCTGTTGTGGCTGCCGCATTATGCCCAGCTGCAGCACGCAGGCCGTAAGCGTAAAACTGAACATCTGGCC |
| | | GGTCGCATTGCGGCAGTGTATGCCCTGCGCGAGTACGGCTACAAATGCGTGCCGGCCATTGGTGAACTGC |
| | | GTCAACCGGTTTGGCCGGCAGAAGTTTACGGTTCCATCTCCCACTGCGGTACTACCGCGTTGGCGGTTGT |
| | | GTCTCGCCAGCCGATCGGTATTGATATTGAAGAGATATTCTCTGTCCAGACGGCACGCGAGCTGACGGAC |
| | | AACATCATTACCCCGGCAGAGCACGAGCGTCTGGCGGACTGTGGTCTGGCGTTCAGCCTGGCGCTGACCC |
| | | TGGCATTCAGCGCAAAAGAGAGCGCGTTCAAGGCTTCCGAGATCCAAACCGATGCGGGCTTCCTGGATTA |
| | | TCAAATCATCAGCTGGAACAAGCAACAGGTTATCATTCACCGTGAGAATGAGATGTTTGCCGTCCATTGG |
| | | CAGATTAAAGAGAAAATCGTTATCACCCTGTGCCAGCACGACTGAGAATTCGGTTTTCCGTCCTGTCTTG |
| | | ATTTTCAAGCAAACAATGCCTCCGATTTCTAATCGGAGGCATTTGT1TTTGTTTATTGCAAAAACAAAAA |
| | | ATATTGTTACAAATTTTTACAGGCTATTAAGCCTACCGTCATAAATAATTTGCCATTTACTAGTTTTTAA |
| | | TTAACCAGAACCTTGACCGAACGCAGCGGTGGTAACGGCGCAGTGGCGGTTTTCATGGCTTGTTATGACT |
| | | GTTTTTTTGGGGTACAGTCTATGCCTCGGGCATCCAAGCAGCAAGCGCGTTACGCCGTGGGTCGATGTTT |
| | | GATGTTATGGAGCAGCAACGATGTTACGCAGCAGGGCAGTCGCCCTAAAACAAAGTTAAACATCATGAGG |
| | | GAAGCGGTGATCGCCGAAGTATCGACTCAACTATCAGAGGTAGTTGGCGTCATCGAGCGCCATCTCGAAC |
| | | CGACGTTGCTGGCCGTACATTTGTACGGCTCCGCAGTGGATGGCGGCCTGAAGCCACACAGTGATATTGA |
| | | TTTGCTGGTTACGGTGACCGTAAGGCTTGATGAAACAACGCGGCGAGCTTTGATCAACGACCTTTTGGAA |
| | | ACTTCGGCTTCCCCTGGAGAGAGCGAGATTCTCCGCGCTGTAGAAGTCACCATTGTTGTGCACGACGACA |
| | | TCATTCCGTGGCGTTATCCAGCTAAGCGCGAACTGCAATTTGGAGAATGGCAGCGCAATGACATTCTTGC |
| | | AGGTATCTTCGAGCCAGCCACGATCGACATTGATCTGGCTATCTTGCTGACAAAAGCAAGAGAACATAGC |
| | | GTTGCCTTGGTAGGTCCAGCGGCGGAGGAACTCTTTGATCCGGTTCCTGAACAGGATCTATTTGAGGCGC |
| | | TAAATGAAACCTTAACGCTATGGAACTCGCCGCCCGACTGGGCTGGCGATGAGCGAAATGTAGTGCTTAC |
| | | GTTGTCCCGCATTTGGTACAGCGCAGTAACCGGCAAAATCGCGCCGAAGGATGTCGCTGCCGACTGGGCA |
| | | ATGGAGCGCCTGCCGGCCCAGTATCAGCCCGTCATACTTGAAGCTAGACAGGCTTATCTTGGACAAGAAG |
| | | AAGATCGCTTGGCCTCGCGCGCAGATCAGTTGGAAGAATTTGTCCACTACGTGAAAGGCGAGATCACCAA |
| | | GGTAGTCGGCAAATAATGTCTAACAATTCGTTCAAGCCGACGCCGCTTCGCGGCGCGGCTTAACTCAAGC |
| | | GTTAGATGCACTAAGCACATAATTGCTCACAGCCAAACTATCAGGTCAAGTCTGCTTTTATTATTTTTAA |
| | | GCGTGCATAATAAGCCCTACACAAATTGGGAGATATATCATGAGGCGCGCCACGAGAAAGAGTTATGACA |
| | | AATTAAAATTCTGACTCTTAGATTATTTCCAGAGAGGCTGATTTTCCCAATCTTTGGGAAAGCCTAAGTT |
| | | TTTAGATTCTATTTCTGGATACATCTCAAAAGTTCTTTTTAAATGCTGTGCAAAATTATGCTCTGGTTTA |
| | | ATTCTGTCTAAGAGATACTGAATACAACATAAGCCAGTGAAAATTTTACGGCTGTTTCTTTGATTAATAT |
| | | CCTCCAATACTTCTCTAGAGAGCCATTTTCCTTTTAACCTATCAGGCAATTTAGGTGATTCTCCTAGCTG |
| | | TATATTCCAGAGCCTTGAATGATGAGCGCAAATATTTCTAATATGCGACAAAGACCGTAACCAAGATATA |
| | | AAAAACTTGTTAGGTAATTGGAAATGAGTATGTATTTTTTGTCGTGTCTTAGATGGTAATAAATTTGTGT |
| | | ACATTCTAGATAACTGCCCAAAGGCGATTATCTCCAAAGCCATATATGACGGCGGTAGTAGAGGATTTGT |
| | | GTACTTGTTTCGATAATGCCCGATAAATTCTTCTACTTTTTTAGATTGGCAATATTGAGTAATCGAATCG |
| | | ATTAATTCTTGATGCTTCCCAGTGTCATAAAATAAACTTTTATTCAGATACCAATGAGGATCATAATCAT |
| | | GGGAGTAGTGATAAATCATTTGAGTTCTGACTGCTACTTCTATCGACTCCGTAGCATTAAAAATAAGCAT |
| | | TCTCAAGGATTTATCAAACTTGTATAGATTTGGCCGGCCCGTCAAAAGGGCGACACCCCATAATTAGCCC |
| | | GGGCGAAAGGCCCAGTCTTTCGACTGAGCCTTTCGTTTTATTTGATGCCTGGCAGTTCCCTACTCTCGCA |
| | | TGGGGAGTCCCCACACTACCATCGGCGCTACGGCGTTTCACTTCTGAGTTCGGCATGGGGTCAGGTGGGA |
| | | CCACCGCGCTACTGCCGCCAGGCAAACAAGGGGTGTTATGAGCCATATTCAGGTATAAATGGGCTCGCGA |
| | | TAATGTTCAGAATTGGTTAATTGGTTGTAACACTGACCCCTATTTGTTTATTTTTCTAAATACATTCAAA |
| | | TATGTATCCGCTCATGAGACAATAACCCTGATAAATGCTTCAATAATATTGAAAAAGGAAGAATATGAGT |
| | | ATTCAACATTTCCGTGTCGCCCTTATTCCCTTTTTTGCGGCATTTTGCCTTCCTGTTTTTGCTCACCCAG |
| | | AAACGCTGGTGAAAGTAAAAGATGCTGAAGATCAGTTGGGTGCACGAGTGGGTTACATCGAACTGGATCT |
| | | CAACAGCGGTAAGATCCTTGAGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACTCGGTCGCCGCATACACTATT |
| | | CTCAGAATGACTTGGTTGAGTACTCACCAGTCACAGAAAAGCATCTTACGGATGGCATGACAGTAAGAGA |
| | | ATTATGCAGTGCTGCCATAACCATGAGTGATAACACTGCGGCCAACTTACTTCTGACAACGATCGGAGGA |
| | | CCGAAGGAGCTAACCGCTTTTTTGCACAACATGGGGGATCATGTAACTCGCCTTGATCGTTGGGAACCGG |
| | | AGCTGAATGAAGCCATACCAAACGACGAGCGTGACACCACGATGCCTGTAGCGATGGCAACAACGTTGCG |
| | | CAAACTATTAACTGGCGAACTACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATTGCTGATAAATCCGGAGCCG |
| | | GTGAGCGTGGTTCTCGCGGTATCATCGCAGCGCTGGGGCCAGATGGTAAGCCCTCCCGTATCGTAGTTAT |
| | | CTACACGACGGGGAGTCAGGCAACTATGGATGAACGAAATAGACAGATCGCTGAGATAGGTGCCTCACTG |
| | | ATTAAGCATTGGT |
| 24 | carboxylic | ATGACCAGCGATGTTCACGACGCCACAGACGGCGTCACCGAAACCGCACTCGACGACGAGCAGTCGACCC |
| | acid | GCCGCATCGCCGAGCTGTACGCCACCGATCCCGAGTTCGCCGCCGCCGCACCGTTGCCCGCCGTGGTCGA |
| | reductase | CGCGGCGCACAAACCCGGGCTGCGGCTGGCAGAGATCCTGCAGACCCTGTTCACCGGCTACGGTGACCGC |
| | amplified | CCGGCGCTGGGATACCGCGCCCGTGAACTGGCCACCGACGAGGGCGGGCGCACCGTGACGCGTCTGCTGC |
| | from | CGCGGTTCGACACCCTCACCTACGCCCAGGTGTGGTCGCGCGTGCAAGCGGTCGCCGCGGCCCTGCGCCA |
| | Mycobacterium | CAACTTCGCGCAGCCGATCTACCCCGGCGACGCCGTCGCGACGATCGGTTTCGCGAGTCCCGATTACCTG |
| | smegmatis. | ACGCTGGATCTCGTATGCGCCTACCTGGGCCTCGTGAGTGTTCCGCTGCAGCACAACGCACCGGTCAGCC |
| | | GGCTCGCCCCGATCCTGGCCGAGGTCGAACCGCGGATCCTCACCGTGAGCGCCGAATACCTCGACCTCGC |
| | | AGTCGAATCCGTGCGGGACGTCAACTCGGTGTCGCAGCTCGTGGTGTTCGACCATCACCCCGAGGTCGAC |
| | | GACCACCGCGACGCACTGGCCCGCGCGCGTGAACAACTCGCCGGCAAGGGCATCGCCGTCACCACCCTGG |
| | | ACGCGATCGCCGACGAGGGCGCCGGGCTGCCGGCCGAACCGATCTACACCGCCGACCATGATCAGCGCCT |
| | | CGCGATGATCCTGTACACCTCGGGTTCCACCGGCGCACCCAAGGGTGCGATGTACACCGAGGCGATGGTG |
| | | GCGCGGCTGTGGACCATGTCGTTCATCACGGGTGACCCCACGCCGGTCATCAACGTCAACTTCATGCCGC |
| | | TCAACCACCTGGGCGGGCGCATCCCCATTTCCACCGCCGTGCAGAACGGTGGAACCAGTTACTTCGTACC |
| | | GGAATCCGACATGTCCACGCTGTTCGAGGATCTCGCGCTGGTGCGCCCGACCGAACTCGGCCTGGTTCCG |
| | | CGCGTCGCCGACATGCTCTACCAGCACCACCTCGCCACCGTCGACCGCCTGGTCACGCAGGGCGCCGACG |
| | | AACTGACCGCCGAGAAGCAGGCCGGTGCCGAACTGCGTGAGCAGGTGCTCGGCGGACGCGTGATCACCGG |
| | | ATTCGTCAGCACCGCACCGCTGGCCGCGGAGATGAGGGCGTTCCTCGACATCACCCTGGGCGCACACATC |
| | | GTCGACGGCTACGGGCTCACCGAGACCGGCGCCGTGACACGCGACGGTGTGATCGTGCGGCCACCGGTGA |
| | | TCGACTACAAGCTGATCGACGTTCCCGAACTCGGCTACTTCAGCACCGACAAGCCCTACCCGCGTGGCGA |
| | | ACTGCTGGTCAGGTCGCAAACGCTGACTCCCGGGTACTACAAGCGCCCCGAGGTCACCGCGAGCGTCTTC |
| | | GACCGGGACGGCTACTACCACACCGGCGACGTCATGGCCGAGACCGCACCCGACCACCTGGTGTACGTGG |
| | | ACCGTCGCAACAACGTCCTCAAACTCGCGCAGGGCGAGTTCGTGGCGGTCGCCAACCTGGAGGCGGTGTT |
| | | CTCCGGCGCGGCGCTGGTGCGCCAGATCTTCGTGTACGGCAACAGCGAGCGCAGTTTCCTTCTGGCCGTG |
| | | GTGGTCCCGACGCCGGAGGCGCTCGAGCAGTACGATCCGGCCGCGCTCAAGGCCGCGCTGGCCGACTCGC |
| | | TGCAGCGCACCGCACGCGACGCCGAACTGCAATCCTACGAGGTGCCGGCCGATTTCATCGTCGAGACCGA |
| | | GCCGTTCAGCGCCGCCAACGGGCTGCTGTCGGGTGTCGGAAAACTGCTGCGGCCCAACCTCAAAGACCGC |
| | | TACGGGCAGCGCCTGGAGCAGATGTACGCCGATATCGCGGCCACGCAGGCCAACCAGTTGCGCGAACTGC |
| | | GGCGCGCGGCCGCCACACAACCGGTGATCGACACCCTCACCCAGGCCGCTGCCACGATCCTCGGCACCGG |
| | | GAGCGAGGTGGCATCCGACGCCCACTTCACCGACCTGGGCGGGGATTCCCTGTCGGCGCTGACACTTTCG |
| | | AACCTGCTGAGCGATTTCTTCGGTTTCGAAGTTCCCGTCGGCACCATCGTGAACCCGGCCACCAACCTCG |
| | | CCCAACTCGCCCAGCACATCGAGGCGCAGCGCACCGCGGGTGACCGCAGGCCGAGTTTCACCACCGTGCA |
| | | CGGCGCGGACGCCACCGAGATCCGGGCGAGTGAGCTGACCCTGGACAAGTTCATCGACGCCGAAACGCTC |
| | | CGGGCCGCACCGGGTCTGCCCAAGGTCACCACCGAGCCACGGACGGTGTTGCTCTCGGGCGCCAACGGCT |
| | | GGCTGGGCCGGTTCCTCACGTTGCAGTGGCTGGAACGCCTGGCACCTGTCGGCGGCACCCTCATCACGAT |
| | | CGTGCGGGGCCGCGACGACGCCGCGGCCCGCGCACGGCTGACCCAGGCCTACGACACCGATCCCGAGTTG |
| | | TCCCGCCGCTTCGCCGAGCTGGCCGACCGCCACCTGCGGGTGGTCGCCGGTGACATCGGCGACCCGAATC |
| | | TGGGCCTCACACCCGAGATCTGGCACCGGCTCGCCGCCGAGGTCGACCTGGTGGTGCATCCGGCAGCGCT |
| | | GGTCAACCACGTGCTCCCCTACCGGCAGCTGTTCGGCCCCAACGTCGTGGGCACGGCCGAGGTGATCAAG |
| | | CTGGCCCTCACCGAACGGATCAAGCCCGTCACGTACCTGTCCACCGTGTCGGTGGCCATGGGGATCCCCG |
| | | ACTTCGAGGAGGACGGCGACATCCGGACCGTGAGCCCGGTGCGCCCGCTCGACGGCGGATACGCCAACGG |
| | | CTACGGCAACAGCAAGTGGGCCGGCGAGGTGCTGCTGCGGGAGGCCCACGATCTGTGCGGGCTGCCCGTG |
| | | GCGACGTTCCGCTCGGACATGATCCTGGCGCATCCGCGCTACCGCGGTCAGGTCAACGTGCCAGACATGT |
| | | TCACGCGACTCCTGTTGAGCCTCTTGATCACCGGCGTCGCGCCGCGGTCGTTCTACATCGGAGACGGTGA |
| | | GCGCCCGCGGGCGCACTACCCCGGCCTGACGGTCGATTTCGTGGCCGAGGCGGTCACGACGCTCGGCGCG |
| | | CAGCAGCGCGAGGGATACGTGTCCTACGACGTGATGAACCCGCACGACGACGGGATCTCCCTGGATGTGT |
| | | TCGTGGACTGGCTGATCCGGGCGGGCCATCCGATCGACCGGGTCGACGACTACGACGACTGGGTGCGTCG |
| | | GTTCGAGACCGCGTTGACCGCGCTTCCCGAGAAGCGCCGCGCACAGACCGTACTGCCGCTGCTGCACGCG |
| | | TTCCGCGCTCCGCAGGCACCGTTGCGCGGCGCACCCGAACCCACGGAGGTGTTCCACGCCGCGGTGCGCA |
| | | CCGCGAAGGTGGGCCCGGGAGACATCCCGCACCTCGACGAGGCGCTGATCGACAAGTACATACGCGATCT |
| | | GCGTGAGTTCGGTCTGATCTGA |
| 25 | codon- | ATGCAGCAACTGACCGATCAAAGCAAAGAACTGGACTTCAAGAGCGAGACGTACAAAGACGCCTATAGCC |
| | optimized | GCATTAACGCGATCGTCATTGAAGGCGAACAAGAGGCGCATGAAAACTACATCACCCTGGCGCAGCTGCT |
| | Nostoc | GCCTGAGAGCCACGACGAACTGATTCGCCTGAGCAAAATGGAGAGCCGTCACAAGAAAGGTTTTGAGGCG |
| | punctiforme | TGTGGCCGCAATCTGGCGGTGACCCCGGACCTGCAATTTGCGAAGGAGTTCTTTAGCGGTCTGCACCAGA |
| | adm. | ATTTCCAGACGGCCGCAGCCGAGGGCAAAGTCGTCACTTGTTTGTTGATCCAGAGCCTGATTATTGAATG |
| | | CTTTGCTATTGCGGCGTACAACATTTACATTCCGGTCGCCGATGACTTTGCGCGTAAAATCACGGAAGGT |
| | | GTTGTCAAAGAGGAGTATTCCCACCTGAATTTCGGTGAAGTGTGGTTGAAGGAACATTTTGCGGAATCTA |
| | | AAGCCGAATTGGAACTGGCAAATCGCCAGAACCTGCCGATCGTTTGGAAGATGCTGAACCAAGTGGAAGG |
| | | TGATGCACATACGATGGCGATGGAGAAGGACGCATTGGTTGAGGACTTTATGATTCAGTATGGCGAAGCA |
| | | CTGTCCAATATCGGTTTCAGCACCCGTGATATCATGCGTCTGAGCGCCTATGGCCTGATCGGTGCCTAA |
| 26 | codon- | ATGGAGTGGAAACCAAAACCGAAACTGCCTCAGCTGCTGGATGACCACTTCGGTCTGCACGGCCTGGTTT |
| | optimized | TCCGTCGTACCTTCGCTATCCGTTCTTACGAAGTCGGCCCTGATCGCTCCACCTCCATCCTGGCGGTAAT |
| | Umbellularia | GAACCACATGCAGGAAGCAACTCTGAACCATGCGAAAAGCGTAGGTATCCTGGGCGATGGTTTCGGCACT |
| | californicia | ACTCTGGAGATGTCCAAACGTGATCTGATGTGGGTTGTTCGCCGTACCCATGTCGCGGTTGAACGCTACC |
| | fatBₘ (without | CGACCTGGGGCGATACGGTTGAAGTGGAATGCTGGATCGGCGCGTCCGGCAACAACGGCATGCGTCGCGA |
| | leader | TTTCCTGGTTCGCGATTGTAAGACGGGCGAGATTCTGACCCGTTGCACGTCCCTGAGCGTTCTGATGAAT |
| | sequence). | ACCCGTACCCGTCGTCTGAGCACCATCCCGGACGAAGTTCGCGGTGAAATTGGCCCGGCATTCATCGATA |
| | | ACGTTGCAGTAAAAGACGATGAAATCAAGAAACTGCAGAAACTGAATGACTCTACCGCGGACTACATCCA |
| | | GGGTGGTCTGACCCCGCGCTGGAACGACCTGGACGTGAACCAGCACGTCAACAACCTGAAATACGTAGCT |
| | | TGGGTATTCGAAACGGTCCCGGATTCTATCTTCGAATCTCACCACATCAGCTCCTTCACCCTGGAATACC |
| | | GTCGTGAGTGTACCCGTGACTCCGTTCTGCGCTCTCTGACCACGGTATCCGGCGGTAGCTCTGAAGCCGG |
| | | TCTGGTTTGCGATCACCTGCTGCAGCTGGAAGGCGGCAGCGAGGTTCTGCGTGCTCGTACTGAGTGGCGT |
| | | CCGAAGCTGACTGACTCTTTCCGCGGCATCTCTGTTATCCCGGCAGAGCCTCGTGTGTAA |
| 27 | codon- | ATGGACCGTAAAAGCAAGCGTCCGGACATGCTGGTTGATTCCTTTGGTCTGGAAAGCACCGTGCAGGACG |
| | optimized | GTCTGGTTTTCCGTCAGTCTTTCTCCATTCGTAGCTATGAGATTGGTACTGATCGTACCGCCTCTATCGA |
| | Cuphea | AACCCTGATGAATCACCTGCAAGAAACCTCTCTGAACCATTGTAAGTCTACTGGCATCCTGCTGGACGGT |
| | hookeriana | TTCGGTCGTACCCTGGAGATGTGCAAACGCGACCTGATTTGGGTAGTGATCAAAATGCAGATCAAAGTTA |
| | fatB2ₘ | ACCGTTATCCGGCATGGGGTGATACCGTTGAAATCAACACCCGCTTTTCTCGTCTGGGCAAAATCGGTAT |
| | (without | GGGCCGTGACTGGCTGATCTCTGACTGTAACACTGGTGAAATTCTGGTTCGTGCTACTAGCGCATACGCG |
| | leader | ATGATGAACCAGAAAACCCGTCGCCTGAGCAAGCTGCCGTACGAGGTCCACCAGGAGATTGTTCCGCTGT |
| | sequence). | TTGTAGACAGCCCAGTGATTGAGGATTCTGACCTGAAAGTGCATAAATTCAAAGTGAAGACCGGTGACAG |
| | | CATCCAAAAAGGCCTGACCCCAGGTTGGAACGATCTGGACGTTAACCAGCACGTTTCCAACGTGAAGTAT |
| | | ATCGGTTGGATTCTGGAGAGCATGCCGACCGAGGTCCTGGAAACCCAGGAGCTGTGTTCCCTGGCGCTGG |
| | | AGTACCGCCGTGAGTGCGGCCGTGACAGCGTGCTGGAGTCTGTGACCGCTATGGACCCAAGCAAAGTTGG |
| | | TGTTCGTAGCCAGTACCAGCACCTGCTGCGTCTGGAAGACGGTACTGCTATCGTGAACGGTGCAACTGAA |
| | | TGGCGTCCTAAAAACGCGGGTGCAAACGGTGCTATCAGCACCGGTAAAACCTCTAACGGTAACTCCGTGA |
| | | GCTAA |
| 28 | codon- | ATGAAAACGACCCACACCAGCTTACCATTTGCCGGCCACACGTTACATTTCGTCGAATTTGATCCGGCGA |
| | optimized | ACTTTTGTGAACAAGACCTGTTGTGGCTGCCGCATTATGCCCAGCTGCAGCACGCAGGCCGTAAGCGTAA |
| | E. coli entD. | AACTGAACATCTGGCCGGTCGCATTGCGGCAGTGTATGCCCTGCGCGAGTACGGCTACAAATGCGTGCCG |
| | | GCCATTGGTGAACTGCGTCAACCGGTTTGGCCGGCAGAAGTTTACGGTTCCATCTCCCACTGCGGTACTA |
| | | CCGCGTTGGCGGTTGTGTCTCGCCAGCCGATCGGTATTGATATTGAAGAGATATTCTCTGTCCAGACGGC |
| | | ACGCGAGCTGACGGACAACATCATTACCCCGGCAGAGCACGAGCGTCTGGCGGACTGTGGTCTGGCGTTC |
| | | AGCCTGGCGCTGACCCTGGCATTCAGCGCAAAAGAGAGCGCGTTCAAGGCTTCCGAGATCCAAACCGATG |
| | | CGGGCTTCCTGGATTATCAAATCATCAGCTGGAACAAGCAACAGGTTATCATTCACCGTGAGAATGAGAT |
| | | GTTTGCCGTCCATTGGCAGATTAAAGAGAAAATCGTTATCACCCTGTGCCAGCACGACTGA |
| 29 | plasmid | TAGAAAAACTCATCGAGCATCAAATGAAACTGCAATTTATTCATATCAGGATTATCAATACCATATTTTT |
| | pAQ4::P(cpcB)- | GAAAAAGCCGTTTCTGTAATGAAGGAGAAAACTCACCGAGGCAGTTCCATAGGATGGCAAGATCCTGGTA |
| | adm_{Npu}-ermC. | TCGGTCTGCGATTCCGACTCGTCCAACATCAATACAACCTATTAATTTCCCCTCGTCAAAAATAAGGTTA |
| | | TCAAGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATTCAT |
| | | TCGTGATTGCGCCTGAGCGAGGCGAAATACGCGATCGCTGTTAAAAGGACAATTACAAACAGGAATCGAG |
| | | TGCAACCGGCGCAGGAACACTGCCAGCGCATCAACAATATTTTCACCTGAATCAGGATATTCTTCTAATA |
| | | CCTGGAACGCTGTTTTTCCGGGGATCGCAGTGGTGAGTAACCATGCATCATCAGGAGTACGGATAAAATG |
| | | CTTGATGGTCGGAAGTGGCATAAATTCCGTCAGCCAGTTTAGTCTGACCATCTCATCTGTAACATCATTG |
| | | GCAACGCTACCTTTGCCATGTTTCAGAAACAACTCTGGCGCATCGGGCTTCCCATACAAGCGATAGATTG |
| | | TCGCACCTGATTGCCCGACATTATCGCGAGCCCATTTATACCCATATAAATCAGCATCCATGTTGGAATT |
| | | TAATCGCGGCCTCGACGTTTCCCGTTGAATATGGCTCATATTCTTCCTTTTTCAATATTATTGAAGCATT |
| | | TATCAGGGTTATTGTCTCATGAGCGGATACATATTTGAATGTATTTAGAAAAATAAACAAATAGGGGTCA |
| | | GTGTTACAACCAATTAACCAATTCTGAACATTATCGCGAGCCCATTTATACCTGAATATGGCTCATAACA |
| | | CCCCTTGTTTGCCTGGCGGCAGTAGCGCGGTGGTCCCACCTGACCCCATGCCGAACTCAGAAGTGAAACG |
| | | CCGTAGCGCCGATGGTAGTGTGGGGACTCCCCATGCGAGAGTAGGGAACTGCCAGGCATCAAATAAAACG |
| | | AAAGGCTCAGTCGAAAGACTGGGCCTTTCGCCCGGGCTAATTAGGGGGTGTCGCCCTTTACACGTACTTA |
| | | GTCGCTGAAGGCCTCACTGGCCCCTGCAGGGATGGTGGAATGCTGGTTATCTGGTGGGGATTAAGTGGTG |
| | | TTTTACTAAAGCTTGAACAACTCAAGAAAGATTATATTCGCAATAACTGCCAATAATCCCAGCATCTTGA |
| | | GAAAATCCAGCAAACCGGGGGCAAAACACCAGCAAGAAGCCAGCAGACTATCACCAAATCCCCAGCGTAC |
| | | AGCTAGAAATAACTGAGCAGTTGTATTCAATTACCTTCTGGTCAAGCCGAGGAAATTTCCCCACACCTTA |
| | | TACACCTCTGGAAGGTTTTTTTGACGAAGCGCAAAATATCCACAATCGGCTGGGGACTTCTTCTGTCAGA |
| | | AAATGGCAGAAATTTTTGAATGTGTTGGCGATCGCCCTCATCAATGATTATTAGAGAACTTTTGTCCCTG |
| | | ATGTTGGGAATACTCTTGATGACAATTGTGATTGCTCAAAGAAGAAAGAAATTTGGAGTAAATCTCTAAA |
| | | AGGGGACTGAAATATTTGTATGGTCAGCATGACCACTGAAATGGAGAGAAGTCTAAGACAGTAGATGTCT |
| | | TAGATATAAGCCTCATTAGAAGCCATGCCATAAAACAGATTTTGTGGATGAAACAACTTGAAATAGTTCA |
| | | GTTGTAGACCATGTTATAAACATTTATTCTTAACACAGTGACACATTAATGACTCATATATCCGTCCAAA |
| | | AAAAACTAAAATGTTTGTAAATTTAGTTTTGCGGCCGCGTCGACTTCGTTATAAAATAAACTTAACAAAT |
| | | CTATACCCACCTGTAGAGAAGAGTCCCTGAATATCAAAATGGTGGGATAAAAAGCTCAAAAAGGAAAGTA |
| | | GGCTGTGGTTCCCTAGGCAACAGTCTTCCCTACCCCACTGGAAACTAAAAAAACGAGAAAAGTTCGCACC |
| | | GAACATCAATTGCATAATTTTAGCCCTAAAACATAAGCTGAACGAAACTGGTTGTCTTCCCTTCCCAATC |
| | | CAGGACAATCTGAGAATCCCCTGCAACATTACTTAACAAAAAAGCAGGAATAAAATTAACAAGATGTAAC |
| | | AGACATAAGTCCCATCACCGTTGTATAAAGTTAACTGTGGGATTGCAAAAGCATTCAAGCCTAGGCGCTG |
| | | AGCTGTTTGAGCATCCCGGTGGCCCTTGTCGCTGCCTCCGTGTTTCTCCCTGGATTTATTTAGGTAATAT |
| | | CTCTCATAAATCCCCGGGTAGTTAACGAAAGTTAATGGAGATCAGTAACAATAACTCTAGGGTCATTACT |
| | | TTGGACTCCCTCAGTTTATCCGGGGGAATTGTGTTTAAGAAAATCCCAACTCATAAAGTCAAGTAGGAGA |
| | | TTAATCATATGCAGCAACTGACCGATCAAAGCAAAGAACTGGACTTCAAGAGCGAGACGTACAAAGACGC |
| | | CTATAGCCGCATTAACGCGATCGTCATTGAAGGCGAACAAGAGGCGCATGAAAACTACATCACCCTGGCG |
| | | CAGCTGCTGCCTGAGAGCCACGACGAACTGATTCGCCTGAGCAAAATGGAGAGCCGTCACAAGAAAGGTT |
| | | TTGAGGCGTGTGGCCGCAATCTGGCGGTGACCCCGGACCTGCAATTTGCGAAGGAGTTCTTTAGCGGTCT |
| | | GCACCAGAATTTCCAGACGGCCGCAGCCGAGGGCAAAGTCGTCACTTGTTTGTTGATCCAGAGCCTGATT |
| | | ATTGAATGCTTTGCTATTGCGGCGTACAACATTTACATTCCGGTCGCCGATGACTTTGCGCGTAAAATCA |
| | | CGGAAGGTGTTGTCAAAGAGGAGTATTCCCACCTGAATTTCGGTGAAGTGTGGTTGAAGGAACATTTTGC |
| | | GGAATCTAAAGCCGAATTGGAACTGGCAAATCGCCAGAACCTGCCGATCGTTTGGAAGATGCTGAACCAA |
| | | GTGGAAGGTGATGCACATACGATGGCGATGGAGAAGGACGCATTGGTTGAGGACTTTATGATTCAGTATG |
| | | GCGAAGCACTGTCCAATATCGGTTTCAGCACCCGTGATATCATGCGTCTGAGCGCCTATGGCCTGATCGG |
| | | TGCCTAACTCGAGCAATTCGGTTTTCCGTCCTGTCTTGATTTTCAAGCAAACAATGCCTCCGATTTCTAA |
| | | TCGGAGGCATTTGTTTTTGTTTATTGCAAAAACAAAAAATATTGTTACAAATTTTTACAGGCTATTAAGC |
| | | CTACCGTCATAAATAATTTGCCATTTACTAGTTTTAATTAACGTGCTATAATTATACTAATTTTATAAGG |
| | | AGGAAAAAATATGGGCATTTTTAGTATTTTTGTAATCAGCACAGTTCATTATCAACCAAACAAAAAATAA |
| | | GTGGTTATAATGAATCGTTAATAAGCAAAATTCATATAACCAAATTAAAGAGGGTTATAATGAACGAGAA |
| | | AAATATAAAACACAGTCAAAACTTTATTACTTCAAAACATAATATAGATAAAATAATGACAAATATAAGA |
| | | TTAAATGAACATGATAATATCTTTGAAATCGGCTCAGGAAAAGGCCATTTTACCCTTGAATTAGTAAAGA |
| | | GGTGTAATTTCGTAACTGCCATTGAAATAGACCATAAATTATGCAAAACTACAGAAAATAAACTTGTTGA |
| | | TCACGATAATTTCCAAGTTTTAAACAAGGATATATTGCAGTTTAAATTTCCTAAAAACCAATCCTATAAA |
| | | ATATATGGTAATATACCTTATAACATAAGTACGGATATAATACGCAAAATTGTTTTTGATAGTATAGCTA |
| | | ATGAGATTTATTTAATCGTGGAATACGGGTTTGCTAAAAGATTATTAAATACAAAACGCTCATTGGCATT |
| | | ACTTTTAATGGCAGAAGTTGATATTTCTATATTAAGTATGGTTCCAAGAGAATATTTTCATCCTAAACCT |
| | | AAAGTGAATAGCTCACTTATCAGATTAAGTAGAAAAAAATCAAGAATATCACACAAAGATAAACAAAAGT |
| | | ATAATTATTTCGTTATGAAATGGGTTAACAAAGAATACAAGAAAATATTTACAAAAAATCAATTTAACAA |
| | | TTCCTTAAAACATGCAGGAATTGACGATTTAAACAATATTAGCTTTGAACAATTCTTATCTCTTTTCAAT |
| | | AGCTATAAATTATTTAATAAGTAAGTTAAGGGATGCATAAACTGCATCCCTTAACTTGTTTTTCGTGTGC |
| | | CTATTTTTTGTGGCGCGCCCAGTTTCCTTTACTGGCCCTAAAGTCGCTGTGGCTAGGGTTCCGAAGGGGC |
| | | ATTATTGGCTCGCGGCTTTACAACCTTGATAAGGAGAGAGATGACAGTTTTTTTTCTCTTTTGCTTAGTA |
| | | AAACAGCAAATTTAAGGCATGTTAAAGAGCAGTAGAACGAAATGGTTGAGCCGGCCTCGATACACTCAAT |
| | | TAACTACTAATAGCTTCAATAAATTTTGGGACGATTGAAGCTATTTTTTTGAAAATCAACTCTTAATATC |
| | | TCCTGTCTCAAAAGAGTTAATTGCTAAACAAAAGCCAGTTTCAGCGAAAAATCTAGAGTTTTATAGGTTC |
| | | GTTCTCAGTACAGGACAAAAAGTTTGAAAAGGATAGAGGGAGAGGGTTTGATGGAAATAAGCACAAATCA |
| | | ATCAAGCCCTCATGAATCAGATTAGCGAAATTCGCCGCCAATTGCGACCTCATCTCGGATGGCATGGAGC |
| | | CAGACTGTCATTTATCGCCCTCTTCCTGGTGGCACTGTTCCGAGCAAAAACCGTCAATCTCGCCAAACTC |
| | | GCCACCGTCTGGGGAGGCAATGCAGCAGAAGAGTCTAATTACAAACGCATGCAGCGATTCTTTCAGTCCT |
| | | TTGACGTCAACATGGACAAAATCGCCAGGATGGTAATGAATATCGCGGCTATCCCGCAACCTTGGGTCTT |
| | | AAGCATCGACCGCACCAACGGCCGGCCTACATGGCCCGTCAATCGAAGGGCGACACAAAATTTATTCTAA |
| | | ATGCATAATAAATACTGATAACATCTTATAGTTTGTATTATATTTTGTATTATCGTTGACATGTATAATT |
| | | TTGATATCAAAAACTGATTTTCCCTTTATTATTTTCGAGATTTATTTTCTTAATTCTCTTTAACAAACTA |
| | | GAAATATTGTATATACAAAAAATCATAAATAATAGATGAATAGTTTAATTATAGGTGTTCATCAATCGAA |
| | | AAAGCAACGTATCTTATTTAAAGTGCGTTGCTTTTTTCTCATTTATAAGGTTAAATAATTCTCATATATC |
| | | AAGCAAAGTGACAGGCGCCCTTAAATATTCTGACAAATGCTCTTTCCCTAAACTCCCCCCATAAAAAAAC |
| | | CCGCCGAAGCGGGTTTTTACGTTATTTGCGGATTAACGATTACTCGTTATCAGAACCGCCCAGGGGGCCC |
| | | GAGCTTAAGACTGGCCGTCGTTTTACAACACAGAAAGAGTTTGTAGAAACGCAAAAAGGCCATCCGTCAG |
| | | GGGCCTTCTGCTTAGTTTGATGCCTGGCAGTTCCCTACTCTCGCCTTCCGCTTCCTCGCTCACTGACTCG |
| | | CTGCGCTCGGTCGTTCGGCTGCGGCGAGCGGTATCAGCTCACTCAAAGGCGGTAATACGGTTATCCACAG |
| | | AATCAGGGGATAACGCAGGAAAGAACATGTGAGCAAAAGGCCAGCAAAAGGCCAGGAACCGTAAAAAGGC |
| | | CGCGTTGCTGGCGTTTTTCCATAGGCTCCGCCCCCCTGACGAGCATCACAAAAATCGACGCTCAAGTCAG |
| | | AGGTGGCGAAACCCGACAGGACTATAAAGATACCAGGCGTTTCCCCCTGGAAGCTCCCTCGTGCGCTCTC |
| | | CTGTTCCGACCCTGCCGCTTACCGGATACCTGTCCGCCTTTCTCCCTTCGGGAAGCGTGGCGCTTTCTCA |
| | | TAGCTCACGCTGTAGGTATCTCAGTTCGGTGTAGGTCGTTCGCTCCAAGCTGGGCTGTGTGCACGAACCC |
| | | CCCGTTCAGCCCGACCGCTGCGCCTTATCCGGTAACTATCGTCTTGAGTCCAACCCGGTAAGACACGACT |
| | | TATCGCCACTGGCAGCAGCCACTGGTAACAGGATTAGCAGAGCGAGGTATGTAGGCGGTGCTACAGAGTT |
| | | CTTGAAGTGGTGGGCTAACTACGGCTACACTAGAAGAACAGTATTTGGTATCTGCGCTCTGCTGAAGCCA |
| | | GTTACCTTCGGAAAAAGAGTTGGTAGCTCTTGATCCGGCAAACAAACCACCGCTGGTAGCGGTGGTTTTT |
| | | TTGTTTGCAAGCAGCAGATTACGCGCAGAAAAAAAGGATCTCAAGAAGATCCTTTGATCTTTTCTACGGG |
| | | GTCTGACGCTCAGTGGAACGACGCGCGCGTAACTCACGTTAAGGGATTTTGGTCATGAGCTTGCGCCGTC |
| | | CCGTCAAGTCAGCGTAATGCTCTGCTTT |
| 30 | plasmid | AAAAGCAGAGCATTACGCTGACTTGACGGGACGGCGCAAGCTCATGACCAAAATCCCTTAACGTGAGTTA |
| | pAQ3::P(nir07)- | CGCGCGCGTCGTTCCACTGAGCGTCAGACCCCGTAGAAAAGATCAAAGGATCTTCTTGAGATCCTTTTTT |
| | fatBₘ-carB- | TCTGCGCGTAATCTGCTGCTTGCAAACAAAAAAACCACCGCTACCAGCGGTGGTTTGTTTGCCGGATCAA |
| | entD-SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTG<+CACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GeTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | G<+AATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | GAGTGGAAACCAAAACCGAAACTGCCTCAGCTGCTGGATGACCACTTCGGTCTGCACGGCCTGGTTTTCC |
| | | GTCGTACCTTCGCTATCCGTTCTTACGAAGTCGGCCCTGATCGCTCCACCTCCATCCTGGCGGTAATGAA |
| | | CCACATGCAGGAAGCAACTCTGAACCATGCGAAAAGCGTAGGTATCCTGGGCGATGGTTTCGGCACTACT |
| | | CTGGAGATGTCCAAACGTGATCTGATGTGGGTTGTTCGCCGTACCCATGTCGCGGTTGAACGCTACCCGA |
| | | CCTGGGGCGATACGGTTGAAGTGGAATGCTGGATCGGCGCGTCCGGCAACAACGGCATGCGTCGCGATTT |
| | | CCTGGTTCGCGATTGTAAGACGGGCGAGATTCTGACCCGTTGCACGTCCCTGAGCGTTCTGATGAATACC |
| | | CGTACCCGTCGTCTGAGCA{XATCCCGGACGAAGTTCGCGGTGAAATTGGCCCGGCATTCATCGATAACG |
| | | TTGCAGTAAAAGACGATGAAATCAAGAAACTGCAGAAACTGAATGACTCTACCGCGGACTACATCCAGGG |
| | | TGGTCTGACCCCGCGCTGGAACGACCTGGACGTGAACCAGCACGTCAACAACCTGAAATACGTAGCTTGG |
| | | GTATTCGAAACGGTCCCGGATTCTATCTTCGAATCTCACCACATCAGCTCCTTCACCCTGGAATACCGTC |
| | | GTGAGTGTACCCGTGACTCCGTTCTGCGCTCTCTGACCACGGTATCCGGCGGTAGCTCTGAAGCCGGTCT |
| | | GGTTTGCGATCACCTGCTGCAGCTGGAAGGCGGCAGCGAGGTTCTGCGTGCTCGTACTGAGTGGCGTCCG |
| | | AAGCTGACTGACTCTTTCCGCGGCATCTCTGTTATCCCGGCAGAGCCTCGTGTGTAAGAGCTCGAGGAGG |
| | | TTTTTACAATGACCAGCGATGTTCACGACGCCACAGACGGCGTCACCGAAACCGCACTCGACGACGAGCA |
| | | GTCGACCCGCCGCATCGCCGAGCTGTACGCCACCGATCCCGAGTTCGCCGCCGCCGCACCGTTGCCCGCC |
| | | GTGGTCGACGCGGCGCACAAACCCGGGCTGCGGCTGGCAGAGATCCTGCAGACCCTGTTCACCGGCTACG |
| | | GTGACCGCCCGGCGCTGGGATACCGCGCCCGTGAACTGGCCACCGACGAGGGCGGGCGCACCGTGACGCG |
| | | TCTGCTGCCGCGGTTCGACACCCTCACCTACGCCCAGGTGTGGTCGCGCGTGCAAGCGGTCGCCGCGGCC |
| | | CTGCGCCACAACTTCGCGCAGCCGATCTACCCCGGCGACGCCGTCGCGACGATCGGTTTCGCGAGTCCCG |
| | | ATTACCTGACGCTGGATCTCGTATGCGCCTACCTGGGCCTCGTGAGTGTTCCGCTGCAGCACAACGCACC |
| | | GGTCAGCCGGCTCGCCCCGATCCTGGCCGAGGTCGAACCGCGGATCCTCACCGTGAGCGCCGAATACCTC |
| | | GACCTCGCAGTCGAATCCGTGCGGGACGTCAACTCGGTGTCGCAGCTCGTGGTGTTCGACCATCACCCCG |
| | | AGGTCGACGACCACCGCGACGCACTGGCCCGCGCGCGTGAACAACTCGCCGGCAAGGGCATCGCCGTCAC |
| | | CACCCTGGACGCGATCGCCGACGAGGGCGCCGGGCTGCCGGCCGAACCGATCTACACCGCCGACCATGAT |
| | | CAGCGCCTCGCGATGATCCTGTACACCTCGGGTTCCACCGGCGCACCCAAGGGTGCGATGTACACCGAGG |
| | | CGATGGTGGCGCGGCTGTGGACCATGTCGTTCATCACGGGTGACCCCACGCCGGTCATCAACGTCAACTT |
| | | CATGCCGCTCAACCACCTGGGCGGGCGCATCCCCATTTCCACCGCCGTGCAGAACGGTGGAACCAGTTAC |
| | | TTCGTACCGGAATCCGACATGTCCACGCTGTTCGAGGATCTCGCGCTGGTGCGCCCGACCGAACTCGGCC |
| | | TGGTTCCGCGCGTCGCCGACATGCTCTACCAGCACCACCTCGCCACCGTCGACCGCCTGGTCACGCAGGG |
| | | CGCCGACGAACTGACCGCCGAGAAGCAGGCCGGTGCCGAACTGCGTGAGCAGGTGCTCGGCGGACGCGTG |
| | | ATCACCGGATTCGTCAGCACCGCACCGCTGGCCGCGGAGATGAGGGCGTTCCTCGACATCACCCTGGGCG |
| | | CACACATCGTCGACGGCTACGGGCTCACCGAGACCGGCGCCGTGACACGCGACGGTGTGATCGTGCGGCC |
| | | ACCGGTGATCGACTACAAGCTGATCGACGTTCCCGAACTCGGCTACTTCAGCACCGACAAGCCCTACCCG |
| | | CGTGGCGAACTGCTGGTCAGGTCGCAAACGCTGACTCCCGGGTACTACAAGCGCCCCGAGGTCACCGCGA |
| | | GCGTCTTCGACCGGGACGGCTACTACCACACCGGCGACGTCATGGCCGAGACCGCACCCGACCACCTGGT |
| | | GTACGTGGACCGTCGCAACAACGTCCTCAAACTCGCGCAGGGCGAGTTCGTGGCGGTCGCCAACCTGGAG |
| | | GCGGTGTTCTCCGGCGCGGCGCTGGTGCGCCAGATCTTCGTGTACGGCAACAGCGAGCGCAGTTTCCTTC |
| | | TGGCCGTGGTGGTCCCGACGCCGGAGGCGCTCGAGCAGTACGATCCGGCCGCGCTCAAGGCCGCGCTGGC |
| | | CGACTCGCTGCAGCGCACCGCACGCGACGCCGAACTGCAATCCTACGAGGTGCCGGCCGATTTCATCGTC |
| | | GAGACCGAGCCGTTCAGCGCCGCCAACGGGCTGCTGTCGGGTGTCGGAAAACTGCTGCGGCCCAACCTCA |
| | | AAGACCGCTACGGGCAGCGCCTGGAGCAGATGTACGCCGATATCGCGGCCACGCAGGCCAACCAGTTGCG |
| | | CGAACTGCGGCGCGCGGCCGCCACACAACCGGTGATCGACACCCTCACCCAGGCCGCTGCCACGATCCTC |
| | | GGCACCGGGAGCGAGGTGGCATCCGACGCCCACTTCACCGACCTGGGCGGGGATTCCCTGTCGGCGCTGA |
| | | CACTTTCGAACCTGCTGAGCGATTTCTTCGGTTTCGAAGTTCCCGTCGGCACCATCGTGAACCCGGCCAC |
| | | CAACCTCGCCCAACTCGCCCAGCACATCGAGGCGCAGCGCACCGCGGGTGACCGCAGGCCGAGTTTCACC |
| | | ACCGTGCACGGCGCGGACGCCACCGAGATCCGGGCGAGTGAGCTGACCCTGGACAAGTTCATCGACGCCG |
| | | AAACGCTCCGGGCCGCACCGGGTCTGCCCAAGGTCACCACCGAGCCACGGACGGTGTTGCTCTCGGGCGC |
| | | CAACGGCTGGCTGGGCCGGTTCCTCACGTTGCAGTGGCTGGAACGCCTGGCACCTGTCGGCGGCACCCTC |
| | | ATCACGATCGTGCGGGGCCGCGACGACGCCGCGGCCCGCGCACGGCTGACCCAGGCCTACGACACCGATC |
| | | CCGAGTTGTCCCGCCGCTTCGCCGAGCTGGCCGACCGCCACCTGCGGGTGGTCGCCGGTGACATCGGCGA |
| | | CCCGAATCTGGGCCTCACACCCGAGATCTGGCACCGGCTCGCCGCCGAGGTCGACCTGGTGGTGCATCCG |
| | | GCAGCGCTGGTCAACCACGTGCTCCCCTACCGGCAGCTGTTCGGCCCCAACGTCGTGGGCACGGCCGAGG |
| | | TGATCAAGCTGGCCCTCACCGAACGGATCAAGCCCGTCACGTACCTGTCCACCGTGTCGGTGGCCATGGG |
| | | GATCCCCGACTTCGAGGAGGACGGCGACATCCGGACCGTGAGCCCGGTGCGCCCGCTCGACGGCGGATAC |
| | | GCCAACGGCTACGGCAACAGCAAGTGGGCCGGCGAGGTGCTGCTGCGGGAGGCCCACGATCTGTGCGGGC |
| | | TGCCCGTGGCGACGTTCCGCTCGGACATGATCCTGGCGCATCCGCGCTACCGCGGTCAGGTCAACGTGCC |
| | | AGACATGTTCACGCGACTCCTGTTGAGCCTCTTGATCACCGGCGTCGCGCCGCGGTCGTTCTACATCGGA |
| | | GACGGTGAGCGCCCGCGGGCGCACTACCCCGGCCTGACGGTCGATTTCGTGGCCGAGGCGGTCACGACGC |
| | | TCGGCGCGCAGCAGCGCGAGGGATACGTGTCCTACGACGTGATGAACCCGCACGACGACGGGATCTCCCT |
| | | GGATGTGTTCGTGGACTGGCTGATCCGGGCGGGCCATCCGATCGACCGGGTCGACGACTACGACGACTGG |
| | | GTGCGTCGGTTCGAGACCGCGTTGACCGCGCTTCCCGAGAAGCGCCGCGCACAGACCGTACTGCCGCTGC |
| | | TGCACGCGTTCCGCGCTCCGCAGGCACCGTTGCGCGGCGCACCCGAACCCACGGAGGTGTTCCACGCCGC |
| | | GGTGCGCACCGCGAAGGTGGGCCCGGGAGACATCCCGCACCTCGACGAGGCGCTGATCGACAAGTACATA |
| | | CGCGATCTGCGTGAGTTCGGTCTGATCTGAGGTACCCACAAGGAGGTTTTTACAATGAAAACGACCCACA |
| | | CCAGCTTACCATTTGCCGGCCACACGTTACATTTCGTCGAATTTGATCCGGCGAACTTTTGTGAACAAGA |
| | | CCTGTTGTGGCTGCCGCATTATGCCCAGCTGCAGCACGCAGGCCGTAAGCGTAAAACTGAACATCTGGCC |
| | | GGTCGCATTGCGGCAGTGTATGCCCTGCGCGAGTACGGCTACAAATGCGTGCCGGCCATTGGTGAACTGC |
| | | GTCAACCGGTTTGGCCGGCAGAAGTTTACGGTTCCATCTCCCACTGCGGTACTACCGCGTTGGCGGTTGT |
| | | GTCTCGCCAGCCGATCGGTATTGATATTGAAGAGATATTCTCTGTCCAGACGGCACGCGAGCTGACGGAC |
| | | AACATCATTACCCCGGCAGAGCACGAGCGTCTGGCGGACTGTGGTCTGGCGTTCAGCCTGGCGCTGACCC |
| | | TGGCATTCAGCGCAAAAGAGAGCGCGTTCAAGGCTTCCGAGATCCAAACCGATGCGGGCTTCCTGGATTA |
| | | TCAAATCATCAGCTGGAACAAGCAACAGGTTATCATTCACCGTGAGAATGAGATGTTTGCCGTCCATTGG |
| | | CAGATTAAAGAGAAAATCGTTATCACCCTGTGCCAGCACGACTGAGAATTCGGTTTTCCGTCCTGTCTTG |
| | | ATTTTCAAGCAAACAATGCCTCCGATTTCTAATCGGAGGCATTTGTTTTTGTTTATTGCAAAAACAAAAA |
| | | ATATTGTTACAAATTTTTACAGGCTATTAAGCCTACCGTCATAAATAATTTGCCATTTACTAGTTTTTAA |
| | | TTAACCAGAACCTTGACCGAACGCAGCGGTGGTAACGGCGCAGTGGCGGTTTTCATGGCTTGTTATGACT |
| | | GTTTTTTTGGGGTACAGTCTATGCCTCGGGCATCCAAGCAGCAAGCGCGTTACGCCGTGGGTCGATGTTT |
| | | GATGTTATGGAGCAGCAACGATGTTACGCAGCAGGGCAGTCGCCCTAAAACAAAGTTAAACATCATGAGG |
| | | GAAGCGGTGATCGCCGAAGTATCGACTCAACTATCAGAGGTAGTTGGCGTCATCGAGCGCCATCTCGAAC |
| | | CGACGTTGCTGGCCGTACATTTGTACGGCTCCGCAGTGGATGGCGGCCTGAAGCCACACAGTGATATTGA |
| | | TTTGCTGGTTACGGTGACCGTAAGGCTTGATGAAACAACGCGGCGAGCTTTGATCAACGACCTTTTGGAA |
| | | ACTTCGGCTTCCCCTGGAGAGAGCGAGATTCTCCGCGCTGTAGAAGTCACCATTGTTGTGCACGACGACA |
| | | TCATTCCGTGGCGTTATCCAGCTAAGCGCGAACTGCAATTTGGAGAATGGCAGCGCAATGACATTCTTGC |
| | | AGGTATCTTCGAGCCAGCCACGATCGACATTGATCTGGCTATCTTGCTGACAAAAGCAAGAGAACATAGC |
| | | GTTGCCTTGGTAGGTCCAGCGGCGGAGGAACTCTTTGATCCGGTTCCTGAACAGGATCTATTTGAGGCGC |
| | | TAAATGAAACCTTAACGCTATGGAACTCGCCGCCCGACTGGGCTGGCGATGAGCGAAATGTAGTGCTTAC |
| | | GTTGTCCCGCATTTGGTACAGCGCAGTAACCGGCAAAATCGCGCCGAAGGATGTCGCTGCCGACTGGGCA |
| | | ATGGAGCGCCTGCCGGCCCAGTATCAGCCCGTCATACTTGAAGCTAGACAGGCTTATCTTGGACAAGAAG |
| | | AAGATCGCTTGGCCTCGCGCGCAGATCAGTTGGAAGAATTTGTCCACTACGTGAAAGGCGAGATCACCAA |
| | | GGTAGTCGGCAAATAATGTCTAACAATTCGTTCAAGCCGACGCCGCTTCGCGGCGCGGCTTAACTCAAGC |
| | | GTTAGATGCACTAAGCACATAATTGCTCACAGCCAAACTATCAGGTCAAGTCTGCTTTTATTATTTTTAA |
| | | GCGTGCATAATAAGCCCTACACAAATTGGGAGATATATCATGAGGCGCGCCACGAGAAAGAGTTATGACA |
| | | AATTAAAATTCTGACTCTTAGATTATTTCCAGAGAGGCTGATTTTCCCAATCTTTGGGAAAGCCTAAGTT |
| | | TTTAGATTCTATTTCTGGATACATCTCAAAAGTTCTTTTTAAATGCTGTGCAAAATTATGCTCTGGTTTA |
| | | ATTCTGTCTAAGAGATACTGAATACAACATAAGCCAGTGAAAATTTTACGGCTGTTTCTTTGATTAATAT |
| | | CCTCCAATACTTCTCTAGAGAGCCATTTTCCTTTTAACCTATCAGGCAATTTAGGTGATTCTCCTAGCTG |
| | | TATATTCCAGAGCCTTGAATGATGAGCGCAAATATTTCTAATATGCGACAAAGACCGTAACCAAGATATA |
| | | AAAAACTTGTTAGGTAATTGGAAATGAGTATGTATTTTTTGTCGTGTCTTAGATGGTAATAAATTTGTGT |
| | | ACATTCTAGATAACTGCCCAAAGGCGATTATCTCCAAAGCCATATATGACGGCGGTAGTAGAGGATTTGT |
| | | GTACTTGTTTCGATAATGCCCGATAAATTCTTCTACTTTTTTAGATTGGCAATATTGAGTAATCGAATCG |
| | | ATTAATTCTTGATGCTTCCCAGTGTCATAAAATAAACTTTTATTCAGATACCAATGAGGATCATAATCAT |
| | | GGGAGTAGTGATAAATCATTTGAGTTCTGACTGCTACTTCTATCGACTCCGTAGCATTAAAAATAAGCAT |
| | | TCTCAAGGATTTATCAAACTTGTATAGATTTGGCCGGCCCGTCAAAAGGGCGACACCCCATAATTAGCCC |
| | | GGGCGAAAGGCCCAGTCTTTCGACTGAGCCTTTCGTTTTATTTGATGCCTGGCAGTTCCCTACTCTCGCA |
| | | TGGGGAGTCCCCACACTACCATCGGCGCTACGGCGTTTCACTTCTGAGTTCGGCATGGGGTCAGGTGGGA |
| | | CCACCGCGCTACTGCCGCCAGGCAAACAAGGGGTGTTATGAGCCATATTCAGGTATAAATGGGCTCGCGA |
| | | TAATGTTCAGAATTGGTTAATTGGTTGTAACACTGACCCCTATTTGTTTATTTTTCTAAATACATTCAAA |
| | | TATGTATCCGCTCATGAGACAATAACCCTGATAAATGCTTCAATAATATTGAAAAAGGAAGAATATGAGT |
| | | ATTCAACATTTCCGTGTCGCCCTTATTCCCTTTTTTGCGGCATTTTGCCTTCCTGTTTTTGCTCACCCAG |
| | | AAACGCTGGTGAAAGTAAAAGATGCTGAAGATCAGTTGGGTGCACGAGTGGGTTACATCGAACTGGATCT |
| | | CAACAGCGGTAAGATCCTTGAGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACTCGGTCGCCGCATACACTATT |
| | | CTCAGAATGACTTGGTTGAGTACTCACCAGTCACAGAAAAGCATCTTACGGATGGCATGACAGTAAGAGA |
| | | ATTATGCAGTGCTGCCATAACCATGAGTGATAACACTGCGGCCAACTTACTTCTGACAACGATCGGAGGA |
| | | CCGAAGGAGCTAACCGCTTTTTTGCACAACATGGGGGATCATGTAACTCGCCTTGATCGTTGGGAACCGG |
| | | AGCTGAATGAAGCCATACCAAACGACGAGCGTGACACCACGATGCCTGTAGCGATGGCAACAACGTTGCG |
| | | CAAACTATTAACTGGCGAACTACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATTGCTGATAAATCCGGAGCCG |
| | | GTGAGCGTGGTTCTCGCGGTATCATCGCAGCGCTGGGGCCAGATGGTAAGCCCTCCCGTATCGTAGTTAT |
| | | CTACACGACGGGGAGTCAGGCAACTATGGATGAACGAAATAGACAGATCGCTGAGATAGGTGCCTCACTG |
| | | ATTAAGCATTGGT |
| 31 | plasmid | AAAAGCAGAGCATTACGCTGACTTGACGGGACGGCGCAAGCTCATGACCAAAATCCCTTAACGTGAGTTA |
| | pAQ3::P(nir07)- | CGCGCGCGTCGTTCCACTGAGCGTCAGACCCCGTAGAAAAGATCAAAGGATCTTCTTGAGATCCTTTTTT |
| | fatB2ₘ-carB- | TCTGCGCGTAATCTGCTGCTTGCAAACAAAAAAACCACCGCTACCAGCGGTGGTTTGTTTGCCGGATCAA |
| | entD-SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | GACCGTAAAAGCAAGCGTCCGGACATGCTGGTTGATTCCTTTGGTCTGGAAAGCACCGTGCAGGACGGTC |
| | | TGGTTTTCCGTCAGTCTTTCTCCATTCGTAGCTATGAGATTGGTACTGATCGTACCGCCTCTATCGAAAC |
| | | CCTGATGAATCACCTGCAAGAAACCTCTCTGAACCATTGTAAGTCTACTGGCATCCTGCTGGACGGTTTC |
| | | GGTCGTACCCTGGAGATGTGCAAACGCGACCTGATTTGGGTAGTGATCAAAATGCAGATCAAAGTTAACC |
| | | GTTATCCGGCATGGGGTGATACCGTTGAAATCAACACCCGCTTTTCTCGTCTGGGCAAAATCGGTATGGG |
| | | CCGTGACTGGCTGATCTCTGACTGTAACACTGGTGAAATTCTGGTTCGTGCTACTAGCGCATACGCGATG |
| | | ATGAACCAGAAAACCCGTCGCCTGAGCAAGCTGCCGTACGAGGTCCACCAGGAGATTGTTCCGCTGTTTG |
| | | TAGACAGCCCAGTGATTGAGGATTCTGACCTGAAAGTGCATAAATTCAAAGTGAAGACCGGTGACAGCAT |
| | | CCAAAAAGGCCTGACCCCAGGTTGGAACGATCTGGACGTTAACCAGCACGTTTCCAACGTGAAGTATATC |
| | | GGTTGGATTCTGGAGAGCATGCCGACCGAGGTCCTGGAAACCCAGGAGCTGTGTTCCCTGGCGCTGGAGT |
| | | ACCGCCGTGAGTGCGGCCGTGACAGCGTGCTGGAGTCTGTGACCGCTATGGACCCAAGCAAAGTTGGTGT |
| | | TCGTAGCCAGTACCAGCACCTGCTGCGTCTGGAAGACGGTACTGCTATCGTGAACGGTGCAACTGAATGG |
| | | CGTCCTAAAAACGCGGGTGCAAACGGTGCTATCAGCACCGGTAAAACCTCTAACGGTAACTCCGTGAGCT |
| | | AAGAGCTCGAGGAGGTTTTTACAATGACCAGCGATGTTCACGACGCCACAGACGGCGTCACCGAAACCGC |
| | | ACTCGACGACGAGCAGTCGACCCGCCGCATCGCCGAGCTGTACGCCACCGATCCCGAGTTCGCCGCCGCC |
| | | GCACCGTTGCCCGCCGTGGTCGACGCGGCGCACAAACCCGGGCTGCGGCTGGCAGAGATCCTGCAGACCC |
| | | TGTTCACCGGCTACGGTGACCGCCCGGCGCTGGGATACCGCGCCCGTGAACTGGCCACCGACGAGGGCGG |
| | | GCGCACCGTGACGCGTCTGCTGCCGCGGTTCGACACCCTCACCTACGCCCAGGTGTGGTCGCGCGTGCAA |
| | | GCGGTCGCCGCGGCCCTGCGCCACAACTTCGCGCAGCCGATCTACCCCGGCGACGCCGTCGCGACGATCG |
| | | GTTTCGCGAGTCCCGATTACCTGACGCTGGATCTCGTATGCGCCTACCTGGGCCTCGTGAGTGTTCCGCT |
| | | GCAGCACAACGCACCGGTCAGCCGGCTCGCCCCGATCCTGGCCGAGGTCGAACCGCGGATCCTCACCGTG |
| | | AGCGCCGAATACCTCGACCTCGCAGTCGAATCCGTGCGGGACGTCAACTCGGTGTCGCAGCTCGTGGTGT |
| | | TCGACCATCACCCCGAGGTCGACGACCACCGCGACGCACTGGCCCGCGCGCGTGAACAACTCGCCGGCAA |
| | | GGGCATCGCCGTCACCACCCTGGACGCGATCGCCGACGAGGGCGCCGGGCTGCCGGCCGAACCGATCTAC |
| | | ACCGCCGACCATGATCAGCGCCTCGCGATGATCCTGTACACCTCGGGTTCCACCGGCGCACCCAAGGGTG |
| | | CGATGTACACCGAGGCGATGGTGGCGCGGCTGTGGACCATGTCGTTCATCACGGGTGACCCCACGCCGGT |
| | | CATCAACGTCAACTTCATGCCGCTCAACCACCTGGGCGGGCGCATCCCCATTTCCACCGCCGTGCAGAAC |
| | | GGTGGAACCAGTTACTTCGTACCGGAATCCGACATGTCCACGCTGTTCGAGGATCTCGCGCTGGTGCGCC |
| | | CGACCGAACTCGGCCTGGTTCCGCGCGTCGCCGACATGCTCTACCAGCACCACCTCGCCACCGTCGACCG |
| | | CCTGGTCACGCAGGGCGCCGACGAACTGACCGCCGAGAAGCAGGCCGGTGCCGAACTGCGTGAGCAGGTG |
| | | CTCGGCGGACGCGTGATCACCGGATTCGTCAGCACCGCACCGCTGGCCGCGGAGATGAGGGCGTTCCTCG |
| | | ACATCACCCTGGGCGCACACATCGTCGACGGCTACGGGCTCACCGAGACCGGCGCCGTGACACGCGACGG |
| | | TGTGATCGTGCGGCCACCGGTGATCGACTACAAGCTGATCGACGTTCCCGAACTCGGCTACTTCAGCACC |
| | | GACAAGCCCTACCCGCGTGGCGAACTGCTGGTCAGGTCGCAAACGCTGACTCCCGGGTACTACAAGCGCC |
| | | CCGAGGTCACCGCGAGCGTCTTCGACCGGGACGGCTACTACCACACCGGCGACGTCATGGCCGAGACCGC |
| | | ACCCGACCACCTGGTGTACGTGGACCGTCGCAACAACGTCCTCAAACTCGCGCAGGGCGAGTTCGTGGCG |
| | | GTCGCCAACCTGGAGGCGGTGTTCTCCGGCGCGGCGCTGGTGCGCCAGATCTTCGTGTACGGCAACAGCG |
| | | AGCGCAGTTTCCTTCTGGCCGTGGTGGTCCCGACGCCGGAGGCGCTCGAGCAGTACGATCCGGCCGCGCT |
| | | CAAGGCCGCGCTGGCCGACTCGCTGCAGCGCACCGCACGCGACGCCGAACTGCAATCCTACGAGGTGCCG |
| | | GCCGATTTCATCGTCGAGACCGAGCCGTTCAGCGCCGCCAACGGGCTGCTGTCGGGTGTCGGAAAACTGC |
| | | TGCGGCCCAACCTCAAAGACCGCTACGGGCAGCGCCTGGAGCAGATGTACGCCGATATCGCGGCCACGCA |
| | | GGCCAACCAGTTGCGCGAACTGCGGCGCGCGGCCGCCACACAACCGGTGATCGACACCCTCACCCAGGCC |
| | | GCTGCCACGATCCTCGGCACCGGGAGCGAGGTGGCATCCGACGCCCACTTCACCGACCTGGGCGGGGATT |
| | | CCCTGTCGGCGCTGACACTTTCGAACCTGCTGAGCGATTTCTTCGGTTTCGAAGTTCCCGTCGGCACCAT |
| | | CGTGAACCCGGCCACCAACCTCGCCCAACTCGCCCAGCACATCGAGGCGCAGCGCACCGCGGGTGACCGC |
| | | AGGCCGAGTTTCACCACCGTGCACGGCGCGGACGCCACCGAGATCCGGGCGAGTGAGCTGACCCTGGACA |
| | | AGTTCATCGACGCCGAAACGCTCCGGGCCGCACCGGGTCTGCCCAAGGTCACCACCGAGCCACGGACGGT |
| | | GTTGCTCTCGGGCGCCAACGGCTGGCTGGGCCGGTTCCTCACGTTGCAGTGGCTGGAACGCCTGGCACCT |
| | | GTCGGCGGCACCCTCATCACGATCGTGCGGGGCCGCGACGACGCCGCGGCCCGCGCACGGCTGACCCAGG |
| | | CCTACGACACCGATCCCGAGTTGTCCCGCCGCTTCGCCGAGCTGGCCGACCGCCACCTGCGGGTGGTCGC |
| | | CGGTGACATCGGCGACCCGAATCTGGGCCTCACACCCGAGATCTGGCACCGGCTCGCCGCCGAGGTCGAC |
| | | CTGGTGGTGCATCCGGCAGCGCTGGTCAACCACGTGCTCCCCTACCGGCAGCTGTTCGGCCCCAACGTCG |
| | | TGGGCACGGCCGAGGTGATCAAGCTGGCCCTCACCGAACGGATCAAGCCCGTCACGTACCTGTCCACCGT |
| | | GTCGGTGGCCATGGGGATCCCCGACTTCGAGGAGGACGGCGACATCCGGACCGTGAGCCCGGTGCGCCCG |
| | | CTCGACGGCGGATACGCCAACGGCTACGGCAACAGCAAGTGGGCCGGCGAGGTGCTGCTGCGGGAGGCCC |
| | | ACGATCTGTGCGGGCTGCCCGTGGCGACGTTCCGCTCGGACATGATCCTGGCGCATCCGCGCTACCGCGG |
| | | TCAGGTCAACGTGCCAGACATGTTCACGCGACTCCTGTTGAGCCTCTTGATCACCGGCGTCGCGCCGCGG |
| | | TCGTTCTACATCGGAGACGGTGAGCGCCCGCGGGCGCACTACCCCGGCCTGACGGTCGATTTCGTGGCCG |
| | | AGGCGGTCACGACGCTCGGCGCGCAGCAGCGCGAGGGATACGTGTCCTACGACGTGATGAACCCGCACGA |
| | | CGACGGGATCTCCCTGGATGTGTTCGTGGACTGGCTGATCCGGGCGGGCCATCCGATCGACCGGGTCGAC |
| | | GACTACGACGACTGGGTGCGTCGGTTCGAGACCGCGTTGACCGCGCTTCCCGAGAAGCGCCGCGCACAGA |
| | | CCGTACTGCCGCTGCTGCACGCGTTCCGCGCTCCGCAGGCACCGTTGCGCGGCGCACCCGAACCCACGGA |
| | | GGTGTTCCACGCCGCGGTGCGCACCGCGAAGGTGGGCCCGGGAGACATCCCGCACCTCGACGAGGCGCTG |
| | | ATCGACAAGTACATACGCGATCTGCGTGAGTTCGGTCTGATCTGAGGTACCCACAAGGAGGTTTTTACAA |
| | | TGAAAACGACCCACACCAGCTTACCATTTGCCGGCCACACGTTACATTTCGTCGAATTTGATCCGGCGAA |
| | | CTTTTGTGAACAAGACCTGTTGTGGCTGCCGCATTATGCCCAGCTGCAGCACGCAGGCCGTAAGCGTAAA |
| | | ACTGAACATCTGGCCGGTCGCATTGCGGCAGTGTATGCCCTGCGCGAGTACGGCTACAAATGCGTGCCGG |
| | | CCATTGGTGAACTGCGTCAACCGGTTTGGCCGGCAGAAGTTTACGGTTCCATCTCCCACTGCGGTACTAC |
| | | CGCGTTGGCGGTTGTGTCTCGCCAGCCGATCGGTATTGATATTGAAGAGATATTCTCTGTCCAGACGGCA |
| | | CGCGAGCTGACGGACAACATCATTACCCCGGCAGAGCACGAGCGTCTGGCGGACTGTGGTCTGGCGTTCA |
| | | GCCTGGCGCTGACCCTGGCATTCAGCGCAAAAGAGAGCGCGTTCAAGGCTTCCGAGATCCAAACCGATGC |
| | | GGGCTTCCTGGATTATCAAATCATCAGCTGGAACAAGCAACAGGTTATCATTCACCGTGAGAATGAGATG |
| | | TTTGCCGTCCATTGGCAGATTAAAGAGAAAATCGTTATCACCCTGTGCCAGCACGACTGAGAATTCGGTT |
| | | TTCCGTCCTGTCTTGATTTTCAAGCAAACAATGCCTCCGATTTCTAATCGGAGGCATTTGTTTTTGTTTA |
| | | TTGCAAAAACAAAAAATATTGTTACAAATTTTTACAGGCTATTAAGCCTACCGTCATAAATAATTTGCCA |
| | | TTTACTAGTTTTTAATTAACCAGAACCTTGACCGAACGCAGCGGTGGTAACGGCGCAGTGGCGGTTTTCA |
| | | TGGCTTGTTATGACTGTTTTTTTGGGGTACAGTCTATGCCTCGGGCATCCAAGCAGCAAGCGCGTTACGC |
| | | CGTGGGTCGATGTTTGATGTTATGGAGCAGCAACGATGTTACGCAGCAGGGCAGTCGCCCTAAAACAAAG |
| | | TTAAACATCATGAGGGAAGCGGTGATCGCCGAAGTATCGACTCAACTATCAGAGGTAGTTGGCGTCATCG |
| | | AGCGCCATCTCGAACCGACGTTGCTGGCCGTACATTTGTACGGCTCCGCAGTGGATGGCGGCCTGAAGCC |
| | | ACACAGTGATATTGATTTGCTGGTTACGGTGACCGTAAGGCTTGATGAAACAACGCGGCGAGCTTTGATC |
| | | AACGACCTTTTGGAAACTTCGGCTTCCCCTGGAGAGAGCGAGATTCTCCGCGCTGTAGAAGTCACCATTG |
| | | TTGTGCACGACGACATCATTCCGTGGCGTTATCCAGCTAAGCGCGAACTGCAATTTGGAGAATGGCAGCG |
| | | CAATGACATTCTTGCAGGTATCTTCGAGCCAGCCACGATCGACATTGATCTGGCTATCTTGCTGACAAAA |
| | | GCAAGAGAACATAGCGTTGCCTTGGTAGGTCCAGCGGCGGAGGAACTCTTTGATCCGGTTCCTGAACAGG |
| | | ATCTATTTGAGGCGCTAAATGAAACCTTAACGCTATGGAACTCGCCGCCCGACTGGGCTGGCGATGAGCG |
| | | AAATGTAGTGCTTACGTTGTCCCGCATTTGGTACAGCGCAGTAACCGGCAAAATCGCGCCGAAGGATGTC |
| | | GCTGCCGACTGGGCAATGGAGCGCCTGCCGGCCCAGTATCAGCCCGTCATACTTGAAGCTAGACAGGCTT |
| | | ATCTTGGACAAGAAGAAGATCGCTTGGCCTCGCGCGCAGATCAGTTGGAAGAATTTGTCCACTACGTGAA |
| | | AGGCGAGATCACCAAGGTAGTCGGCAAATAATGTCTAACAATTCGTTCAAGCCGACGCCGCTTCGCGGCG |
| | | CGGCTTAACTCAAGCGTTAGATGCACTAAGCACATAATTGCTCACAGCCAAACTATCAGGTCAAGTCTGC |
| | | TTTTATTATTTTTAAGCGTGCATAATAAGCCCTACACAAATTGGGAGATATATCATGAGGCGCGCCACGA |
| | | GAAAGAGTTATGACAAATTAAAATTCTGACTCTTAGATTATTTCCAGAGAGGCTGATTTTCCCAATCTTT |
| | | GGGAAAGCCTAAGTTTTTAGATTCTATTTCTGGATACATCTCAAAAGTTCTTTTTAAATGCTGTGCAAAA |
| | | TTATGCTCTGGTTTAATTCTGTCTAAGAGATACTGAATACAACATAAGCCAGTGAAAATTTTACGGCTGT |
| | | TTCTTTGATTAATATCCTCCAATACTTCTCTAGAGAGCCATTTTCCTTTTAACCTATCAGGCAATTTAGG |
| | | TGATTCTCCTAGCTGTATATTCCAGAGCCTTGAATGATGAGCGCAAATATTTCTAATATGCGACAAAGAC |
| | | CGTAACCAAGATATAAAAAACTTGTTAGGTAATTGGAAATGAGTATGTATTTTTTGTCGTGTCTTAGATG |
| | | GTAATAAATTTGTGTACATTCTAGATAACTGCCCAAAGGCGATTATCTCCAAAGCCATATATGACGGCGG |
| | | TAGTAGAGGATTTGTGTACTTGTTTCGATAATGCCCGATAAATTCTTCTACTTTTTTAGATTGGCAATAT |
| | | TGAGTAATCGAATCGATTAATTCTTGATGCTTCCCAGTGTCATAAAATAAACTTTTATTCAGATACCAAT |
| | | GAGGATCATAATCATGGGAGTAGTGATAAATCATTTGAGTTCTGACTGCTACTTCTATCGACTCCGTAGC |
| | | ATTAAAAATAAGCATTCTCAAGGATTTATCAAACTTGTATAGATTTGGCCGGCCCGTCAAAAGGGCGACA |
| | | CCCCATAATTAGCCCGGGCGAAAGGCCCAGTCTTTCGACTGAGCCTTTCGTTTTATTTGATGCCTGGCAG |
| | | TTCCCTACTCTCGCATGGGGAGTCCCCACACTACCATCGGCGCTACGGCGTTTCACTTCTGAGTTCGGCA |
| | | TGGGGTCAGGTGGGACCACCGCGCTACTGCCGCCAGGCAAACAAGGGGTGTTATGAGCCATATTCAGGTA |
| | | TAAATGGGCTCGCGATAATGTTCAGAATTGGTTAATTGGTTGTAACACTGACCCCTATTTGTTTATTTTT |
| | | CTAAATACATTCAAATATGTATCCGCTCATGAGACAATAACCCTGATAAATGCTTCAATAATATTGAAAA |
| | | AGGAAGAATATGAGTATTCAACATTTCCGTGTCGCCCTTATTCCCTTTTTTGCGGCATTTTGCCTTCCTG |
| | | TTTTTGCTCACCCAGAAACGCTGGTGAAAGTAAAAGATGCTGAAGATCAGTTGGGTGCACGAGTGGGTTA |
| | | CATCGAACTGGATCTCAACAGCGGTAAGATCCTTGAGAGTTTTCGCCCCGAAGAACGTTTTCCAATGATG |
| | | AGCACTTTTAAAGTTCTGCTATGTGGCGCGGTATTATCCCGTATTGACGCCGGGCAAGAGCAACTCGGTC |
| | | GCCGCATACACTATTCTCAGAATGACTTGGTTGAGTACTCACCAGTCACAGAAAAGCATCTTACGGATGG |
| | | CATGACAGTAAGAGAATTATGCAGTGCTGCCATAACCATGAGTGATAACACTGCGGCCAACTTACTTCTG |
| | | ACAACGATCGGAGGACCGAAGGAGCTAACCGCTTTTTTGCACAACATGGGGGATCATGTAACTCGCCTTG |
| | | ATCGTTGGGAACCGGAGCTGAATGAAGCCATACCAAACGACGAGCGTGACACCACGATGCCTGTAGCGAT |
| | | GGCAACAACGTTGCGCAAACTATTAACTGGCGAACTACTTACTCTAGCTTCCCGGCAACAATTAATAGAC |
| | | TGGATGGAGGCGGATAAAGTTGCAGGACCACTTCTGCGCTCGGCCCTTCCGGCTGGCTGGTTTATTGCTG |
| | | ATAAATCCGGAGCCGGTGAGCGTGGTTCTCGCGGTATCATCGCAGCGCTGGGGCCAGATGGTAAGCCCTC |
| | | CCGTATCGTAGTTATCTACACGACGGGGAGTCAGGCAACTATGGATGAACGAAATAGACAGATCGCTGAG |
| | | ATAGGTGCCTCACTGATTAAGCATTGGT |
| 32 | carboxylic | GAGCTCGAGGAGGTTTTTACAATGACCAGCGATGTTCACGACGCCACAGACGGCGTCACCGAAACCGCAC |
| | acid | TCGACGACGAGCAGTCGACCCGCCGCATCGCCGAGCTGTACGCCACCGATCCCGAGTTCGCCGCCGCCGC |
| | reductase | ACCGTTGCCCGCCGTGGTCGACGCGGCGCACAAACCCGGGCTGCGGCTGGCAGAGATCCTGCAGACCCTG |
| | amplified | TTCACCGGCTACGGTGACCGCCCGGCGCTGGGATACCGCGCCCGTGAACTGGCCACCGACGAGGGCGGGC |
| | from | GCACCGTGACGCGTCTGCTGCCGCGGTTCGACACCCTCACCTACGCCCAGGTGTGGTCGCGCGTGCAAGC |
| | Mycobacterium | GGTCGCCGCGGCCCTGCGCCACAACTTCGCGCAGCCGATCTACCCCGGCGACGCCGTCGCGACGATCGGT |
| | smegmatis. | TTCGCGAGTCCCGATTACCTGACGCTGGATCTCGTATGCGCCTACCTGGGCCTCGTGAGTGTTCCGCTGC |
| | | AGCACAACGCACCGGTCAGCCGGCTCGCCCCGATCCTGGCCGAGGTCGAACCGCGGATCCTCACCGTGAG |
| | | CGCCGAATACCTCGACCTCGCAGTCGAATCCGTGCGGGACGTCAACTCGGTGTCGCAGCTCGTGGTGTTC |
| | | GACCATCACCCCGAGGTCGACGACCACCGCGACGCACTGGCCCGCGCGCGTGAACAACTCGCCGGCAAGG |
| | | GCATCGCCGTCACCACCCTGGACGCGATCGCCGACGAGGGCGCCGGGCTGCCGGCCGAACCGATCTACAC |
| | | CGCCGACCATGATCAGCGCCTCGCGATGATCCTGTACACCTCGGGTTCCACCGGCGCACCCAAGGGTGCG |
| | | ATGTACACCGAGGCGATGGTGGCGCGGCTGTGGACCATGTCGTTCATCACGGGTGACCCCACGCCGGTCA |
| | | TCAACGTCAACTTCATGCCGCTCAACCACCTGGGCGGGCGCATCCCCATTTCCACCGCCGTGCAGAACGG |
| | | TGGAACCAGTTACTTCGTACCGGAATCCGACATGTCCACGCTGTTCGAGGATCTCGCGCTGGTGCGCCCG |
| | | ACCGAACTCGGCCTGGTTCCGCGCGTCGCCGACATGCTCTACCAGCACCACCTCGCCACCGTCGACCGCC |
| | | TGGTCACGCAGGGCGCCGACGAACTGACCGCCGAGAAGCAGGCCGGTGCCGAACTGCGTGAGCAGGTGCT |
| | | CGGCGGACGCGTGATCACCGGATTCGTCAGCACCGCACCGCTGGCCGCGGAGATGAGGGCGTTCCTCGAC |
| | | ATCACCCTGGGCGCACACATCGTCGACGGCTACGGGCTCACCGAGACCGGCGCCGTGACACGCGACGGTG |
| | | TGATCGTGCGGCCACCGGTGATCGACTACAAGCTGATCGACGTTCCCGAACTCGGCTACTTCAGCACCGA |
| | | CAAGCCCTACCCGCGTGGCGAACTGCTGGTCAGGTCGCAAACGCTGACTCCCGGGTACTACAAGCGCCCC |
| | | GAGGTCACCGCGAGCGTCTTCGACCGGGACGGCTACTACCACACCGGCGACGTCATGGCCGAGACCGCAC |
| | | CCGACCACCTGGTGTACGTGGACCGTCGCAACAACGTCCTCAAACTCGCGCAGGGCGAGTTCGTGGCGGT |
| | | CGCCAACCTGGAGGCGGTGTTCTCCGGCGCGGCGCTGGTGCGCCAGATCTTCGTGTACGGCAACAGCGAG |
| | | CGCAGTTTCCTTCTGGCCGTGGTGGTCCCGACGCCGGAGGCGCTCGAGCAGTACGATCCGGCCGCGCTCA |
| | | AGGCCGCGCTGGCCGACTCGCTGCAGCGCACCGCACGCGACGCCGAACTGCAATCCTACGAGGTGCCGGC |
| | | CGATTTCATCGTCGAGACCGAGCCGTTCAGCGCCGCCAACGGGCTGCTGTCGGGTGTCGGAAAACTGCTG |
| | | CGGCCCAACCTCAAAGACCGCTACGGGCAGCGCCTGGAGCAGATGTACGCCGATATCGCGGCCACGCAGG |
| | | CCAACCAGTTGCGCGAACTGCGGCGCGCGGCCGCCACACAACCGGTGATCGACACCCTCACCCAGGCCGC |
| | | TGCCACGATCCTCGGCACCGGGAGCGAGGTGGCATCCGACGCCCACTTCACCGACCTGGGCGGGGATTCC |
| | | CTGTCGGCGCTGACACTTTCGAACCTGCTGAGCGATTTCTTCGGTTTCGAAGTTCCCGTCGGCACCATCG |
| | | TGAACCCGGCCACCAACCTCGCCCAACTCGCCCAGCACATCGAGGCGCAGCGCACCGCGGGTGACCGCAG |
| | | GCCGAGTTTCACCACCGTGCACGGCGCGGACGCCACCGAGATCCGGGCGAGTGAGCTGACCCTGGACAAG |
| | | TTCATCGACGCCGAAACGCTCCGGGCCGCACCGGGTCTGCCCAAGGTCACCACCGAGCCACGGACGGTGT |
| | | TGCTCTCGGGCGCCAACGGCTGGCTGGGCCGGTTCCTCACGTTGCAGTGGCTGGAACGCCTGGCACCTGT |
| | | CGGCGGCACCCTCATCACGATCGTGCGGGGCCGCGACGACGCCGCGGCCCGCGCACGGCTGACCCAGGCC |
| | | TACGACACCGATCCCGAGTTGTCCCGCCGCTTCGCCGAGCTGGCCGACCGCCACCTGCGGGTGGTCGCCG |
| | | GTGACATCGGCGACCCGAATCTGGGCCTCACACCCGAGATCTGGCACCGGCTCGCCGCCGAGGTCGACCT |
| | | GGTGGTGCATCCGGCAGCGCTGGTCAACCACGTGCTCCCCTACCGGCAGCTGTTCGGCCCCAACGTCGTG |
| | | GGCACGGCCGAGGTGATCAAGCTGGCCCTCACCGAACGGATCAAGCCCGTCACGTACCTGTCCACCGTGT |
| | | CGGTGGCCATGGGGATCCCCGACTTCGAGGAGGACGGCGACATCCGGACCGTGAGCCCGGTGCGCCCGCT |
| | | CGACGGCGGATACGCCAACGGCTACGGCAACAGCAAGTGGGCCGGCGAGGTGCTGCTGCGGGAGGCCCAC |
| | | GATCTGTGCGGGCTGCCCGTGGCGACGTTCCGCTCGGACATGATCCTGGCGCATCCGCGCTACCGCGGTC |
| | | AGGTCAACGTGCCAGACATGTTCACGCGACTCCTGTTGAGCCTCTTGATCACCGGCGTCGCGCCGCGGTC |
| | | GTTCTACATCGGAGACGGTGAGCGCCCGCGGGCGCACTACCCCGGCCTGACGGTCGATTTCGTGGCCGAG |
| | | GCGGTCACGACGCTCGGCGCGCAGCAGCGCGAGGGATACGTGTCCTACGACGTGATGAACCCGCACGACG |
| | | ACGGGATCTCCCTGGATGTGTTCGTGGACTGGCTGATCCGGGCGGGCCATCCGATCGACCGGGTCGACGA |
| | | CTACGACGACTGGGTGCGTCGGTTCGAGACCGCGTTGACCGCGCTTCCCGAGAAGCGCCGCGCACAGACC |
| | | GTACTGCCGCTGCTGCACGCGTTCCGCGCTCCGCAGGCACCGTTGCGCGGCGCACCCGAACCCACGGAGG |
| | | TGTTCCACGCCGCGGTGCGCACCGCGAAGGTGGGCCCGGGAGACATCCCGCACCTCGACGAGGCGCTGAT |
| | | CGACAAGTACATACGCGATCTGCGTGAGTTCGGTCTGATCTGAGGTACC |
| 33 | codon- | CATATGCAAGAACTGGCCCTGAGAAGCGAGCTGGACTTCAATAGCGAAACCTATAAAGATGCGTATAGCC |
| | optimized | GTATTAACGCCATTGTGATCGAAGGCGAGCAAGAAGCATACCAAAACTACCTGGACATGGCGCAACTGCT |
| | Cyanothece | GCCGGAGGACGAGGCTGAGCTGATTCGTTTGAGCAAGATGGAGAACCGTCACAAAAAGGGTTTTCAAGCG |
| | adm. | TGCGGCAAGAACCTCAATGTGACTCCGGATATGGATTATGCACAGCAGTTCTTTGCGGAGCTGCACGGCA |
| | | ATTTTCAGAAGGCTAAAGCCGAGGGTAAGATTGTTACCTGCCTGCTCATCCAAAGCCTGATCATCGAGGC |
| | | GTTTGCGATTGCAGCCTACAACATTTACATTCCAGTGGCTGATCCGTTTGCACGTAAAATCACCGAGGGT |
| | | GTCGTCAAGGATGAGTATACCCACCTGAATTTCGGCGAAGTTTGGTTGAAGGAACATTTTGAAGCAAGCA |
| | | AGGCGGAGTTGGAGGACGCCAACAAAGAGAACTTACCGCTGGTCTGGCAGATGTTGAACCAGGTCGAAAA |
| | | GGATGCCGAAGTGCTGGGTATGGAGAAAGAGGCTCTGGTGGAGGACTTTATGATTAGCTATGGTGAGGCA |
| | | CTGAGCAACATCGGCTTTTCTACGAGAGAAATCATGAAGATGAGCGCGTACGGTCTGCGTGCAGCATAAG |
| | | AGCTC |
| 34 | codon- | GAGCTCGAGGAGGTTTTTACAATGACCAGCGATGTTCACGACGCCACAGACGGCGTCACCGAAACCGCAC |
| | Optimized E. | TCGACGACGAGCAGTCGACCCGCCGCATCGCCGAGCTGTACGCCACCGATCCCGAGTTCGCCGCCGCCGC |
| | coli tesAₘ and | ACCGTTGCCCGCCGTGGTCGACGCGGCGCACAAACCCGGGCTGCGGCTGGCAGAGATCCTGCAGACCCTG |
| | E. coli entD | TTCACCGGCTACGGTGACCGCCCGGCGCTGGGATACCGCGCCCGTGAACTGGCCACCGACGAGGGCGGGC |
| | genes. | GCACCGTGACGCGTCTGCTGCCGCGGTTCGACACCCTCACCTACGCCCAGGTGTGGTCGCGCGTGCAAGC |
| | | GGTCGCCGCGGCCCTGCGCCACAACTTCGCGCAGCCGATCTACCCCGGCGACGCCGTCGCGACGATCGGT |
| | | TTCGCGAGTCCCGATTACCTGACGCTGGATCTCGTATGCGCCTACCTGGGCCTCGTGAGTGTTCCGCTGC |
| | | AGCACAACGCACCGGTCAGCCGGCTCGCCCCGATCCTGGCCGAGGTCGAACCGCGGATCCTCACCGTGAG |
| | | CGCCGAATACCTCGACCTCGCAGTCGAATCCGTGCGGGACGTCAACTCGGTGTCGCAGCTCGTGGTGTTC |
| | | GACCATCACCCCGAGGTCGACGACCACCGCGACGCACTGGCCCGCGCGCGTGAACAACTCGCCGGCAAGG |
| | | GCATCGCCGTCACCACCCTGGACGCGATCGCCGACGAGGGCGCCGGGCTGCCGGCCGAACCGATCTACAC |
| | | CGCCGACCATGATCAGCGCCTCGCGATGATCCTGTACACCTCGGGTTCCACCGGCGCACCCAAGGGTGCG |
| | | ATGTACACCGAGGCGATGGTGGCGCGGCTGTGGACCATGTCGTTCATCACGGGTGACCCCACGCCGGTCA |
| | | TCAACGTCAACTTCATGCCGCTCAACCACCTGGGCGGGCGCATCCCCATTTCCACCGCCGTGCAGAACGG |
| | | TGGAACCAGTTACTTCGTACCGGAATCCGACATGTCCACGCTGTTCGAGGATCTCGCGCTGGTGCGCCCG |
| | | ACCGAACTCGGCCTGGTTCCGCGCGTCGCCGACATGCTCTACCAGCACCACCTCGCCACCGTCGACCGCC |
| | | TGGTCACGCAGGGCGCCGACGAACTGACCGCCGAGAAGCAGGCCGGTGCCGAACTGCGTGAGCAGGTGCT |
| | | CGGCGGACGCGTGATCACCGGATTCGTCAGCACCGCACCGCTGGCCGCGGAGATGAGGGCGTTCCTCGAC |
| | | ATCACCCTGGGCGCACACATCGTCGACGGCTACGGGCTCACCGAGACCGGCGCCGTGACACGCGACGGTG |
| | | TGATCGTGCGGCCACCGGTGATCGACTACAAGCTGATCGACGTTCCCGAACTCGGCTACTTCAGCACCGA |
| | | CAAGCCCTACCCGCGTGGCGAACTGCTGGTCAGGTCGCAAACGCTGACTCCCGGGTACTACAAGCGCCCC |
| | | GAGGTCACCGCGAGCGTCTTCGACCGGGACGGCTACTACCACACCGGCGACGTCATGGCCGAGACCGCAC |
| | | CCGACCACCTGGTGTACGTGGACCGTCGCAACAACGTCCTCAAACTCGCGCAGGGCGAGTTCGTGGCGGT |
| | | CGCCAACCTGGAGGCGGTGTTCTCCGGCGCGGCGCTGGTGCGCCAGATCTTCGTGTACGGCAACAGCGAG |
| | | CGCAGTTTCCTTCTGGCCGTGGTGGTCCCGACGCCGGAGGCGCTCGAGCAGTACGATCCGGCCGCGCTCA |
| | | AGGCCGCGCTGGCCGACTCGCTGCAGCGCACCGCACGCGACGCCGAACTGCAATCCTACGAGGTGCCGGC |
| | | CGATTTCATCGTCGAGACCGAGCCGTTCAGCGCCGCCAACGGGCTGCTGTCGGGTGTCGGAAAACTGCTG |
| | | CGGCCCAACCTCAAAGACCGCTACGGGCAGCGCCTGGAGCAGATGTACGCCGATATCGCGGCCACGCAGG |
| | | CCAACCAGTTGCGCGAACTGCGGCGCGCGGCCGCCACACAACCGGTGATCGACACCCTCACCCAGGCCGC |
| | | TGCCACGATCCTCGGCACCGGGAGCGAGGTGGCATCCGACGCCCACTTCACCGACCTGGGCGGGGATTCC |
| | | CTGTCGGCGCTGACACTTTCGAACCTGCTGAGCGATTTCTTCGGTTTCGAAGTTCCCGTCGGCACCATCG |
| | | TGAACCCGGCCACCAACCTCGCCCAACTCGCCCAGCACATCGAGGCGCAGCGCACCGCGGGTGACCGCAG |
| | | GCCGAGTTTCACCACCGTGCACGGCGCGGACGCCACCGAGATCCGGGCGAGTGAGCTGACCCTGGACAAG |
| | | TTCATCGACGCCGAAACGCTCCGGGCCGCACCGGGTCTGCCCAAGGTCACCACCGAGCCACGGACGGTGT |
| | | TGCTCTCGGGCGCCAACGGCTGGCTGGGCCGGTTCCTCACGTTGCAGTGGCTGGAACGCCTGGCACCTGT |
| | | CGGCGGCACCCTCATCACGATCGTGCGGGGCCGCGACGACGCCGCGGCCCGCGCACGGCTGACCCAGGCC |
| | | TACGACACCGATCCCGAGTTGTCCCGCCGCTTCGCCGAGCTGGCCGACCGCCACCTGCGGGTGGTCGCCG |
| | | GTGACATCGGCGACCCGAATCTGGGCCTCACACCCGAGATCTGGCACCGGCTCGCCGCCGAGGTCGACCT |
| | | GGTGGTGCATCCGGCAGCGCTGGTCAACCACGTGCTCCCCTACCGGCAGCTGTTCGGCCCCAACGTCGTG |
| | | GGCACGGCCGAGGTGATCAAGCTGGCCCTCACCGAACGGATCAAGCCCGTCACGTACCTGTCCACCGTGT |
| | | CGGTGGCCATGGGGATCCCCGACTTCGAGGAGGACGGCGACATCCGGACCGTGAGCCCGGTGCGCCCGCT |
| | | CGACGGCGGATACGCCAACGGCTACGGCAACAGCAAGTGGGCCGGCGAGGTGCTGCTGCGGGAGGCCCAC |
| | | GATCTGTGCGGGCTGCCCGTGGCGACGTTCCGCTCGGACATGATCCTGGCGCATCCGCGCTACCGCGGTC |
| | | AGGTCAACGTGCCAGACATGTTCACGCGACTCCTGTTGAGCCTCTTGATCACCGGCGTCGCGCCGCGGTC |
| | | GTTCTACATCGGAGACGGTGAGCGCCCGCGGGCGCACTACCCCGGCCTGACGGTCGATTTCGTGGCCGAG |
| | | GCGGTCACGACGCTCGGCGCGCAGCAGCGCGAGGGATACGTGTCCTACGACGTGATGAACCCGCACGACG |
| | | ACGGGATCTCCCTGGATGTGTTCGTGGACTGGCTGATCCGGGCGGGCCATCCGATCGACCGGGTCGACGA |
| | | CTACGACGACTGGGTGCGTCGGTTCGAGACCGCGTTGACCGCGCTTCCCGAGAAGCGCCGCGCACAGACC |
| | | GTACTGCCGCTGCTGCACGCGTTCCGCGCTCCGCAGGCACCGTTGCGCGGCGCACCCGAACCCACGGAGG |
| | | TGTTCCACGCCGCGGTGCGCACCGCGAAGGTGGGCCCGGGAGACATCCCGCACCTCGACGAGGCGCTGAT |
| | | CGACAAGTACATACGCGATCTGCGTGAGTTCGGTCTGATCTGAGGTACCAGGAGGTTTTTACAATGGCTG |
| | | ATACTTTGTTGATTTTGGGTGATTCTCTCTCTGCAGGCTACCGTATGTCCGCGAGCGCGGCATGGCCGGC |
| | | TCTGCTGAACGATAAGTGGCAGAGCAAGACCAGCGTGGTCAATGCGAGCATCAGCGGCGATACCAGCCAG |
| | | CAGGGTCTGGCACGTCTGCCAGCGCTGCTGAAGCAACACCAGCCGCGTTGGGTGCTGGTTGAACTGGGCG |
| | | GCAATGACGGTCTGCGTGGTTTTCAGCCGCAGCAGACCGAACAAACGTTGCGTCAGATTCTGCAGGACGT |
| | | CAAGGCGGCTAACGCGGAACCGCTGCTGATGCAAATTCGCCTGCCGGCGAATTATGGTCGTCGTTACAAC |
| | | GAGGCTTTCAGCGCCATTTATCCTAAACTGGCTAAAGAGTTTGACGTGCCGCTGCTGCCGTTCTTCATGG |
| | | AAGAGGTCTACCTGAAACCGCAATGGATGCAAGACGACGGTATTCATCCGAATCGTGATGCACAACCTTT |
| | | CATCGCGGATTGGATGGCGAAGCAATTGCAACCGCTGGTGAACCATGACTCGTAAAAGCTTGTTGCTGCA |
| | | TGCAGGAGGTTTTTACAATGAAAACGACCCACACCAGCTTACCATTTGCCGGCCACACGTTACATTTCGT |
| | | CGAATTTGATCCGGCGAACTTTTGTGAACAAGACCTGTTGTGGCTGCCGCATTATGCCCAGCTGCAGCAC |
| | | GCAGGCCGTAAGCGTAAAACTGAACATCTGGCCGGTCGCATTGCGGCAGTGTATGCCCTGCGCGAGTACG |
| | | GCTACAAATGCGTGCCGGCCATTGGTGAACTGCGTCAACCGGTTTGGCCGGCAGAAGTTTACGGTTCCAT |
| | | CTCCCACTGCGGTACTACCGCGTTGGCGGTTGTGTCTCGCCAGCCGATCGGTATTGATATTGAAGAGATA |
| | | TTCTCTGTCCAGACGGCACGCGAGCTGACGGACAACATCATTACCCCGGCAGAGCACGAGCGTCTGGCGG |
| | | ACTGTGGTCTGGCGTTCAGCCTGGCGCTGACCCTGGCATTCAGCGCAAAAGAGAGCGCGTTCAAGGCTTC |
| | | CGAGATCCAAACCGATGCGGGCTTCCTGGATTATCAAATCATCAGCTGGAACAAGCAACAGGTTATCATT |
| | | CACCGTGAGAATGAGATGTTTGCCGTCCATTGGCAGATTAAAGAGAAAATCGTTATCACCCTGTGCCAGC |
| | | ACGACTGAGAATTC |
| 35 | plasmid | AAAAGCAGAGCATTACGCTGACTTGACGGGACGGCGCAAGCTCATGACCAAAATCCCTTAACGTGAGTTA |
| | pAQ3::P(nir07)- | CGCGCGCGTCGTTCCACTGAGCGTCAGACCCCGTAGAAAAGATCAAAGGATCTTCTTGAGATCCTTTTTT |
| | adm-carB- | TCTGCGCGTAATCTGCTGCTTGCAAACAAAAAAACCACCGCTACCAGCGGTGGTTTGTTTGCCGGATCAA |
| | tesAm-entD- | GAGCTACCAACTCTTTTTCCGAAGGTAACTGGCTTCAGCAGAGCGCAGATACCAAATACTGTTCTTCTAG |
| | SpecR. | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | CAAGAACTGGCCCTGAGAAGCGAGCTGGACTTCAATAGCGAAACCTATAAAGATGCGTATAGCCGTATTA |
| | | ACGCCATTGTGATCGAAGGCGAGCAAGAAGCATACCAAAACTACCTGGACATGGCGCAACTGCTGCCGGA |
| | | GGACGAGGCTGAGCTGATTCGTTTGAGCAAGATGGAGAACCGTCACAAAAAGGGTTTTCAAGCGTGCGGC |
| | | AAGAACCTCAATGTGACTCCGGATATGGATTATGCACAGCAGTTCTTTGCGGAGCTGCACGGCAATTTTC |
| | | AGAAGGCTAAAGCCGAGGGTAAGATTGTTACCTGCCTGCTCATCCAAAGCCTGATCATCGAGGCGTTTGC |
| | | GATTGCAGCCTACAACATTTACATTCCAGTGGCTGATCCGTTTGCACGTAAAATCACCGAGGGTGTCGTC |
| | | AAGGATGAGTATACCCACCTGAATTTCGGCGAAGTTTGGTTGAAGGAACATTTTGAAGCAAGCAAGGCGG |
| | | AGTTGGAGGACGCCAACAAAGAGAACTTACCGCTGGTCTGGCAGATGTTGAACCAGGTCGAAAAGGATGC |
| | | CGAAGTGCTGGGTATGGAGAAAGAGGCTCTGGTGGAGGACTTTATGATTAGCTATGGTGAGGCACTGAGC |
| | | AACATCGGCTTTTCTACGAGAGAAATCATGAAGATGAGCGCGTACGGTCTGCGTGCAGCATAAGAGCTCG |
| | | AGGAGGTTTTTACAATGACCAGCGATGTTCACGACGCCACAGACGGCGTCACCGAAACCGCACTCGACGA |
| | | CGAG<+GTCGACCCGCCGCATCGCCGAGCTGTACGCCACCGATCCCGAGTTCGCCGCCGCCGCACCGTTG |
| | | CCCGCCGTGGTCGACGCGGCGCACAAACCCGGGCTGCGGCTGGCAGAGATCCTGCAGACCCTGTTCACCG |
| | | GCTACGGTGACCGCCCGGCGCTGGGATACCGCGCCCGTGAACTGGCCACCGACGAGGGCGGGCGCACCGT |
| | | GACGCGTCTGCTGCCGCGGTTCGACACCCTCACCTACGCCCAGGTGTGGTCGCGCGTGCAAGCGGTCGCC |
| | | GCGGCCCTGCGCCACAACTTCGCGCAGCCGATCTACCCCGGCGACGCCGTCGCGACGATCGGTTTCGCGA |
| | | GTCCCGATTACCTGACGCTGGATCTCGTATGCGCCTACCTGGGCCTCGTGAGTGTTCCGCTGCAGCACAA |
| | | CGCACCGGTCAGCCGGCTCGCCCCGATCCTGGCCGAGGTCGAACCGCGGATCCTCACCGTGAGCGCCGAA |
| | | TACCTCGACCTCGCAGTCGAATCCGTGCGGGACGTCAACTCGGTGTCGCAGCTCGTGGTGTTCGACCATC |
| | | ACCCCGAGGTCGACGACCACCGCGACGCACTGGCCCGCGCGCGTGAACAACTCGCCGGCAAGGGCATCGC |
| | | CGTCACCACCCTGGACGCGATCGCCGACGAGGGCGCCGGGCTGCCGGCCGAACCGATCTACACCGCCGAC |
| | | CATGATCAGCGCCTCGCGATGATCCTGTACACCTCGGGTTCCACCGGCGCACCCAAGGGTGCGATGTACA |
| | | CCGAGGCGATGGTGGCGCGGCTGTGGACCATGTCGTTCATCACGGGTGACCCCACGCCGGTCATCAACGT |
| | | CAACTTCATGCCGCTCAACCACCTGGGCGGGCGCATCCCCATTTCCACCGCCGTGCAGAACGGTGGAACC |
| | | AGTTACTTCGTACCGGAATCCGACATGTCCACGCTGTTCGAGGATCTCGCGCTGGTGCGCCCGACCGAAC |
| | | TCGGCCTGGTTCCGCGCGTCGCCGACATGCTCTACCAGCACCACCTCGCCACCGTCGACCGCCTGGTCAC |
| | | GCAGGGCGCCGACGAACTGACCGCCGAGAAGCAGGCCGGTGCCGAACTGCGTGAGCAGGTGCTCGGCGGA |
| | | CGCGTGATCACCGGATTCGTCAGCACCGCACCGCTGGCCGCGGAGATGAGGGCGTTCCTCGACATCACCC |
| | | TGGGCGCACACATCGTCGACGGCTACGGGCTCACCGAGACCGGCGCCGTGACACGCGACGGTGTGATCGT |
| | | GCGGCCACCGGTGATCGACTACAAGCTGATCGACGTTCCCGAACTCGGCTACTTCAGCACCGACAAGCCC |
| | | TACCCGCGTGGCGAACTGCTGGTCAGGTCGCAAACGCTGACTCCCGGGTACTACAAGCGCCCCGAGGTCA |
| | | CCGCGAGCGTCTTCGACCGGGACGGCTACTACCACACCGGCGACGTCATGGCCGAGACCGCACCCGACCA |
| | | CCTGGTGTACGTGGACCGTCGCAACAACGTCCTCAAACTCGCGCAGGGCGAGTTCGTGGCGGTCGCCAAC |
| | | CTGGAGGCGGTGTTCTCCGGCGCGGCGCTGGTGCGCCAGATCTTCGTGTACGGCAACAGCGAGCGCAGTT |
| | | TCCTTCTGGCCGTGGTGGTCCCGACGCCGGAGGCGCTCGAGCAGTACGATCCGGCCGCGCTCAAGGCCGC |
| | | GCTGGCCGACTCGCTGCAGCGCACCGCACGCGACGCCGAACTGCAATCCTACGAGGTGCCGGCCGATTTC |
| | | ATCGTCGAGACCGAGCCGTTCAGCGCCGCCAACGGGCTGCTGTCGGGTGTCGGAAAACTGCTGCGGCCCA |
| | | ACCTCAAAGACCGCTACGGGCAGCGCCTGGAGCAGATGTACGCCGATATCGCGGCCACGCAGGCCAACCA |
| | | GTTGCGCGAACTGCGGCGCGCGGCCGCCACACAACCGGTGATCGACACCCTCACCCAGGCCGCTGCCACG |
| | | ATCCTCGGCACCGGGAGCGAGGTGGCATCCGACGCCCACTTCACCGACCTGGGCGGGGATTCCCTGTCGG |
| | | CGCTGACACTTTCGAACCTGCTGAGCGATTTCTTCGGTTTCGAAGTTCCCGTCGGCACCATCGTGAACCC |
| | | GGCCACCAACCTCGCCCAACTCGCCCAGCACATCGAGGCGCAGCGCACCGCGGGTGACCGCAGGCCGAGT |
| | | TTCACCACCGTGCACGGCGCGGACGCCACCGAGATCCGGGCGAGTGAGCTGACCCTGGACAAGTTCATCG |
| | | ACGCCGAAACGCTCCGGGCCGCACCGGGTCTGCCCAAGGTCACCACCGAGCCACGGACGGTGTTGCTCTC |
| | | GGGCGCCAACGGCTGGCTGGGCCGGTTCCTCACGTTGCAGTGGCTGGAACGCCTGGCACCTGTCGGCGGC |
| | | ACCCTCATCACGATCGTGCGGGGCCGCGACGACGCCGCGGCCCGCGCACGGCTGACCCAGGCCTACGACA |
| | | CCGATCCCGAGTTGTCCCGCCGCTTCGCCGAGCTGGCCGACCGCCACCTGCGGGTGGTCGCCGGTGACAT |
| | | CGGCGACC<MAATCTG<WCCTCACACCCGAGATCTGGCACCGGCTCGCCGCCGAGGTCGACCTGGTGGTG |
| | | CATCCGGCAGCGCTGGTCAACCACGTGCTCCCCTACCGGCAGCTGTTCGGCCCCAACGTCGTGGGCACGG |
| | | CCGAGGTGATCAAGCTGGCCCTCACCGAACGGATCAAGCCCGTCACGTACCTGTCCACCGTGTCGGTGGC |
| | | CATGGGGATCCCCGACTTCGAGGAGGACGGCGACATCCGGACCGTGAGCCCGGTGCGCCCGCTCGACGGC |
| | | GGATACGCCAACGGCTACGGCAACAGCAAGTGGGCCGGCGAGGTGCTGCTGCGGGAGGCCCACGATCTGT |
| | | GCGGGCTGCCCGTGGCGACGTTCCGCTCGGACATGATCCTGGCGCATCCGCGCTACCGCGGTCAGGTCAA |
| | | CGTGCCAGACATGTTCACGCGACTCCTGTTGAGCCTCTTGATCACCGGCGTCGCGCCGCGGTCGTTCTAC |
| | | ATCGGAGACGGTGAGCGCCCGCGGGCGCACTACCCCGGCCTGACGGTCGATTTCGTGGCCGAGGCGGTCA |
| | | CGACGCTCGGCGCGCAGCAGCGCGAGGGATACGTGTCCTACGACGTGATGAACCCGCACGACGACGGGAT |
| | | CTCCCTGGATGTGTTCGTGGACTGGCTGATCCGGGCGGGCCATCCGATCGACCGGGTCGACGACTACGAC |
| | | GACTGGGTGCGTCGGTTCGAGACCGCGTTGACCGCGCTTCCCGAGAAGCGCCGCGCACAGACCGTACTGC |
| | | CGCTGCTGCACGCGTTCCGCGCTCCGCAGGCACCGTTGCGCGGCGCACCCGAACCCACGGAGGTGTTCCA |
| | | CGCCGCGGTGCGCACCGCGAAGGTGGGCCCGGGAGACATCCCGCACCTCGACGAGGCGCTGATCGACAAG |
| | | TACATACGCGATCTGCGTGAGTTCGGTCTGATCTGAGGTACCAGGAGGTTTTTACAATGGCTGATACTTT |
| | | GTTGATTTTGGGTGATTCTCTCTCTGCAGGCTACCGTATGTCCGCGAGCGCGGCATGGCCGGCTCTGCTG |
| | | AACGATAAGTGGCAGAGCAAGACCAGCGTGGTCAATGCGAGCATCAGCGGCGATACCAGCCAGCAGGGTC |
| | | TGGCACGTCTGCCAGCGCTGCTGAAGCAACACCAGCCGCGTTGGGTGCTGGTTGAACTGGGCGGCAATGA |
| | | CGGTCTGCGTGGTTTTCAGCCGCAGCAGACCGAACAAACGTTGCGTCAGATTCTGCAGGACGTCAAGGCG |
| | | GCTAACGCGGAACCGCTGCTGATGCAAATTCGCCTGCCGGCGAATTATGGTCGTCGTTACAACGAGGCTT |
| | | TCAGCGCCATTTATCCTAAACTGGCTAAAGAGTTTGACGTGCCGCTGCTGCCGTTCTTCATGGAAGAGGT |
| | | CTACCTGAAACCGCAATGGATGCAAGACGACGGTATTCATCCGAATCGTGATGCACAACCTTTCATCGCG |
| | | GATTGGATGGCGAAGCAATTGCAACCGCTGGTGAACCATGACTCGTAAAAGCTTGTTGCTGCATGCAGGA |
| | | GGTTTTTACAATGAAAACGACCCACACCAGCTTACCATTTGCCGGCCACACGTTACATTTCGTCGAATTT |
| | | GATCCGGCGAACTTTTGTGAACAAGACCTGTTGTGGCTGCCGCATTATGCCCAGCTGCAGCACGCAGGCC |
| | | GTAAGCGTAAAACTGAACATCTGGCCGGTCGCATTGCGGCAGTGTATGCCCTGCGCGAGTACGGCTACAA |
| | | ATGCGTGCCGGCCATTGGTGAACTGCGTCAACCGGTTTGGCCGGCAGAAGTTTACGGTTCCATCTCCCAC |
| | | TGCGGTACTACCGCGTTGGCGGTTGTGTCTCGCCAGCCGATCGGTATTGATATTGAAGAGATATTCTCTG |
| | | TCCAGACGGCACGCGAGCTGACGGACAACATCATTACCCCGGCAGAGCACGAGCGTCTGGCGGACTGTGG |
| | | TCTGGCGTTCAGCCTGGCGCTGACCCTGGCATTCAGCGCAAAAGAGAGCGCGTTCAAGGCTTCCGAGATC |
| | | CAAACCGATGCGGGCTTCCTGGATTATCAAATCATCAGCTGGAACAAGCAACAGGTTATCATTCACCGTG |
| | | AGAATGAGATGTTTGCCGTCCATTGGCAGATTAAAGAGAAAATCGTTATCACCCTGTGCCAGCACGACTG |
| | | AGAATTCGGTTTTCCGTCCTGTCTTGATTTTCAAGCAAACAATGCCTCCGATTTCTAATCGGAGGCATTT |
| | | GTTTTTGTTTATTGCAAAAACAAAAAATATTGTTACAAATTTTTACAGGCTATTAAGCCTACCGTCATAA |
| | | ATAATTTGCCATTTACTAGTTTTTAATTAACCAGAACCTTGACCGAACGCAGCGGTGGTAACGGCGCAGT |
| | | GGCGGTTTTCATGGCTTGTTATGACTGTTTTTTTGGGGTACAGTCTATGCCTCGGGCATCCAAGCAGCAA |
| | | GCGCGTTACGCCGTGGGTCGATGTTTGATGTTATGGAGCAGCAACGATGTTACGCAGCAGGGCAGTCGCC |
| | | CTAAAACAAAGTTAAACATCATGAGGGAAGCGGTGATCGCCGAAGTATCGACTCAACTATCAGAGGTAGT |
| | | TGGCGTCATCGAGCGCCATCTCGAACCGACGTTGCTGGCCGTACATTTGTACGGCTCCGCAGTGGATGGC |
| | | GGCCTGAAGCCACACAGTGATATTGATTTGCTGGTTACGGTGACCGTAAGGCTTGATGAAACAACGCGGC |
| | | GAGCTTTGATCAACGACCTTTTGGAAACTTCGGCTTCCCCTGGAGAGAGCGAGATTCTCCGCGCTGTAGA |
| | | AGTCACCATTGTTGTGCACGACGACATCATTCCGTGGCGTTATCCAGCTAAGCGCGAACTGCAATTTGGA |
| | | GAATGGCAGCGCAATGACATTCTTGCAGGTATCTTCGAGCCAGCCACGATCGACATTGATCTGGCTATCT |
| | | TGCTGACAAAAGCAAGAGAACATAGCGTTGCCTTGGTAGGTCCAGCGGCGGAGGAACTCTTTGATCCGGT |
| | | TCCTGAACAGGATCTATTTGAGGCGCTAAATGAAACCTTAACGCTATGGAACTCGCCGCCCGACTGGGCT |
| | | GGCGATGAGCGAAATGTAGTGCTTACGTTGTCCCGCATTTGGTACAGCGCAGTAACCGGCAAAATCGCGC |
| | | CGAAGGATGTCGCTGCCGACTGGGCAATGGAGCGCCTGCCGGCCCAGTATCAGCCCGTCATACTTGAAGC |
| | | TAGACAGGCTTATCTTGGACAAGAAGAAGATCGCTTGGCCTCGCGCGCAGATCAGTTGGAAGAATTTGTC |
| | | CACTACGTGAAAGGCGAGATCACCAAGGTAGTCGGCAAATAATGTCTAACAATTCGTTCAAGCCGACGCC |
| | | GCTTCGCGGCGCGGCTTAACTCAAGCGTTAGATGCACTAAGCACATAATTGCTCACAGCCAAACTATCAG |
| | | GTCAAGTCTGCTTTTATTATTTTTAAGCGTGCATAATAAGCCCTACACAAATTGGGAGATATATCATGAG |
| | | GCGCGCCACGAGAAAGAGTTATGACAAATTAAAATTCTGACTCTTAGATTATTTCCAGAGAGGCTGATTT |
| | | TCCCAATCTTTGGGAAAGCCTAAGTTTTTAGATTCTATTTCTGGATACATCTCAAAAGTTCTTTTTAAAT |
| | | GCTGTGCAAAATTATGCTCTGGTTTAATTCTGTCTAAGAGATACTGAATACAACATAAGCCAGTGAAAAT |
| | | TTTACGGCTGTTTCTTTGATTAATATCCTCCAATACTTCTCTAGAGAGCCATTTTCCTTTTAACCTATCA |
| | | GGCAATTTAGGTGATTCTCCTAGCTGTATATTCCAGAGCCTTGAATGATGAGCGCAAATATTTCTAATAT |
| | | GCGACAAAGACCGTAACCAAGATATAAAAAACTTGTTAGGTAATTGGAAATGAGTATGTATTTTTTGTCG |
| | | TGTCTTAGATGGTAATAAATTTGTGTACATTCTAGATAACTGCCCAAAGGCGATTATCTCCAAAGCCATA |
| | | TATGACGGCGGTAGTAGAGGATTTGTGTACTTGTTTCGATAATGCCCGATAAATTCTTCTACTTTTTTAG |
| | | ATTGGCAATATTGAGTAATCGAATCGATTAATTCTTGATGCTTCCCAGTGTCATAAAATAAACTTTTATT |
| | | CAGATACCAATGAGGATCATAATCATGGGAGTAGTGATAAATCATTTGAGTTCTGACTGCTACTTCTATC |
| | | GACTCCGTAGCATTAAAAATAAGCATTCTCAAGGATTTATCAAACTTGTATAGATTTGGCCGGCCCGTCA |
| | | AAAGGGCGACACCCCATAATTAGCCCGGGCGAAAGGCCCAGTCTTTCGACTGAGCCTTTCGTTTTATTTG |
| | | ATGCCTGGCAGTTCCCTACTCTCGCATGGGGAGTCCCCACACTACCATCGGCGCTACGGCGTTTCACTTC |
| | | TGAGTTCGGCATGGGGTCAGGTGGGACCACCGCGCTACTGCCGCCAGGCAAACAAGGGGTGTTATGAGCC |
| | | ATATTCAGGTATAAATGGGCTCGCGATAATGTTCAGAATTGGTTAATTGGTTGTAACACTGACCCCTATT |
| | | TGTTTATTTTTCTAAATACATTCAAATATGTATCCGCTCATGAGACAATAACCCTGATAAATGCTTCAAT |
| | | AATATTGAAAAAGGAAGAATATGAGTATTCAACATTTCCGTGTCGCCCTTATTCCCTTTTTTGCGGCATT |
| | | TTGCCTTCCTGTTTTTGCTCACCCAGAAACGCTGGTGAAAGTAAAAGATGCTGAAGATCAGTTGGGTGCA |
| | | CGAGTGGGTTACATCGAACTGGATCTCAACAGCGGTAAGATCCTTGAGAGTTTTCGCCCCGAAGAACGTT |
| | | TTCCAATGATGAGCACTTTTAAAGTTCTGCTATGTGGCGCGGTATTATCCCGTATTGACGCCGGGCAAGA |
| | | GCAACTCGGTCGCCGCATACACTATTCTCAGAATGACTTGGTTGAGTACTCACCAGTCACAGAAAAGCAT |
| | | CTTACGGATGGCATGACAGTAAGAGAATTATGCAGTGCTGCCATAACCATGAGTGATAACACTGCGGCCA |
| | | ACTTACTTCTGACAACGATCGGAGGACCGAAGGAGCTAACCGCTTTTTTGCACAACATGGGGGATCATGT |
| | | AACTCGCCTTGATCGTTGGGAACCGGAGCTGAATGAAGCCATACCAAACGACGAGCGTGACACCACGATG |
| | | CCTGTAGCGATGGCAACAACGTTGCGCAAACTATTAACTGGCGAACTACTTACTCTAGCTTCCCGGCAAC |
| | | AATTAATAGACTGGATGGAGGCGGATAAAGTTGCAGGACCACTTCTGCGCTCGGCCCTTCCGGCTGGCTG |
| | | GTTTATTGCTGATAAATCCGGAGCCGGTGAGCGTGGTTCTCGCGGTATCATCGCAGCGCTGGGGCCAGAT |
| | | GGTAAGCCCTCCCGTATCGTAGTTATCTACACGACGGGGAGTCAGGCAACTATGGATGAACGAAATAGAC |
| | | AGATCGCTGAGATAGGTGCCTCACTGATTAAGCATTGGT |
| 36 | N. | MQQLTDQSKELDFKSETYKDAYSRINAIVIEGEQEAHENYITLAQLLPESHDELIRLSKMESRHKKGFEA |
| | punctiforme | CGRNLAVTPDLQFAKEFFSGLHQNFQTAAAEGKVVTCLLIQSLIIECFAIAAYNIYIPVADDFARKITEG |
| | Adm sequence | VVKEEYSHLNEGEVWLKEHFAESKAELELANRQNLPIVWKMLNQVEGDAHTMAMEKDALVEDFMIQYGEA |
| | (polypeptide) | LSNIGFSTRDIMRLSAYGLIGA |
| 37 | N. | ATGCAGCAACTGACCGATCAAAGCAAAGAACTGGACTTCAAGAGCGAGACGTACAAAGACGCCTATAGCC |
| | punctiforme | GCATTAACGCGATCGTCATTGAAGGCGAACAAGAGGCGCATGAAAACTACATCACCCTGGCGCAGCTGCT |
| | adm sequence | GCCTGAGAGCCACGACGAACTGATTCGCCTGAGCAAAATGGAGAGCCGTCACAAGAAAGGTTTTGAGGCG |
| | (nucleotide) , | TGTGGCCGCAATCTGGCGGTGACCCCGGACCTGCAATTTGCGAAGGAGTTCTTTAGCGGTCTGCACCAGA |
| | codon- | ATTTCCAGACGGCCGCAGCCGAGGGCAAAGTCGTCACTTGTTTGTTGATCCAGAGCCTGATTATTGAATG |
| | optimized for | CTTTGCTATTGCGGCGTACAACATTTACATTCCGGTCGCCGATGACTTTGCGCGTAAAATCACGGAAGGT |
| | E. coli | GTTGTCAAAGAGGAGTATTCCCACCTGAATTTCGGTGAAGTGTGGTTGAAGGAACATTTTGCGGAATCTA |
| | | AAGCCGAATTGGAACTGGCAAATCGCCAGAACCTGCCGATCGTTTGGAAGATGCTGAACCAAGTGGAAGG |
| | | TGATGCACATACGATGGCGATGGAGAAGGACGCATTGGTTGAGGACTTTATGATTCAGTATGGCGAAGCA |
| | | CTGTCCAATATCGGTTTCAGCACCCGTGATATCATGCGTCTGAGCGCCTATGGCCTGATCGGTGCCTAA |
| 38 | N. | MGSSHHHHHHSQDPMQQLTDQSKELDFKSETYKDAYSRINAIVIEGEQEA |
| | Imnctiforme | HENYITLAQLLPESHDELIRLSKMESRHKKGFEACGRNLAVTPDLQFAKE |
| | His-Tagged | FFSGLHQNFQTAAAEGKVVTCLLIQSLIIECFAIAAYNIYIPVADDFARK |
| | Adm sequence | ITEGVVKEEYSHLNFGEVWLKEHFAESKAELELANRQNLPIVWKMLNQVE |
| | (polypeptide) | GDAHTMAMEKDALVEDFMIQYGEALSNIGFSTRDIMRLSAYGLIGA |
| 39 | N. | ATGGGCAGCAGCCATCACCATCATCACCACAGCCAGGATCCGATGCAGCAACTGACCGATCAAAGCAAAG |
| | Imnctiforme | AACTGGACTTCAAGAGCGAGACGTACAAAGACGCCTATAGCCGCATTAACGCGATCGTCATTGAAGGCGA |
| | adm sequence | ACAAGAGGCGCATGAAAACTACATCACCCTGGCGCAGCTGCTGCCTGAGAGCCACGACGAACTGATTCGC |
| | (His-Tagged) | CTGAGCAAAATGGAGAGCCGTCACAAGAAAGGTTTTGAGGCGTGTGGCCGCAATCTGGCGGTGACCCCGG |
| | (nucleotide) | ACCTGCAATTTGCGAAGGAGTTCTTTAGCGGTCTGCACCAGAATTTCCAGACGGCCGCAGCCGAGGGCAA |
| | | AGTCGTCACTTGTTTGTTGATCCAGAGCCTGATTATTGAATGCTTTGCTATTGCGGCGTACAACATTTAC |
| | | ATTCCGGTCGCCGATGACTTTGCGCGTAAAATCACGGAAGGTGTTGTCAAAGAGGAGTATTCCCACCTGA |
| | | ATTTCGGTGAAGTGTGGTTGAAGGAACATTTTGCGGAATCTAAAGCCGAATTGGAACTGGCAAATCGCCA |
| | | GAACCTGCCGATCGTTTGGAAGATGCTGAACCAAGTGGAAGGTGATGCACATACGATGGCGATGGAGAAG |
| | | GACGCATTGGTTGAGGACTTTATGATTCAGTATGGCGAAGCACTGTCCAATATCGGTTTCAGCACCCGTG |
| | | ATATCATGCGTCTGAGCGCCTATGGCCTGATCGGTGCC |
| 40 | N. | 5'-CATCACCACAGCCAGGATCCGATGCAGCAACTGACCGATCAAAGCAAA |
| | punctiforme | GAACTGGACTTC-3' |
| | adm Primer | |
| | UN19 | |
| 41 | N. | 5'-CGGCCCGCCAAGCTTTTAGGCACCGATCAGGCCATAGGCGCTCAGACG |
| | punctiforme | CATGATATC-3' |
| | adm Primer | |
| | UN20 | |
| 42 | Plasmid pCDF- | GGGGAATTGTGAGCGGATAACAATTCCCCTGTAGAAATAATTTTGTTTAACTTTAATAAGGAGATATACC |
| | npu (Table 5 | ATGGGCAGCAGCCATCACCATCATCACCACAGCCAGGATCCGATGCAGCAACTGACCGATCAAAGCAAAG |
| | for key) | AACTGGACTTCAAGAGCGAGACGTACAAAGACGCCTATAGCCGCATTAACGCGATCGTCATTGAAGGCGA |
| | | ACAAGAGGCGCATGAAAACTACATCACCCTGGCGCAGCTGCTGCCTGAGAGCCACGACGAACTGATTCGC |
| | | CTGAGCAAAATGGAGAGCCGTCACAAGAAAGGTTTTGAGGCGTGTGGCCGCAATCTGGCGGTGACCCCGG |
| | | ACCTGCAATTTGCGAAGGAGTTCTTTAGCGGTCTGCACCAGAATTTCCAGACGGCCGCAGCCGAGGGCAA |
| | | AGTCGTCACTTGTTTGTTGATCCAGAGCCTGATTATTGAATGCTTTGCTATTGCGGCGTACAACATTTAC |
| | | ATTCCGGTCGCCGATGACTTTGCGCGTAAAATCACGGAAGGTGTTGTCAAAGAGGAGTATTCCCACCTGA |
| | | ATTTCGGTGAAGTGTGGTTGAAGGAACATTTTGCGGAATCTAAAGCCGAATTGGAACTGGCAAATCGCCA |
| | | GAACCTGCCGATCGTTTGGAAGATGCTGAACCAAGTGGAAGGTGATGCACATACGATGGCGATGGAGAAG |
| | | GACGCATTGGTTGAGGACTTTATGATTCAGTATGGCGAAGCACTGTCCAATATCGGTTTCAGCACCCGTG |
| | | ATATCATGCGTCTGAGCGCCTATGGCCTGATCGGTGCCTAAAGCTTGCGGCCGCATAATGCTTAAGTCGA |
| | | ACAGAAAGTAATCGTATTGTACACGGCCGCATAATCGAAATTAATACGACTCACTATAGGGGAATTGTGA |
| | | GCGGATAACAATTCCCCATCTTAGTATATTAGTTAAGTATAAGAAGGAGATATACATATGGCAGATCTCA |
| | | ATTGGATATCGGCCGGCCACGCGATCGCTGACGTCGGTACCCTCGAGTCTGGTAAAGAAACCGCTGCTGC |
| | | GAAATTTGAACGCCAGCACATGGACTCGTCTACTAGCGCAGCTTAATTAACCTAGGCTGCTGCCACCGCT |
| | | GAGCAATAACTAGCATAACCCCTTGGGGCCTCTAAACGGGTCTTGAGGGGTTTTTTGCTGAAACCTCAGG |
| | | CATTTGAGAAGCACACGGTCACACTGCTTCCGGTAGTCAATAAACCGGTAAACCAGCAATAGACATAAGC |
| | | GGCTATTTAACGACCCTGCCCTGAACCGACGACCGGGTCATCGTGGCCGGATCTTGCGGCCCCTCGGCTT |
| | | GAACGAATTGTTAGACATTATTTGCCGACTACCTTGGTGATCTCGCCTTTCACGTAGTGGACAAATTCTT |
| | | CCAACTGATCTGCGCGCGAGGCCAAGCGATCTTCTTCTTGTCCAAGATAAGCCTGTCTAGCTTCAAGTAT |
| | | GACGGGCTGATACTGGGCCGGCAGGCGCTCCATTGCCCAGTCGGCAGCGACATCCTTCGGCGCGATTTTG |
| | | CCGGTTACTGCGCTGTACCAAATGCGGGACAACGTAAGCACTACATTTCGCTCATCGCCAGCCCAGTCGG |
| | | GCGGCGAGTTCCATAGCGTTAAGGTTTCATTTAGCGCCTCAAATAGATCCTGTTCAGGAACCGGATCAAA |
| | | GAGTTCCTCCGCCGCTGGACCTACCAAGGCAACGCTATGTTCTCTTGCTTTTGTCAGCAAGATAGCCAGA |
| | | TCAATGTCGATCGTGGCTGGCTCGAAGATACCTGCAAGAATGTCATTGCGCTGCCATTCTCCAAATTGCA |
| | | GTTCGCGCTTAGCTGGATAACGCCACGGAATGATGTCGTCGTGCACAACAATGGTGACTTCTACAGCGCG |
| | | GAGAATCTCGCTCTCTCCAGGGGAAGCCGAAGTTTCCAAAAGGTCGTTGATCAAAGCTCGCCGCGTTGTT |
| | | TCATCAAGCCTTACGGTCACCGTAACCAGCAAATCAATATCACTGTGTGGCTTCAGGCCGCCATCCACTG |
| | | CGGAGCCGTACAAATGTACGGCCAGCAACGTCGGTTCGAGATGGCGCTCGATGACGCCAACTACCTCTGA |
| | | TAGTTGAGTCGATACTTCGGCGATCACCGCTTCCCTCATACTCTTCCTTTTTCAATATTATTGAAGCATT |
| | | TATCAGGGTTATTGTCTCATGAGCGGATACATATTTGAATGTATTTAGAAAAATAAACAAATAGCTAGCT |
| | | CACTCGGTCGCTACGCTCCGGGCGTGAGACTGCGGCGGGCGCTGCGGACACATACAAAGTTACCCACAGA |
| | | TTCCGTGGATAAGCAGGGGACTAACATGTGAGGCAAAACAGCAGGGCCGCGCCGGTGGCGTTTTTCCATA |
| | | GGCTCCGCCCTCCTGCCAGAGTTCACATAAACAGACGCTTTTCCGGTGCATCTGTGGGAGCCGTGAGGCT |
| | | CAACCATGAATCTGACAGTACGGGCGAAACCCGACAGGACTTAAAGATCCCCACCGTTTCCGGCGGGTCG |
| | | CTCCCTCTTGCGCTCTCCTGTTCCGACCCTGCCGTTTACCGGATACCTGTTCCGCCTTTCTCCCTTACGG |
| | | GAAGTGTGGCGCTTTCTCATAGCTCACACACTGGTATCTCGGCTCGGTGTAGGTCGTTCGCTCCAAGCTG |
| | | GGCTGTAAGCAAGAACTCCCCGTTCAGCCCGACTGCTGCGCCTTATCCGGTAACTGTTCACTTGAGTCCA |
| | | ACCCGGAAAAGCACGGTAAAACGCCACTGGCAGCAGCCATTGGTAACTGGGAGTTCGCAGAGGATTTGTT |
| | | TAGCTAAACACGCGGTTGCTCTTGAAGTGTGCGCCAAAGTCCGGCTACACTGGAAGGACAGATTTGGTTG |
| | | CTGTGCTCTGCGAAAGCCAGTTACCACGGTTAAGCAGTTCCCCAACTGACTTAACCTTCGATCAAACCAC |
| | | CTCCCCAGGTGGTTTTTTCGTTTACAGGGCAAAAGATTACGCGCAGAAAAAAAGGATCTCAAGAAGATCC |
| | | TTTGATCTTTTCTACTGAACCGCTCTAGATTTCAGTGCAATTTATCTCTTCAAATGTAGCACCTGAAGTC |
| | | AGCCCCATACGATATAAGTTGTAATTCTCATGTTAGTCATGCCCCGCGCCCACCGGAAGGAGCTGACTGG |
| | | GTTGAAGGCTCTCAAGGGCATCGGTCGAGATCCCGGTGCCTAATGAGTGAGCTAACTTACATTAATTGCG |
| | | TTGCGCTCACTGCCCGCTTTCCAGTCGGGAAACCTGTCGTGCCAGCTGCATTAATGAATCGGCCAACGCG |
| | | CGGGGAGAGGCGGTTTGCGTATTGGGCGCCAGGGTGGTTTTTCTTTTCACCAGTGAGACGGGCAACAGCT |
| | | GATTGCCCTTCACCGCCTGGCCCTGAGAGAGTTGCAGCAAGCGGTCCACGCTGGTTTGCCCCAGCAGGCG |
| | | AAAATCCTGTTTGATGGTGGTTAACGGCGGGATATAACATGAGCTGTCTTCGGTATCGTCGTATCCCACT |
| | | ACCGAGATGTCCGCACCAACGCGCAGCCCGGACTCGGTAATGGCGCGCATTGCGCCCAGCGCCATCTGAT |
| | | CGTTGGCAACCAGCATCGCAGTGGGAACGATGCCCTCATTCAGCATTTGCATGGTTTGTTGAAAACCGGA |
| | | CATGGCACTCCAGTCGCCTTCCCGTTCCGCTATCGGCTGAATTTGATTGCGAGTGAGATATTTATGCCAG |
| | | CCAGCCAGACGCAGACGCGCCGAGACAGAACTTAATGGGCCCGCTAACAGCGCGATTTGCTGGTGACCCA |
| | | ATGCGACCAGATGCTCCACGCCCAGTCGCGTACCGTCTTCATGGGAGAAAATAATACTGTTGATGGGTGT |
| | | CTGGTCAGAGACATCAAGAAATAACGCCGGAACATTAGTGCAGGCAGCTTCCACAGCAATGGCATCCTGG |
| | | TCATCCAGCGGATAGTTAATGATCAGCCCACTGACGCGTTGCGCGAGAAGATTGTGCACCGCCGCTTTAC |
| | | AGGCTTCGACGCCGCTTCGTTCTACCATCGACACCACCACGCTGGCACCCAGTTGATCGGCGCGAGATTT |
| | | AATCGCCGCGACAATTTGCGACGGCGCGTGCAGGGCCAGACTGGAGGTGGCAACGCCAATCAGCAACGAC |
| | | TGTTTGCCCGCCAGTTGTTGTGCCACGCGGTTGGGAATGTAATTCAGCTCCGCCATCGCCGCTTCCACTT |
| | | TTTCCCGCGTTTTCGCAGAAACGTGGCTGGCCTGGTTCACCACGCGGGAAACGGTCTGATAAGAGACACC |
| | | GGCATACTCTGCGACATCGTATAACGTTACTGGTTTCACATTCACCACCCTGAATTGACTCTCTTCCGGG |
| | | CGCTATCATGCCATACCGCGAAAGGTTTTGCGCCATTCGATGGTGTCCGGGATCTCGACGCTCTCCCTTA |
| | | TGCGACTCCTGCATTAGGAAATTAATACGACTCACTATA |
| 43 | ketoacid | MYTVGDYLLDRLHELGIEEIFGVPGDYNLQFLDQIISRKDMKWVGNANELNASYMADGYARTKKAAAFLT |
| | decarboxylase | TFGVGELSAVNGLAGSYAENLPVVEIVGSPTSKVQNEGKEVHHTLADGDFKHFMKMHEPVTAARTLLTAE |
| | KivD | NATVEIDRVLSALLKERKPVYINLPVDVAAAKAEKPSLPLKKENPTSNTSDQEILNKIQESLKNAKKPIV |
| | (ADA65057) | ITGHEIISFGLENTVTQFISKTKLPITTLNFGKSSVDETLPSFLGIYNGKLSEPNLKEFVESADFILMLG |
| | from | VKLTDSSTGAFTHHLNENKMISLNIDEGKIFNESIQNFDFESLISSLLDLSGIEYKGKYIDKKQEDFVPS |
| | Lactococcus | NALLSQDRLWQAVENLTQSNETIVAEQGTSFFGASSIFLKPKSHFIGQPLWGSIGYTFPAALGSQIADKE |
| | lactis subsp. | SRHLLFIGDGSLQLTVQELGLAIREKINPICFIINNDGYTVEREIHGPNQSYNDIPMWNYSKLPESFGAT |
| | lactis KF147 | EERVVSKIVRTENEFVSVMKEAQADPNRMYWIELVLAKEDAPKVLKKMGKLFAEQNKS |
| | (polypeptide) | |
| 44 | ketoacid | MAPVTIEKEVNQEERHLVSNRSATIPFGEYIFKRLLSIDTKSVFGVPGDFNLSLLEYLYSPSVESAGLRW |
| | decarboxylase | VGTCNELNAAYAADGYSRYSNKIGCLITTYGVGELSALNGIAGSFAENVKVLHIVGVAKSIDSRSSNFSD |
| | ARO10 | RNLHHLVPQLHDSNFKGPNHKVYHDMVKDRVACSVAYLEDIETACDQVDNVIRDIYKYSKPGYIFVPADF |
| | (NP_010668) | ADMSVTCDNLVNVPRISQQDCIVYPSENQLSDIINKITSWIYSSKTPAILGDVLTDRYGVSNFLNKLICK |
| | from | TGIWNESTVMGKSVIDESNPTYMGQYNGKEGLKQVYEHFELCDLVLHFGVDINEINNGHYTFTYKPNAKI |
| | Saccharomyces | IQFHPNYIRLVDTRQGNEQMFKGINFAPILKELYKRIDVSKLSLQYDSNVTQYTNETMRLEDPTNGQSSI |
| | cerevisiae | ITQVHLQKTMPKFLNPGDVVVCETGSFQFSVRDFAFPSQLKYISQGFFLSIGMALPAALGVGIAMQDHSN |
| | S288c | AHINGGNVKEDYKPRLILFEGDGAAQMTIQELSTILKCNIPLEVIIWNNNGYTIERAIMGPTRSYNDVMS |
| | (polypeptide) | WKWTKLFEAFGDFDGKYTNSTLIQCPSKLALKLEELKNSNKRSGIELLEVKLGELDFPEQLKCMVEAAAL |
| | | KRNKK |
| 45 | pdc(Z. | ATGAGTTATACTGTCGGTACCTATTTAGCGGAGCGGCTTGTCCAGATTGGTCTCAAGCATCACTTCGCAG |
| | mobilis) | TCGCGGGCGACTACAACCTCGTCCTTCTTGACAACCTGCTTTTGAACAAAAACATGGAGCAGGTTTATTG |
| | nucleotide | CTGTAACGAACTGAACTGCGGTTTCAGTGCAGAAGGTTATGCTCGTGCCAAAGGCGCAGCAGCAGCCGTC |
| | sequence | GTTACCTACAGCGTCGGTGCGCTTTCCGCATTTGATGCTATCGGTGGCGCCTATGCAGAAAACCTTCCGG |
| | | TTATCCTGATCTCCGGTGCTCCGAACAACAATGATCACGCTGCTGGTCACGTGTTGCATCACGCTCTTGG |
| | | CAAAACCGACTATCACTATCAGTTGGAAATGGCCAAGAACATCACGGCCGCAGCTGAAGCGATTTACACC |
| | | CCAGAAGAAGCTCCGGCTAAAATCGATCACGTGATTAAAACTGCTCTTCGTGAGAAGAAGCCGGTTTATC |
| | | TCGAAATCGCTTGCAACATTGCTTCCATGCCCTGCGCCGCTCCTGGACCGGCAAGCGCATTGTTCAATGA |
| | | CGAAGCCAGCGACGAAGCTTCTTTGAATGCAGCGGTTGAAGAAACCCTGAAATTCATCGCCAACCGCGAC |
| | | AAAGTTGCCGTCCTCGTCGGCAGCAAGCTGCGCGCAGCTGGTGCTGAAGAAGCTGCTGTCAAATTTGCTG |
| | | ATGCTCTCGGTGGCGCAGTTGCTACCATGGCTGCTGCAAAAAGCTTCTTCCCAGAAGAAAACCCGCATTA |
| | | CATCGGTACCTCATGGGGTGAAGTCAGCTATCCGGGCGTTGAAAAGACGATGAAAGAAGCCGATGCGGTT |
| | | ATCGCTCTGGCTCCTGTCTTCAACGACTACTCCACCACTGGTTGGACGGATATTCCTGATCCTAAGAAAC |
| | | TGGTTCTCGCTGAACCGCGTTCTGTCGTCGTTAACGGCGTTCGCTTCCCCAGCGTTCATCTGAAAGACTA |
| | | TCTGACCCGTTTGGCTCAGAAAGTTTCCAAGAAAACCGGTGCTTTGGACTTCTTCAAATCCCTCAATGCA |
| | | GGTGAACTGAAGAAAGCCGCTCCGGCTGATCCGAGTGCTCCGTTGGTCAACGCAGAAATCGCCCGTCAGG |
| | | TCGAAGCTCTTCTGACCCCGAACACGACGGTTATTGCTGAAACCGGTGACTCTTGGTTCAATGCTCAGCG |
| | | CATGAAGCTCCCGAACGGTGCTCGCGTTGAATATGAAATGCAGTGGGGTCACATCGGTTGGTCCGTTCCT |
| | | GCCGCCTTCGGTTATGCCGTCGGTGCTCCGGAACGTCGCAACATCCTCATGGTTGGTGATGGTTCCTTCC |
| | | AGCTGACGGCTCAGGAAGTCGCTCAGATGGTTCGCCTGAAACTGCCGGTTATCATCTTCTTGATCAATAA |
| | | CTATGGTTACACCATCGAAGTTATGATCCATGATGGTCCGTACAACAACATCAAGAACTGGGATTATGCC |
| | | GGTCTGATGGAAGTGTTCAACGGTAACGGTGGTTATGACAGCGGTGCTGGTAAAGGCCTGAAGGCTAAAA |
| | | CCGGTGGCGAACTGGCAGAAGCTATCAAGGTTGCTCTGGCAAACACCGACGGCCCAACCCTGATCGAATG |
| | | CTTCATCGGTCGTGAAGACTGCACTGAAGAATTGGTCAAATGGGGTAAGCGCGTTGCTGCCGCCAACAGC |
| | | CGTAAGCCTGTTAACAAGCTCCTCTAG |
| 46 | Pdc(Z. | MSYTVGTYLAERLVQIGLKHHFAVAGDYNLVLLDNLLLNKNMEQVYCCNELNCGESAEGYARAKGAAAAV |
| | mobilis) | VTYSVGALSAFDAIGGAYAENLPVILISGAPNNNDHAAGHVLHHALGKTDYHYQLEMAKNITAAAEAIYT |
| | protein | PEEAPAKIDHVIKTALREKKPVYLEIACNIASMPCAAPGPASALENDEASDEASLNAAVEETLKFIANRD |
| | sequence | KVAVLVGSKLRAAGAEEAAVKFADALGGAVATMAAAKSFFPEENPHYIGTSWGEVSYPGVEKTMKEADAV |
| | | IALAPVENDYSTTGWTDIPDPKKLVLAEPRSVVVNGVRFPSVHLKDYLTRLAQKVSKKTGALDFFKSLNA |
| | | GELKKAAPADPSAPLVNAEIARQVEALLTPNTTVIAETGDSWENAQRMKLPNGARVEYEMQWGHIGWSVP |
| | | AAFGYAVGAPERRNILMVGDGSFQLTAQEVAQMVRLKLPVIIFLINNYGYTIEVMIHDGPYNNIKNWDYA |
| | | GLMEVFNGNGGYDSGAGKGLKAKTGGELAEAIKVALANTDGPTLIECFIGREDCTEELVKWGKRVAAANS |
| | | RKPVNKLL |
| 47 | carboxylic | ATGACCAGCGATGTTCACGACGCCACAGACGGCGTCACCGAAACCGCACTCGACGACGAGCAGTCGACCC |
| | acid | GCCGCATCGCCGAGCTGTACGCCACCGATCCCGAGTTCGCCGCCGCCGCACCGTTGCCCGCCGTGGTCGA |
| | reductase | CGCGGCGCACAAACCCGGGCTGCGGCTGGCAGAGATCCTGCAGACCCTGTTCACCGGCTACGGTGACCGC |
| | amplified | CCGGCGCTGGGATACCGCGCCCGTGAACTGGCCACCGACGAGGGCGGGCGCACCGTGACGCGTCTGCTGC |
| | from | CGCGGTTCGACACCCTCACCTACGCCCAGGTGTGGTCGCGCGTGCAAGCGGTCGCCGCGGCCCTGCGCCA |
| | Mycobacterium | CAACTTCGCGCAGCCGATCTACCCCGGCGACGCCGTCGCGACGATCGGTTTCGCGAGTCCCGATTACCTG |
| | smegmatis | ACGCTGGATCTCGTATGCGCCTACCTGGGCCTCGTGAGTGTTCCGCTGCAGCACAACGCACCGGTCAGCC |
| | | GGCTCGCCCCGATCCTGGCCGAGGTCGAACCGCGGATCCTCACCGTGAGCGCCGAATACCTCGACCTCGC |
| | | AGTCGAATCCGTGCGGGACGTCAACTCGGTGTCGCAGCTCGTGGTGTTCGACCATCACCCCGAGGTCGAC |
| | | GACCACCGCGACGCACTGGCCCGCGCGCGTGAACAACTCGCCGGCAAGGGCATCGCCGTCACCACCCTGG |
| | | ACGCGATCGCCGACGAGGGCGCCGGGCTGCCGGCCGAACCGATCTACACCGCCGACCATGATCAGCGCCT |
| | | CGCGATGATCCTGTACACCTCGGGTTCCACCGGCGCACCCAAGGGTGCGATGTACACCGAGGCGATGGTG |
| | | GCGCGGCTGTGGACCATGTCGTTCATCACGGGTGACCCCACGCCGGTCATCAACGTCAACTTCATGCCGC |
| | | TCAACCACCTGGGCGGGCGCATCCCCATTTCCACCGCCGTGCAGAACGGTGGAACCAGTTACTTCGTACC |
| | | GGAATCCGACATGTCCACGCTGTTCGAGGATCTCGCGCTGGTGCGCCCGACCGAACTCGGCCTGGTTCCG |
| | | CGCGTCGCCGACATGCTCTACCAGCACCACCTCGCCACCGTCGACCGCCTGGTCACGCAGGGCGCCGACG |
| | | AACTGACCGCCGAGAAGCAGGCCGGTGCCGAACTGCGTGAGCAGGTGCTCGGCGGACGCGTGATCACCGG |
| | | ATTCGTCAGCACCGCACCGCTGGCCGCGGAGATGAGGGCGTTCCTCGACATCACCCTGGGCGCACACATC |
| | | GTCGACGGCTACGGGCTCACCGAGACCGGCGCCGTGACACGCGACGGTGTGATCGTGCGGCCACCGGTGA |
| | | TCGACTACAAGCTGATCGACGTTCCCGAACTCGGCTACTTCAGCACCGACAAGCCCTACCCGCGTGGCGA |
| | | ACTGCTGGTCAGGTCGCAAACGCTGACTCCCGGGTACTACAAGCGCCCCGAGGTCACCGCGAGCGTCTTC |
| | | GACCGGGACGGCTACTACCACACCGGCGACGTCATGGCCGAGACCGCACCCGACCACCTGGTGTACGTGG |
| | | ACCGTCGCAACAACGTCCTCAAACTCGCGCAGGGCGAGTTCGTGGCGGTCGCCAACCTGGAGGCGGTGTT |
| | | CTCCGGCGCGGCGCTGGTGCGCCAGATCTTCGTGTACGGCAACAGCGAGCGCAGTTTCCTTCTGGCCGTG |
| | | GTGGTCCCGACGCCGGAGGCGCTCGAGCAGTACGATCCGGCCGCGCTCAAGGCCGCGCTGGCCGACTCGC |
| | | TGCAGCGCACCGCACGCGACGCCGAACTGCAATCCTACGAGGTGCCGGCCGATTTCATCGTCGAGACCGA |
| | | GCCGTTCAGCGCCGCCAACGGGCTGCTGTCGGGTGTCGGAAAACTGCTGCGGCCCAACCTCAAAGACCGC |
| | | TACGGGCAGCGCCTGGAGCAGATGTACGCCGATATCGCGGCCACGCAGGCCAACCAGTTGCGCGAACTGC |
| | | GGCGCGCGGCCGCCACACAACCGGTGATCGACACCCTCACCCAGGCCGCTGCCACGATCCTCGGCACCGG |
| | | GAGCGAGGTGGCATCCGACGCCCACTTCACCGACCTGGGCGGGGATTCCCTGTCGGCGCTGACACTTTCG |
| | | AACCTGCTGAGCGATTTCTTCGGTTTCGAAGTTCCCGTCGGCACCATCGTGAACCCGGCCACCAACCTCG |
| | | CCCAACTCGCCCAGCACATCGAGGCGCAGCGCACCGCGGGTGACCGCAGGCCGAGTTTCACCACCGTGCA |
| | | CGGCGCGGACGCCACCGAGATCCGGGCGAGTGAGCTGACCCTGGACAAGTTCATCGACGCCGAAACGCTC |
| | | CGGGCCGCACCGGGTCTGCCCAAGGTCACCACCGAGCCACGGACGGTGTTGCTCTCGGGCGCCAACGGCT |
| | | GGCTGGGCCGGTTCCTCACGTTGCAGTGGCTGGAACGCCTGGCACCTGTCGGCGGCACCCTCATCACGAT |
| | | CGTGCGGGGCCGCGACGACGCCGCGGCCCGCGCACGGCTGACCCAGGCCTACGACACCGATCCCGAGTTG |
| | | TCCCGCCGCTTCGCCGAGCTGGCCGACCGCCACCTGCGGGTGGTCGCCGGTGACATCGGCGACCCGAATC |
| | | TGGGCCTCACACCCGAGATCTGGCACCGGCTCGCCGCCGAGGTCGACCTGGTGGTGCATCCGGCAGCGCT |
| | | GGTCAACCACGTGCTCCCCTACCGGCAGCTGTTCGGCCCCAACGTCGTGGGCACGGCCGAGGTGATCAAG |
| | | CTGGCCCTCACCGAACGGATCAAGCCCGTCACGTACCTGTCCACCGTGTCGGTGGCCATGGGGATCCCCG |
| | | ACTTCGAGGAGGACGGCGACATCCGGACCGTGAGCCCGGTGCGCCCGCTCGACGGCGGATACGCCAACGG |
| | | CTACGGCAACAGCAAGTGGGCCGGCGAGGTGCTGCTGCGGGAGGCCCACGATCTGTGCGGGCTGCCCGTG |
| | | GCGACGTTCCGCTCGGACATGATCCTGGCGCATCCGCGCTACCGCGGTCAGGTCAACGTGCCAGACATGT |
| | | TCACGCGACTCCTGTTGAGCCTCTTGATCACCGGCGTCGCGCCGCGGTCGTTCTACATCGGAGACGGTGA |
| | | GCGCCCGCGGGCGCACTACCCCGGCCTGACGGTCGATTTCGTGGCCGAGGCGGTCACGACGCTCGGCGCG |
| | | CAGCAGCGCGAGGGATACGTGTCCTACGACGTGATGAACCCGCACGACGACGGGATCTCCCTGGATGTGT |
| | | TCGTGGACTGGCTGATCCGGGCGGGCCATCCGATCGACCGGGTCGACGACTACGACGACTGGGTGCGTCG |
| | | GTTCGAGACCGCGTTGACCGCGCTTCCCGAGAAGCGCCGCGCACAGACCGTACTGCCGCTGCTGCACGCG |
| | | TTCCGCGCTCCGCAGGCACCGTTGCGCGGCGCACCCGAACCCACGGAGGTGTTCCACGCCGCGGTGCGCA |
| | | CCGCGAAGGTGGGCCCGGGAGACATCCCGCACCTCGACGAGGCGCTGATCGACAAGTACATACGCGATCT |
| | | GCGTGAGTTCGGTCTGATCTGA |
| 48 | codon- | ATGCACCATCACCACCATCATGGAGGCGGACAGCAACTGACCGATCAAAGCAAAGAACTGGACTTCAAGA |
| | optimized | GCGAGACGTACAAAGACGCCTATAGCCGCATTAACGCGATCGTCATTGAAGGCGAACAAGAGGCGCATGA |
| | hexahistidine- | AAACTACATCACCCTGGCGCAGCTGCTGCCTGAGAGCCACGACGAACTGATTCGCCTGAGCAAAATGGAG |
| | tagged | AGCCGTCACAAGAAAGGTTTTGAGGCGTGTGGCCGCAATCTGGCGGTGACCCCGGACCTGCAATTTGCGA |
| | Nostoc | AGGAGTTCTTTAGCGGTCTGCACCAGAATTTCCAGACGGCCGCAGCCGAGGGCAAAGTCGTCACTTGTTT |
| | punctiforme | GTTGATCCAGAGCCTGATTATTGAATGCTTTGCTATTGCGGCGTACAACATTTACATTCCGGTCGCCGAT |
| | adm. | GACTTTGCGCGTAAAATCACGGAAGGTGTTGTCAAAGAGGAGTATTCCCACCTGAATTTCGGTGAAGTGT |
| | | GGTTGAAGGAACATTTTGCGGAATCTAAAGCCGAATTGGAACTGGCAAATCGCCAGAACCTGCCGATCGT |
| | | TTGGAAGATGCTGAACCAAGTGGAAGGTGATGCACATACGATGGCGATGGAGAAGGACGCATTGGTTGAG |
| | | GACTTTATGATTCAGTATGGCGAAGCACTGTCCAATATCGGTTTCAGCACCCGTGATATCATGCGTCTGA |
| | | GCGCCTATGGCCTGATCGGTGCCTAA |
| 49 | codon- | ATGGAGTGGAAACCAAAACCGAAACTGCCTCAGCTGCTGGATGACCACTTCGGTCTGCACGGCCTGGTTT |
| | optimized | TCCGTCGTACCTTCGCTATCCGTTCTTACGAAGTCGGCCCTGATCGCTCCACCTCCATCCTGGCGGTAAT |
| | Umbellularia | GAACCACATGCAGGAAGCAACTCTGAACCATGCGAAAAGCGTAGGTATCCTGGGCGATGGTTTCGGCACT |
| | californica | ACTCTGGAGATGTCCAAACGTGATCTGATGTGGGTTGTTCGCCGTACCCATGTCGCGGTTGAACGCTACC |
| | fatBₘ (without | CGACCTGGGGCGATACGGTTGAAGTGGAATGCTGGATCGGCGCGTCCGGCAACAACGGCATGCGTCGCGA |
| | leader | TTTCCTGGTTCGCGATTGTAAGACGGGCGAGATTCTGACCCGTTGCACGTCCCTGAGCGTTCTGATGAAT |
| | sequence). | ACCCGTACCCGTCGTCTGAGCACCATCCCGGACGAAGTTCGCGGTGAAATTGGCCCGGCATTCATCGATA |
| | | ACGTTGCAGTAAAAGACGATGAAATCAAGAAACTGCAGAAACTGAATGACTCTACCGCGGACTACATCCA |
| | | GGGTGGTCTGACCCCGCGCTGGAACGACCTGGACGTGAACCAGCACGTCAACAACCTGAAATACGTAGCT |
| | | TGGGTATTCGAAACGGTCCCGGATTCTATCTTCGAATCTCACCACATCAGCTCCTTCACCCTGGAATACC |
| | | GTCGTGAGTGTACCCGTGACTCCGTTCTGCGCTCTCTGACCACGGTATCCGGCGGTAGCTCTGAAGCCGG |
| | | TCTGGTTTGCGATCACCTGCTGCAGCTGGAAGGCGGCAGCGAGGTTCTGCGTGCTCGTACTGAGTGGCGT |
| | | CCGAAGCTGACTGACTCTTTCCGCGGCATCTCTGTTATCCCGGCAGAGCCTCGTGTGTAA |
| 50 | codon- | ATGAAAACGACCCACACCAGCTTACCATTTGCCGGCCACACGTTACATTTCGTCGAATTTGATCCGGCGA |
| | Optimized E. | ACTTTTGTGAACAAGACCTGTTGTGGCTGCCGCATTATGCCCAGCTGCAGCACGCAGGCCGTAAGCGTAA |
| | coli entD. | AACTGAACATCTGGCCGGTCGCATTGCGGCAGTGTATGCCCTGCGCGAGTACGGCTACAAATGCGTGCCG |
| | | GCCATTGGTGAACTGCGTCAACCGGTTTGGCCGGCAGAAGTTTACGGTTCCATCTCCCACTGCGGTACTA |
| | | CCGCGTTGGCGGTTGTGTCTCGCCAGCCGATCGGTATTGATATTGAAGAGATATTCTCTGTCCAGACGGC |
| | | ACGCGAGCTGACGGACAACATCATTACCCCGGCAGAGCACGAGCGTCTGGCGGACTGTGGTCTGGCGTTC |
| | | AGCCTGGCGCTGACCCTGGCATTCAGCGCAAAAGAGAGCGCGTTCAAGGCTTCCGAGATCCAAACCGATG |
| | | CGGGCTTCCTGGATTATCAAATCATCAGCTGGAACAAGCAACAGGTTATCATTCACCGTGAGAATGAGAT |
| | | GTTTGCCGTCCATTGGCAGATTAAAGAGAAAATCGTTATCACCCTGTGCCAGCACGACTGA |
| 51 | plasmid | TAGAAAAACTCATCGAGCATCAAATGAAACTGCAATTTATTCATATCAGGATTATCAATACCATATTTTT |
| | pAQ4::P(cpcB)- | GAAAAAGCCGTTTCTGTAATGAAGGAGAAAACTCACCGAGGCAGTTCCATAGGATGGCAAGATCCTGGTA |
| | Nhistag_adm | TCGGTCTGCGATTCCGACTCGTCCAACATCAATACAACCTATTAATTTCCCCTCGTCAAAAATAAGGTTA |
| | (Npu)-ErmC | TCAAGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATTCAT |
| | | TCGTGATTGCGCCTGAGCGAGGCGAAATACGCGATCGCTGTTAAAAGGACAATTACAAACAGGAATCGAG |
| | | TGCAACCGGCGCAGGAACACTGCCAGCGCATCAACAATATTTTCACCTGAATCAGGATATTCTTCTAATA |
| | | CCTGGAACGCTGTTTTTCCGGGGATCGCAGTGGTGAGTAACCATGCATCATCAGGAGTACGGATAAAATG |
| | | CTTGATGGTCGGAAGTGGCATAAATTCCGTCAGCCAGTTTAGTCTGACCATCTCATCTGTAACATCATTG |
| | | GCAACGCTACCTTTGCCATGTTTCAGAAACAACTCTGGCGCATCGGGCTTCCCATACAAGCGATAGATTG |
| | | TCGCACCTGATTGCCCGACATTATCGCGAGCCCATTTATACCCATATAAATCAGCATCCATGTTGGAATT |
| | | TAATCGCGGCCTCGACGTTTCCCGTTGAATATGGCTCATATTCTTCCTTTTTCAATATTATTGAAGCATT |
| | | TATCAGGGTTATTGTCTCATGAGCGGATACATATTTGAATGTATTTAGAAAAATAAACAAATAGGGGTCA |
| | | GTGTTACAACCAATTAACCAATTCTGAACATTATCGCGAGCCCATTTATACCTGAATATGGCTCATAACA |
| | | CCCCTTGTTTGCCTGGCGGCAGTAGCGCGGTGGTCCCACCTGACCCCATGCCGAACTCAGAAGTGAAACG |
| | | CCGTAGCGCCGATGGTAGTGTGGGGACTCCCCATGCGAGAGTAGGGAACTGCCAGGCATCAAATAAAACG |
| | | AAAGGCTCAGTCGAAAGACTGGGCCTTTCGCCCGGGCTAATTAGGGGGTGTCGCCCTTTACACGTACTTA |
| | | GTCGCTGAAGGCCTCACTGGCCCCTGCAGGGATGGTGGAATGCTGGTTATCTGGTGGGGATTAAGTGGTG |
| | | TTTTACTAAAGCTTGAACAACTCAAGAAAGATTATATTCGCAATAACTGCCAATAATCCCAGCATCTTGA |
| | | GAAAATCCAGCAAACCGGGGGCAAAACACCAGCAAGAAGCCAGCAGACTATCACCAAATCCCCAGCGTAC |
| | | AGCTAGAAATAACTGAGCAGTTGTATTCAATTACCTTCTGGTCAAGCCGAGGAAATTTCCCCACACCTTA |
| | | TACACCTCTGGAAGGTTTTTTTGACGAAGCGCAAAATATCCACAATCGGCTGGGGACTTCTTCTGTCAGA |
| | | AAATGGCAGAAATTTTTGAATGTGTTGGCGATCGCCCTCATCAATGATTATTAGAGAACTTTTGTCCCTG |
| | | ATGTTGGGAATACTCTTGATGACAATTGTGATTGCTCAAAGAAGAAAGAAATTTGGAGTAAATCTCTAAA |
| | | AGGGGACTGAAATATTTGTATGGTCAGCATGACCACTGAAATGGAGAGAAGTCTAAGACAGTAGATGTCT |
| | | TAGATATAAGCCTCATTAGAAGCCATGCCATAAAACAGATTTTGTGGATGAAACAACTTGAAATAGTTCA |
| | | GTTGTAGACCATGTTATAAACATTTATTCTTAACACAGTGACACATTAATGACTCATATATCCGTCCAAA |
| | | AAAAACTAAAATGTTTGTAAATTTAGTTTTGCGGCCGCGTCGACTTCGTTATAAAATAAACTTAACAAAT |
| | | CTATACCCACCTGTAGAGAAGAGTCCCTGAATATCAAAATGGTGGGATAAAAAGCTCAAAAAGGAAAGTA |
| | | GGCTGTGGTTCCCTAGGCAACAGTCTTCCCTACCCCACTGGAAACTAAAAAAACGAGAAAAGTTCGCACC |
| | | GAACATCAATTGCATAATTTTAGCCCTAAAACATAAGCTGAACGAAACTGGTTGTCTTCCCTTCCCAATC |
| | | CAGGACAATCTGAGAATCCCCTGCAACATTACTTAACAAAAAAGCAGGAATAAAATTAACAAGATGTAAC |
| | | AGACATAAGTCCCATCACCGTTGTATAAAGTTAACTGTGGGATTGCAAAAGCATTCAAGCCTAGGCGCTG |
| | | AGCTGTTTGAGCATCCCGGTGGCCCTTGTCGCTGCCTCCGTGTTTCTCCCTGGATTTATTTAGGTAATAT |
| | | CTCTCATAAATCCCCGGGTAGTTAACGAAAGTTAATGGAGATCAGTAACAATAACTCTAGGGTCATTACT |
| | | TTGGACTCCCTCAGTTTATCCGGGGGAATTGTGTTTAAGAAAATCCCAACTCATAAAGTCAAGTAGGAGA |
| | | TTAATCATATGCACCATCACCACCATCATGGAGGCGGACAGCAACTGACCGATCAAAGCAAAGAACTGGA |
| | | CTTCAAGAGCGAGACGTACAAAGACGCCTATAGCCGCATTAACGCGATCGTCATTGAAGGCGAACAAGAG |
| | | GCGCATGAAAACTACATCACCCTGGCGCAGCTGCTGCCTGAGAGCCACGACGAACTGATTCGCCTGAGCA |
| | | AAATGGAGAGCCGTCACAAGAAAGGTTTTGAGGCGTGTGGCCGCAATCTGGCGGTGACCCCGGACCTGCA |
| | | ATTTGCGAAGGAGTTCTTTAGCGGTCTGCACCAGAATTTCCAGACGGCCGCAGCCGAGGGCAAAGTCGTC |
| | | ACTTGTTTGTTGATCCAGAGCCTGATTATTGAATGCTTTGCTATTGCGGCGTACAACATTTACATTCCGG |
| | | TCGCCGATGACTTTGCGCGTAAAATCACGGAAGGTGTTGTCAAAGAGGAGTATTCCCACCTGAATTTCGG |
| | | TGAAGTGTGGTTGAAGGAACATTTTGCGGAATCTAAAGCCGAATTGGAACTGGCAAATCGCCAGAACCTG |
| | | CCGATCGTTTGGAAGATGCTGAACCAAGTGGAAGGTGATGCACATACGATGGCGATGGAGAAGGACGCAT |
| | | TGGTTGAGGACTTTATGATTCAGTATGGCGAAGCACTGTCCAATATCGGTTTCAGCACCCGTGATATCAT |
| | | GCGTCTGAGCGCCTATGGCCTGATCGGTGCCTAAGAGCTCCTCGAGGAATTCGGTTTTCCGTCCTGTCTT |
| | | GATTTTCAAGCAAACAATGCCTCCGATTTCTAATCGGAGGCATTTGTTTTTGTTTATTGCAAAAACAAAA |
| | | AATATTGTTACAAATTTTTACAGGCTATTAAGCCTACCGTCATAAATAATTTGCCATTTACTAGTTTTAA |
| | | TTAACGTGCTATAATTATACTAATTTTATAAGGAGGAAAAAATATGGGCATTTTTAGTATTTTTGTAATC |
| | | AGCACAGTTCATTATCAACCAAACAAAAAATAAGTGGTTATAATGAATCGTTAATAAGCAAAATTCATAT |
| | | AACCAAATTAAAGAGGGTTATAATGAACGAGAAAAATATAAAACACAGTCAAAACTTTATTACTTCAAAA |
| | | CATAATATAGATAAAATAATGACAAATATAAGATTAAATGAACATGATAATATCTTTGAAATCGGCTCAG |
| | | GAAAAGGCCATTTTACCCTTGAATTAGTAAAGAGGTGTAATTTCGTAACTGCCATTGAAATAGACCATAA |
| | | ATTATGCAAAACTACAGAAAATAAACTTGTTGATCACGATAATTTCCAAGTTTTAAACAAGGATATATTG |
| | | CAGTTTAAATTTCCTAAAAACCAATCCTATAAAATATATGGTAATATACCTTATAACATAAGTACGGATA |
| | | TAATACGCAAAATTGTTTTTGATAGTATAGCTAATGAGATTTATTTAATCGTGGAATACGGGTTTGCTAA |
| | | AAGATTATTAAATACAAAACGCTCATTGGCATTACTTTTAATGGCAGAAGTTGATATTTCTATATTAAGT |
| | | ATGGTTCCAAGAGAATATTTTCATCCTAAACCTAAAGTGAATAGCTCACTTATCAGATTAAGTAGAAAAA |
| | | AATCAAGAATATCACACAAAGATAAACAAAAGTATAATTATTTCGTTATGAAATGGGTTAACAAAGAATA |
| | | CAAGAAAATATTTACAAAAAATCAATTTAACAATTCCTTAAAACATGCAGGAATTGACGATTTAAACAAT |
| | | ATTAGCTTTGAACAATTCTTATCTCTTTTCAATAGCTATAAATTATTTAATAAGTAAGTTAAGGGATGCA |
| | | TAAACTGCATCCCTTAACTTGTTTTTCGTGTGCCTATTTTTTGTGGCGCGCCCAGTTTCCTTTACTGGCC |
| | | CTAAAGTCGCTGTGGCTAGGGTTCCGAAGGGGCATTATTGGCTCGCGGCTTTACAACCTTGATAAGGAGA |
| | | GAGATGACAGTTTTTTTTCTCTTTTGCTTAGTAAAACAGCAAATTTAAGGCATGTTAAAGAGCAGTAGAA |
| | | CGAAATGGTTGAGCCGGCCTCGATACACTCAATTAACTACTAATAGCTTCAATAAATTTTGGGACGATTG |
| | | AAGCTATTTTTTTGAAAATCAACTCTTAATATCTCCTGTCTCAAAAGAGTTAATTGCTAAACAAAAGCCA |
| | | GTTTCAGCGAAAAATCTAGAGTTTTATAGGTTCGTTCTCAGTACAGGACAAAAAGTTTGAAAAGGATAGA |
| | | GGGAGAGGGTTTGATGGAAATAAGCACAAATCAATCAAGCCCTCATGAATCAGATTAGCGAAATTCGCCG |
| | | CCAATTGCGACCTCATCTCGGATGGCATGGAGCCAGACTGTCATTTATCGCCCTCTTCCTGGTGGCACTG |
| | | TTCCGAGCAAAAACCGTCAATCTCGCCAAACTCGCCACCGTCTGGGGAGGCAATGCAGCAGAAGAGTCTA |
| | | ATTACAAACGCATGCAGCGATTCTTTCAGTCCTTTGACGTCAACATGGACAAAATCGCCAGGATGGTAAT |
| | | GAATATCGCGGCTATCCCGCAACCTTGGGTCTTAAGCATCGACCGCACCAACGGCCGGCCTACATGGCCC |
| | | GTCAATCGAAGGGCGACACAAAATTTATTCTAAATGCATAATAAATACTGATAACATCTTATAGTTTGTA |
| | | TTATATTTTGTATTATCGTTGACATGTATAATTTTGATATCAAAAACTGATTTTCCCTTTATTATTTTCG |
| | | AGATTTATTTTCTTAATTCTCTTTAACAAACTAGAAATATTGTATATACAAAAAATCATAAATAATAGAT |
| | | GAATAGTTTAATTATAGGTGTTCATCAATCGAAAAAGCAACGTATCTTATTTAAAGTGCGTTGCTTTTTT |
| | | CTCATTTATAAGGTTAAATAATTCTCATATATCAAGCAAAGTGACAGGCGCCCTTAAATATTCTGACAAA |
| | | TGCTCTTTCCCTAAACTCCCCCCATAAAAAAACCCGCCGAAGCGGGTTTTTACGTTATTTGCGGATTAAC |
| | | GATTACTCGTTATCAGAACCGCCCAGGGGGCCCGAGCTTAAGACTGGCCGTCGTTTTACAACACAGAAAG |
| | | AGTTTGTAGAAACGCAAAAAGGCCATCCGTCAGGGGCCTTCTGCTTAGTTTGATGCCTGGCAGTTCCCTA |
| | | CTCTCGCCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTGAGCAAA |
| | | AGGCCAGCAAAAGGCCAGGAACCGTAAAAAGGCCGCGTTGCTGGCGTTTTTCCATAGGCTCCGCCCCCCT |
| | | GACGAGCATCACAAAAATCGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCTTACCGGATACCTGTCCGC |
| | | CTTTCTCCCTTCGGGAAGCGTGGCGCTTTCTCATAGCTCACGCTGTAGGTATCTCAGTTCGGTGTAGGTC |
| | | GTTCGCTCCAAGCTGGGCTGTGTGCACGAACCCCCCGTTCAGCCCGACCGCTGCGCCTTATCCGGTAACT |
| | | ATCGTCTTGAGTCCAACCCGGTAAGACACGACTTATCGCCACTGGCAGCAGCCACTGGTAACAGGATTAG |
| | | CAGAGCGAGGTATGTAGGCGGTGCTACAGAGTTCTTGAAGTGGTGGGCTAACTACGGCTACACTAGAAGA |
| | | ACAGTATTTGGTATCTGCGCTCTGCTGAAGCCAGTTACCTTCGGAAAAAGAGTTGGTAGCTCTTGATCCG |
| | | GCAAACAAACCACCGCTGGTAGCGGTGGTTTTTTTGTTTGCAAGCAGCAGATTACGCGCAGAAAAAAAGG |
| | | ATCTCAAGAAGATCCTTTGATCTTTTCTACGGGGTCTGACGCTCAGTGGAACGACGCGCGCGTAACTCAC |
| | | GTTAAGGGATTTTGGTCATGAGCTTGCGCCGTCCCGTCAAGTCAGCGTAATGCTCTGCTTT |
| 52 | plasmid | AAAAGCAGAGCATTACGCTGACTTGACGGGACGGCGCAAGCTCATGACCAAAATCCCTTAACGTGAGTTA |
| | pAQ3::P(nir07)- | CGCGCGCGTCGTTCCACTGAGCGTCAGACCCCGTAGAAAAGATCAAAGGATCTTCTTGAGATCCTTTTTT |
| | fatBₘ-carB- | TCTGCGCGTAATCTGCTGCTTGCAAACAAAAAAACCACCGCTACCAGCGGTGGTTTGTTTGCCGGATCAA |
| | entD-SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCTTGTAGCAATTGC |
| | | TACTAAAAACTGCGATCGCTGCTGAAATGAGCTGGAATTTTGTCCCTCTCAGCTCAAAAAGTATCAATGA |
| | | TTACTTAATGTTTGTTCTGCGCAAACTTCTTGCAGAACATGCATGATTTACAAAAAGTTGTAGTTTCTGT |
| | | TACCAATTGCGAATCGAGAACTGCCTAATCTGCCGAGTATGCGATCCTTTAGCAGGAGGAAAACCATATG |
| | | GAGTGGAAACCAAAACCGAAACTGCCTCAGCTGCTGGATGACCACTTCGGTCTGCACGGCCTGGTTTTCC |
| | | GTCGTACCTTCGCTATCCGTTCTTACGAAGTCGGCCCTGATCGCTCCACCTCCATCCTGGCGGTAATGAA |
| | | CCACATGCAGGAAGCAACTCTGAACCATGCGAAAAGCGTAGGTATCCTGGGCGATGGTTTCGGCACTACT |
| | | CTGGAGATGTCCAAACGTGATCTGATGTGGGTTGTTCGCCGTACCCATGTCGCGGTTGAACGCTACCCGA |
| | | CCTGGGGCGATACGGTTGAAGTGGAATGCTGGATCGGCGCGTCCGGCAACAACGGCATGCGTCGCGATTT |
| | | CCTGGTTCGCGATTGTAAGACGGGCGAGATTCTGACCCGTTGCACGTCCCTGAGCGTTCTGATGAATACC |
| | | CGTACCCGTCGTCTGAGCACCATCCCGGACGAAGTTCGCGGTGAAATTGGCCCGGCATTCATCGATAACG |
| | | TTGCAGTAAAAGACGATGAAATCAAGAAACTGCAGAAACTGAATGACTCTACCGCGGACTACATCCAGGG |
| | | TGGTCTGACCCCGCGCTGGAACGACCTGGACGTGAACCAGCACGTCAACAACCTGAAATACGTAGCTTGG |
| | | GTATTCGAAACGGTCCCGGATTCTATCTTCGAATCTCACCACATCAGCTCCTTCACCCTGGAATACCGTC |
| | | GTGAGTGTACCCGTGACTCCGTTCTGCGCTCTCTGACCACGGTATCCGGCGGTAGCTCTGAAGCCGGTCT |
| | | GGTTTGCGATCACCTGCTGCAGCTGGAAGGCGGCAGCGAGGTTCTGCGTGCTCGTACTGAGTGGCGTCCG |
| | | AAGCTGACTGACTCTTTCCGCGGCATCTCTGTTATCCCGGCAGAGCCTCGTGTGTAAGAGCTCGAGGAGG |
| | | TTTTTACAATGACCAGCGATGTTCACGACGCCACAGACGGCGTCACCGAAACCGCACTCGACGACGAGCA |
| | | GTCGACCCGCCGCATCGCCGAGCTGTACGCCACCGATCCCGAGTTCGCCGCCGCCGCACCGTTGCCCGCC |
| | | GTGGTCGACGCGGCGCACAAACCCGGGCTGCGGCTGGCAGAGATCCTGCAGACCCTGTTCACCGGCTACG |
| | | GTGACCGCCCGGCGCTGGGATACCGCGCCCGTGAACTGGCCACCGACGAGGGCGGGCGCACCGTGACGCG |
| | | TCTGCTGCCGCGGTTCGACACCCTCACCTACGCCCAGGTGTGGTCGCGCGTGCAAGCGGTCGCCGCGGCC |
| | | CTGCGCCACAACTTCGCGCAGCCGATCTACCCCGGCGACGCCGTCGCGACGATCGGTTTCGCGAGTCCCG |
| | | ATTACCTGACGCTGGATCTCGTATGCGCCTACCTGGGCCTCGTGAGTGTTCCGCTGCAGCACAACGCACC |
| | | GGTCAGCCGGCTCGCCCCGATCCTGGCCGAGGTCGAACCGCGGATCCTCACCGTGAGCGCCGAATACCTC |
| | | GACCTCGCAGTCGAATCCGTGCGGGACGTCAACTCGGTGTCGCAGCTCGTGGTGTTCGACCATCACCCCG |
| | | AGGTCGACGACCACCGCGACGCACTGGCCCGCGCGCGTGAACAACTCGCCGGCAAGGGCATCGCCGTCAC |
| | | CACCCTGGACGCGATCGCCGACGAGGGCGCCGGGCTGCCGGCCGAACCGATCTACACCGCCGACCATGAT |
| | | CAGCGCCTCGCGATGATCCTGTACACCTCGGGTTCCACCGGCGCACCCAAGGGTGCGATGTACACCGAGG |
| | | CGATGGTGGCGCGGCTGTGGACCATGTCGTTCATCACGGGTGACCCCACGCCGGTCATCAACGTCAACTT |
| | | CATGCCGCTCAACCACCTGGGCGGGCGCATCCCCATTTCCACCGCCGTGCAGAACGGTGGAACCAGTTAC |
| | | TTCGTACCGGAATCCGACATGTCCACGCTGTTCGAGGATCTCGCGCTGGTGCGCCCGACCGAACTCGGCC |
| | | TGGTTCCGCGCGTCGCCGACATGCTCTACCAGCACCACCTCGCCACCGTCGACCGCCTGGTCACGCAGGG |
| | | CGCCGACGAACTGACCGCCGAGAAGCAGGCCGGTGCCGAACTGCGTGAGCAGGTGCTCGGCGGACGCGTG |
| | | ATCACCGGATTCGTCAGCACCGCACCGCTGGCCGCGGAGATGAGGGCGTTCCTCGACATCACCCTGGGCG |
| | | CACACATCGTCGACGGCTACGGGCTCACCGAGACCGGCGCCGTGACACGCGACGGTGTGATCGTGCGGCC |
| | | ACCGGTGATCGACTACAAGCTGATCGACGTTCCCGAACTCGGCTACTTCAGCACCGACAAGCCCTACCCG |
| | | CGTGGCGAACTGCTGGTCAGGTCGCAAACGCTGACTCCCGGGTACTACAAGCGCCCCGAGGTCACCGCGA |
| | | GCGTCTTCGACCGGGACGGCTACTACCACACCGGCGACGTCATGGCCGAGACCGCACCCGACCACCTGGT |
| | | GTACGTGGACCGTCGCAACAACGTCCTCAAACTCGCGCAGGGCGAGTTCGTGGCGGTCGCCAACCTGGAG |
| | | GCGGTGTTCTCCGGCGCGGCGCTGGTGCGCCAGATCTTCGTGTACGGCAACAGCGAGCGCAGTTTCCTTC |
| | | TGGCCGTGGTGGTCCCGACGCCGGAG<+GCTCGAGCAGTACGATCCGGCCGCGCTCAAGGCCGCGCTGGC |
| | | CGACTCGCTGCAGCGCACCGCACGCGACGCCGAACTGCAATCCTACGAGGTGCCGGCCGATTTCATCGTC |
| | | GAGACCGAGCCGTTCAGCGCCGCCAACGGGCTGCTGTCGGGTGTCGGAAAACTGCTGCGGCCCAACCTCA |
| | | AAGACCGCTACGGGCAGCGCCTGGAGCAGATGTACGCCGATATCGCGGCCACGCAGGCCAACCAGTTGCG |
| | | CGAACTGCGGCGCGCGGCCGCCACACAACCGGTGATCGACACCCTCACCCAGGCCGCTGCCACGATCCTC |
| | | GGCACCGGGAGCGAGGTGGCATCCGACGCCCACTTCACCGACCTGGGCGGGGATTCCCTGTCGGCGCTGA |
| | | CACTTTCGAACCTGCTGAGCGATTTCTTCGGTTTCGAAGTTCCCGTCGGCACCATCGTGAACCCGGCCAC |
| | | CAACCTCGCCCAACTCGCCCAGCACATCGAGGCGCAGCGCACCGCGGGTGACCGCAGGCCGAGTTTCACC |
| | | ACCGTGCACGGCGCGGACGCCACCGAGATCCGGGCGAGTGAGCTGACCCTGGACAAGTTCATCGACGCCG |
| | | AAACGCTCCGGGCCGCACCGGGTCTGCCCAAGGTCACCACCGAGCCACGGACGGTGTTGCTCTCGGGCGC |
| | | CAACGGCTGGCTGGGCCGGTTCCTCACGTTGCAGTGGCTGGAACGCCTGGCACCTGTCGGCGGCACCCTC |
| | | ATCACGATCGTGCGGGGCCGCGACGACGCCGCGGCCCGCGCACGGCTGACCCAGGCCTACGACACCGATC |
| | | CCGAGTTGTCCCGCCGCTTCGCCGAGCTGGCCGACCGCCACCTGCGGGTGGTCGCCGGTGACATCGGCGA |
| | | CCCGAATCTGGGCCTCACACCCGAGATCTGGCACCGGCTCGCCGCCGAGGTCGACCTGGTGGTGCATCCG |
| | | GCAGCGCTGGTCAACCACGTGCTCCCCTACCGG<+GCTGTTCGGCCCCAACGTCGTGGGCACGGCCGAGG |
| | | TGATCAAGCTGGCCCTCACCGAACGGATCAAGCCCGTCACGTACCTGTCCACCGTGTCGGTGGCCATGGG |
| | | GATCCCCGACTTCGAGGAGGACGGCGACATCCGGACCGTGAGCCCGGTGCGCCCGCTCGACGGCGGATAC |
| | | GCCAACGGCTACGGCAACAGCAAGTGGGCCGGCGAGGTGCTGCTGCGGGAGGCCCACGATCTGTGCGGGC |
| | | TGCCCGTGGCGACOTTCCGCTCGGACATGATCCTGGCGCATCCGCGCTACCGCGGTCAGGTCAACGTGCC |
| | | AGACATGTTCACGCGACTCCTGTTGAGCCTCTTGATCACCGGCGTCGCGCCGCGGTCGTTCTACATCGGA |
| | | GACGGTGAGCGCCCGCGGGCGCACTACCCCGGCCTGACGGTCGATTTCGTGGCCGAGGCGGTCACGACGC |
| | | TCGGCGCGCAGCAGCGCGAGGGATACGTGTCCTACGACGTGATGAACCCGCACGACGACGGGATCTCCCT |
| | | GGATGTGTTCGTGGACTGGCTGATCCGGGCGGGCCATCCGATCGACCGGGTCGACGACTACGACGACTGG |
| | | GTGCGTCGGTTCGAGACCGCGTTGACCGCGCTTCCCGAGAAGCGCCGCGCACAGACCGTACTGCCGCTGC |
| | | TGCACGCGTTCCGCGCTCCGCAGGCACCGTTGCGCGGCGCACCCGAACCCACGGAGGTGTTCCACGCCGC |
| | | GGTGCGCACCGCGAAGGTGGGCCCGGGAGACATCCCGCACCTCGACGAGGCGCTGATCGACAAGTACATA |
| | | CGCGATCTGCGTGAGTTCGGTCTGATCTGAGGTACCCACAAGGAGGTTTTTACAATGAAAACGACCCACA |
| | | CCAGCTTA<XATTTGCCGGCCACACGTTACATTTCGTCGAATTTGATCCGGCGAACTTTTGTGAACAAGA |
| | | CCTGTTGTGGCTGCCGCATTATGCCCAGCTGCAGCACGCAGGCCGTAAGCGTAAAACTGAACATCTGGCC |
| | | GGTCGCATTGCGGCAGTGTATGCCCTGCGCGAGTACGGCTACAAATGCGTGCCGGCCATTGGTGAACTGC |
| | | GTCAACCGGTTTGGCCGGCAGAAGTTTACGGTTCCATCTCCCACTGCGGTACTACCGCGTTGGCGGTTGT |
| | | GTCTCGCCAGCCGATCGGTATTGATATTGAAGAGATATTCTCTGTCCAGACGGCACGCGAGCTGACGGAC |
| | | AACATCATTACCCCGGCAGAGCACGAGCGTCTGGCGGACTGTGGTCTGGCGTTCAGCCTGGCGCTGACCC |
| | | TGGCATTCAGCGCAAAAGAGAGCGCGTTCAAGGCTTCCGAGATCCAAACCGATGCGGGCTTCCTGGATTA |
| | | TCAAATCATCAGCTGGAACAAGCAACAGGTTATCATTCACCGTGAGAATGAGATGTTTGCCGTCCATTGG |
| | | CAGATTAAAGAGAAAATCGTTATCACCCTGTGCCAGCACGACTGAGAATTCGGTTTTCCGTCCTGTCTTG |
| | | ATTTTCAAGCAAACAATGCCTCCGATTTCTAATCGGAGGCATTTGTTTTTGTTTATTGCAAAAACAAAAA |
| | | ATATTGTTACAAATTTTTACAGGCTATTAAGCCTACCGTCATAAATAATTTGCCATTTACTAGTTTTTAA |
| | | TTAACCAGAACCTTGACCGAACGCAGCGGTGGTAACGGCGCAGTGGCGGTTTTCATGGCTTGTTATGACT |
| | | GTTTTTTTGGGGTACAGTCTATGCCTCGGGCATCCAAGCAGCAAGCGCGTTACGCCGTGGGTCGATGTTT |
| | | GATGTTATGGAGCAGCAACGATGTTACGCAGCAGGGCAGTCGCCCTAAAACAAAGTTAAACATCATGAGG |
| | | GAAGCGGTGATCGCCGAAGTATCGACTCAACTATCAGAGGTAGTTGGCGTCATCGAGCGCCATCTCGAAC |
| | | CGACGTTGCTGGCCGTACATTTGTACGGCTCCGCAGTGGATGGCGGCCTGAAGCCACACAGTGATATTGA |
| | | TTTGCTGGTTACGGTGACCGTAAGGCTTGATGAAACAACGCGGCGAGCTTTGATCAACGACCTTTTGGAA |
| | | ACTTCGGCTTCCCCTGGAGAGAGCGAGATTCTCCGCGCTGTAGAAGTCACCATTGTTGTGCACGACGACA |
| | | TCATTCCGTGGCGTTATCCAGCTAAGCGCGAACTGCAATTTGGAGAATGGCAGCGCAATGACATTCTTGC |
| | | AGGTATCTTCGAGCCAGCCACGATCGACATTGATCTGGCTATCTTGCTGACAAAAGCAAGAGAACATAGC |
| | | GTTGCCTTGGTAGGTCCAGCGGCGGAGGAACTCTTTGATCCGGTTCCTGAACAGGATCTATTTGAGGCGC |
| | | TAAATGAAACCTTAACGCTATGGAACTCGCCGCCCGACTGGGCTGGCGATGAGCGAAATGTAGTGCTTAC |
| | | GTTGTCCCGCATTTGGTACAGCGCAGTAACCGGCAAAATCGCGCCGAAGGATGTCGCTGCCGACTGGGCA |
| | | ATGGAGCGCCTGCCGGCCCAGTATCAGCCCGTCATACTTGAAGCTAGACAGGCTTATCTTGGACAAGAAG |
| | | AAGATCGCTTGGCCTCGCGCGCAGATCAGTTGGAAGAATTTGTCCACTACGTGAAAGGCGAGATCACCAA |
| | | GGTAGTCGGCAAATAATGTCTAACAATTCGTTCAAGCCGACGCCGCTTCGCGGCGCGGCTTAACTCAAGC |
| | | GTTAGATGCACTAAGCACATAATTGCTCACAGCCAAACTATCAGGTCAAGTCTGCTTTTATTATTTTTAA |
| | | GCGTGCATAATAAGCCCTACACAAATTGGGAGATATATCATGAGGCGCGCCACGAGAAAGAGTTATGACA |
| | | AATTAAAATTCTGACTCTTAGATTATTTCCAGAGAGGCTGATTTTCCCAATCTTTGGGAAAGCCTAAGTT |
| | | TTTAGATTCTATTTCTGGATACATCTCAAAAGTTCTTTTTAAATGCTGTGCAAAATTATGCTCTGGTTTA |
| | | ATTCTGTCTAAGAGATACTGAATACAACATAAGCCAGTGAAAATTTTACGGCTGTTTCTTTGATTAATAT |
| | | CCTCCAATACTTCTCTAGAGAGCCATTTTCCTTTTAACCTATCAGGCAATTTAGGTGATTCTCCTAGCTG |
| | | TATATTCCAGAGCCTTGAATGATGAGCGCAAATATTTCTAATATGCGACAAAGACCGTAACCAAGATATA |
| | | AAAAACTTGTTAGGTAATTGGAAATGAGTATGTATTTTTTGTCGTGTCTTAGATGGTAATAAATTTGTGT |
| | | ACATTCTAGATAACTGCCCAAAGGCGATTATCTCCAAAGCCATATATGACGGCGGTAGTAGAGGATTTGT |
| | | GTACTTGTTTCGATAATGCCCGATAAATTCTTCTACTTTTTTAGATTGGCAATATTGAGTAATCGAATCG |
| | | ATTAATTCTTGATGCTTCCCAGTGTCATAAAATAAACTTTTATTCAGATACCAATGAGGATCATAATCAT |
| | | GGGAGTAGTGATAAATCATTTGAGTTCTGACTGCTACTTCTATCGACTCCGTAGCATTAAAAATAAGCAT |
| | | TCTCAAGGATTTATCAAACTTGTATAGATTTGGCCGGCCCGTCAAAAGGGCGACACCCCATAATTAGCCC |
| | | GGGCGAAAGGCCCAGTCTTTCGACTGAGCCTTTCGTTTTATTTGATGCCTGGCAGTTCCCTACTCTCGCA |
| | | TGGGGAGTCCCCACACTACCATCGGCGCTACGGCGTTTCACTTCTGAGTTCGGCATGGGGTCAGGTGGGA |
| | | CCACCGCGCTACTGCCGCCAGGCAAACAAGGGGTGTTATGAGCCATATTCAGGTATAAATGGGCTCGCGA |
| | | TAATGTTCAGAATTGGTTAATTGGTTGTAACACTGACCCCTATTTGTTTATTTTTCTAAATACATTCAAA |
| | | TATGTATCCGCTCATGAGACAATAACCCTGATAAATGCTTCAATAATATTGAAAAAGGAAGAATATGAGT |
| | | ATTCAACATTTCCGTGTCGCCCTTATTCCCTTTTTTGCGGCATTTTGCCTTCCTGTTTTTGCTCACCCAG |
| | | AAACGCTGGTGAAAGTAAAAGATGCTGAAGATCAGTTGGGTGCACGAGTGGGTTACATCGAACTGGATCT |
| | | CAACAGCGGTAAGATCCTTGAGAGTTTTCGCCCCGAAGAACGTTTTCCAATGATGAGCACTTTTAAAGTT |
| | | CTGCTATGTGGCGCGGTATTATCCCGTATTGACGCCGGGCAAGAGCAACTCGGTCGCCGCATACACTATT |
| | | CTCAGAATGACTTGGTTGAGTACTCACCAGTCACAGAAAAGCATCTTACGGATGGCATGACAGTAAGAGA |
| | | ATTATGCAGTGCTGCCATAACCATGAGTGATAACACTGCGGCCAACTTACTTCTGACAACGATCGGAGGA |
| | | CCGAAGGAGCTAACCGCTTTTTTGCACAACATGGGGGATCATGTAACTCGCCTTGATCGTTGGGAACCGG |
| | | AGCTGAATGAAGCCATACCAAACGACGAGCGTGACACCACGATGCCTGTAGCGATGGCAACAACGTTGCG |
| | | CAAACTATTAACTGGCGAACTACTTACTCTAGCTTCCCGGCAACAATTAATAGACTGGATGGAGGCGGAT |
| | | AAAGTTGCAGGACCACTTCTGCGCTCGGCCCTTCCGGCTGGCTGGTTTATTGCTGATAAATCCGGAGCCG |
| | | GTGAGCGTGGTTCTCGCGGTATCATCGCAGCGCTGGGGCCAGATGGTAAGCCCTCCCGTATCGTAGTTAT |
| | | CTACACGACGGGGAGTCAGGCAACTATGGATGAACGAAATAGACAGATCGCTGAGATAGGTGCCTCACTG |
| | | ATTAAGCATTGGT |
| 53 | carboxylic | ATGACCAGCGATGTTCACGACGCCACAGACGGCGTCACCGAAACCGCACTCGACGACGAGCAGTCGACCC |
| | acid | GCCGCATCGCCGAGCTGTACGCCACCGATCCCGAGTTCGCCGCCGCCGCACCGTTGCCCGCCGTGGTCGA |
| | reductase | CGCGGCGCACAAACCCGGGCTGCGGCTGGCAGAGATCCTGCAGACCCTGTTCACCGGCTACGGTGACCGC |
| | amplified | CCGGCGCTGGGATACCGCGCCCGTGAACTGGCCACCGACGAGGGCGGGCGCACCGTGACGCGTCTGCTGC |
| | from | CGCGGTTCGACACCCTCACCTACGCCCAGGTGTGGTCGCGCGTGCAAGCGGTCGCCGCGGCCCTGCGCCA |
| | Mycobacterium | CAACTTCGCGCAGCCGATCTACCCCGGCGACGCCGTCGCGACGATCGGTTTCGCGAGTCCCGATTACCTG |
| | smegmatis. | ACGCTGGATCTCGTATGCGCCTACCTGGGCCTCGTGAGTGTTCCGCTGCAGCACAACGCACCGGTCAGCC |
| | | GGCTCGCCCCGATCCTGGCCGAGGTCGAACCGCGGATCCTCACCGTGAGCGCCGAATACCTCGACCTCGC |
| | | AGTCGAATCCGTGCGGGACGTCAACTCGGTGTCGCAGCTCGTGGTGTTCGACCATCACCCCGAGGTCGAC |
| | | GACCACCGCGACGCACTGGCCCGCGCGCGTGAACAACTCGCCGGCAAGGGCATCGCCGTCACCACCCTGG |
| | | ACGCGATCGCCGACGAGGGCGCCGGGCTGCCGGCCGAACCGATCTACACCGCCGACCATGATCAGCGCCT |
| | | CGCGATGATCCTGTACACCTCGGGTTCCACCGGCGCACCCAAGGGTGCGATGTACACCGAGGCGATGGTG |
| | | GCGCGGCTGTGGACCATGTCGTTCATCACGGGTGACCCCACGCCGGTCATCAACGTCAACTTCATGCCGC |
| | | TCAACCACCTGGGCGGGCGCATCCCCATTTCCACCGCCGTGCAGAACGGTGGAACCAGTTACTTCGTACC |
| | | GGAATCCGACATGTCCACGCTGTTCGAGGATCTCGCGCTGGTGCGCCCGACCGAACTCGGCCTGGTTCCG |
| | | CGCGTCGCCGACATGCTCTACCAGCACCACCTCGCCACCGTCGACCGCCTGGTCACGCAGGGCGCCGACG |
| | | AACTGACCGCCGAGAAGCAGGCCGGTGCCGAACTGCGTGAGCAGGTGCTCGGCGGACGCGTGATCACCGG |
| | | ATTCGTCAGCACCGCACCGCTGGCCGCGGAGATGAGGGCGTTCCTCGACATCACCCTGGGCGCACACATC |
| | | GTCGACGGCTACGGGCTCACCGAGACCGGCGCCGTGACACGCGACGGTGTGATCGTGCGGCCACCGGTGA |
| | | TCGACTACAAGCTGATCGACGTTCCCGAACTCGGCTACTTCAGCACCGACAAGCCCTACCCGCGTGGCGA |
| | | ACTGCTGGTCAGGTCGCAAACGCTGACTCCCGGGTACTACAAGCGCCCCGAGGTCACCGCGAGCGTCTTC |
| | | GACCGGGACGGCTACTACCACACCGGCGACGTCATGGCCGAGACCGCACCCGACCACCTGGTGTACGTGG |
| | | ACCGTCGCAACAACGTCCTCAAACTCGCGCAGGGCGAGTTCGTGGCGGTCGCCAACCTGGAGGCGGTGTT |
| | | CTCCGGCGCGGCGCTGGTGCGCCAGATCTTCGTGTACGGCAACAGCGAGCGCAGTTTCCTTCTGGCCGTG |
| | | GTGGTCCCGACGCCGGAGGCGCTCGAGCAGTACGATCCGGCCGCGCTCAAGGCCGCGCTGGCCGACTCGC |
| | | TGCAGCGCACCGCACGCGACGCCGAACTGCAATCCTACGAGGTGCCGGCCGATTTCATCGTCGAGACCGA |
| | | GCCGTTCAGCGCCGCCAACGGGCTGCTGTCGGGTGTCGGAAAACTGCTGCGGCCCAACCTCAAAGACCGC |
| | | TACGGGCAGCGCCTGGAGCAGATGTACGCCGATATCGCGGCCACGCAGGCCAACCAGTTGCGCGAACTGC |
| | | GGCGCGCGGCCGCCACACAACCGGTGATCGACACCCTCACCCAGGCCGCTGCCACGATCCTCGGCACCGG |
| | | GAGCGAGGTGGCATCCGACGCCCACTTCACCGACCTGGGCGGGGATTCCCTGTCGGCGCTGACACTTTCG |
| | | AACCTGCTGAGCGATTTCTTCGGTTTCGAAGTTCCCGTCGGCACCATCGTGAACCCGGCCACCAACCTCG |
| | | CCCAACTCGCCCAGCACATCGAGGCGCAGCGCACCGCGGGTGACCGCAGGCCGAGTTTCACCACCGTGCA |
| | | CGGCGCGGACGCCACCGAGATCCGGGCGAGTGAGCTGACCCTGGACAAGTTCATCGACGCCGAAACGCTC |
| | | CGGGCCGCACCGGGTCTGCCCAAGGTCACCACCGAGCCACGGACGGTGTTGCTCTCGGGCGCCAACGGCT |
| | | GGCTGGGCCGGTTCCTCACGTTGCAGTGGCTGGAACGCCTGGCACCTGTCGGCGGCACCCTCATCACGAT |
| | | CGTGCGGGGCCGCGACGACGCCGCGGCCCGCGCACGGCTGACCCAGGCCTACGACACCGATCCCGAGTTG |
| | | TCCCGCCGCTTCGCCGAGCTGGCCGACCGCCACCTGCGGGTGGTCGCCGGTGACATCGGCGACCCGAATC |
| | | TGGGCCTCACACCCGAGATCTGGCACCGGCTCGCCGCCGAGGTCGACCTGGTGGTGCATCCGGCAGCGCT |
| | | GGTCAACCACGTGCTCCCCTACCGGCAGCTGTTCGGCCCCAACGTCGTGGGCACGGCCGAGGTGATCAAG |
| | | CTGGCCCTCACCGAACGGATCAAGCCCGTCACGTACCTGTCCACCGTGTCGGTGGCCATGGGGATCCCCG |
| | | ACTTCGAGGAGGACGGCGACATCCGGACCGTGAGCCCGGTGCGCCCGCTCGACGGCGGATACGCCAACGG |
| | | CTACGGCAACAGCAAGTGGGCCGGCGAGGTGCTGCTGCGGGAGGCCCACGATCTGTGCGGGCTGCCCGTG |
| | | GCGACGTTCCGCTCGGACATGATCCTGGCGCATCCGCGCTACCGCGGTCAGGTCAACGTGCCAGACATGT |
| | | TCACGCGACTCCTGTTGAGCCTCTTGATCACCGGCGTCGCGCCGCGGTCGTTCTACATCGGAGACGGTGA |
| | | GCGCCCGCGGGCGCACTACCCCGGCCTGACGGTCGATTTCGTGGCCGAGGCGGTCACGACGCTCGGCGCG |
| | | CAGCAGCGCGAGGGATACGTGTCCTACGACGTGATGAACCCGCACGACGACGGGATCTCCCTGGATGTGT |
| | | TCGTGGACTGGCTGATCCGGGCGGGCCATCCGATCGACCGGGTCGACGACTACGACGACTGGGTGCGTCG |
| | | GTTCGAGACCGCGTTGACCGCGCTTCCCGAGAAGCGCCGCGCACAGACCGTACTGCCGCTGCTGCACGCG |
| | | TTCCGCGCTCCGCAGGCACCGTTGCGCGGCGCACCCGAACCCACGGAGGTGTTCCACGCCGCGGTGCGCA |
| | | CCGCGAAGGTGGGCCCGGGAGACATCCCGCACCTCGACGAGGCGCTGATCGACAAGTACATACGCGATCT |
| | | GCGTGAGTTCGGTCTGATCTGA |
| 54 | codon- | ATGCACCATCACCACCATCATGGAGGCGGACAGCAACTGACCGATCAAAGCAAAGAACTGGACTTCAAGA |
| | optimized | GCGAGACGTACAAAGACGCCTATAGCCGCATTAACGCGATCGTCATTGAAGGCGAACAAGAGGCGCATGA |
| | hexahistidine- | AAACTACATCACCCTGGCGCAGCTGCTGCCTGAGAGCCACGACGAACTGATTCGCCTGAGCAAAATGGAG |
| | tagged | AGCCGTCACAAGAAAGGTTTTGAGGCGTGTGGCCGCAATCTGGCGGTGACCCCGGACCTGCAATTTGCGA |
| | Nostoc | AGGAGTTCTTTAGCGGTCTGCACCAGAATTTCCAGACGGCCGCAGCCGAGGGCAAAGTCGTCACTTGTTT |
| | punctiforme | GTTGATCCAGAGCCTGATTATTGAATGCTTTGCTATTGCGGCGTACAACATTTACATTCCGGTCGCCGAT |
| | adm. | GACTTTGCGCGTAAAATCACGGAAGGTGTTGTCAAAGAGGAGTATTCCCACCTGAATTTCGGTGAAGTGT |
| | | GGTTGAAGGAACATTTTGCGGAATCTAAAGCCGAATTGGAACTGGCAAATCGCCAGAACCTGCCGATCGT |
| | | TTGGAAGATGCTGAACCAAGTGGAAGGTGATGCACATACGATGGCGATGGAGAAGGACGCATTGGTTGAG |
| | | GACTTTATGATTCAGTATGGCGAAGCACTGTCCAATATCGGTTTCAGCACCCGTGATATCATGCGTCTGA |
| | | GCGCCTATGGCCTGATCGGTGCCTAA |
| 55 | codon- | ATGAAAACGACCCACACCAGCTTACCATTTGCCGGCCACACGTTACATTTCGTCGAATTTGATCCGGCGA |
| | Optimized E. | ACTTTTGTGAACAAGACCTGTTGTGGCTGCCGCATTATGCCCAGCTGCAGCACGCAGGCCGTAAGCGTAA |
| | coli entD. | AACTGAACATCTGGCCGGTCGCATTGCGGCAGTGTATGCCCTGCGCGAGTACGGCTACAAATGCGTGCCG |
| | | GCCATTGGTGAACTGCGTCAACCGGTTTGGCCGGCAGAAGTTTACGGTTCCATCTCCCACTGCGGTACTA |
| | | CCGCGTTGGCGGTTGTGTCTCGCCAGCCGATCGGTATTGATATTGAAGAGATATTCTCTGTCCAGACGGC |
| | | ACGCGAGCTGACGGACAACATCATTACCCCGGCAGAGCACGAGCGTCTGGCGGACTGTGGTCTGGCGTTC |
| | | AGCCTGGCGCTGACCCTGGCATTCAGCGCAAAAGAGAGCGCGTTCAAGGCTTCCGAGATCCAAACCGATG |
| | | CGGGCTTCCTGGATTATCAAATCATCAGCTGGAACAAGCAACAGGTTATCATTCACCGTGAGAATGAGAT |
| | | GTTTGCCGTCCATTGGCAGATTAAAGAGAAAATCGTTATCACCCTGTGCCAGCACGACTGA |
| 56 | plasmid | AAAAGCAGAGCATTACGCTGACTTGACGGGACGGCGCAAGCTCATGACCAAAATCCCTTAACGTGAGTTA |
| | pAQ3::P(cpcB)- | CGCGCGCGTCGTTCCACTGAGCGTCAGACCCCGTAGAAAAGATCAAAGGATCTTCTTGAGATCCTTTTTT |
| | Nhistag_adm | TCTGCGCGTAATCTGCTGCTTGCAAACAAAAAAACCACCGCTACCAGCGGTGGTTTGTTTGCCGGATCAA |
| | (Npu)-SpecR. | GAGCTACCAACTCTTTTTCCGAAGGTAACTGGCTTCAGCAGAGCGCAGATACCAAATACTGTTCTTCTAG |
| | | TGTAGCCGTAGTTAGCCCACCACTTCAAGAACTCTGTAGCACCGCCTACATACCTCGCTCTGCTAATCCT |
| | | GTTACCAGTGGCTGCTGCCAGTGGCGATAAGTCGTGTCTTACCGGGTTGGACTCAAGACGATAGTTACCG |
| | | GATAAGGCGCAGCGGTCGGGCTGAACGGGGGGTTCGTGCACACAGCCCAGCTTGGAGCGAACGACCTACA |
| | | CCGAACTGAGATACCTACAGCGTGAGCTATGAGAAAGCGCCACGCTTCCCGAAGGGAGAAAGGCGGACAG |
| | | GTATCCGGTAAGCGGCAGGGTCGGAACAGGAGAGCGCACGAGGGAGCTTCCAGGGGGAAACGCCTGGTAT |
| | | CTTTATAGTCCTGTCGGGTTTCGCCACCTCTGACTTGAGCGTCGATTTTTGTGATGCTCGTCAGGGGGGC |
| | | GGAGCCTATGGAAAAACGCCAGCAACGCGGCCTTTTTACGGTTCCTGGCCTTTTGCTGGCCTTTTGCTCA |
| | | CATGTTCTTTCCTGCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAAGGCGAGAGTAGGGAAC |
| | | TGCCAGGCATCAAACTAAGCAGAAGGCCCCTGACGGATGGCCTTTTTGCGTTTCTACAAACTCTTTCTGT |
| | | GTTGTAAAACGACGGCCAGTCTTAAGCTCGGGCCCCCTGGGCGGTTCTGATAACGAGTAATCGTTAATCC |
| | | GCAAATAACGTAAAAACCCGCTTCGGCGGGTTTTTTTATGGGGGGAGTTTAGGGAAAGAGCATTTGTCAG |
| | | AATATTTAAGGGCGCCTGTCACTTTGCTTGATATATGAGAATTATTTAACCTTATAAATGAGAAAAAAGC |
| | | AACGCACTTTAAATAAGATACGTTGCTTTTTCGATTGATGAACACCTATAATTAAACTATTCATCTATTA |
| | | TTTATGATTTTTTGTATATACAATATTTCTAGTTTGTTAAAGAGAATTAAGAAAATAAATCTCGAAAATA |
| | | ATAAAGGGAAAATCAGTTTTTGATATCAAAATTATACATGTCAACGATAATACAAAATATAATACAAACT |
| | | ATAAGATGTTATCAGTATTTATTATGCATTTAGAATAAATTTTGTGTCGCCCTTCGCTGAACCTGCAGGC |
| | | GAGCATTTCAACGATGATGAATGGGACGGCGAACCCACTGAACCCGTCGCCATTGACCCAGAACCGCGCA |
| | | AAGAACGGGAAAAAATTGATCTCGATCTGGAGGATGAACCAGAGGAAAACCGCAAACCGCAAAAAATCAA |
| | | AGTGAAGTTAGCCGATGGGAAAGAGCGGGAACTCGCCCATACTCAAACCACAACTTTTTGGGATGCTGAT |
| | | GGTAAACCCATTTCCGCCCAAGAATTTATCGAAAAGCTATTTGGCGACCTGCCCGACCTCTTCAAGGATG |
| | | AAGCCGAACTACGCACCATCTGGGGGAAACCCGATACCCGTAAATCGTTCCTGACCGGACTCGCGGAAAA |
| | | AGGCTACGGTGACACCCAACTGAAGGCGATCGCACGCATTGCCGAAGCGGAAAAAAGTGATGTCTATGAT |
| | | GTCCTGACTTGGGTTGCCTACAACACCAAACCCATTAGCAGAGAAGAGCGAGTAATTAAGCATCGAGATC |
| | | TGATTTTCTCGAAGTACACCGGAAAGCAGCAAGAATTTTTAGATTTTGTCCTAGACCAATACATTCGAGA |
| | | AGGAGTGGAGGAACTTGATCGGGGGAAACTGCCTACCCTCATCGAAATCAAATACCAAACCGTTAATGAA |
| | | GGTTTAGTGATCTTGGGTCAGGATATCGGTCAAGTATTCGCAGATTTTCAGGCGGATTTATATACCGAAG |
| | | ATGTGGCATAAAAAAGGACGGCGATCGCCGGGGGCGTTGCCTGCCTTGAGCGGCCGCGTCGACTTCGTTA |
| | | TAAAATAAACTTAACAAATCTATACCCACCTGTAGAGAAGAGTCCCTGAATATCAAAATGGTGGGATAAA |
| | | AAGCTCAAAAAGGAAAGTAGGCTGTGGTTCCCTAGGCAACAGTCTTCCCTACCCCACTGGAAACTAAAAA |
| | | AACGAGAAAAGTTCGCACCGAACATCAATTGCATAATTTTAGCCCTAAAACATAAGCTGAACGAAACTGG |
| | | TTGTCTTCCCTTCCCAATCCAGGACAATCTGAGAATCCCCTGCAACATTACTTAACAAAAAAGCAGGAAT |
| | | AAAATTAACAAGATGTAACAGACATAAGTCCCATCACCGTTGTATAAAGTTAACTGTGGGATTGCAAAAG |
| | | CATTCAAGCCTAGGCGCTGAGCTGTTTGAGCATCCCGGTGGCCCTTGTCGCTGCCTCCGTGTTTCTCCCT |
| | | GGATTTATTTAGGTAATATCTCTCATAAATCCCCGGGTAGTTAACGAAAGTTAATGGAGATCAGTAACAA |
| | | TAACTCTAGGGTCATTACTTTGGACTCCCTCAGTTTATCCGGGGGAATTGTGTTTAAGAAAATCCCAACT |
| | | CATAAAGTCAAGTAGGAGATTAATCATATGCACCATCACCACCATCATGGAGGCGGACAGCAACTGACCG |
| | | ATCAAAGCAAAGAACTGGACTTCAAGAGCGAGACGTACAAAGACGCCTATAGCCGCATTAACGCGATCGT |
| | | CATTGAAGGCGAACAAGAGGCGCATGAAAACTACATCACCCTGGCGCAGCTGCTGCCTGAGAGCCACGAC |
| | | GAACTGATTCGCCTGAGCAAAATGGAGAGCCGTCACAAGAAAGGTTTTGAGGCGTGTGGCCGCAATCTGG |
| | | CGGTGACCCCGGACCTGCAATTTGCGAAGGAGTTCTTTAGCGGTCTGCACCAGAATTTCCAGACGGCCGC |
| | | AGCCGAGGGCAAAGTCGTCACTTGTTTGTTGATCCAGAGCCTGATTATTGAATGCTTTGCTATTGCGGCG |
| | | TACAACATTTACATTCCGGTCGCCGATGACTTTGCGCGTAAAATCACGGAAGGTGTTGTCAAAGAGGAGT |
| | | ATTCCCACCTGAATTTCGGTGAAGTGTGGTTGAAGGAACATTTTGCGGAATCTAAAGCCGAATTGGAACT |
| | | GGCAAATCGCCAGAACCTGCCGATCGTTTGGAAGATGCTGAACCAAGTGGAAGGTGATGCACATACGATG |
| | | GCGATGGAGAAGGACGCATTGGTTGAGGACTTTATGATTCAGTATGGCGAAGCACTGTCCAATATCGGTT |
| | | TCAGCACCCGTGATATCATGCGTCTGAGCGCCTATGGCCTGATCGGTGCCTAAGAGCTCCTCGAGGAATT |
| | | CGGTTTTCCGTCCTGTCTTGATTTTCAAGCAAACAATGCCTCCGATTTCTAATCGGAGGCATTTGTTTTT |
| | | GTTTATTGCAAAAACAAAAAATATTGTTACAAATTTTTACAGGCTATTAAGCCTACCGTCATAAATAATT |
| | | TGCCATTTACTAGTTTTTAATTAACCAGAACCTTGACCGAACGCAGCGGTGGTAACGGCGCAGTGGCGGT |
| | | TTTCATGGCTTGTTATGACTGTTTTTTTGGGGTACAGTCTATGCCTCGGGCATCCAAGCAGCAAGCGCGT |
| | | TACGCCGTGGGTCGATGTTTGATGTTATGGAGCAGCAACGATGTTACGCAGCAGGGCAGTCGCCCTAAAA |
| | | CAAAGTTAAACATCATGAGGGAAGCGGTGATCGCCGAAGTATCGACTCAACTATCAGAGGTAGTTGGCGT |
| | | CATCGAGCGCCATCTCGAACCGACGTTGCTGGCCGTACATTTGTACGGCTCCGCAGTGGATGGCGGCCTG |
| | | AAGCCACACAGTGATATTGATTTGCTGGTTACGGTGACCGTAAGGCTTGATGAAACAACGCGGCGAGCTT |
| | | TGATCAACGACCTTTTGGAAACTTCGGCTTCCCCTGGAGAGAGCGAGATTCTCCGCGCTGTAGAAGTCAC |
| | | CATTGTTGTGCACGACGACATCATTCCGTGGCGTTATCCAGCTAAGCGCGAACTGCAATTTGGAGAATGG |
| | | CAGCGCAATGACATTCTTGCAGGTATCTTCGAGCCAGCCACGATCGACATTGATCTGGCTATCTTGCTGA |
| | | CAAAAGCAAGAGAACATAGCGTTGCCTTGGTAGGTCCAGCGGCGGAGGAACTCTTTGATCCGGTTCCTGA |
| | | ACAGGATCTATTTGAGGCGCTAAATGAAACCTTAACGCTATGGAACTCGCCGCCCGACTGGGCTGGCGAT |
| | | GAGCGAAATGTAGTGCTTACGTTGTCCCGCATTTGGTACAGCGCAGTAACCGGCAAAATCGCGCCGAAGG |
| | | ATGTCGCTGCCGACTGGGCAATGGAGCGCCTGCCGGCCCAGTATCAGCCCGTCATACTTGAAGCTAGACA |
| | | GGCTTATCTTGGACAAGAAGAAGATCGCTTGGCCTCGCGCGCAGATCAGTTGGAAGAATTTGTCCACTAC |
| | | GTGAAAGGCGAGATCACCAAGGTAGTCGGCAAATAATGTCTAACAATTCGTTCAAGCCGACGCCGCTTCG |
| | | CGGCGCGGCTTAACTCAAGCGTTAGATGCACTAAGCACATAATTGCTCACAGCCAAACTATCAGGTCAAG |
| | | TCTGCTTTTATTATTTTTAAGCGTGCATAATAAGCCCTACACAAATTGGGAGATATATCATGAGGCGCGC |
| | | CACGAGAAAGAGTTATGACAAATTAAAATTCTGACTCTTAGATTATTTCCAGAGAGGCTGATTTTCCCAA |
| | | TCTTTGGGAAAGCCTAAGTTTTTAGATTCTATTTCTGGATACATCTCAAAAGTTCTTTTTAAATGCTGTG |
| | | CAAAATTATGCTCTGGTTTAATTCTGTCTAAGAGATACTGAATACAACATAAGCCAGTGAAAATTTTACG |
| | | GCTGTTTCTTTGATTAATATCCTCCAATACTTCTCTAGAGAGCCATTTTCCTTTTAACCTATCAGGCAAT |
| | | TTAGGTGATTCTCCTAGCTGTATATTCCAGAGCCTTGAATGATGAGCGCAAATATTTCTAATATGCGACA |
| | | AAGACCGTAACCAAGATATAAAAAACTTGTTAGGTAATTGGAAATGAGTATGTATTTTTTGTCGTGTCTT |
| | | AGATGGTAATAAATTTGTGTACATTCTAGATAACTGCCCAAAGGCGATTATCTCCAAAGCCATATATGAC |
| | | GGCGGTAGTAGAGGATTTGTGTACTTGTTTCGATAATGCCCGATAAATTCTTCTACTTTTTTAGATTGGC |
| | | AATATTGAGTAATCGAATCGATTAATTCTTGATGCTTCCCAGTGTCATAAAATAAACTTTTATTCAGATA |
| | | CCAATGAGGATCATAATCATGGGAGTAGTGATAAATCATTTGAGTTCTGACTGCTACTTCTATCGACTCC |
| | | GTAGCATTAAAAATAAGCATTCTCAAGGATTTATCAAACTTGTATAGATTTGGCCGGCCCGTCAAAAGGG |
| | | CGACACCCCATAATTAGCCCGGGCGAAAGGCCCAGTCTTTCGACTGAGCCTTTCGTTTTATTTGATGCCT |
| | | GGCAGTTCCCTACTCTCGCATGGGGAGTCCCCACACTACCATCGGCGCTACGGCGTTTCACTTCTGAGTT |
| | | CGGCATGGGGTCAGGTGGGACCACCGCGCTACTGCCGCCAGGCAAACAAGGGGTGTTATGAGCCATATTC |
| | | AGGTATAAATGGGCTCGCGATAATGTTCAGAATTGGTTAATTGGTTGTAACACTGACCCCTATTTGTTTA |
| | | TTTTTCTAAATACATTCAAATATGTATCCGCTCATGAGACAATAACCCTGATAAATGCTTCAATAATATT |
| | | GAAAAAGGAAGAATATGAGTATTCAACATTTCCGTGTCGCCCTTATTCCCTTTTTTGCGGCATTTTGCCT |
| | | TCCTGTTTTTGCTCACCCAGAAACGCTGGTGAAAGTAAAAGATGCTGAAGATCAGTTGGGTGCACGAGTG |
| | | GGTTACATCGAACTGGATCTCAACAGCGGTAAGATCCTTGAGAGTTTTCGCCCCGAAGAACGTTTTCCAA |
| | | TGATGAGCACTTTTAAAGTTCTGCTATGTGGCGCGGTATTATCCCGTATTGACGCCGGGCAAGAGCAACT |
| | | CGGTCGCCGCATACACTATTCTCAGAATGACTTGGTTGAGTACTCACCAGTCACAGAAAAGCATCTTACG |
| | | GATGGCATGACAGTAAGAGAATTATGCAGTGCTGCCATAACCATGAGTGATAACACTGCGGCCAACTTAC |
| | | TTCTGACAACGATCGGAGGACCGAAGGAGCTAACCGCTTTTTTGCACAACATGGGGGATCATGTAACTCG |
| | | CCTTGATCGTTGGGAACCGGAGCTGAATGAAGCCATACCAAACGACGAGCGTGACACCACGATGCCTGTA |
| | | GCGATGGCAACAACGTTGCGCAAACTATTAACTGGCGAACTACTTACTCTAGCTTCCCGGCAACAATTAA |
| | | TAGACTGGATGGAGGCGGATAAAGTTGCAGGACCACTTCTGCGCTCGGCCCTTCCGGCTGGCTGGTTTAT |
| | | TGCTGATAAATCCGGAGCCGGTGAGCGTGGTTCTCGCGGTATCATCGCAGCGCTGGGGCCAGATGGTAAG |
| | | CCCTCCCGTATCGTAGTTATCTACACGACGGGGAGTCAGGCAACTATGGATGAACGAAATAGACAGATCG |
| | | CTGAGATAGGTGCCTCACTGATTAAGCATTGGT |
| 57 | plasmid | TAGAAAAACTCATCGAGCATCAAATGAAACTGCAATTTATTCATATCAGGATTATCAATACCATATTTTT |
| | pAQ4::P(cpcB)- | GAAAAAGCCGTTTCTGTAATGAAGGAGAAAACTCACCGAGGCAGTTCCATAGGATGGCAAGATCCTGGTA |
| | Nhistag_adm | TCGGTCTGCGATTCCGACTCGTCCAACATCAATACAACCTATTAATTTCCCCTCGTCAAAAATAAGGTTA |
| | (Npu)-ErmC. | TCAAGTGAGAAATCACCATGAGTGACGACTGAATCCGGTGAGAATGGCAAAAGTTTATGCATTTCTTTCC |
| | | AGACTTGTTCAACAGGCCAGCCATTACGCTCGTCATCAAAATCACTCGCATCAACCAAACCGTTATTCAT |
| | | TCGTGATTGCGCCTGAGCGAGGCGAAATACGCGATCGCTGTTAAAAGGACAATTACAAACAGGAATCGAG |
| | | TGCAACCGGCGCAGGAACACTGCCAGCGCATCAACAATATTTTCACCTGAATCAGGATATTCTTCTAATA |
| | | CCTGGAACGCTGTTTTTCCGGGGATCGCAGTGGTGAGTAACCATGCATCATCAGGAGTACGGATAAAATG |
| | | CTTGATGGTCGGAAGTGGCATAAATTCCGTCAGCCAGTTTAGTCTGACCATCTCATCTGTAACATCATTG |
| | | GCAACGCTACCTTTGCCATGTTTCAGAAACAACTCTGGCGCATCGGGCTTCCCATACAAGCGATAGATTG |
| | | TCGCACCTGATTGCCCGACATTATCGCGAGCCCATTTATACCCATATAAATCAGCATCCATGTTGGAATT |
| | | TAATCGCGGCCTCGACGTTTCCCGTTGAATATGGCTCATATTCTTCCTTTTTCAATATTATTGAAGCATT |
| | | TATCAGGGTTATTGTCTCATGAGCGGATACATATTTGAATGTATTTAGAAAAATAAACAAATAGGGGTCA |
| | | GTGTTACAACCAATTAACCAATTCTGAACATTATCGCGAGCCCATTTATACCTGAATATGGCTCATAACA |
| | | CCCCTTGTTTGCCTGGCGGCAGTAGCGCGGTGGTCCCACCTGACCCCATGCCGAACTCAGAAGTGAAACG |
| | | CCGTAGCGCCGATGGTAGTGTGGGGACTCCCCATGCGAGAGTAGGGAACTGCCAGGCATCAAATAAAACG |
| | | AAAGGCTCAGTCGAAAGACTGGGCCTTTCGCCCGGGCTAATTAGGGGGTGTCGCCCTTTACACGTACTTA |
| | | GTCGCTGAAGGCCTCACTGGCCCCTGCAGGGATGGTGGAATGCTGGTTATCTGGTGGGGATTAAGTGGTG |
| | | TTTTACTAAAGCTTGAACAACTCAAGAAAGATTATATTCGCAATAACTGCCAATAATCCCAGCATCTTGA |
| | | GAAAATCCAGCAAACCGGGGGCAAAACACCAGCAAGAAGCCAGCAGACTATCACCAAATCCCCAGCGTAC |
| | | AGCTAGAAATAACTGAGCAGTTGTATTCAATTACCTTCTGGTCAAGCCGAGGAAATTTCCCCACACCTTA |
| | | TACACCTCTGGAAGGTTTTTTTGACGAAGCGCAAAATATCCACAATCGGCTGGGGACTTCTTCTGTCAGA |
| | | AAATGGCAGAAATTTTTGAATGTGTTGGCGATCGCCCTCATCAATGATTATTAGAGAACTTTTGTCCCTG |
| | | ATGTTGGGAATACTCTTGATGACAATTGTGATTGCTCAAAGAAGAAAGAAATTTGGAGTAAATCTCTAAA |
| | | AGGGGACTGAAATATTTGTATGGTCAGCATGACCACTGAAATGGAGAGAAGTCTAAGACAGTAGATGTCT |
| | | TAGATATAAGCCTCATTAGAAGCCATGCCATAAAACAGATTTTGTGGATGAAACAACTTGAAATAGTTCA |
| | | GTTGTAGACCATGTTATAAACATTTATTCTTAACACAGTGACACATTAATGACTCATATATCCGTCCAAA |
| | | AAAAACTAAAATGTTTGTAAATTTAGTTTTGCGGCCGCGTCGACTTCGTTATAAAATAAACTTAACAAAT |
| | | CTATACCCACCTGTAGAGAAGAGTCCCTGAATATCAAAATGGTGGGATAAAAAGCTCAAAAAGGAAAGTA |
| | | GGCTGTGGTTCCCTAGGCAACAGTCTTCCCTACCCCACTGGAAACTAAAAAAACGAGAAAAGTTCGCACC |
| | | GAACATCAATTGCATAATTTTAGCCCTAAAACATAAGCTGAACGAAACTGGTTGTCTTCCCTTCCCAATC |
| | | CAGGACAATCTGAGAATCCCCTGCAACATTACTTAACAAAAAAGCAGGAATAAAATTAACAAGATGTAAC |
| | | AGACATAAGTCCCATCACCGTTGTATAAAGTTAACTGTGGGATTGCAAAAGCATTCAAGCCTAGGCGCTG |
| | | AGCTGTTTGAGCATCCCGGTGGCCCTTGTCGCTGCCTCCGTGTTTCTCCCTGGATTTATTTAGGTAATAT |
| | | CTCTCATAAATCCCCGGGTAGTTAACGAAAGTTAATGGAGATCAGTAACAATAACTCTAGGGTCATTACT |
| | | TTGGACTCCCTCAGTTTATCCGGGGGAATTGTGTTTAAGAAAATCCCAACTCATAAAGTCAAGTAGGAGA |
| | | TTAATCATATGCACCATCACCACCATCATGGAGGCGGACAGCAACTGACCGATCAAAGCAAAGAACTGGA |
| | | CTTCAAGAGCGAGACGTACAAAGACGCCTATAGCCGCATTAACGCGATCGTCATTGAAGGCGAACAAGAG |
| | | GCGCATGAAAACTACATCACCCTGGCGCAGCTGCTGCCTGAGAGCCACGACGAACTGATTCGCCTGAGCA |
| | | AAATGGAGAGCCGTCACAAGAAAGGTTTTGAGGCGTGTGGCCGCAATCTGGCGGTGACCCCGGACCTGCA |
| | | ATTTGCGAAGGAGTTCTTTAGCGGTCTGCACCAGAATTTCCAGACGGCCGCAGCCGAGGGCAAAGTCGTC |
| | | ACTTGTTTGTTGATCCAGAGCCTGATTATTGAATGCTTTGCTATTGCGGCGTACAACATTTACATTCCGG |
| | | TCGCCGATGACTTTGCGCGTAAAATCACGGAAGGTGTTGTCAAAGAGGAGTATTCCCACCTGAATTTCGG |
| | | TGAAGTGTGGTTGAAGGAACATTTTGCGGAATCTAAAGCCGAATTGGAACTGGCAAATCGCCAGAACCTG |
| | | CCGATCGTTTGGAAGATGCTGAACCAAGTGGAAGGTGATGCACATACGATGGCGATGGAGAAGGACGCAT |
| | | TGGTTGAGGACTTTATGATTCAGTATGGCGAAGCACTGTCCAATATCGGTTTCAGCACCCGTGATATCAT |
| | | GCGTCTGAGCGCCTATGGCCTGATCGGTGCCTAAGAGCTCCTCGAGGAATTCGGTTTTCCGTCCTGTCTT |
| | | GATTTTCAAGCAAACAATGCCTCCGATTTCTAATCGGAGGCATTTGTTTTTGTTTATTGCAAAAACAAAA |
| | | AATATTGTTACAAATTTTTACAGGCTATTAAGCCTACCGTCATAAATAATTTGCCATTTACTAGTTTTAA |
| | | TTAACGTGCTATAATTATACTAATTTTATAAGGAGGAAAAAATATGGGCATTTTTAGTATTTTTGTAATC |
| | | AGCACAGTTCATTATCAACCAAACAAAAAATAAGTGGTTATAATGAATCGTTAATAAGCAAAATTCATAT |
| | | AACCAAATTAAAGAGGGTTATAATGAACGAGAAAAATATAAAACACAGTCAAAACTTTATTACTTCAAAA |
| | | CATAATATAGATAAAATAATGACAAATATAAGATTAAATGAACATGATAATATCTTTGAAATCGGCTCAG |
| | | GAAAAGGCCATTTTACCCTTGAATTAGTAAAGAGGTGTAATTTCGTAACTGCCATTGAAATAGACCATAA |
| | | ATTATGCAAAACTACAGAAAATAAACTTGTTGATCACGATAATTTCCAAGTTTTAAACAAGGATATATTG |
| | | CAGTTTAAATTTCCTAAAAACCAATCCTATAAAATATATGGTAATATACCTTATAACATAAGTACGGATA |
| | | TAATACGCAAAATTGTTTTTGATAGTATAGCTAATGAGATTTATTTAATCGTGGAATACGGGTTTGCTAA |
| | | AAGATTATTAAATACAAAACGCTCATTGGCATTACTTTTAATGGCAGAAGTTGATATTTCTATATTAAGT |
| | | ATGGTTCCAAGAGAATATTTTCATCCTAAACCTAAAGTGAATAGCTCACTTATCAGATTAAGTAGAAAAA |
| | | AATCAAGAATATCACACAAAGATAAACAAAAGTATAATTATTTCGTTATGAAATGGGTTAACAAAGAATA |
| | | CAAGAAAATATTTACAAAAAATCAATTTAACAATTCCTTAAAACATGCAGGAATTGACGATTTAAACAAT |
| | | ATTAGCTTTGAACAATTCTTATCTCTTTTCAATAGCTATAAATTATTTAATAAGTAAGTTAAGGGATGCA |
| | | TAAACTGCATCCCTTAACTTGTTTTTCGTGTGCCTATTTTTTGTGGCGCGCCCAGTTTCCTTTACTGGCC |
| | | CTAAAGTCGCTGTGGCTAGGGTTCCGAAGGGGCATTATTGGCTCGCGGCTTTACAACCTTGATAAGGAGA |
| | | GAGATGACAGTTTTTTTTCTCTTTTGCTTAGTAAAACAGCAAATTTAAGGCATGTTAAAGAGCAGTAGAA |
| | | CGAAATGGTTGAGCCGGCCTCGATACACTCAATTAACTACTAATAGCTTCAATAAATTTTGGGACGATTG |
| | | AAGCTATtTTTTTGAAAATCAACTCTTAATATCTCCTGTCTCAAAAGAGTTAATTGCTAAACAAAAGCCA |
| | | GTTTCAGCGAAAAATCTAGAGTTTTATAGGTTCGTTCTCAGTACAGGACAAAAAGTTTGAAAAGGATAGA |
| | | GGGAGAGGGTTTGATGGAAATAAGCACAAATCAATCAAGCCCTCATGAATCAGATTAGCGAAATTCGCCG |
| | | CCAATTGCGACCTCATCTCGGATGGCATGGAGCCAGACTGTCATTTATCGCCCTCTTCCTGGTGGCACTG |
| | | TTCCGAGCAAAAACCGTCAATCTCGCCAAACTCGCCACCGTCTGGGGAGGCAATGCAGCAGAAGAGTCTA |
| | | ATTACAAACGCATGCAGCGATTCTTTCAGTCCTTTGACGTCAACATGGACAAAATCGCCAGGATGGTAAT |
| | | GAATATCGCGGCTATCCCGCAACCTTGGGTCTTAAGCATCGACCGCACCAACGGCCGGCCTACATGGCCC |
| | | GTCAATCGAAGGGCGACACAAAATTTATTCTAAATGCATAATAAATACTGATAACATCTTATAGTTTGTA |
| | | TTATATTTTGTATTATCGTTGACATGTATAATTTTGATATCAAAAACTGATTTTCCCTTTATTATTTTCG |
| | | AGATTTATTTTCTTAATTCTCTTTAACAAACTAGAAATATTGTATATACAAAAAATCATAAATAATAGAT |
| | | GAATAGTTTAATTATAGGTGTTCATCAATCGAAAAAGCAACGTATCTTATTTAAAGTGCGTTGCTTTTTT |
| | | CTCATTTATAAGGTTAAATAATTCTCATATATCAAGCAAAGTGACAGGCGCCCTTAAATATTCTGACAAA |
| | | TGCTCTTTCCCTAAACTCCCCCCATAAAAAAACCCGCCGAAGCGGGTTTTTACGTTATTTGCGGATTAAC |
| | | GATTACTCGTTATCAGAACCGCCCAGGGGGCCCGAGCTTAAGACTGGCCGTCGTTTTACAACACAGAAAG |
| | | AGTTTGTAGAAACGCAAAAAGGCCATCCGTCAGGGGCCTTCTGCTTAGTTTGATGCCTGGCAGTTCCCTA |
| | | CTCTCGCCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTGAGCAAA |
| | | AGGCCAGCAAAAGGCCAGGAACCGTAAAAAGGCCGCGTTGCTGGCGTTTTTCCATAGGCTCCGCCCCCCT |
| | | GACGAGCATCACAAAAATCGACGCTCAAGTCAGAGGTGGCGAAACCCGACAGGACTATAAAGATACCAGG |
| | | CGTTTCCCCCTGGAAGCTCCCTCGTGCGCTCTCCTGTTCCGACCCTGCCGCTTACCGGATACCTGTCCGC |
| | | CTTTCTCCCTTCGGGAAGCGTGGCGCTTTCTCATAGCTCACGCTGTAGGTATCTCAGTTCGGTGTAGGTC |
| | | GTTCGCTCCAAGCTGGGCTGTGTGCACGAACCCCCCGTTCAGCCCGACCGCTGCGCCTTATCCGGTAACT |
| | | ATCGTCTTGAGTCCAACCCGGTAAGACACGACTTATCGCCACTGGCAGCAGCCACTGGTAACAGGATTAG |
| | | CAGAGCGAGGTATGTAGGCGGTGCTACAGAGTTCTTGAAGTGGTGGGCTAACTACGGCTACACTAGAAGA |
| | | ACAGTATTTGGTATCTGCGCTCTGCTGAAGCCAGTTACCTTCGGAAAAAGAGTTGGTAGCTCTTGATCCG |
| | | GCAAACAAACCACCGCTGGTAGCGGTGGTTTTTTTGTTTGCAAGCAGCAGATTACGCGCAGAAAAAAAGG |
| | | ATCTCAAGAAGATCCTTTGATCTTTTCTACGGGGTCTGACGCTCAGTGGAACGACGCGCGCGTAACTCAC |
| | | GTTAAGGGATTTTGGTCATGAGCTTGCGCCGTCCCGTCAAGTCAGCGTAATGCTCTGCTTT |
| 58 | plasmid | ACCAATGCTTAATCAGTGAGGCACCTATCTCAGCGATCTGTCTATTTCGTTCATCCATAGTTGCCTGACT |
| | pAQ7::P(nir07)- | CCCCGTCGTGTAGATAACTACGATACGGGAGGGCTTACCATCTGGCCCCAGCGCTGCGATGATACCGCGA |
| | carB-entD- | GAACCACGCTCACCGGCTCCGGATTTATCAGCAATAAACCAGCCAGCCGGAAGGGCCGAGCGCAGAAGTG |
| | KanR | GTCCTGCAACTTTATCCGCCTCCATCCAGTCTATTAATTGTTGCCGGGAAGCTAGAGTAAGTAGTTCGCC |
| | | AGTTAATAGTTTGCGCAACGTTGTTGCCATCGCTACAGGCATCGTGGTGTCACGCTCGTCGTTTGGTATG |
| | | GCTTCATTCAGCTCCGGTTCCCAACGATCAAGGCGAGTTACATGATCCCCCATGTTGTGCAAAAAAGCGG |
| | | TTAGCTCCTTCGGTCCTCCGATCGTTGTCAGAAGTAAGTTGGCCGCAGTGTTATCACTCATGGTTATGGC |
| | | AGCACTGCATAATTCTCTTACTGTCATGCCATCCGTAAGATGCTTTTCTGTGACTGGTGAGTACTCAACC |
| | | AAGTCATTCTGAGAATAGTGTATGCGGCGACCGAGTTGCTCTTGCCCGGCGTCAATACGGGATAATACCG |
| | | CGCCACATAGCAGAACTTTAAAAGTGCTCATCATTGGAAAACGTTCTTCGGGGCGAAAACTCTCAAGGAT |
| | | CTTACCGCTGTTGAGATCCAGTTCGATGTAACCCACTCGTGCACCCAACTGATCTTCAGCATCTTTTACT |
| | | TTCACCAGCGTTTCTGGGTGAGCAAAAACAGGAAGGCAAAATGCCGCAAAAAAGGGAATAAGGGCGACAC |
| | | GGAAATGTTGAATACTCATATTCTTCCTTTTTCAATATTATTGAAGCATTTATCAGGGTTATTGTCTCAT |
| | | GAGCGGATACATATTTGAATGTATTTAGAAAAATAAACAAATAGGGGTCAGTGTTACAACCAATTAACCA |
| | | ATTCTGAACATTATCGCGAGCCCATTTATACCTGAATATGGCTCATAACACCCCTTGTTTGCCTGGCGGC |
| | | AGTAGCGCGGTGGTCCCACCTGACCCCATGCCGAACTCAGAAGTGAAACGCCGTAGCGCCGATGGTAGTG |
| | | TGGGGACTCCCCATGCGAGAGTAGGGAACTGCCAGGCATCAAATAAAACGAAAGGCTCAGTCGAAAGACT |
| | | GGGCCTTTCGCCCGGGCTAATTATGGGGTGTCGCCCTTATTCGACTCTATAGTGAAGTTCCTATTCTCTA |
| | | GAAAGTATAGGAACTTCTGAAGTGGGGCCTGCAGGGCCACCACAGCCAAATTCATCGTTAATGTGGACTT |
| | | GCCGACGCCCCCTTTTCGACTAACAATCGCAATTTTTTTCATAGACATTTCCCACAGACCACATCAAATT |
| | | ACAGCAATTGATCTAGCTGAAAGTTTAACCCACTTCCCCCCAGACCCAGAAGACCAGAGGCGCTTAAGCT |
| | | TCCCCGAACAAACTCAACTGACCGAGGGGGAGGGAGCCGTAGCGGCGTTGGTGTTGGCGTAAATGACAGG |
| | | CCGAGCAAAGAGCGATGAGATTTTCCCGACGATTGTCTTCGGGGATGTAATTTTTGTGGTGGACGCTTAA |
| | | GGTTAAAACAGCCCGCAGGTGACGATCAATGCCTTTGACCTTCACATCCGACGGAATACAAACCAAGCCA |
| | | CAGAGTTCACAGCGCCAGTCTGCATCCTCTTTTACTTGTAAGGCGATCGCCTGCCAATCATCAGAATATC |
| | | GAGAAGAATGTTTCATCTAAACCTAGCGCCGCAAGATAATCCTGAAATCGCTACAGTATTAAAAAATTCT |
| | | GGCCAACATCACAGCCAATACTGCGGCCGCTACTCATTAGTTAAGTGTAATGCAGAAAACGCATATTCTC |
| | | TATTAAACTTACGCATTAATACGAGAATTTTGTAGCTACTTATACTATTTTACCTGAGATCCCGACATAA |
| | | CCTTAGAAGTATCGAAATCGTTACATAAACATTCACACAAACCACTTGACAAATTTAGCCAATGTAAAAG |
| | | ACTACAGTTTCTCCCCGGTTTAGTTCTAGAGTTACCTTCAGTGAAACATCGGCGGCGTGTCAGTCATTGA |
| | | AGTAGCATAAATCAATTCAAAATACCCTGCGGGAAGGCTGCGCCAACAAAATTAAATATTTGGTTTTTCA |
| | | CTATTAGAGCATCGATTCATTAATCAAAAACCTTACCCCCCAGCCCCCTTCCCTTGTAGGGAAGTGGGAG |
| | | CCAAACTCCCCTCTCCGCGTCGGAGCGAAAAGTCTGAGCGGAGGTTTCCTCCGAACAGAACTTTTAAAGA |
| | | GAGAGGGGTTGGGGGAGAGGTTCTTTCAAGATTACTAAATTGCTATCACTAGACCTCGTAGAACTAGCAA |
| | | AGACTACGGGTGGATTGATCTTGAGCAAAAAAACTTTATGAGAACTTTAGCAGGAGGAAAACCATATGAC |
| | | CAGCGATGTTCACGACGCCACAGACGGCGTCACCGAAACCGCACTCGACGACGAGCAGTCGACCCGCCGC |
| | | ATCGCCGAGCTGTACGCCACCGATCCCGAGTTCGCCGCCGCCGCACCGTTGCCCGCCGTGGTCGACGCGG |
| | | CGCACAAACCCGGGCTGCGGCTGGCAGAGATCCTGCAGACCCTGTTCACCGGCTACGGTGACCGCCCGGC |
| | | GCTGGGATACCGCGCCCGTGAACTGGCCACCGACGAGGGCGGGCGCACCGTGACGCGTCTGCTGCCGCGG |
| | | TTCGACACCCTCACCTACGCCCAGGTGTGGTCGCGCGTGCAAGCGGTCGCCGCGGCCCTGCGCCACAACT |
| | | TCGCGCAGCCGATCTACCCCGGCGACGCCGTCGCGACGATCGGTTTCGCGAGTCCCGATTACCTGACGCT |
| | | GGATCTCGTATGCGCCTACCTGGGCCTCGTGAGTGTTCCGCTGCAGCACAACGCACCGGTCAGCCGGCTC |
| | | GCCCCGATCCTGGCCGAGGTCGAACCGCGGATCCTCACCGTGAGCGCCGAATACCTCGACCTCGCAGTCG |
| | | AATCCGTGCGGGACGTCAACTCGGTGTCGCAGCTCGTGGTGTTCGACCATCACCCCGAGGTCGACGACCA |
| | | CCGCGACGCACTGGCCCGCGCGCGTGAACAACTCGCCGGCAAGGGCATCGCCGTCACCACCCTGGACGCG |
| | | ATCGCCGACGAGGGCGCCGGGCTGCCGGCCGAACCGATCTACACCGCCGACCATGATCAGCGCCTCGCGA |
| | | TGATCCTGTACACCTCGGGTTCCACCGGCGCACCCAAGGGTGCGATGTACACCGAGGCGATGGTGGCGCG |
| | | GCTGTGGACCATGTCGTTCATCACGGGTGACCCCACGCCGGTCATCAACGTCAACTTCATGCCGCTCAAC |
| | | CACCTGGGCGGGCGCATCCCCATTTCCACCGCCGTGCAGAACGGTGGAACCAGTTACTTCGTACCGGAAT |
| | | CCGACATGTCCACGCTGTTCGAGGATCTCGCGCTGGTGCGCCCGACCGAACTCGGCCTGGTTCCGCGCGT |
| | | CGCCGACATGCTCTACCAGCACCACCTCGCCACCGTCGACCGCCTGGTCACGCAGGGCGCCGACGAACTG |
| | | ACCGCCGAGAAGCAGGCCGGTGCCGAACTGCGTGAGCAGGTGCTCGGCGGACGCGTGATCACCGGATTCG |
| | | TCAGCACCGCACCGCTGGCCGCGGAGATGAGGGCGTTCCTCGACATCACCCTGGGCGCACACATCGTCGA |
| | | CGGCTACGGGCTCACCGAGACCGGCGCCGTGACACGCGACGGTGTGATCGTGCGGCCACCGGTGATCGAC |
| | | TACAAGCTGATCGACGTTCCCGAACTCGGCTACTTCAGCACCGACAAGCCCTACCCGCGTGGCGAACTGC |
| | | TGGTCAGGTCGCAAACGCTGACTCCCGGGTACTACAAGCGCCCCGAGGTCACCGCGAGCGTCTTCGACCG |
| | | GGACGGCTACTACCACACCGGCGACGTCATGGCCGAGACCGCACCCGACCACCTGGTGTACGTGGACCGT |
| | | CGCAACAACGTCCTCAAACTCGCGCAGGGCGAGTTCGTGGCGGTCGCCAACCTGGAGGCGGTGTTCTCCG |
| | | GCGCGGCGCTGGTGCGCCAGATCTTCGTGTACGGCAACAGCGAGCGCAGTTTCCTTCTGGCCGTGGTGGT |
| | | CCCGACGCCGGAGGCGCTCGAGCAGTACGATCCGGCCGCGCTCAAGGCCGCGCTGGCCGACTCGCTGCAG |
| | | CGCACCGCACGCGACGCCGAACTGCAATCCTACGAGGTGCCGGCCGATTTCATCGTCGAGACCGAGCCGT |
| | | TCAGCGCCGCCAACGGGCTGCTGTCGGGTGTCGGAAAACTGCTGCGGCCCAACCTCAAAGACCGCTACGG |
| | | GCAGCGCCTGGAGCAGATGTACGCCGATATCGCGGCCACGCAGGCCAACCAGTTGCGCGAACTGCGGCGC |
| | | GCGGCCGCCACACAACCGGTGATCGACACCCTCACCCAGGCCGCTGCCACGATCCTCGGCACCGGGAGCG |
| | | AGGTGGCATCCGACGCCCACTTCACCGACCTGGGCGGGGATTCCCTGTCGGCGCTGACACTTTCGAACCT |
| | | GCTGAGCGATTTCTTCGGTTTCGAAGTTCCCGTCGGCACCATCGTGAACCCGGCCACCAACCTCGCCCAA |
| | | CTCGCCCAGCACATCGAGGCGCAGCGCACCGCGGGTGACCGCAGGCCGAGTTTCACCACCGTGCACGGCG |
| | | CGGACGCCACCGAGATCCGGGCGAGTGAGCTGACCCTGGACAAGTTCATCGACGCCGAAACGCTCCGGGC |
| | | CGCACCGGGTCTGCCCAAGGTCACCACCGAGCCACGGACGGTGTTGCTCTCGGGCGCCAACGGCTGGCTG |
| | | GGCCGGTTCCTCACGTTGCAGTGGCTGGAACGCCTGGCACCTGTCGGCGGCACCCTCATCACGATCGTGC |
| | | GGGGCCGCGACGACGCCGCGGCCCGCGCACGGCTGACCCAGGCCTACGACACCGATCCCGAGTTGTCCCG |
| | | CCGCTTCGCCGAGCTGGCCGACCGCCACCTGCGGGTGGTCGCCGGTGACATCGGCGACCCGAATCTGGGC |
| | | CTCACACCCGAGATCTGGCACCGGCTCGCCGCCGAGGTCGACCTGGTGGTGCATCCGGCAGCGCTGGTCA |
| | | ACCACGTGCTCCCCTACCGGCAGCTGTTCGGCCCCAACGTCGTGGGCACGGCCGAGGTGATCAAGCTGGC |
| | | CCTCACCGAACGGATCAAGCCCGTCACGTACCTGTCCACCGTGTCGGTGGCCATGGGGATCCCCGACTTC |
| | | GAGGAGGACGGCGACATCCGGACCGTGAGCCCGGTGCGCCCGCTCGACGGCGGATACGCCAACGGCTACG |
| | | GCAACAGCAAGTGGGCCGGCGAGGTGCTGCTGCGGGAGGCCCACGATCTGTGCGGGCTGCCCGTGGCGAC |
| | | GTTCCGCTCGGACATGATCCTGGCGCATCCGCGCTACCGCGGTCAGGTCAACGTGCCAGACATGTTCACG |
| | | CGACTCCTGTTGAGCCTCTTGATCACCGGCGTCGCGCCGCGGTCGTTCTACATCGGAGACGGTGAGCGCC |
| | | CGCGGGCGCACTACCCCGGCCTGACGGTCGATTTCGTGGCCGAGGCGGTCACGACGCTCGGCGCGCAGCA |
| | | GCGCGAGGGATACGTGTCCTACGACGTGATGAACCCGCACGACGACGGGATCTCCCTGGATGTGTTCGTG |
| | | GACTGGCTGATCCGGGCGGGCCATCCGATCGACCGGGTCGACGACTACGACGACTGGGTGCGTCGGTTCG |
| | | AGACCGCGTTGACCGCGCTTCCCGAGAAGCGCCGCGCACAGACCGTACTGCCGCTGCTGCACGCGTTCCG |
| | | CGCTCCGCAGGCACCGTTGCGCGGCGCACCCGAACCCACGGAGGTGTTCCACGCCGCGGTGCGCACCGCG |
| | | AAGGTGGGCCCGGGAGACATCCCGCACCTCGACGAGGCGCTGATCGACAAGTACATACGCGATCTGCGTG |
| | | AGTTCGGTCTGATCTCGAGCTCGTGAGGTACCCACAAGGAGGTTTTTACAATGAAAACGACCCACACCAG |
| | | CTTACCATTTGCCGGCCACACGTTACATTTCGTCGAATTTGATCCGGCGAACTTTTGTGAACAAGACCTG |
| | | TTGTGGCTGCCGCATTATGCCCAGCTGCAGCACGCAGGCCGTAAGCGTAAAACTGAACATCTGGCCGGTC |
| | | GCATT6CGGCAGTGTATGCCCTGCGCGAGTACGGCTACAAATGCGTGCCGGCCATTGGTGAACTGCGTCA |
| | | ACCGGTTTGGCCGGCAGAAGTTTACGGTTCCATCTCCCACTGCGGTACTACCGCGTTGGCGGTTGTGTCT |
| | | CGCCAGCCGATCGGTATTGATATTGAAGAGATATTCTCTGTCCAGACGGCACGCGAGCTGACGGACAACA |
| | | TCATTACCCCGGCAGAGCACGAGCGTCTGGCGGACTGTGGTCTGGCGTTCAGCCTGGCGCTGACCCTGGC |
| | | ATTCAGCGCAAAAGAGAGCGCGTTCAAGGCTTCCGAGATCCAAACCGATGCGGGCTTCCTGGATTATCAA |
| | | ATCATCAGCTGGAACAAGCAACAGGTTATCATTCACCGTGAGAATGAGATGTTTGCCGTCCATTGGCAGA |
| | | TTAAAGAGAAAATCGTTATCACCCTGTGCCAGCACGACTGAGAATTCGGTTTTCCGTCCTGTCTTGATTT |
| | | TCAAGCAAACAATGCCTCCGATTTCTAATCGGAGGCATTTGTTTTTGTTTATTGCAAAAACAAAAAATAT |
| | | TGTTACAAATTTTTACAGGCTATTAAGCCTACCGTCATAAATAATTTGCCATTTACTAGTTTTTAATTAA |
| | | ACCCCTATTTGTTTATTTTTCTAAATACATTCAAATATGTATCCGCTCATGAGACAATAACCCTGATAAA |
| | | TGCTTCAATAATATTGAAAAAGGAAGAGTATGATTGAACAAGATGGCCTGCATGCTGGTTCTCCGGCTGC |
| | | TTGGGTGGAACGCCTGTTTGGTTACGACTGGGCTCAGCTGACTATTGGCTGTAGCGATGCAGCGGTTTTC |
| | | CGTCTGTCTGCACAGGGTCGTCCGGTTCTGTTTGTGAAAACCGACCTGTCCGGCGCACTGAACGAACTGC |
| | | AGGACGAAGCGGCCCGTCTGTCCTGGCTCGCGACGACTGGTGTTCCGTGCGCGGCAGTTCTGGACGTAGT |
| | | TACTGAAGCCGGTCGCGATTGGCTGCTGCTGGGTGAAGTTCCGGGTCAGGATCTGCTGAGCAGCCACCTC |
| | | GCTCCGGCAGAAAAAGTTTCCATCATGGCGGACGCGATGCGCCGTCTGCACACCCTGGACCCGGCAACTT |
| | | GCCCGTTTGACCATCAGGCTAAACACCGTATTGAACGTGCACGCACTCGTATGGAAGCGGGTCTGGTTGA |
| | | TCAGGACGACCTGGATGAAGAGCACCAGGGCCTCGCACCGGCGGAACTGTTTGCACGTCTGAAAGCCCGC |
| | | ATGCCGGACGGCGAAGACCTGGTGGTAACGCATGGCGACGCTTGTCTGCCAAACATTATGGTGGAAAACG |
| | | GCCGCTTCTCTGGTTTTATTGACTGTGGCCGTCTGGGTGTAGCTGATCGCTATCAGGATATCGCCCTCGC |
| | | TACCCGCGATATTGCAGAAGAACTGGGTGGTGAATGGGCTGACCGTTTCCTGGTGCTGTACGGTATCGCA |
| | | GCGCCGGATTCTCAGCGCATTGCCTTCTACCGTCTGCTGGATGAGTTCTTCTAAGGCGCGCCGAAACTGC |
| | | GCCAAGAATAGCTCACTTCAAATCAGTCACGGTTTTGTTTAGGGCTTGTCTGGCGATTTTGGTGACATAG |
| | | ACAGTCACAGCAACAGTAGCCACAAAACCAAGAATCCGGATCGACCACTGGGCAATGGGGTTGGCGCTGG |
| | | TGCTTTCTGTGCCGAGGGTCGCAAGATTTCCGGCCAGGGAGCCAATGTAGACATACATGATGGTGCCAGG |
| | | GATCATCCCCACAGAGCCGAGGACATAGTCTTTTAGGGAAACGCCCGTGACCCCATAGGCATAGTTAAGC |
| | | AGATTAAAGGGAAATACAGGTGAGAGACGCGTCAGGAGAACAATCTTCAGGCCTTCCTTGCCCACAGCTT |
| | | CGTCGATGGCGCGAAATTTCGGGTTGTCGGCGATTTTTTGGCTCACCCATTGGCGGGCCAGATAACGACC |
| | | CACTAGGAAAGCAGCGATCGCTCCTAGGGTTGCGCCAACAAAGACGTAAATTGATCCTAAAGCGACACCA |
| | | AAAACAACCCCGGCTCCCAAGGTCAGAATCGACCCCGGTAGAAAAGCCACCGTCGCCACCACATAAAGCA |
| | | CCATAAAGGCGATGGCCGGCCAAAATGAAGTGAAGTTCCTATACTTTCTAGAGAATAGGAACTTCTATAG |
| | | TGAGTCGAATAAGGGCGACACAAAATTTATTCTAAATGCATAATAAATACTGATAACATCTTATAGTTTG |
| | | TATTATATTTTGTATTATCGTTGACATGTATAATTTTGATATCAAAAACTGATTTTCCCTTTATTATTTT |
| | | CGAGATTTATTTTCTTAATTCTCTTTAACAAACTAGAAATATTGTATATACAAAAAATCATAAATAATAG |
| | | ATGAATAGTTTAATTATAGGTGTTCATCAATCGAAAAAGCAACGTATCTTATTTAAAGTGCGTTGCTTTT |
| | | TTCTCATTTATAAGGTTAAATAATTCTCATATATCAAGCAAAGTGACAGGCGCCCTTAAATATTCTGACA |
| | | AATGCTCTTTCCCTAAACTCCCCCCATAAAAAAACCCGCCGAAGCGGGTTTTTACGTTATTTGCGGATTA |
| | | ACGATTACTCGTTATCAGAACCGCCCAGGGGGCCCGAGCTTAAGACTGGCCGTCGTTTTACAACACAGAA |
| | | AGAGTTTGTAGAAACGCAAAAAGGCCATCCGTCAGGGGCCTTCTGCTTAGTTTGATGCCTGGCAGTTCCC |
| | | TACTCTCGCCTTCCGCTTCCTCGCTCACTGACTCGCTGCGCTCGGTCGTTCGGCTGCGGCGAGCGGTATC |
| | | AGCTCACTCAAAGGCGGTAATACGGTTATCCACAGAATCAGGGGATAACGCAGGAAAGAACATGTGAGCA |
| | | AAAGGCCAGCAAAAGGCCAGGAACCGTAAAAAGGCCGCGTTGCTGGCGTTTTTCCATAGGCTCCGCCCCC |
| | | CTGACGAGCATCACAAAAATCGACGCTCAAGTCAGAGGTGGCGAAACCCGACAGGACTATAAAGATACCA |
| | | GGCGTTTCCCCCTGGAAGCTCCCTCGTGCGCTCTCCTGTTCCGACCCTGCCGCTTACCGGATACCTGTCC |
| | | GCCTTTCTCCCTTCGGGAAGCGTGGCGCTTTCTCATAGCTCACGCTGTAGGTATCTCAGTTCGGTGTAGG |
| | | TCGTTCGCTCCAAGCTGGGCTGTGTGCACGAACCCCCCGTTCAGCCCGACCGCTGCGCCTTATCCGGTAA |
| | | CTATCGTCTTGAGTCCAACCCGGTAAGACACGACTTATCGCCACTGGCAGCAGCCACTGGTAACAGGATT |
| | | AGCAGAGCGAGGTATGTAGGCGGTGCTACAGAGTTCTTGAAGTGGTGGGCTAACTACGGCTACACTAGAA |
| | | GAACAGTATTTGGTATCTGCGCTCTGCTGAAGCCAGTTACCTTCGGAAAAAGAGTTGGTAGCTCTTGATC |
| | | CGGCAAACAAACCACCGCTGGTAGCGGTGGTTTTTTTGTTTGCAAGCAGCAGATTACGCGCAGAAAAAAA |
| | | GGATCTCAAGAAGATCCTTTGATCTTTTCTACGGGGTCTGACGCTCAGTGGAACGACGCGCGCGTAACTC |
| | | ACGTTAAGGGATTTTGGTCATGAGCTTGCGCCGTCCCGTCAAGTCAGCGTAATGCTCTGCTTTT |

TABLE 2

| | General | | | | | | |
|---|---|---|---|---|---|---|---|
| | Enzyme | Enzyme | | | Gene | | Accession |
| | Activity | Activity | Enzyme | EC # | Name | Organism | Number |
| 1 | Alkane | An aldehyde + | alkane | 4.1.99.5 | adm | Cyanothece sp. | YP_001802195 |
| | deformylative | O2 + 2 | deformylative | | | ATCC 51142 | |
| | monooxygenase | NADPH + 2 | monooxygenase | | adm | Nostoc | YP_001865325 |
| | activity | H+ = an (n − 1) | | | | punctiforme | |
| | | alkane + | | | adm | Prochlorococcus | YP_397029 |
| | | formate + H2O + | | | | marinus MIT | |
| | | 2 NADP+ | | | | 9312 | |
| | | | | | adm | Thermosynechococcus | NP_682103 |
| | | | | | | elongatus | |
| | | | | | | BP-1 | |
| 2 | Carboxylic acid | An aldehyde + | carboxylic acid | 1.2.99.6 | carB | Mycobacterium | YP_889972 |
| | reductase | acceptor + | reductase | | | smegmatis str. | |
| | activity | H2O = a | | | | MC2 155 | |
| | | carboxylate + | | | car | Nocardia | AAR91681 |
| | | reduced | | | | iowensis | |
| | | acceptor | | | fadD9 | Mycobacterium | YP_001850422 |
| | | | | | | marinum M | |
| 3 | Phospho- | CoA-[4′- | phosphopanthetheinyl | 2.7.8.7 | entD | Escherichia coli | NP_415115 |
| | panthetheinyl | phosphopantetheine] + | transferase | | sfp | Bacillus subtilis | ZP_12673024 |
| | transferase | apo- | | | | subsp. subtilis | |
| | activity | [acyl-carrier | | | | str. SC-8 | |
| | | protein] = | | | | | |
| | | adenosine 3′,5′- | | | | | |
| | | bisphosphate + | | | | | |
| | | holo-[acyl- | | | | | |
| | | carrier protein] | | | | | |
| 4 | Thioesterase | A fatty acyl- | thioesterase | 3.1.2.14 | fatB2 | Cuphea | AAC49269 |
| | activity | [acyl-carrier | | | | hookeriana | |
| | | protein] + H2O = | | | tesA | Escherichia coli | NP_415027 |
| | | [acyl-carrier | | | FatB3 | Cocos nucifera | AEM72521 |
| | | protein] + a | | | Ua- | Ulmus | AAB71731 |
| | | fatty acid | | | FatB1 | americana | |
| 5 | Long-chain | An aldehyde + | long-chain acyl- | 1.2.1.50 | acrM | Acinetobacter | BAB85476 |
| | acyl-CoA | CoA + NADP + = | CoA reductase | | | sp. M-1 | |
| | reductase | an acyl-CoA + | | | ucpA | Escherichia coli | NP_416921 |
| | activity | NADPH + | | | ybbO | Escherichia coli | NP_415026 |
| | | H+ | | | luxC | Photorhabdus | NP_929340 |
| | | | | | | luminescens | |
| | | | | | | subsp. laumondii | |
| | | | | | | TTO1 | |
| | | | | | acr1 | Acinetobacter | YP_047869 |
| | | | | | | sp. ADP-1 | |
| 6 | Long-chain fatty | ATP + a long- | long-chain fatty | 6.2.1.3 | fadD | Escherichia coli | NP_416319 |
| | acid CoA-ligase | chain fatty acid + | acid CoA-ligase | | fadD | Synechococcus | YP_001733936 |
| | activity | CoA = AMP + | | | | elongatus | |
| | | diphosphate + | | | TTC0079 | Thermus | YP_004054 |
| | | an acyl-CoA | | | | thermophilus | |
| | | | | | | HB27 | |

TABLE 5
Key to sequences on pCDF-npu plasmid

| Location (nt) | Direction | Feature |
|---|---|---|
| 5-25 | forward | lac operator |
| 58-63 | forward | adm ribosome binding site |
| 71-811 | forward | His-tagged Nostoc punctiforme adm |
| 882-898 | forward | T7 promoter |
| 903-923 | forward | lac operator |
| 954-959 | forward | ribosome binding site |
| 965-1106 | forward | multiple cloning site |
| 1130-1177 | forward | T7 terminator |
| 1351-2139 | complement | streptomycin resistance (SmR) gene |
| 2279-3017 | complement | CloDF13 origin |
| 3227-4309 | complement | lac repressor (lacI) |
| 4433-4449 | forward | T7 promoter |

What is claimed is:

1. An engineered microorganism, wherein said engineered microorganism comprises one or more recombinant nucleic acid sequences encoding one or more enzymes having enzyme activities which catalyze the production of alkanes, wherein the enzyme activities comprise an alkane deformylative monooxygenase activity and a thioesterase activity, a carboxylic acid reductase activity, and a phosphopanthetheinyl transferase activity; or a thioesterase activity, a long-chain fatty acid CoA-ligase activity, and a long-chain acyl-CoA reductase activity.

2. The engineered microorganism of claim 1, wherein the enzymes comprise an alkane deformylative monooxygenase, a thioesterase, a carboxylic acid reductase, and a phosphopanthetheinyl transferase.

3. The engineered microorganism of claim 2, wherein the alkane deformylative monooxygenase has EC number 4.1.99.5, the thioesterase has EC number 3.1.2.14, the carboxylic acid reductase has EC number 1.2.99.6, and the phosphopanthetheinyl transferase has EC number 2.7.8.7.

4. The engineered microorganism of claim 2, wherein the alkane deformylative monooxygenase is encoded by adm, the thioesterase is encoded by tesA, fatB, or fatB2, the carboxylic acid reductase is encoded by carB, and the phosphopanthetheinyl transferase is encoded by entD.

5. The engineered microorganism of claim 1, wherein the enzyme having alkane deformylative monooxygenase activity has EC number 4.1.99.5, the enzyme having thioesterase activity has EC number 3.1.2.14, the enzyme having carboxylic acid reductase activity has EC number 1.2.99.6, and the enzyme having phosphopanthetheinyl transferase activity has EC number 2.7.8.7.

6. The engineered microorganism of claim 1, wherein said microorganism is a cyanobacterium, a thermotolerant cyanobacterium, or a Synechococcus species.

7. The engineered microorganism of claim 1, wherein expression of an operon comprising one or more of the recombinant genes is controlled by a recombinant promoter, and wherein the promoter is constitutive or inducible.

8. The engineered photosynthetic microorganism of claim 1, wherein said microorganism produces alkanes 7, 8, 9, 10, or 11 carbon atoms in length.

9. The engineered photosynthetic microorganism of claim 1, wherein the microorganism produces alkanes 9 carbon atoms in length.

10. The engineered photosynthetic microorganism of claim 1, wherein the microorganism produces alkanes 11 carbon atoms in length.

11. The engineered photosynthetic microorganism of claim 1, wherein the microorganism produces alkanes 9 and 11 carbon atoms in length.

12. The engineered microorganism of claim 1, wherein the engineered microorganism produces at least one of heptane, nonane, and undecane in an amount greater than an otherwise identical microorganism, cultured under identical conditions, but lacking the recombinant nucleic acid sequences.

13. The engineered microorganism of claim 1, wherein the engineered microorganism produces at least one of heptane, nonane, and undecane in an amount at least two times greater than an otherwise identical microorganism, cultured under identical conditions for twelve hours, but lacking the recombinant nucleic acid sequences.

14. A cell culture comprising a culture medium and the microorganism of claim 1.

15. A method for producing hydrocarbons, comprising: culturing an engineered microorganism of claim 1 in a culture medium, wherein said engineered microorganism produces increased amounts of alkanes relative to an otherwise identical microorganism, cultured under identical conditions, but lacking said recombinant genes.

16. The method of claim 15, wherein the alkanes produced are 9 and/or 11 carbons in length.

17. A composition comprising alkanes, wherein said alkanes are produced by the method of claim 15.

18. A method for producing hydrocarbons, comprising: (i) culturing an engineered microorganism of claim 1 in a culture medium; and (ii) exposing said engineered microorganism to light and inorganic carbon, wherein said exposure results in the conversion of said inorganic carbon by said microorganism into alkanes, wherein said alkanes are produced in an amount greater than that produced by an otherwise identical microorganism, cultured under identical conditions, but lacking said recombinant genes.

19. The method of claim 18, wherein the alkanes produced are 9 and/or 11 carbons in length.

20. A composition comprising alkanes, wherein said alkanes are produced by the method of claim 18.
